(19) United States
(12) Patent Application Publication
(10) Pub. No.: US 2024/0043828 A1
(43) Pub. Date: Feb. 8, 2024

(54) T-DNA MEDIATED GENETIC MODIFICATION

(71) Applicants: THE BROAD INSTITUTE, INC., Cambridge, MA (US); MASSACHUSETTS INSTITUTE OF TECHNOLOGY, Cambridge, MA (US)

(72) Inventors: Feng Zhang, Cambridge, MA (US); Alim Ladha, Cambridge, MA (US)

(21) Appl. No.: 18/030,324

(22) PCT Filed: Oct. 5, 2021
(86) PCT No.: PCT/US2021/053588

§ 371 Date: Apr. 5, 2023

Related U.S. Application Data
(60) Provisional application No. 63/088,420, filed on Oct. 6, 2020.

(51) Int. Cl. C12N 15/10; C12N 9/22; C12N 15/11; C12N 9/12; C07K 14/195

(52) U.S. Cl. CPC C12N 15/102; C12N 9/22; C12N 15/11; C12N 9/1252; C12Y 207/07007; C07K 14/195; C12N 2310/20

(57) ABSTRACT

Described in several embodiments herein are compositions, systems, and methods for targeted gene modification, targeted insertion, perturbation of gene transcripts, and nucleic acid editing. Described in several embodiments are nucleic acid targeting systems that include components of CRISPR systems, VirD polypeptide(s), and DNA polymerase(s).

### CROSS-REFERENCE TO RELATED APPLICATIONS

This application claims the benefit of U.S. Provisional Application No. 63/088,420, filed Oct. 6, 2020. The entire contents of the above-identified applications are hereby fully incorporated herein by reference.

### STATEMENT REGARDING FEDERALLY SPONSORED RESEARCH

This invention was made with government support under Grant No. HL141201 awarded by National Institutes of Health. The government has certain rights in the invention.

### REFERENCE TO AN ELECTRONIC SEQUENCE LISTING

The contents of the electronic sequence listing (“BROD-5280WP_ST25.txt”; Size is 27,013 bytes (29 KB on disk) and it was created on Oct. 5, 2021) is herein incorporated by reference in its entirety.

### TECHNICAL FIELD

The subject matter disclosed herein is generally directed to systems, methods and compositions used for inter alia targeted gene modification, targeted insertion, perturbation of gene transcripts, and nucleic acid editing. Aspects of the subject matter disclosed herein involve components of Clustered Regularly Interspaced Short Palindromic Repeats (CRISPR) systems and agrobacterium elements.

### BACKGROUND

Recent advances in genome sequencing techniques and analysis methods have significantly accelerated the ability to catalog and map genetic factors associated with a diverse range of biological functions and diseases. Precise genome targeting technologies are needed to enable systematic reverse engineering of causal genetic variations by allowing selective perturbation of individual genetic elements, as well as to advance synthetic biology, biotechnological, and medical applications. Although genome-editing techniques such as designer zinc fingers, transcription activator-like effectors (TALEs), or homing meganucleases are available for producing targeted genome perturbations, there remains a need for new genome engineering technologies that employ new and/or improved strategies and molecular mechanisms and are affordable, easy to set up, scalable, and amenable to targeting multiple positions within the eukaryotic genome. This would provide a major resource for new applications in genome engineering and biotechnology.

Citation or identification of any document in this application is not an admission that such document is available as prior art to the present invention.

### SUMMARY

Described in exemplary embodiments herein are engineered or non-naturally occurring compositions comprising a site-specific nuclease polypeptide; a DNA polymerase polypeptide connected to or otherwise capable of forming a complex with the site-specific nuclease polypeptide; and a VirD2 polypeptide connected to or otherwise capable of forming a complex with the site-specific nuclease polypeptide, wherein the site-specific nuclease polypeptide directs the DNA polymerase and VirD2 polypeptide to a target sequence in a target polynucleotide.

In certain example embodiments, the engineered or non-naturally occurring compositions further comprise a donor construct capable of forming a complex with the VirD2 polypeptide, and encoding a donor polynucleotide sequence, the donor polynucleotide sequence serving as a template for integration of the donor polynucleotide sequence into the target polynucleotide by the DNA polymerase.

In certain example embodiments, the donor construct comprises a single-stranded donor polynucleotide sequence and a VirD2 binding sequence.

In certain example embodiments, the donor construct is a double-stranded polynucleotide encoding a 5′ and a 3′ boundary sequence and a donor polynucleotide sequence located between the 5′ and the 3′ boundary sequences, and wherein the composition further comprises a VirD1 polypeptide that alone, or in combination with the VirD2 polypeptide, releases a single-stranded donor polynucleotide sequence from the donor construct, resulting in a complex of the single-stranded donor polynucleotide sequence with the VirD2 polypeptide.

In certain example embodiments, the VirD1 polypeptide is connected to the VirD2 polypeptide.

In certain example embodiments, the donor construct further comprises a homology sequence complementary to at least a portion of the target sequence.

In certain example embodiments, the donor polynucleotide sequence is 10 bp to 50 kb bp in length.

In certain example embodiments, the site-specific nuclease polypeptide is a Cas polypeptide, and the composition further comprises a guide polynucleotide capable of forming a complex with the Cas-polypeptide and directing site specific binding of the complex to the target sequence.

In certain example embodiments, the homology sequence is complementary to at least a portion of the guide polynucleotide.

In certain example embodiments, the Cas polypeptide is a nickase that nicks a targeted strand of the target polynucleotide.

In certain example embodiments, the compositions further comprise a second guide molecule capable of forming a complex with the Cas nickase and directing nicking of a non-targeted strand of the target polynucleotide.

In certain example embodiments, the site-specific nuclease is an IscB polypeptide, or a TnpB polypeptide.

In certain example embodiments, the VirD2 and/or VirD1 polypeptides are derived from Agrobacterium tumefaceins or Rhizobium meliloti.

In certain example embodiments, the DNA polymerase is a DNA Pol I polymerase.

In certain example embodiments, the DNA polymerase is an E. coli DNA polymerase.

Described in certain example embodiments herein are vector systems comprising one or more vectors encoding the site-specific nuclease, the DNA polymerase, the virD1 polypeptide, the virD2 polypeptide, and the donor on construct described herein.

Described in certain example embodiments herein are methods of inserting a donor polynucleotide sequence into a target polynucleotide comprising: introducing a non-natural or engineered composition as described herein into a cell or cell population, wherein the complex of the site-specific nuclease polypeptide and the guide directs the single-stranded donor polynucleotide and the DNA polymerase to the target sequence, and wherein the DNA polymerase facilitates incorporation of the donor polynucleotide at or adjacent to the target sequence.

In certain example embodiments, the donor polynucleotide is between 5 bases and 50 kb in length.

In certain example embodiments, the polypeptide and/or nucleic acid components are provided via one or more polynucleotides encoding the polypeptides and/or nucleic acid component(s), and wherein the one or more polynucleotides are operably configured to express the polypeptides and/or nucleic acid component(s).

In certain example embodiments, the donor polynucleotide is inserted to a region on the target polynucleotide that is 5′ of the PAM. In certain other example embodiments, the donor polynucleotide is inserted into a region on the target polynucleotide that is 3′ of the PAM sequence.

In certain example embodiments, the donor polynucleotide introduces one or more mutations to the target polynucleotide, inserts a functional gene or gene fragment at the target polynucleotide, corrects or introduces a premature stop codon in the target polynucleotide, disrupts or restores a splice cite in the target polynucleotide, causes a shift in the open reading frame of the target polynucleotide, or any combination thereof.

In certain example embodiments, the one or more mutations include substitutions, deletions, and insertions.

These and other aspects, objects, features, and advantages of the example embodiments will become apparent to those having ordinary skill in the art upon consideration of the following detailed description of illustrated example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1 shows an exemplary double stranded donor construct and site-specific nucleic acid compositing including a donor polynucleotide sequence.

FIG. 2 shows an exemplary single stranded donor construct and site-specific nucleic acid composition including a donor polynucleotide sequence.

FIG. 3 shows an exemplary method of integration of a donor polynucleotide sequence where the site-specific nuclease is a Cas protein.

FIG. 4 shows an exemplary method of integration of a donor polynucleotide sequence where the site-specific nuclease is a Cas protein and integration further includes nicking of a non-target strand with a nickase.

FIG. 5 shows an exemplary method of integration of a donor polynucleotide sequence where the site-specific nuclease is a wild-type Cas protein.

The figures herein are for illustrative purposes only and are not necessarily drawn to scale.

### DETAILED DESCRIPTION OF THE EXAMPLE EMBODIMENTS

General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R.I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a” “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

The term “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

All publications, published patent documents, and patent applications cited herein are hereby incorporated by reference to the same extent as though each individual publication, published patent document, or patent application was specifically and individually indicated as being incorporated by reference.

The recitation of numerical ranges by endpoints includes all numbers and fractions subsumed within the respective ranges, as well as the recited endpoints.

The term “about” in relation to a reference numerical value and its grammatical equivalents as used herein can include the numerical value itself and a range of values plus or minus 10% from that numerical value. For example, the amount “about 10” includes 10 and any amounts from 9 to 11. For example, the term “about” in relation to a reference numerical value can also include a range of values plus or minus 10%, 9%, 8%, 7%, 6%, 5%, 4%, 3%, 2%, or 1% from that value.

Whereas the terms “one or more” or “at least one” or “X or more”, where X is a number and understand to mean X or increases one by one of X, such as one or more or at least one member(s) or “X or more” of a group of members, is clear per se, by means of further exemplification, the term encompasses inter alia a reference to any one of said members, or to any two or more of said members, such as, e.g., any >3, >4, >5, >6 or >7 etc. of said members, and up to all said members.

The term “functional variant or functional fragment” means that the amino-acid sequence of the polypeptide may not be strictly limited to the sequence observed in nature but may contain additional amino-acids. The term “functional fragment” means that the sequence of the polypeptide may include less amino-acid than the original sequence but still enough amino-acids to confer the enzymatic activity of the original sequence of reference. It is well known in the art that a polypeptide can be modified by substitution, insertion, deletion and/or addition of one or more amino-acids while retaining its enzymatic activity. For example, substitutions of one amino-acid at a given position by chemically equivalent amino-acids that do not affect the functional properties of a protein are common.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

A protein or nucleic acid derived from a species means that the protein or nucleic acid has a sequence identical to an endogenous protein or nucleic acid or a portion thereof in the species. The protein or nucleic acid derived from the species may be directly obtained from an organism of the species (e.g., by isolation), or may be produced, e.g., by recombination production or chemical synthesis.

All references cited in the present specification are hereby incorporated by reference in their entirety. In particular, the teachings of all references herein specifically referred to are incorporated by reference.

### Overview

The present disclosure provides engineered or non-naturally occurring nucleic acid modification systems and methods for modifying a target polynucleotide, for example, by inserting a donor polynucleotide at desired position in the target polynucleotide. In general, exemplary embodiments of the engineered nucleic acid modification systems are composed of one or more components of a site specific nuclease system (e.g., a CRISPR-Cas system) and one or more components of an agrobacterium-based nucleic acid transfer system, and a DNA polymerase. The components of the site specific nuclease system (e.g., a CRISPR-Cas system) may direct the donor polynucleotide sequence to a target nucleic acid sequence. In one aspect, the present disclosure provides a system comprising a Cas polypeptide, a VirD2 polypeptide, a DNA polymerase polypeptide, a T-DNA-based donor polynucleotide, and an optional guide molecule, and/or polynucleotide(s) encoding these components. In some examples, the Cas polypeptide is a nickase or lacks nuclease activity. In some embodiments, the engineered nucleic acid modification system of the present invention can include a VirD1 polypeptide that alone, or in combination with the VirD2 polypeptide, releases a single-stranded donor polynucleotide sequence from the donor construct, resulting in a complex of the single-stranded donor polynucleotide sequence with the VirD2 polypeptide. The present disclosure also includes polynucleotides encoding components of the engineered nucleic acid modification system, and vector systems comprising one or more polynucleotides of or encoding components of the engineered nucleic acid modification systems provided herein. Further provided herein are cells, tissues, organs, and organisms comprising the engineered nucleic acid modification systems or components thereof and/or cells, tissues, organs, and organisms generated using the engineered nucleic acid modification systems described herein.

Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

### Genetic Modification Systems and Compositions

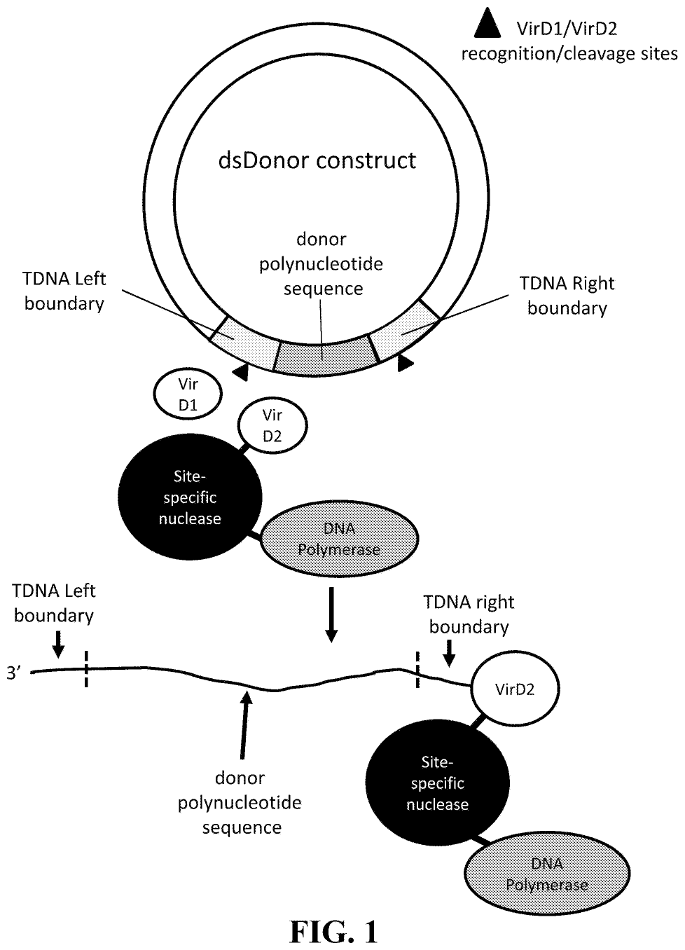

Generally, described in several example embodiments herein are engineered nucleic acid modification systems and compositions that are capable of gene transfer. In several example embodiments, the present disclosure includes systems and compositions that comprise one or more components of an agrobacterium gene transfer system, one or more components of a site-specific nuclease system, such as a CRISPR-Cas system, and a DNA polymerase. In some embodiments, the systems and compositions include a VirD2 polypeptide and optionally a VirD1 polypeptide. In certain embodiments, the systems and compositions also include a DNA polymerase. In some embodiments, the system and compositions include a donor polynucleotide sequence that is coupled to the VirD2. For example, the present disclosure provides a site-specific nuclease polypeptide; a DNA polymerase polypeptide connected to or otherwise capable of forming a complex with the site-specific nuclease polypeptide; and a VirD2 polypeptide connected to or otherwise capable of forming a complex with the site-specific nuclease polypeptide; wherein the site-specific nuclease polypeptide directs the DNA polymerase, VirD2 polypeptide, and donor polynucleotide sequence to a target sequence in a target polynucleotide. The target sequence can be a target insertion site. The target polynucleotide can be double-stranded DNA. The site-specific nuclease may either create a double-strand break or a single-strand nick at the target site. The DNA polymerase polypeptide may then facilitate insertion of the donor polynucleotide sequence into the target polynucleotide. FIGS. 1-4 show exemplary embodiments of the systems and compositions described herein.

The term “fusion protein” is used herein to refer to protein construction comprising the site-specific nuclease connected to the VirD2 polypeptide and/or the DNA polymerase polypeptide for example by a polypeptide linker or other suitable linker. The term is also used to refer to protein construction comprising the VirD2 connected to the VirD1 polypeptide, for example, directly to or via a polypeptide or other suitable linker. It should be understood that the term “fusion protein” includes embodiments where the composition comprises a site-specific nuclease and a VirD2 polypeptide and/or DNA polymerase polypeptide are already connected to one another, where the composition comprises a VirD1 and a VirD2 polypeptide are already connected to one another or embodiments where the site-specific nuclease, the VirD2, VirD1, and/or DNA polymerase comprise two separate components that may come together to form a single complex, for example, through the use of engineered domains on each polypeptide that functions as binding partners to bring the site-specific nuclease VirD2, VirD1, DNA polymerase or combinations thereof together. When the term “fusion protein” is used in connection with or to describe other proteins discussed herein, the same rationale expressed here can be applied in those contexts as well unless otherwise specified herein.

In certain example embodiments, the site specific nuclease may comprise a paired nickase in which each site-specific nuclease in the pair is fused with a VirD2 and/or DNA polymerase and creates a nick on opposing strands of a targeted insertion site and whereby the corresponding DNA polymerase(s) facilitate insertion of the donor polynucleotide from the donor construct.

In certain example embodiments, the site-specific nuclease is a Cas polypeptide and the composition further comprises a guide polynucleotide capable of forming a complex with the Cas-polypeptide and directing site specific binding of the complex to the target sequence.

In some examples, the guide directs the polypeptides (e.g., a complex or fusion protein of the Cas polypeptide, VirD2, and DNA polymerase polypeptide) to a target sequence 5′ or 3′ of the targeted insertion site, and wherein the Cas polypeptide generates a double-strand break at the targeted insertion site.

VirD and other Vir Proteins

The systems and compositions herein can include one or more VirD proteins. VirD2 is an endonuclease that is capable of nicking the two strands of an e.g., Ti plasmid (in the context of an agrobacterium gene transfer system) at two sites (left (3′) and right (5′) boundaries of the T-DNA (see e.g., Yadav et al. 1982. PNAS 79:6322-6326. Doi: 10.1007/BF00425665; Yanofsky et al. 1987 Cell. 47:471-477. Doi: 10.10165/0092-8674(86)90604-5; Nester, E. W. 2015, Front. Plant Sci. 5, art. 730 doi: 10.3389/fpls.2014/00730; Gelvin, S. B. Microbiol. Molec. Biol. Rev. 2003. 67(1):16-37 doi: 10.1128/MMBR.67.1.16-37.2003). These 25 bp right and left boundaries occur as direct repeats and their recognition and cleavage by VirD2 alone or by VirD2 and VirD1 results in the formation of single stranded T-DNA molecule that is covalently attached at the 5′ end to the VirD2 through a phosphotyrosine bond (see e.g., Stachel et. al. 1986. Nature 322:d706-712; Albright et al. 1987 J Bacteriol. 169:1046-1055; Veluthambi et al., 1988. J. Bacteriol. 170:1523-1532; Nester, E. W. 2015, Front. Plant Sci. 5, art. 730 doi: 10.3389/fpls.2014/00730 and Gelvin, S. B. Microbiol. Molec. Biol. Rev. 2003. 67(1):16-37 doi: 10.1128/MMBR.67.1.16-37.2003).

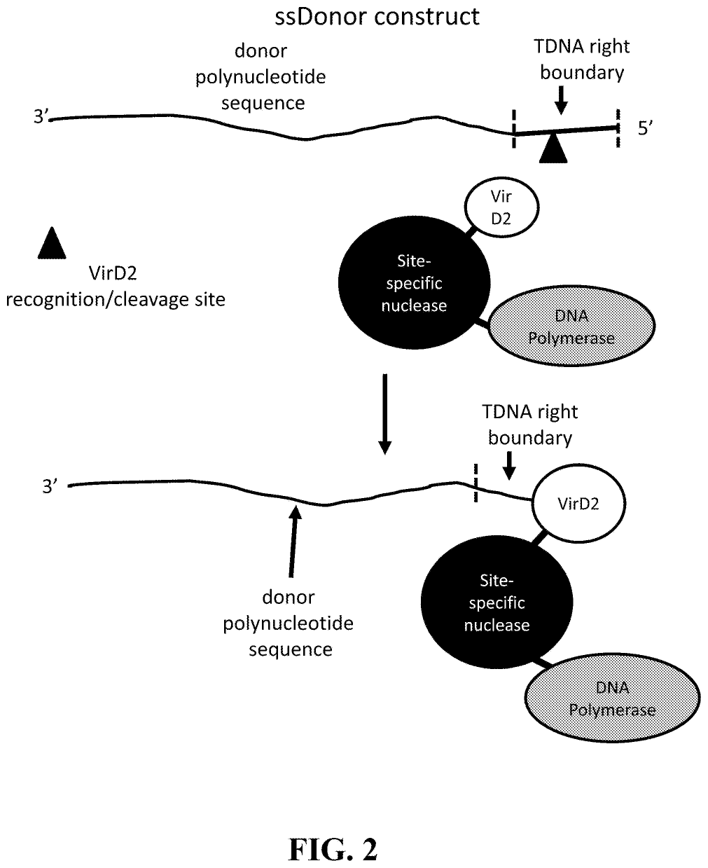

In some embodiments, the VirD2 of the systems and compositions described herein act alone or optionally with a VirD1 and/or other Vir polypeptide on a donor construct such that a donor polynucleotide sequence is coupled to the VirD2 polypeptide through a phosphotyrosine bond in a 5′ T-DNA boundary that flanks the donor polynucleotide sequence. See e.g., FIGS. 1-2.

In some embodiments, the systems and compositions include one or more other Vir proteins, including but not limited to a VirA, a VirB, a VirC, a VirE (e.g., a VirE2), a VirG, a VirH, or any combination thereof.

In some embodiments, the Vir protein (e.g., VirD2, VirD1, VirA, VirB, VirC, VirE (e.g., a VirE2), VirG, VirH) is/are an Agrobacterium tumefaceins or Rhizobium meliloti vir protein(s). In some embodiments, the Vir protein (e.g., VirD2, VirD1, VirA, VirB, VirC, VirE (e.g., a VirE2), VirG, VirH) is/are derived from Agrobacterium tumefaceins or Rhizobium meliloti.

In some embodiments, the VirD protein(s) is/are an Agrobacterium tumefaceins or Rhizobium meliloti VirD protein(s). In some embodiments, the VirD protein(s) is/are derived from an Agrobacterium tumefaceins or Rhizobium meliloti. In some embodiments the VirD2 protein is an Agrobacterium tumefaceins or Rhizobium meliloti VirD2 protein. In some embodiments the VirD2 protein is derived from Agrobacterium tumefaceins or Rhizobium meliloti. In some embodiments the VirD1 protein is an Agrobacterium tumefaceins or Rhizobium meliloti VirD1 protein. In some embodiments the VirD1 protein is derived from Agrobacterium tumefaceins or Rhizobium meliloti.

In some embodiments, the VirD1 protein has a polypeptide sequence that is 80-100 percent identical to SEQ ID NO: 1.

VirD1 Agrobacterium fabrum (strain C58/ATCC 33970)
(SEQ ID NO: 1)
MSQGSRPTSSDIAVNQRECVKVEGFKVVSTRLRSAEYESFSHQARLLGL
SDSMAIRVAVRRIGGFLEIDAETRHRMEAILQSIGTLSSNIAALLSAYA
ENPTMDLEALRAERIAFGKSFADLDGLLRSILSVSRRRIDGCSLLKDAL

In some embodiments, the VirD1 protein includes or is composed entirely of an amino acid sequence that is 80, 81, 82, 83, 84, 85, 86, 87, 88, 89, 90, 91, 92, 93, 94, 95, 96, 97, 98, 99, to/or 100 percent identical to SEQ ID NO: 1. In some embodiments, the VirD1 protein includes or is composed entirely of an amino acid sequence that is 80 to 81, 80 to 82, 80 to 83, 80 to 84, 80 to 85, 80 to 86, 80 to 87, 80 to 88, 80 to 89, 80 to 90, 80 to 91, 80 to 92, 80 to 93, 80 to 94, 80 to 95, 80 to 96, 80 to 97, 80 to 98, 80 to 99, or 80 to 100 percent identical to SEQ ID NO: 1. In some embodiments, the VirD1 protein includes or is composed entirely of an amino acid sequence that is 81 to 82, 81 to 83, 81 to 84, 81 to 85, 81 to 86, 81 to 87, 81 to 88, 81 to 89, 81 to 90, 81 to 91, 81 to 92, 81 to 93, 81 to 94, 81 to 95, 81 to 96, 81 to 97, 81 to 98, 81 to 99, or 81 to 100 percent identical to SEQ ID NO: 1. In some embodiments, the VirD1 protein includes or is composed entirely of an amino acid sequence that is 82 to 83, 82 to 84, 82 to 85, 82 to 86, 82 to 87, 82 to 88, 82 to 89, 82 to 90, 82 to 91, 82 to 92, 82 to 93, 82 to 94, 82 to 95, 82 to 96, 82 to 97, 82 to 98, 82 to 99, or 82 to 100 percent identical to SEQ ID NO: 1. In some embodiments, the VirD1 protein includes or is composed entirely of an amino acid sequence that is 83 to 84, 83 to 85, 83 to 86, 83 to 87, 83 to 88, 83 to 89, 83 to 90, 83 to 91, 83 to 92, 83 to 93, 83 to 94, 83 to 95, 83 to 96, 83 to 97, 83 to 98, 83 to 99, or 83 to 100 percent identical to SEQ ID NO: 1. In some embodiments, the VirD1 protein includes or is composed entirely of an amino acid sequence that is 84 to 85, 84 to 86, 84 to 87, 84 to 88, 84 to 89, 84 to 90, 84 to 91, 84 to 92, 84 to 93, 84 to 94, 84 to 95, 84 to 96, 84 to 97, 84 to 98, 84 to 99, or 84 to 100 percent identical to SEQ ID NO: 1. In some embodiments, the VirD1 protein includes or is composed entirely of an amino acid sequence that is 85 to 86, 85 to 87, 85 to 88, 85 to 89, 85 to 90, 85 to 91, 85 to 92, 85 to 93, 85 to 94, 85 to 95, 85 to 96, 85 to 97, 85 to 98, 85 to 99, or 85 to 100 percent identical to SEQ ID NO: 1. In some embodiments, the VirD1 protein includes or is composed entirely of an amino acid sequence that is 86 to 87, 86 to 88, 86 to 89, 86 to 90, 86 to 91, 86 to 92, 86 to 93, 86 to 94, 86 to 95, 86 to 96, 86 to 97, 86 to 98, 86 to 99, or 86 to 100 percent identical to SEQ ID NO: 1. In some embodiments, the VirD1 protein includes or is composed entirely of an amino acid sequence that is 87 to 88, 87 to 89, 87 to 90, 87 to 91, 87 to 92, 87 to 93, 87 to 94, 87 to 95, 87 to 96, 87 to 97, 87 to 98, 87 to 99, or 87 to 100 percent identical to SEQ ID NO: 1. In some embodiments, the VirD1 protein includes or is composed entirely of an amino acid sequence that is 88 to 89, 88 to 90, 88 to 91, 88 to 92, 88 to 93, 88 to 94, 88 to 95, 88 to 96, 88 to 97, 88 to 98, 88 to 99, or 88 to 100 percent identical to SEQ ID NO: 1. In some embodiments, the VirD1 protein includes or is composed entirely of an amino acid sequence that is 89 to 90, 89 to 91, 89 to 92, 89 to 93, 89 to 94, 89 to 95, 89 to 96, 89 to 97, 89 to 98, 89 to 99, or 89 to 100 percent identical to SEQ ID NO: 1. In some embodiments, the VirD1 protein includes or is composed entirely of an amino acid sequence that is 90 to 91, 90 to 92, 90 to 93, 90 to 94, 90 to 95, 90 to 96, 90 to 97, 90 to 98, 90 to 99, or 90 to 100 percent identical to SEQ ID NO: 1. In some embodiments, the VirD1 protein includes or is composed entirely of an amino acid sequence that is 91 to 92, 91 to 93, 91 to 94, 91 to 95, 91 to 96, 91 to 97, 91 to 98, 91 to 99, or 91 to 100 percent identical to SEQ ID NO: 1. In some embodiments, the VirD1 protein includes or is composed entirely of an amino acid sequence that is 92 to 93, 92 to 94, 92 to 95, 92 to 96, 92 to 97, 92 to 98, 92 to 99, or 92 to 100 percent identical to SEQ ID NO: 1. In some embodiments, the VirD1 protein includes or is composed entirely of an amino acid sequence that is 93 to 94, 93 to 95, 93 to 96, 93 to 97, 93 to 98, 93 to 99, or 93 to 100 percent identical to SEQ ID NO: 1. In some embodiments, the VirD1 protein includes or is composed entirely of an amino acid sequence that is 94 to 95, 94 to 96, 94 to 97, 94 to 98, 94 to 99, or 94 to 100 percent identical to SEQ ID NO: 1. In some embodiments, the VirD1 protein includes or is composed entirely of an amino acid sequence that is 95 to 96, 95 to 97, 95 to 98, 95 to 99, or 95 to 100 percent identical to SEQ ID NO: 1. In some embodiments, the VirD1 protein includes or is composed entirely of an amino acid sequence that is 96 to 97, 96 to 98, 96 to 99, or 96 to 100 percent identical to SEQ ID NO: 1. In some embodiments, the VirD1 protein includes or is composed entirely of an amino acid sequence that is 97 to 98, 97 to 99, or 97 to 100 percent identical to SEQ ID NO: 1. In some embodiments, the VirD1 protein includes or is composed entirely of an amino acid sequence that is 98 to 99, or 98 to 100 percent identical to SEQ ID NO: 1. In some embodiments, the VirD1 protein includes or is composed entirely of an amino acid sequence that is 99 to 100 percent identical to SEQ ID NO: 1.

In some embodiments, the VirD2 protein has a polypeptide sequence that is 80-100 percent identical to SEQ ID NO: 2.

VirD2 Agrobacterium fabrum (strain C58/ATCC 33970)
(SEQ ID NO: 2)
MPDRAQVIIRIVPGGGTKTLQQIINQLEYLSRKGRLELQRSARHLDIPL
PPDQIHELARSWVQETGTYDESQPDEERQQELTTHIIVSFPAGTSQVAA
YAASREWAAEMFGSGAGGGRYNYLTAFHIDRDHPHLHVVVNRRELLGHG
WLKISRRHPQLNYDALRIKMAEISLRHGIALDASRRAERGITERPITYA
QYRRLEREQARQIRFEDADLEQSSPQGDHPEFSQPFDTSPFEASAGGPE
DMPRPNNRQNESQVHLQEPAGVSNEAGVLVRVALETERLAQPFVSETIL
ADDIGSGSSRVAEGRVESANRTPDIPRAATEAATHTTHDRQRRAKRPHD
DDGGPSGAKRVTLEGIAVGPQANAGEQDGSSGPLVRQAGTSRPSPPTAT
TRASTATDSLSATAHLQQRRGVLSKRPREDDDGEPSERKRERDERSKDG
RGGNRR

In some embodiments, the VirD2 protein includes or is composed entirely of an amino acid sequence that is 80, 81, 82, 83, 84, 85, 86, 87, 88, 89, 90, 91, 92, 93, 94, 95, 96, 97, 98, 99, to/or 100 percent identical to SEQ ID NO: 2. In some embodiments, the VirD2 protein includes or is composed entirely of an amino acid sequence that is 80 to 81, 80 to 82, 80 to 83, 80 to 84, 80 to 85, 80 to 86, 80 to 87, 80 to 88, 80 to 89, 80 to 90, 80 to 91, 80 to 92, 80 to 93, 80 to 94, 80 to 95, 80 to 96, 80 to 97, 80 to 98, 80 to 99, or 80 to 100 percent identical to SEQ ID NO: 2. In some embodiments, the VirD2 protein includes or is composed entirely of an amino acid sequence that is 81 to 82, 81 to 83, 81 to 84, 81 to 85, 81 to 86, 81 to 87, 81 to 88, 81 to 89, 81 to 90, 81 to 91, 81 to 92, 81 to 93, 81 to 94, 81 to 95, 81 to 96, 81 to 97, 81 to 98, 81 to 99, or 81 to 100 percent identical to SEQ ID NO: 2. In some embodiments, the VirD2 protein includes or is composed entirely of an amino acid sequence that is 82 to 83, 82 to 84, 82 to 85, 82 to 86, 82 to 87, 82 to 88, 82 to 89, 82 to 90, 82 to 91, 82 to 92, 82 to 93, 82 to 94, 82 to 95, 82 to 96, 82 to 97, 82 to 98, 82 to 99, or 82 to 100 percent identical to SEQ ID NO: 2. In some embodiments, the VirD2 protein includes or is composed entirely of an amino acid sequence that is 83 to 84, 83 to 85, 83 to 86, 83 to 87, 83 to 88, 83 to 89, 83 to 90, 83 to 91, 83 to 92, 83 to 93, 83 to 94, 83 to 95, 83 to 96, 83 to 97, 83 to 98, 83 to 99, or 83 to 100 percent identical to SEQ ID NO: 2. In some embodiments, the VirD2 protein includes or is composed entirely of an amino acid sequence that is 84 to 85, 84 to 86, 84 to 87, 84 to 88, 84 to 89, 84 to 90, 84 to 91, 84 to 92, 84 to 93, 84 to 94, 84 to 95, 84 to 96, 84 to 97, 84 to 98, 84 to 99, or 84 to 100 percent identical to SEQ ID NO: 2. In some embodiments, the VirD2 protein includes or is composed entirely of an amino acid sequence that is 85 to 86, 85 to 87, 85 to 88, 85 to 89, 85 to 90, 85 to 91, 85 to 92, 85 to 93, 85 to 94, 85 to 95, 85 to 96, 85 to 97, 85 to 98, 85 to 99, or 85 to 100 percent identical to SEQ ID NO: 2. In some embodiments, the VirD2 protein includes or is composed entirely of an amino acid sequence that is 86 to 87, 86 to 88, 86 to 89, 86 to 90, 86 to 91, 86 to 92, 86 to 93, 86 to 94, 86 to 95, 86 to 96, 86 to 97, 86 to 98, 86 to 99, or 86 to 100 percent identical to SEQ ID NO: 2. In some embodiments, the VirD2 protein includes or is composed entirely of an amino acid sequence that is 87 to 88, 87 to 89, 87 to 90, 87 to 91, 87 to 92, 87 to 93, 87 to 94, 87 to 95, 87 to 96, 87 to 97, 87 to 98, 87 to 99, or 87 to 100 percent identical to SEQ ID NO: 2. In some embodiments, the VirD2 protein includes or is composed entirely of an amino acid sequence that is 88 to 89, 88 to 90, 88 to 91, 88 to 92, 88 to 93, 88 to 94, 88 to 95, 88 to 96, 88 to 97, 88 to 98, 88 to 99, or 88 to 100 percent identical to SEQ ID NO: 2. In some embodiments, the VirD2 protein includes or is composed entirely of an amino acid sequence that is 89 to 90, 89 to 91, 89 to 92, 89 to 93, 89 to 94, 89 to 95, 89 to 96, 89 to 97, 89 to 98, 89 to 99, or 89 to 100 percent identical to SEQ ID NO: 2. In some embodiments, the VirD2 protein includes or is composed entirely of an amino acid sequence that is 90 to 91, 90 to 92, 90 to 93, 90 to 94, 90 to 95, 90 to 96, 90 to 97, 90 to 98, 90 to 99, or 90 to 100 percent identical to SEQ ID NO: 2. In some embodiments, the VirD2 protein includes or is composed entirely of an amino acid sequence that is 91 to 92, 91 to 93, 91 to 94, 91 to 95, 91 to 96, 91 to 97, 91 to 98, 91 to 99, or 91 to 100 percent identical to SEQ ID NO: 2. In some embodiments, the VirD2 protein includes or is composed entirely of an amino acid sequence that is 92 to 93, 92 to 94, 92 to 95, 92 to 96, 92 to 97, 92 to 98, 92 to 99, or 92 to 100 percent identical to SEQ ID NO: 2. In some embodiments, the VirD2 protein includes or is composed entirely of an amino acid sequence that is 93 to 94, 93 to 95, 93 to 96, 93 to 97, 93 to 98, 93 to 99, or 93 to 100 percent identical to SEQ ID NO: 2. In some embodiments, the VirD2 protein includes or is composed entirely of an amino acid sequence that is 94 to 95, 94 to 96, 94 to 97, 94 to 98, 94 to 99, or 94 to 100 percent identical to SEQ ID NO: 2. In some embodiments, the VirD2 protein includes or is composed entirely of an amino acid sequence that is 95 to 96, 95 to 97, 95 to 98, 95 to 99, or 95 to 100 percent identical to SEQ ID NO: 2. In some embodiments, the VirD2 protein includes or is composed entirely of an amino acid sequence that is 96 to 97, 96 to 98, 96 to 99, or 96 to 100 percent identical to SEQ ID NO: 2. In some embodiments, the VirD2 protein includes or is composed entirely of an amino acid sequence that is 97 to 98, 97 to 99, or 97 to 100 percent identical to SEQ ID NO: 2. In some embodiments, the VirD2 protein includes or is composed entirely of an amino acid sequence that is 98 to 99, or 98 to 100 percent identical to SEQ ID NO: 2. In some embodiments, the VirD2 protein includes or is composed entirely of an amino acid sequence that is 99 to 100 percent identical to SEQ ID NO: 2.

Also provided herein are encoding polynucleotides for the Vir polypeptides, including encoding polynucleotides for VirD1, VirD2, and any other Vir proteins (e.g., VirA, VirB, VirC, VirE (e.g., a VirE2), VirG, VirH) included in the compositions and systems described herein. In some embodiments, the encoding polynucleotides are codon optimized for expression in certain organism such as human, non-human primates, non-human animals (e.g. chicken, rat, mouse, dog, cat, horse, pig, etc.), or plants.

In some embodiments, the VirA protein is an Agrobacterium tumefaciens VirA protein. Exemplary Agrobacterium tumefaciens VirA are GenBank Accession No. AF001781, M18059, AAZ50512, U41447, and AAF77159. In some embodiments, the VirA protein is 80-100% identical to GenBank Accession No. AF001781, M18059, AAZ50512, U41447, or AAF77159.

In some embodiments, the VirB protein is an Agrobacterium tumefaciens VirB protein. Exemplary Agrobacterium tumefaciens VirB are GenBank Accession No. CAA68594. In some embodiments, the VirB protein is 80-100% identical to GenBank Accession No. CAA68594.

In some embodiments, the VirC protein is an Agrobacterium tumefaciens VirC protein. Exemplary Agrobacterium tumefaciens VirC are GenBank Accession No. CAA68597, In some embodiments, the VirC protein is 80-100% identical to GenBank Accession No. CAA68597.

In some embodiments, the VirE protein is an Agrobacterium tumefaciens VirE protein. Exemplary Agrobacterium tumefaciens VirE are GenBank Accession No. CVI25582. In some embodiments, the VirE protein is 80-100% identical to GenBank Accession No. CVI25582.

In some embodiments, the VirG protein is an Agrobacterium tumefaciens VirG protein. Exemplary Agrobacterium tumefaciens VirG are GenBank Accession No. CAA68595. In some embodiments, the VirG protein is 80-100% identical to GenBank Accession No. CAA68595.

In some embodiments, the VirH protein is an Agrobacterium tumefaciens VirH protein. Exemplary Agrobacterium tumefaciens VirH are GenBank Accession No. In some embodiments, the VirH protein is 80-100% identical to GenBank Accession No.

In some embodiments the left and right boundary sequences recognized by the VirD1 and/or VirD2, which can be included in a donor construct (discussed in greater detail elsewhere herein) are as follows: 5′ GTTTACCCGCCAATATATCCTGTCA 3′ (SEQ ID NO: 4).

In some embodiments, the VirD2 polypeptide is connected to or otherwise capable of forming a complex with the site-specific nuclease polypeptide of the systems and compositions of the present invention.

In some embodiments, the VirD1 polypeptide is connected to or otherwise capable of forming a complex with the VirD2 polypeptide of the systems and compositions of the present invention.

### DNA Polymerase

In some embodiments, the engineered nucleic acid modification systems and compositions described herein include a DNA polymerase. Generally, DNA polymerases are enzymes that are capable of synthesizing DNA molecules from nucleoside triphosphates and template single stranded DNA.

In some embodiments, the DNA polymerase is connected to, coupled to, associated with, or otherwise capable of forming a complex with the site-specific nuclease polypeptide. In some embodiments, the DNA polymerase can facilitate integration of a donor polynucleotide into a target polynucleotide.

The DNA polymerase included in the systems and compositions described herein can be any suitable DNA polymerase. In some embodiments, the DNA polymerase is a prokaryotic polymerase. In some embodiments, the DNA polymerase is an E. coli DNA polymerase. In some embodiments, the DNA polymerase is a Pol I DNA polymerase. In some embodiments, the DNA polymerase is a Pol I DNA polymerase (see e.g., Maga et al. 2010. DNA Polymerases: Discovery Characterization Functions in Cellular DNA Transactions. World Scientific publishing Company. ISBN 978-981-4288-16-9). In some embodiments, the DNA polymerase is a Pol II DNA polymerase (see e.g., Banach-Orlowska M, Fijalkowska I J, Schaaper R M, Jonczyk P (October 2005). “DNA polymerase II as a fidelity factor in chromosomal DNA synthesis in Escherichia coli”. Molecular Microbiology. 58 (1): 61-70. doi:10.1111/j.1365-2958.2005.04805.x). In some embodiments, the DNA polymerase is a Pol III DNA polymerase (Olson M W, Dallmann H G, McHenry C S (December 1995). “DnaX complex of Escherichia coli DNA polymerase III holoenzyme. The chi psi complex functions by increasing the affinity of tau and gamma for delta.delta′ to a physiologically relevant range”. J. Biol. Chem. 270 (49): 29570-7. doi:10.1074/jbc.270.49.29570). In some embodiments, the DNA polymerase is a Pol IV DNA polymerase (see e.g., Goodman M F (2002). “Error-prone repair DNA polymerases in prokaryotes and eukaryotes”. Annual Review of Biochemistry. 71: 17-50. doi:10.1146/annurev.biochem.71.083101.124707). In some embodiments, the DNA polymerase is a Pol V DNA polymerase (see e.g., Patel M, Jiang Q, Woodgate R, Cox M M, Goodman M F (June 2010). “A new model for SOS-induced mutagenesis: how RecA protein activates DNA polymerase V”. Critical Reviews in Biochemistry and Molecular Biology. 45 (3): 171-84. doi:10.3109/10409238.2010.480968). In some embodiments, the DNA polymerase is a family D DNA polymerase (see e.g., Raia et al. 2019, PLOS Biol. 17(1): e3000122 doi:10.1371/journal.pbio.3000122).

In some embodiments, the DNA polymerase is a eukaryotic polymerase. In some embodiments the DNA polymerase is polymerase beta, polymerase lambda, polymerase sigma, polymerase mu, or polymerase TdT (see e.g., Yamtich J, Sweasy J B (May 2010). “DNA polymerase family X: function, structure, and cellular roles”. Biochimica et Biophysica Acta (BBA)—Proteins and Proteomics. 1804 (5): 1136-50. doi:10.1016/j.bbapap.2009.07.008). In some embodiments, the DNA polymerase is DNA polymerase alpha, polymerase delta, or polymerase epsilon (see e.g., Lujan S A, Williams J S, Kunkel T A (September 2016). “DNA Polymerases Divide the Labor of Genome Replication”. Trends in Cell Biology. 26 (9): 640-654. doi:10.1016/j.tcb.2016.04.012). In some embodiments, the DNA polymerase is polymerase eta, polymerase iota, or polymerase kappa (Ohmori H, Hanafusa T, Ohashi E, Vaziri C (2009). Separate roles of structured and unstructured regions of Y-family DNA polymerases. Advances in Protein Chemistry and Structural Biology. 78. pp. 99-146. doi:10.1016/S1876-1623(08)78004-0). In some embodiments, the DNA polymerase is polymerase Rev1 or polymerase zeta (Gan G N, Wittschieben J P, Wittschieben B O, Wood R D (January 2008). “DNA polymerase zeta (pol zeta) in higher eukaryotes”. Cell Research. 18 (1): 174-83. doi:10.1038/cr.2007.117). In some embodiments, the polymerase is a polymerase gamma or a polymerase nu (see e.g., Zhang L, Chan S S, Wolff D J (July 2011). “Mitochondrial disorders of DNA polymerase 7 dysfunction: from anatomic to molecular pathology diagnosis”. Archives of Pathology & Laboratory Medicine. 135 (7): 925-34. doi:10.1043/2010-0356-RAR.1; Hogg M, Sauer-Eriksson A E, Johansson E (March 2012). “Promiscuous DNA synthesis by human DNA polymerase 0”. Nucleic Acids Research. 40 (6): 2611-22. doi:10.1093/nar/gkr1102; Cupp J D, Nielsen B L (November 2014). “Minireview: DNA replication in plant mitochondria”. Mitochondrion. 19 Pt B: 231-7. doi:10.1016/j.mito.2014.03.008; and UniProtKB—Q7Z5Q5 (DPOLN_HUMAN)”. Uniprot. Retrieved 5 Jul. 2018).

In some embodiments, the DNA polymerase is a telomerase. In some embodiments the polymerase is a reverse transcriptase. In some embodiments, the DNA polymerase is a thermo-stable DNA polymerase.

In some embodiments, the DNA polymerase has an amino acid sequence that is 80-100 percent identical to the following amino acid sequence:

DNA polymerase I Escherichia coli (strain K12)
(SEQ ID NO: 3)
MVQIPQNPLILVDGSSYLYRAYHAFPPLTNSAGEPTGAMYGVLNMLRSL
IMQYKPTHAAVVFDAKGKTFRDELFEHYKSHRPPMPDDLRAQIEPLHAM
VKAMGLPLLAVSGVEADDVIGTLAREAEKAGRPVLISTGDKDMAQLVTP
NITLINTMTNTILGPEEVVNKYGVPPELIIDFLALMGDSSDNIPGVPGV
GEKTAQALLQGLGGLDTLYAEPEKIAGLSFRGAKTMAAKLEQNKEVAYL
SYQLATIKTDVELELTCEQLEVQQPAAEELLGLFKKYEFKRWTADVEAG
KWLQAKGAKPAAKPQETSVADEAPEVTATVISYDNYVTILDEETLKAWI
AKLEKAPVFAFDTETDSLDNISANLVGLSFAIEPGVAAYIPVAHDYLDA
PDQISRERALELLKPLLEDEKALKVGQNLKYDRGILANYGIELRGIAFD
TMLESYILNSVAGRHDMDSLAERWLKHKTITFEEIAGKGKNQLTFNQIA
LEEAGRYAAEDADVTLQLHLKMWPDLQKHKGPLNVFENIEMPLVPVLSR
IERNGVKIDPKVLHNHSEELTLRLAELEKKAHEIAGEEFNLSSTKQLQT
ILFEKQGIKPLKKTPGGAPSTSEEVLEELALDYPLPKVILEYRGLAKLK
STYTDKLPLMINPKTGRVHTSYHQAVTATGRLSSTDPNLQNIPVRNEEG
RRIRQAFIAPEDYVIVSADYSQIELRIMAHLSRDKGLLTAFAEGKDIHR
ATAAEVFGLPLETVTSEQRRSAKAINFGLIYGMSAFGLARQLNIPRKEA
QKYMDLYFERYPGVLEYMERTRAQAKEQGYVETLDGRRLYLPDIKSSNG
ARRAAAERAAINAPMQGTAADIIKRAMIAVDAWLQAEQPRVRMIMQVHD
ELVFEVHKDDVDAVAKQIHQLMENCTRLDVPLLVEVGSGENWDQAH

In some embodiments, the DNA polymerase includes or is composed entirely of an amino acid sequence that is 80, 81, 82, 83, 84, 85, 86, 87, 88, 89, 90, 91, 92, 93, 94, 95, 96, 97, 98, 99, to/or 100 percent identical to SEQ ID NO: 3. In some embodiments, the DNA polymerase includes or is composed entirely of an amino acid sequence that is 80 to 81, 80 to 82, 80 to 83, 80 to 84, 80 to 85, 80 to 86, 80 to 87, 80 to 88, 80 to 89, 80 to 90, 80 to 91, 80 to 92, 80 to 93, 80 to 94, 80 to 95, 80 to 96, 80 to 97, 80 to 98, 80 to 99, or 80 to 100 percent identical to SEQ ID NO: 3. In some embodiments, the DNA polymerase includes or is composed entirely of an amino acid sequence that is 81 to 82, 81 to 83, 81 to 84, 81 to 85, 81 to 86, 81 to 87, 81 to 88, 81 to 89, 81 to 90, 81 to 91, 81 to 92, 81 to 93, 81 to 94, 81 to 95, 81 to 96, 81 to 97, 81 to 98, 81 to 99, or 81 to 100 percent identical to SEQ ID NO: 3. In some embodiments, the DNA polymerase includes or is composed entirely of an amino acid sequence that is 82 to 83, 82 to 84, 82 to 85, 82 to 86, 82 to 87, 82 to 88, 82 to 89, 82 to 90, 82 to 91, 82 to 92, 82 to 93, 82 to 94, 82 to 95, 82 to 96, 82 to 97, 82 to 98, 82 to 99, or 82 to 100 percent identical to SEQ ID NO: 3. In some embodiments, the DNA polymerase includes or is composed entirely of an amino acid sequence that is 83 to 84, 83 to 85, 83 to 86, 83 to 87, 83 to 88, 83 to 89, 83 to 90, 83 to 91, 83 to 92, 83 to 93, 83 to 94, 83 to 95, 83 to 96, 83 to 97, 83 to 98, 83 to 99, or 83 to 100 percent identical to SEQ ID NO: 3. In some embodiments, the DNA polymerase includes or is composed entirely of an amino acid sequence that is 84 to 85, 84 to 86, 84 to 87, 84 to 88, 84 to 89, 84 to 90, 84 to 91, 84 to 92, 84 to 93, 84 to 94, 84 to 95, 84 to 96, 84 to 97, 84 to 98, 84 to 99, or 84 to 100 percent identical to SEQ ID NO: 3. In some embodiments, the DNA polymerase includes or is composed entirely of an amino acid sequence that is 85 to 86, 85 to 87, 85 to 88, 85 to 89, 85 to 90, 85 to 91, 85 to 92, 85 to 93, 85 to 94, 85 to 95, 85 to 96, 85 to 97, 85 to 98, 85 to 99, or 85 to 100 percent identical to SEQ ID NO: 3. In some embodiments, the DNA polymerase includes or is composed entirely of an amino acid sequence that is 86 to 87, 86 to 88, 86 to 89, 86 to 90, 86 to 91, 86 to 92, 86 to 93, 86 to 94, 86 to 95, 86 to 96, 86 to 97, 86 to 98, 86 to 99, or 86 to 100 percent identical to SEQ ID NO: 3. In some embodiments, the DNA polymerase includes or is composed entirely of an amino acid sequence that is 87 to 88, 87 to 89, 87 to 90, 87 to 91, 87 to 92, 87 to 93, 87 to 94, 87 to 95, 87 to 96, 87 to 97, 87 to 98, 87 to 99, or 87 to 100 percent identical to SEQ ID NO: 3. In some embodiments, the DNA polymerase includes or is composed entirely of an amino acid sequence that is 88 to 89, 88 to 90, 88 to 91, 88 to 92, 88 to 93, 88 to 94, 88 to 95, 88 to 96, 88 to 97, 88 to 98, 88 to 99, or 88 to 100 percent identical to SEQ ID NO: 3. In some embodiments, the DNA polymerase includes or is composed entirely of an amino acid sequence that is 89 to 90, 89 to 91, 89 to 92, 89 to 93, 89 to 94, 89 to 95, 89 to 96, 89 to 97, 89 to 98, 89 to 99, or 89 to 100 percent identical to SEQ ID NO: 3. In some embodiments, the DNA polymerase includes or is composed entirely of an amino acid sequence that is 90 to 91, 90 to 92, 90 to 93, 90 to 94, 90 to 95, 90 to 96, 90 to 97, 90 to 98, 90 to 99, or 90 to 100 percent identical to SEQ ID NO: 3. In some embodiments, the DNA polymerase includes or is composed entirely of an amino acid sequence that is 91 to 92, 91 to 93, 91 to 94, 91 to 95, 91 to 96, 91 to 97, 91 to 98, 91 to 99, or 91 to 100 percent identical to SEQ ID NO: 3. In some embodiments, the DNA polymerase includes or is composed entirely of an amino acid sequence that is 92 to 93, 92 to 94, 92 to 95, 92 to 96, 92 to 97, 92 to 98, 92 to 99, or 92 to 100 percent identical to SEQ ID NO: 3. In some embodiments, the DNA polymerase includes or is composed entirely of an amino acid sequence that is 93 to 94, 93 to 95, 93 to 96, 93 to 97, 93 to 98, 93 to 99, or 93 to 100 percent identical to SEQ ID NO: 3. In some embodiments, the DNA polymerase includes or is composed entirely of an amino acid sequence that is 94 to 95, 94 to 96, 94 to 97, 94 to 98, 94 to 99, or 94 to 100 percent identical to SEQ ID NO: 3. In some embodiments, the DNA polymerase includes or is composed entirely of an amino acid sequence that is 95 to 96, 95 to 97, 95 to 98, 95 to 99, or 95 to 100 percent identical to SEQ ID NO: 3. In some embodiments, the DNA polymerase includes or is composed entirely of an amino acid sequence that is 96 to 97, 96 to 98, 96 to 99, or 96 to 100 percent identical to SEQ ID NO: 3. In some embodiments, the DNA polymerase includes or is composed entirely of an amino acid sequence that is 97 to 98, 97 to 99, or 97 to 100 percent identical to SEQ ID NO: 3. In some embodiments, the DNA polymerase includes or is composed entirely of an amino acid sequence that is 98 to 99, or 98 to 100 percent identical to SEQ ID NO: 3. In some embodiments, the DNA polymerase includes or is composed entirely of an amino acid sequence that is 99 to 100 percent identical to SEQ ID NO: 3.

In some embodiments, the DNA polymerase is connected to or otherwise capable of complexing with the site-specific nuclease. In some embodiments the DNA polymerase is capable of complexing with or otherwise interacting with a donor polynucleotide sequence and/or a target polynucleotide.

Also described herein are DNA polymerase encoding polynucleotides that encode one or more DNA polymerases described herein that are included in the compositions and systems described herein. In some embodiments, the encoding polynucleotides are codon optimized for expression in certain organism such as human, non-human primates, non-human animals (e.g., chicken, rat, mouse, dog, cat, horse, pig, etc.), or plants.

### Donor Construct

The engineered nucleic acid modification systems can include one or more donor constructs comprising one or more donor polynucleotide sequences for insertion into a target polynucleotide. In embodiments, the donor polynucleotide sequence is flanked on the 5′ end with a right T-DNA boundary sequence. In some embodiments, the donor polynucleotide sequence is also flanked on the 3′ end with a left T-DNA boundary sequence. As previously discussed, the T-DNA boundary sequences can provide binding/cleavage/interaction sequences for complexing or otherwise interacting with VirD2 and/or VirD1. In certain example embodiments, the donor construct comprises a polynucleotide encoding a 5′ and a 3′ boundary sequence and a donor polynucleotide sequence located between the 5′ and the 3′ boundary sequences. In some embodiments the donor construct comprises a double stranded polynucleotide encoding a 5′ and a 3′ boundary sequence and a donor polynucleotide sequence located between the 5′ and the 3′ boundary sequences. In some embodiments, the donor construct comprises a single stranded polynucleotide encoding a 5′ boundary sequence followed by a donor polynucleotide sequence at the 3′ end of the 5′ boundary sequence. In certain example embodiments, the single stranded polynucleotide donor construct does not contain a 3′ boundary sequence.

A donor polynucleotide sequence may be any type of polynucleotides, including, but not limited to, a gene, a gene fragment, a non-coding polynucleotide, a regulatory polynucleotide, a synthetic polynucleotide, etc.

In some embodiments the donor construct is a double stranded polynucleotide. In some embodiments, the donor construct is or is based on a Ti or Ri plasmid. In some embodiments, the donor construct includes one or more features or polynucleotides from or modified from a Ti or Ri plasmid. In some embodiments, the donor construct is a single-stranded polynucleotide.

The donor construct may further comprise one or more processing element. The processing element is an element that may be added to ensure accurate processing and incorporation of the donor polynucleotide sequence by the fusion proteins disclosed herein.

The donor construct may comprise one or more homology sequence. A homology sequence is a sequence that shares or complete or partial homology with a target sequence at the site the targeted site of insertion and/or guide molecule. The homology sequence may be located on the 5′ end, ′3 end, or on both the 5′ and 3′ end of the donor construct. In certain example embodiments, the homology sequence is only located on the 5′ end of the donor construct and/or donor polynucleotide sequence. In certain example embodiments, the homology sequence is located only on the 3′ end of the donor construct and/or donor polynucleotide sequence. In certain example embodiments, the location of the homology sequence may depend on whether the site-specific nuclease is being directed to create a nick or cut 5′ or 3′ of the targeted insertion site, e.g., a 5′ homology sequence on the donor construct may be used when the site specific nuclease creates a nick or cut 5′ of the targeted insertion site and a 3′ homology sequence may be used when the site-specific nuclease is configured to create a nick or cut 3′ of the targeted insertion site. In certain example embodiments, the homology sequence is included on both the 5′ and 3′ ends of the donor construct and/or donor polynucleotide sequence regardless of whether the site-specific nuclease creates a nick or cut 5′ or 3′ of the targeted insertion site. In certain example embodiments the donor construct may comprise in a 5′ to 3′, a 5′ boundary sequence, and the donor sequence. In certain example embodiments, the donor construct may comprise in a 5′ to 3′ direction a homology sequence, a boundary sequence, and the donor polynucleotide sequence. In certain example embodiments the donor construct may comprise in a 5′ to 3′ direction a homology sequence, a 5′ boundary sequence, the donor polynucleotide sequence, and a 3′ boundary sequence. In certain example embodiments, the donor construct may comprise in a 5′ to 3′ direction a first homology sequence, a first boundary sequence, the donor sequence, and a second homology sequence. In certain example embodiments the donor construct may comprise, in a 5′ to 3′ direction, a first homology sequence, a first boundary sequence, the donor sequence, a second boundary sequence, and a second homology sequence. In certain example embodiments, the donor construct may comprise, in a 5′ to 3′ direction, the donor sequence and a boundary sequence. In certain example embodiments, the donor construct may comprise, in a 5′ to 3′ direction, the donor sequence, a boundary sequence, and a homology sequence.

The homology sequence may have at least 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 130, 140, 150, 175, 200 bases of homology to the target DNA and/or guide molecule. In certain example embodiments, the homology sequence may have between 8, 9, 10, 11, 12, 13, 14, 15, 16, 17, 18, 19, 20, 21, 22, 23, 24, or 25 base pairs of homology to the target sequence and/or guide molecule. In embodiments with a homology sequence on both the 5′ and 3′ end of the donor construct, the size of the homology may be the same or different on each end. In some examples, the homology sequence comprises from 1 to 30, from 4 to 10, or from 10 to 25 nucleotides. For example, the homology sequence comprises from 4 to 10 nucleotides. For example, the homology sequence comprises from 10 to 25 nucleotides. For example, the homology sequence comprises 1 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides. In some embodiments, the homology sequence is 17 nucleotides.

A target polynucleotide may comprise a protospacer adjacent motif (PAM) sequence. An example of the PAM sequence is AT. The donor polynucleotides may be inserted into a recipient target polynucleotide upstream or downstream of the PAM sequence of a target polynucleotide. For example, the donor polynucleotide may be inserted at a position between 10 bases and 200 bases, e.g., between 20 bases and 150 bases, between 30 bases and 100 bases, between 45 bases and 70 bases, between 45 bases and 60 bases, between 55 bases and 70 bases, between 49 bases and 56 bases or between 60 bases and 66 bases, from a PAM sequence on the target polynucleotide. In some cases, the insertion is at a position upstream of the PAM sequence. In some cases, the insertion is at a position downstream of the PAM sequence. In some cases, the insertion is at a position from 49 to 56 bases or base pairs downstream from a PAM sequence. In some cases, the insertion is at a position from 60 to 66 bases or base pairs downstream from a PAM sequence.

In a strand of a polynucleotide, anything towards the 5′ end of a reference point is “upstream” of that point, and anything towards the 3′ end of a reference point is “downstream” of that point. A location upstream of a PAM sequence refers to a location at the 5′ side of the PAM sequence on the PAM-containing strand of the target sequence. A location downstream of a PAM sequence refers to a location at the 3′ side of the PAM sequence on the PAM-containing strand of the target sequence.

The compositions and systems herein may be used to insert a donor polynucleotide with desired orientation. For example, appropriate homology sequence may be selected to control the orientation of insertion on the 5′ or 3′ strand of the target sequence.

The donor polynucleotide comprises a homology sequence of a region of the target sequence. The homology sequence may share at least 50%, at least 60%, at least 70%, at least 80%, at least 90%, at least 95%, or 100% sequence identity with the region of the target sequence. In an example, the homology sequence shares 100% sequence identity with the region of the target sequence.

In some embodiments, the donor polynucleotide may be inserted into the strand on the target polynucleotide that contains the PAM (e.g., the PAM sequence of the site-specific nuclease such as Cas). In such cases, the donor polynucleotide may comprise a homology sequence of a region on the PAM containing strand of the target polynucleotide. Such a region may comprise the PAM sequence. The region may be at the 3′ side of the cleavage site of the site-specific nuclease. In some examples, the homology sequence may comprise from 4 to 10, or from 10 to 25 nucleotides in length.

In some embodiments, the donor polynucleotide may be inserted into the strand on the target polynucleotide that binds to the guide, e.g., the strand that contains a guide-binding sequence. In such cases, the donor polynucleotide may comprise a homology sequence of a region that comprises at least a portion of the guide-binding sequence of the target polynucleotide. In some cases, the region may comprise the entire guide-binding sequence. Such region may further comprise a sequence at the 3′ side of the guide-binding sequence. For example, the region may comprise from 5 to 15 nucleotides, e.g., 5, 6, 7, 8, 9, 10, 11, 12, 13, 14, 15 nucleotides from the 3′ side of the guide-binding sequence. In some cases, the region may be adjacent to the R-loop of the guide. For example, in the cases where the guide forms a RNA-DNA duplex with the guide-binding sequence, the region comprises a sequence at the 3′ side from the RNA-DNA duplex, e.g., from 5 to from 5 to 15 nucleotides, e.g., 5, 6, 7, 8, 9, 10, 11, 12, 13, 14, 15 nucleotides from the 3′ side from the RNA-DNA duplex.

In some examples, the homology sequence is of a region on the target sequence at 3′ side of a PAM-containing strand. In certain examples, the homology sequence is of a region on the target sequence 10 nucleotides from 3′ side of an RNA-DNA duplex formed by a guide molecule and a target sequence. For example, the guide molecule forms an RNA-DNA duplex with the target sequence, and the homology sequence is of a region on the target sequence 5 to 15 nucleotides from 3′ side of the RNA-DNA duplex. In some embodiments, the donor polynucleotide is inserted into a region on the target sequence that is 3′ side of a PAM-containing strand. In some cases, the donor polynucleotide is inserted to a region on the target sequence that is 3′ side of a sequence complementary to the guide molecule.

The donor polynucleotide sequence may be used for editing or otherwise modifying the target polynucleotide. In some cases, the donor polynucleotide comprises one or more mutations to be introduced into the target polynucleotide. Examples of such mutations include substitutions, deletions, insertions, or a combination thereof. The mutations may cause a shift in an open reading frame on the target polynucleotide. In some cases, the donor polynucleotide alters a stop codon in the target polynucleotide. For example, the donor polynucleotide may correct a premature stop codon. The correction may be achieved by deleting the stop codon or introduces one or more mutations to the stop codon. In other example embodiments, the donor polynucleotide addresses loss of function mutations, deletions, or translocations that may occur, for example, in certain disease contexts by inserting or restoring a functional copy of a gene, or functional fragment thereof, or a functional regulatory sequence or functional fragment of a regulatory sequence. A functional fragment refers to less than the entire copy of a gene by providing sufficient nucleotide sequence to restore the functionality of a wild type gene or non-coding regulatory sequence (e.g., sequences encoding long non-coding RNA). In certain example embodiments, the systems disclosed herein may be used to replace a single allele of a defective gene or defective fragment thereof. In another example embodiment, the systems disclosed herein may be used to replace both alleles of a defective gene or defective gene fragment. A “defective gene” or “defective gene fragment” is a gene or portion of a gene that when expressed fails to generate a functioning protein or non-coding RNA with functionality of a the corresponding wild-type gene. In certain example embodiments, these defective genes may be associated with one or more disease phenotypes. In certain example embodiments, the defective gene or gene fragment is not replaced but the systems described herein are used to insert donor polynucleotides that encode gene or gene fragments that compensate for or override defective gene expression such that cell phenotypes associated with defective gene expression are eliminated or changed to a different or desired cellular phenotype.

In certain embodiments, the donor may include, but not be limited to, genes or gene fragments, encoding proteins or RNA transcripts to be expressed, regulatory elements, repair templates, and the like. According to the invention, the donor polynucleotides may comprise left end and right end sequence elements that function with transposition components that mediate insertion.

In certain cases, the donor polynucleotide manipulates a splicing site on the target polynucleotide. In some examples, the donor polynucleotide disrupts a splicing site. The disruption may be achieved by inserting the polynucleotide to a splicing site and/or introducing one or more mutations to the splicing site. In certain examples, the donor polynucleotide may restore a splicing site. For example, the polynucleotide may comprise a splicing site sequence.

The donor polynucleotide to be inserted may has a size from 5 bases to 50 kb in length, e.g., from 50 to 40 kb, from 100 and 30 kb, from 100 bases to 300 bases, from 200 bases to 400 bases, from 300 bases to 500 bases, from 400 bases to 600 bases, from 500 bases to 700 bases, from 600 bases to 800 bases, from 700 bases to 900 bases, from 800 bases to 1000 bases, from 900 bases to from 1100 bases, from 1000 bases to 1200 bases, from 1100 bases to 1300 bases, from 1200 bases to 1400 bases, from 1300 bases to 1500 bases, from 1400 bases to 1600 bases, from 1500 bases to 1700 bases, from 600 bases to 1800 bases, from 1700 bases to 1900 bases, from 1800 bases to 2000 bases, from 1900 bases to 2100 bases, from 2000 bases to 2200 bases, from 2100 bases to 2300 bases, from 2200 bases to 2400 bases, from 2300 bases to 2500 bases, from 2400 bases to 2600 bases, from 2500 bases to 2700 bases, from 2600 bases to 2800 bases, from 2700 bases to 2900 bases, from 2800 bases to 3000 bases, from 2900 bases to 3100 bases, from 3000 bases to 3200 bases, from 3100 bases to 3300 bases, from 3200 bases to 3400 bases, from 3300 bases to 3500 bases, from 3400 bases to 3600 bases, from 3500 bases to 3700 bases, from 3600 bases to 3800 bases, from 3700 bases to 3900 bases, from 3800 bases to 4000 bases, from 3900 bases to 4100 bases, from 4000 bases to 4200 bases, from 4100 bases to 4300 bases, from 4200 bases to 4400 bases, from 4300 bases to 4500 bases, from 4400 bases to 4600 bases, from 4500 bases to 4700 bases, from 4600 bases to 4800 bases, from 4700 bases to 4900 bases, or from 4800 bases to 5000 bases in length.

### CRISPR-Cas Systems and Components Thereof

In some embodiments, the site-specific nuclease system is a CRISPR-Cas system. The DNA polymerase, Vir polypeptide (e.g., a VirD polypeptide), donor construct, or other component of the engineered nucleic acid modification system described herein may be associated with and/or be capable of forming a complex with one or more components of a CRISPR-Cas system, e.g., a Cas protein or polypeptide. The complex of Cas and DNA polymerase, Vir polypeptide, donor construct, or other component of the engineered nucleic acid modification system described herein may be directed to or recruited to a region of a target polynucleotide by sequence-specific binding of a CRISPR-Cas complex. In certain example embodiments, the DNA polymerase, Vir polypeptide (e.g., a VirD polypeptide), donor construct, or other component of the engineered nucleic acid modification system described herein may be connected to, fused or tethered (e.g., by a linker) to, or may otherwise form a complex with one or more components in a CRISPR-Cas system, e.g., Cas protein, guide molecule etc.).

The engineered nucleic acid modification systems herein may comprise one or more components of a CRISPR-Cas system. The one or more components of the CRISPR-Cas system may serve as the nucleotide-binding component in the systems. The nucleotide-binding molecule may be a Cas protein or polypeptide (used interchangeably with CRISPR protein, CRISPR enzyme, Cas effector, CRISPR-Cas protein, CRISPR-Cas enzyme), a fragment thereof, or a mutated form thereof. The Cas protein may have reduced or no nuclease activity. For example, the Cas protein may be an inactive or dead Cas protein (dCas). The dead Cas protein may comprise one or more mutations or truncations. In some examples, the DNA binding domain comprises one or more Class 1 (e.g., Type I, Type III, Type VI) or Class 2 (e.g., Type II, Type V, or Type VI) CRISPR-Cas proteins. In certain embodiments, the sequence-specific nucleotide binding domains directs a donor construct, donor polynucleotide, Vir polypeptide (e.g., a VirD polypeptide), a DNA polymerase, another component of the engineered nucleic acid modification system of the present invention, or any combination thereof to a target site in a target polypeptide comprising a target sequence and the VirD, and/or DNA polymerase directs insertion of a donor polynucleotide sequence at the target site. In certain example embodiments, the transposon component includes, associates with, or forms a complex with a CRISPR-Cas complex. In one example embodiment, the CRISPR-Cas component directs the transposon component and/or transposase(s) to a target insertion site where the transposon component directs insertion of the donor polynucleotide into a target nucleic acid sequence.

In general, a CRISPR-Cas or CRISPR system as used in herein and in documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov et al. (2015) “Discovery and Functional Characterization of Diverse Class 2 CRISPR-Cas Systems”, Molecular Cell, DOI: dx.doi.org/10.1016/j.molcel.2015.10.008.

In certain embodiments, a protospacer adjacent motif (PAM) or PAM-like motif directs binding of the effector protein complex as disclosed herein to the target sequence of interest. In some embodiments, the PAM may be a 5′ PAM (i.e., located upstream of the 5′ end of the protospacer). In other embodiments, the PAM may be a 3′ PAM (i.e., located downstream of the 5′ end of the protospacer). The term “PAM” may be used interchangeably with the term “PFS” or “protospacer flanking site” or “protospacer flanking sequence”.

In a preferred embodiment, the CRISPR effector protein may recognize a 3′ PAM. In certain embodiments, the CRISPR effector protein may recognize a 3′ PAM which is 5′H, wherein H is A, C or U.

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to a RNA polynucleotide being or comprising the target sequence. In other words, the target RNA may be a RNA polynucleotide or a part of a RNA polynucleotide to which a part of the gRNA, i.e. the guide sequence, is designed to have complementarity and to which the effector function mediated by the complex comprising CRISPR effector protein and a gRNA is to be directed. In some embodiments, a target sequence is located in the nucleus or cytoplasm of a cell.

### Cas Proteins and Polypeptides

The CRISPR-Cas systems herein may comprise a Cas protein and a guide molecule. In some embodiments, the system comprises one or more Cas proteins. The Cas proteins may be Type II or V Cas proteins, e.g., Cas proteins of Type II or V CRISPR-Cas systems.

A CRISPR-Cas system or CRISPR system refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system).

In the context of formation of a CRISPR complex, “target sequence” refers to a sequence in a polynucleotide (e.g., a target polynucleotide) to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell. In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, 2 of these criteria may be used, for instance 1 and 2, 2 and 3, or 1 and 3. In some embodiments, all 3 criteria may be used.

Examples of Cas proteins include those of Class 1 (e.g., Type I, Type III, and Type IV) and Class 2 (e.g., Type II, Type V, and Type VI) Cas proteins, e.g., Cas9, Cas12 (e.g., Cas12a, Cas12b, Cas12c, Cas12d), Cas13 (e.g., Cas13a, Cas13b, Cas13c, Cas13d,), CasX, CasY, Cas14, variants thereof (e.g., mutated forms, truncated forms), homologs thereof, and orthologs thereof.

The terms “orthologue” (also referred to as “ortholog” herein) and “homologue” (also referred to as “homolog” herein) are well known in the art. By means of further guidance, a “homologue” of a protein as used herein is a protein of the same species which performs the same or a similar function as the protein it is a homologue of Homologous proteins may but need not be structurally related or are only partially structurally related. An “orthologue” of a protein as used herein is a protein of a different species which performs the same or a similar function as the protein it is an orthologue of Orthologous proteins may but need not be structurally related or are only partially structurally related.

### Class 2 Cas Proteins

In certain example embodiments, the Cas protein is the Cas protein of a Class 2 CRISPR-Cas system (i.e., a Class 2 Cas protein). A Class 2 CRISPR-Cas system may be of a subtype, e.g., Type II-A, Type II-B, Type II-C, Type V-A, Type V-B, Type V-C, or Type V-U CRISPR-Cas system. In certain example embodiments, the Cas protein is Cas9, Cas12a, Cas12b, Cas12c, or Cas12d. In some embodiments, Cas9 may be SpCas9, SaCas9, StCas9 and other Cas9 orthologs. Cas 12 may be Cas12a, Cas12b, and Cas12c, including FnCas12a, or homology or orthologs thereof. The definition and exemplary members of the CRISPR-Cas system include those described in Kira S. Makarova and Eugene V. Koonin, Annotation and Classification of CRISPR-Cas systems, Methods Mol Biol. 2015; 1311: 47-75; and Sergey Shmakov et al., Diversity and evolution of class 2 CRISPR-Cas systems, Nat Rev Microbiol. 2017 March; 15(3): 169-182.

In some examples, the Cas protein comprises at least one RuvC and at least one HNH domain. In some examples, the Cas comprises at least one RuvC domain but does not comprise an HNH domain.

### Class 2 Type II Cas

In some embodiments, the Cas protein may be a Cas protein of a Class 2, Type II CRISPR-Cas system (a Type II Cas protein). In some embodiments, the Cas protein may be a class 2 Type II Cas protein, e.g., Cas9. By “Cas9 (CRISPR associated protein 9)” is meant a polypeptide or fragment thereof having at least about 85% amino acid identity to NCBI Accession No. NP_269215 and having RNA binding activity, DNA binding activity, and/or DNA cleavage activity (e.g., endonuclease or nickase activity). “Cas9 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cas 9 nucleic acid molecule” is meant a polynucleotide encoding a Cas9 polypeptide or fragment thereof. An exemplary Cas9 nucleic acid molecule sequence is provided at NCBI Accession No. NC_002737. In some embodiments, disclosed herein are inhibitors of Cas9, e.g., naturally occurring Cas9 in S. pyogenes (SpCas9) or S. aureus (SaCas9), or variants thereof. Cas9 recognizes foreign DNA using Protospacer Adjacent Motif (PAM) sequence and the base pairing of the target DNA by the guide RNA (gRNA). The relative ease of inducing targeted strand breaks at any genomic loci by Cas9 has enabled efficient genome editing in multiple cell types and organisms. Cas9 derivatives can also be used as transcriptional activators/repressors.

In some examples, the Cas9 may be in a mutated form. Examples of Cas9 mutations include D10A, E762A, H840A, N854A, N863A and D986A in respect of SpCas9. In one example, the Cas9 is Cas9D10A. In another example, the Cas9 is Cas9H840A.

### Class 2 Type V Cas

In certain embodiments, the Cas protein may be a Cas protein of a Class 2, Type V CRISPR-Cas system (a Type V Cas protein). Examples of class 2 Type V Cas proteins include Cas12a (Cpf1), Cas12b (C2c1), Cas12c (C2c3), or Cas12k.

In some examples, the Cas protein is Cpf1. By “Cpf1 (CRISPR associated protein Cpf1)” is meant a polypeptide or fragment thereof having at least about 85% amino acid identity to GenBank Accession No. AJI61006. 1 and having RNA binding activity, DNA binding activity, and/or DNA cleavage activity (e.g., endonuclease or nickase activity). “Cpf1 function” can be defined by any of a number of assays including, but not limited to, fluorescence polarization-based nucleic acid bind assays, fluorescence polarization-based strand invasion assays, transcription assays, EGFP disruption assays, DNA cleavage assays, and/or Surveyor assays, for example, as described herein. By “Cpf1 nucleic acid molecule” is meant a polynucleotide encoding a Cpf1 polypeptide or fragment thereof. An exemplary Cpf1 nucleic acid molecule sequence is provided at GenBank Accession No. CP009633, nucleotides 652838-656740. Cpf1 (CRISPR-associated protein Cpf1, subtype PREFRAN) is a large protein (about 1300 amino acids) that contains a RuvC-like nuclease domain homologous to the corresponding domain of Cas9 along with a counterpart to the characteristic arginine-rich cluster of Cas9. However, Cpf1 lacks the HNH nuclease domain that is present in all Cas9 proteins, and the RuvC-like domain is contiguous in the Cpf1 sequence, in contrast to Cas9 where it contains long inserts including the HNH domain. Accordingly, in particular embodiments, the CRISPR-Cas enzyme comprises only a RuvC-like nuclease domain.

The Cpf1 gene is found in several diverse bacterial genomes, typically in the same locus with cas1, cas2, and cas4 genes and a CRISPR cassette (for example, FNFX1_1431-FNFX1_1428 of Francisella cf. novicida Fx1). Thus, the layout of this putative novel CRISPR-Cas system appears to be similar to that of type II-B. Furthermore, similar to Cas9, the Cpf1 protein contains a readily identifiable C-terminal region that is homologous to the transposon ORF-B and includes an active RuvC-like nuclease, an arginine-rich region, and a Zn finger (absent in Cas9). However, unlike Cas9, Cpf1 is also present in several genomes without a CRISPR-Cas context and its relatively high similarity with ORF-B suggests that it might be a transposon component. It was suggested that if this was a genuine CRISPR-Cas system and Cpf1 is a functional analog of Cas9 it would be a novel CRISPR-Cas type, namely type V (See Annotation and Classification of CRISPR-Cas Systems. Makarova K S, Koonin E V. Methods Mol Biol. 2015; 1311:47-75). However, as described herein, Cpf1 is denoted to be in subtype V-A to distinguish it from C2c1p which does not have an identical domain structure and is hence denoted to be in subtype V-B.

In some examples, the Cas protein is Cc2c1. The C2c1 gene is found in several diverse bacterial genomes, typically in the same locus with cas1, cas2, and cas4 genes and a CRISPR cassette. Thus, the layout of this putative novel CRISPR-Cas system appears to be similar to that of type II-B. Furthermore, similar to Cas9, the C2c1 protein contains an active RuvC-like nuclease, an arginine-rich region, and a Zn finger (absent in Cas9). C2c1 (Cas12b) is derived from a C2c1 locus denoted as subtype V-B. Herein such effector proteins are also referred to as “C2c1p”, e.g., a C2c1 protein (and such effector protein or C2c1 protein or protein derived from a C2c1 locus is also called “CRISPR enzyme”). Presently, the subtype V-B loci encompasses cas1-Cas4 fusion, cas2, a distinct gene denoted C2c1 and a CRISPR array. C2c1 (CRISPR-associated protein C2c1) is a large protein (about 1100-1300 amino acids) that contains a RuvC-like nuclease domain homologous to the corresponding domain of Cas9 along with a counterpart to the characteristic arginine-rich cluster of Cas9. However, C2c1 lacks the HNH nuclease domain that is present in all Cas9 proteins, and the RuvC-like domain is contiguous in the C2c1 sequence, in contrast to Cas9 where it contains long inserts including the HNH domain. Accordingly, in particular embodiments, the CRISPR-Cas enzyme comprises only a RuvC-like nuclease domain.

C2c1 proteins are RNA guided nucleases. Its cleavage relies on a tracr RNA to recruit a guide RNA comprising a guide sequence and a direct repeat, where the guide sequence hybridizes with the target nucleotide sequence to form a DNA/RNA heteroduplex. Based on current studies, C2c1 nuclease activity also requires relies on recognition of PAM sequence. C2c1 PAM sequences may be T-rich sequences. In some embodiments, the PAM sequence is 5′ TTN 3′ or 5′ ATTN 3′, wherein N is any nucleotide. In a particular embodiment, the PAM sequence is 5′ TTC 3′. In a particular embodiment, the PAM is in the sequence of Plasmodium falciparum. C2c1 creates a staggered cut at the target locus, with a 5′ overhang, or a “sticky end” at the PAM distal side of the target sequence. In some embodiments, the 5′ overhang is 7 nt. See Lewis and Ke, Mol Cell. 2017 Feb. 2; 65(3):377-379.

### Nickases

In some embodiments, the Cas protein or polypeptide may be a nickase. The Cas proteins with nickase activity may be a mutated form of a wildtype Cas protein. Mutations can also be made at neighboring residues at amino acids that participate in the nuclease activity. In some embodiments, only the RuvC domain is inactivated, and in other embodiments, another putative nuclease domain is inactivated, wherein the effector protein complex functions as a nickase and cleaves only one DNA strand. In some embodiments, two Cas variants (each a different nickase) are used to increase specificity, two nickase variants are used to cleave DNA at a target (where both nickases cleave a DNA strand, while minimizing or eliminating off-target modifications where only one DNA strand is cleaved and subsequently repaired). In preferred embodiments the Cas protein cleaves sequences associated with or at a target locus of interest as a homodimer comprising two Cas protein molecules. In a preferred embodiment the homodimer may comprise two Cas protein molecules comprising a different mutation in their respective RuvC domains.

The Cas protein may be mutated with respect to a corresponding wild-type enzyme such that the mutated Cas protein lacks the ability to cleave one or both DNA strands of a target locus containing a target sequence. In particular embodiments, one or more catalytic domains of the Cas protein are mutated to produce a mutated Cas protein which cleaves only one DNA strand of a target sequence.

In certain embodiments of the methods provided herein the Cas protein is a mutated Cas protein which cleaves only one DNA strand, i.e., a nickase. More particularly, in the context of the present invention, the nickase ensures cleavage within the non-target sequence, i.e., the sequence which is on the opposite DNA strand of the target sequence and which is 3′ of the PAM sequence. By means of further guidance, and without limitation, an arginine-to-alanine substitution (R911A) in the Nuc domain of C2c1 from Alicyclobacillus acidoterrestris converts C2c1 from a nuclease that cleaves both strands to a nickase (cleaves a single strand). It will be understood by the skilled person that where the enzyme is not AacC2c1, a mutation may be made at a residue in a corresponding position.

In certain embodiments, the Cas protein may be a C2c1 nickase which comprises a mutation in the Nuc domain. In some embodiments, the C2c1 nickase comprises a mutation corresponding to amino acid positions R911, R1000, or R1015 in Alicyclobacillus acidoterrestris C2c1. In some embodiments, the C2c1 nickase comprises a mutation corresponding to R911A, R1000A, or R1015A in Alicyclobacillus acidoterrestris C2c1. In some embodiments, the C2c1 nickase comprises a mutation corresponding to R894A in Bacillus sp. V3-13 C2c1. In certain embodiments, the C2c1 protein recognizes PAMs with increased or decreased specificity as compared with an unmutated or unmodified form of the protein. In some embodiments, the C2c1 protein recognizes altered PAMs as compared with an unmutated or unmodified form of the protein.

In some embodiments, to minimize the level of toxicity and off-target effect, a Cas nickase can be used with a pair of guide RNAs targeting a site of interest. Guide sequences and strategies to minimize toxicity and off-target effects can be as in WO 2014/093622 (PCT/US2013/074667); or, via mutation as described herein.

In some examples, the system may comprise two or more nickases, in particular a dual or double nickase approach. In some embodiments, a single type Cas nickase may be delivered, for example a modified Cas or a modified Cas nickase as described herein. This results in the target DNA being bound by two Cas nickases. In addition, it is also envisaged that different orthologs may be used, e.g., a Cas nickase on one strand (e.g., the coding strand) of the DNA and an ortholog on the non-coding or opposite DNA strand. The ortholog can be, but is not limited to, a Cas nickase. It may be advantageous to use two different orthologs that require different PAMs and may also have different guide requirements, thus allowing a greater deal of control for the user. In certain embodiments, DNA cleavage will involve at least four types of nickases, wherein each type is guided to a different sequence of target DNA, wherein each pair introduces a first nick into one DNA strand and the second introduces a nick into the second DNA strand. In such methods, at least two pairs of single stranded breaks are introduced into the target DNA wherein upon introduction of first and second pairs of single-strand breaks, target sequences between the first and second pairs of single-strand breaks are excised. In certain embodiments, one or both of the orthologs is controllable, i.e., inducible.

### Dead Cas

In certain embodiments, the Cas protein is a catalytically inactive or dead Cas protein (dCas). For example, the Cas protein or polypeptide may lack nuclease activity. In some embodiments, the dCas comprises mutations in the nuclease domain. In some embodiments, the dCas effector protein has been truncated. In some cases, the dead Cas proteins may be fused with one or more functional domains.

### IscB and TnpB

In certain example embodiments, the site specific nuclease may be an IscB or TnpB protein or functional domain thereof. See e.g., Kapitonov et al. J. Bacteriol. 2016. 198(5):797-807.

dCas—Functional Domain

The Cas protein or its variant (e.g., dCas) may be associated (e.g., fused) to one or more functional domains. The association can be by direct linkage of the Cas protein to the functional domain, or by association with the crRNA. In a non-limiting example, the crRNA comprises an added or inserted sequence that can be associated with a functional domain of interest, including, for example, an aptamer or a nucleotide that binds to a nucleic acid binding adapter protein. The functional domain may be a functional heterologous domain.

The functional domain may cleave a DNA sequence or modify transcription or translation of a gene. Examples of functional domains include domains that have methylase activity, demethylase activity, transcription activation activity, transcription repression activity, transcription release factor activity, histone modification activity, RNA cleavage activity, DNA cleavage activity, nucleic acid binding activity, and molecular switches (e.g., light inducible). Preferred domains are Fok1, VP64, P65, HSF1, MyoD1. In the event that Fok1 is provided, multiple Fok1 functional domains may be provided to allow for a functional dimer and that gRNAs are designed to provide proper spacing for functional use (Fok1).

In some cases, the functional domains may be heterologous functional domains. For example, the one or more heterologous functional domains may comprise one or more nuclear localization signal (NLS) domains. The one or more heterologous functional domains may comprise at least two or more NLS domains. The one or more NLS domain(s) may be positioned at or near or in proximity to a terminus of the Cas protein and if two or more NLSs, each of the two may be positioned at or near or in proximity to a terminus of the Cas protein. The one or more heterologous functional domains may comprise one or more transcriptional activation domains. In a preferred embodiment the transcriptional activation domain may comprise VP64. The one or more heterologous functional domains may comprise one or more transcriptional repression domains. In a preferred embodiment the transcriptional repression domain comprises a KRAB domain or a SID domain (e.g., SID4X). The one or more heterologous functional domains may comprise one or more nuclease domains. In a preferred embodiment a nuclease domain comprises Fok1. Other examples of functional domains include translational initiator, translational activator, translational repressor, nucleases, in particular ribonucleases, a spliceosome, beads, a light inducible/controllable domain or a chemically inducible/controllable domain.

The positioning of the one or more functional domain on Cas or dCas protein is one which allows for correct spatial orientation for the functional domain to affect the target with the attributed functional effect. For example, if the functional domain is a transcription activator (e.g., VP64 or p65), the transcription activator is placed in a spatial orientation which allows it to affect the transcription of the target. Likewise, a transcription repressor may be positioned to affect the transcription of the target, and a nuclease (e.g., Fok1) will be advantageously positioned to cleave or partially cleave the target. This may include positions other than the N-/C-terminus of the Cas protein.

The Cas or dCas protein may be associated with the one or more functional domains through one or more adaptor proteins. The adaptor protein may utilize known linkers to attach such functional domains.

The fusion between the adaptor protein and the activator or repressor may include a linker. For example, GlySer linkers GGGS can be used. They can be used in repeats of 3 ((GGGGS)3 (SEQ ID NO: 5)) or 6, 9 or even 12 or more, to provide suitable lengths, as required. Linkers can be used between the guide RNAs and the functional domain (activator or repressor), or between the nucleic acid-targeting effector protein and the functional domain (activator or repressor). The linkers the user to engineer appropriate amounts of “mechanical flexibility”.

The term “linker” as used in reference to a fusion protein refers to a molecule which joins the proteins to form a fusion protein. Generally, such molecules have no specific biological activity other than to join or to preserve some minimum distance or other spatial relationship between the proteins. However, in certain embodiments, the linker may be selected to influence some property of the linker and/or the fusion protein such as the folding, net charge, or hydrophobicity of the linker. Suitable linkers for use in the methods of the present invention are well known to those of skill in the art and include, but are not limited to, straight or branched-chain carbon linkers, heterocyclic carbon linkers, or peptide linkers. However, as used herein the linker may also be a covalent bond (carbon-carbon bond or carbon-heteroatom bond). In particular embodiments, the linker is used to separate the Cas protein and the nucleotide deaminase by a distance sufficient to ensure that each protein retains its required functional property. Preferred peptide linker sequences adopt a flexible extended conformation and do not exhibit a propensity for developing an ordered secondary structure. In certain embodiments, the linker can be a chemical moiety which can be monomeric, dimeric, multimeric or polymeric. Preferably, the linker comprises amino acids. Typical amino acids in flexible linkers include Gly, Asn and Ser. Accordingly, in particular embodiments, the linker comprises a combination of one or more of Gly, Asn and Ser amino acids. Other near neutral amino acids, such as Thr and Ala, also may be used in the linker sequence. Exemplary linkers are disclosed in Maratea et al. (1985), Gene 40: 39-46; Murphy et al. (1986) Proc. Nat'l. Acad. Sci. USA 83: 8258-62; U.S. Pat. Nos. 4,935,233; and 4,751,180. For example, GlySer linkers GGS, GGGS (SEQ ID NO: 6) or GSG can be used. GGS, GSG, GGGS (SEQ ID NO: 6) or GGGGS (SEQ ID NO: 7) linkers can be used in repeats of 3 (such as (GGS)₃ (SEQ ID NO: 8), (GGGGS)₃ (SEQ ID NO: 5)) or 5, 6, 7, 9 or even 12 or more, to provide suitable lengths. In some cases, the linker may be (GGGGS)₃₁₅, For example, in some cases, the linker may be (GGGGS)₃₁₁, e.g., GGGGS (SEQ ID NO: 7), (GGGGS)₂ (SEQ ID NO: 9), (GGGGS)₃ (SEQ ID NO: 5), (GGGGS)₄ (SEQ ID NO: 10), (GGGGS)₅ (SEQ ID NO: 11), (GGGGS)₆ (SEQ ID NO: 12), (GGGGS)₇ (SEQ ID NO: 13), (GGGGS)₈ (SEQ ID NO: 14), (GGGGS)₉ (SEQ ID NO: 15), (GGGGS)₁₀ (SEQ ID NO: 16), or (GGGGS)₁₁ (SEQ ID NO: 17). In particular embodiments, linkers such as (GGGGS)₃ (SEQ ID NO: 5) are preferably used herein. (GGGGS)₆ (SEQ ID NO: 12), (GGGGS)₉ (SEQ ID NO: 15) or (GGGGS)₁₂ (SEQ ID NO: 18) may preferably be used as alternatives. Other preferred alternatives are (GGGGS)₁ (SEQ ID NO: 7), (GGGGS)₂ (SEQ ID NO: 9), (GGGGS)₄ (SEQ ID NO: 10), (GGGGS)₅ (SEQ ID NO: 11), (GGGGS)₇ (SEQ ID NO: 13), (GGGGS)₈ (SEQ ID NO: 14), (GGGGS)₁₀ (SEQ ID NO: 16), or (GGGGS)₁₁ (SEQ ID NO: 17). In yet a further embodiment, LEPGEKPYKCPECGKSFSQSGALTRHQRTHTR (SEQ ID NO: 19) is used as a linker. In yet an additional embodiment, the linker is an XTEN linker. In particular embodiments, the CRISPR-Cas protein is linked to the deaminase protein or its catalytic domain by means of an LEPGEKPYKCPECGKSFSQSGALTRHQRTHTR (SEQ ID NO: 19) linker. In further particular embodiments, the CRISPR-Cas protein is linked C-terminally to the N-terminus of a deaminase protein or its catalytic domain by means of an LEPGEKPYKCPECGKSFSQSGALTRHQRTHTR (SEQ ID NO: 19) linker. In addition, N- and C-terminal NLSs can also function as linker (e.g., PKKKRKVEASSPKKRKVEAS (SEQ ID NO: 20)).

The skilled person will understand that modifications to the guide which allow for binding of the adapter+functional domain but not proper positioning of the adapter+functional domain (e.g., due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and most preferably at both the tetra loop and stem loop 2.

### Guide Molecules

The engineered nucleic acid modification system described herein may comprise one or more guide molecules. The guide molecule(s) may be component(s) of the site-specific nuclease system (e.g., a CRISPR-Cas system) herein. As used herein, the term “guide sequence,” “guide polynucleotide,” and “guide molecule” in the context of a CRISPR-Cas system and generally the engineered nucleic acid modification systems of the present invention, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some embodiments, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example embodiments, the guide molecule comprises a guide sequence that may be designed to have at least one mismatch with the target sequence, such that an RNA duplex formed between the guide sequence and the target sequence. Accordingly, the degree of complementarity is preferably less than 99%. For instance, where the guide sequence consists of 24 nucleotides, the degree of complementarity is more particularly about 96% or less. In particular embodiments, the guide sequence is designed to have a stretch of two or more adjacent mismatching nucleotides, such that the degree of complementarity over the entire guide sequence is further reduced. For instance, where the guide sequence consists of 24 nucleotides, the degree of complementarity is more particularly about 96% or less, more particularly, about 92% or less, more particularly about 88% or less, more particularly about 84% or less, more particularly about 80% or less, more particularly about 76% or less, more particularly about 72% or less, depending on whether the stretch of two or more mismatching nucleotides encompasses 2, 3, 4, 5, 6 or 7 nucleotides, etc. In some embodiments, aside from the stretch of one or more mismatching nucleotides, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. Other assays are possible, and will occur to those skilled in the art. A guide sequence, and hence a nucleic acid-targeting guide RNA may be selected to target any target nucleic acid sequence.

The guide molecule may direct the fusion proteins of the present invention to a target sequence that is 5′ to or 3′ the targeted insertion site. In the case of paired nickase embodiments, one guide molecule be configured to bind to a target sequence on the sense strand of the target polypeptide and a second guide may be configured to bind to the anti-sense strand of the target polynucleotide.

In certain embodiments, the guide sequence or spacer length of the guide molecules is from 15 to 50 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example embodiment, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In some embodiments, the guide sequence is an RNA sequence of between 10 to 50 nt in length, but more particularly of about 20-30 nt advantageously about 20 nt, 23-25 nt or 24 nt. The guide sequence is selected so as to ensure that it hybridizes to the target sequence. This is described more in detail below. Selection can encompass further steps which increase efficacy and specificity.

In some embodiments, the guide sequence has a canonical length (e.g., about 15-30 nt) is used to hybridize with the target RNA or DNA. In some embodiments, a guide molecule is longer than the canonical length (e.g., >30 nt) is used to hybridize with the target RNA or DNA, such that a region of the guide sequence hybridizes with a region of the RNA or DNA strand outside of the Cas-guide target complex. This can be of interest where additional modifications, such deamination of nucleotides is of interest. In alternative embodiments, it is of interest to maintain the limitation of the canonical guide sequence length.

In some embodiments, the sequence of the guide molecule (direct repeat and/or spacer) is selected to reduce the degree secondary structure within the guide molecule. In some embodiments, about or less than about 75%, 50%, 40%, 30%, 25%, 20%, 15%, 10%, 5%, 1%, or fewer of the nucleotides of the nucleic acid-targeting guide RNA participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In some embodiments, a guide molecule is designed or selected to modulate intermolecular interactions among guide molecules, such as among stem-loop regions of different guide molecules. It will be appreciated that nucleotides within a guide that base-pair to form a stem-loop are also capable of base-pairing to form an intermolecular duplex with a second guide and that such an intermolecular duplex would not have a secondary structure compatible with CRISPR complex formation. Accordingly, it is useful to select or design DR sequences in order to modulate stem-loop formation and CRISPR complex formation. In some embodiments, about or less than about 75%, 50%, 40%, 30%, 25%, 20%, 15%, 10%, 5%, 1%, or fewer of nucleic acid-targeting guides are in intermolecular duplexes. It will be appreciated that stem-loop variation will often be within limits imposed by DR-CRISPR effector interactions. One way to modulate stem-loop formation or change the equilibrium between stem-loop and intermolecular duplex is to vary nucleotide pairs in the stem of the stem-loop of a DR. For example, in one embodiment, a G-C pair is replaced by an A-U or U-A pair. In another embodiment, an A-U pair is substituted for a G-C or a C-G pair. In another embodiment, a naturally occurring nucleotide is replaced by a nucleotide analog. Another way to modulate stem-loop formation or change the equilibrium between stem-loop and intermolecular duplex is to modify the loop of the stem-loop of a DR. Without be bound by theory, the loop can be viewed as an intervening sequence flanked by two sequences that are complementary to each other. When that intervening sequence is not self-complementary, its effect will be to destabilize intermolecular duplex formation. The same principle applies when guides are multiplexed: while the targeting sequences may differ, it may be advantageous to modify the stem-loop region in the DRs of the different guides. Moreover, when guides are multiplexed, the relative activities of the different guides can be modulated by balancing the activity of each individual guide. In certain embodiments, the equilibrium between intermolecular stem-loops vs. intermolecular duplexes is determined. The determination may be made by physical or biochemical means and can be in the presence or absence of a CRISPR effector.

In some embodiments, it is of interest to reduce the susceptibility of the guide molecule to RNA cleavage, such as cleavage by a CRISPR system that cleaves RNA. Accordingly, in particular embodiments, the guide molecule is adjusted to avoid cleavage by a CRISPR system or other RNA-cleaving enzymes.

In certain embodiments, the guide molecule comprises non-naturally occurring nucleic acids and/or non-naturally occurring nucleotides and/or nucleotide analogs, and/or chemically modifications. Preferably, these non-naturally occurring nucleic acids and non-naturally occurring nucleotides are located outside the guide sequence. Non-naturally occurring nucleic acids can include, for example, mixtures of naturally and non-naturally occurring nucleotides. Non-naturally occurring nucleotides and/or nucleotide analogs may be modified at the ribose, phosphate, and/or base moiety. In an embodiment of the invention, a guide nucleic acid comprises ribonucleotides and non-ribonucleotides. In one such embodiment, a guide comprises one or more ribonucleotides and one or more deoxyribonucleotides. In an embodiment of the invention, the guide comprises one or more non-naturally occurring nucleotide or nucleotide analog such as a nucleotide with phosphorothioate linkage, a locked nucleic acid (LNA) nucleotides comprising a methylene bridge between the 2′ and 4′ carbons of the ribose ring or bridged nucleic acids (BNA). Other examples of modified nucleotides include 2′-O-methyl analogs, 2′-deoxy analogs, or 2′-fluoro analogs. Further examples of modified bases include, but are not limited to, 2-aminopurine, 5-bromo-uridine, pseudouridine, inosine, 7-methylguanosine. Examples of guide RNA chemical modifications include, without limitation, incorporation of 2′-O-methyl (M), 2′-O-methyl 3′phosphorothioate (MS), S-constrained ethyl(cEt), or 2′-O-methyl 3′thioPACE (MSP) at one or more terminal nucleotides. Such chemically modified guides can comprise increased stability and increased activity as compared to unmodified guides, though on-target vs. off-target specificity is not predictable. (See, Hendel, 2015, Nat Biotechnol. 33(9):985-9, doi: 10.1038/nbt.3290, published online 29 Jun. 2015 Ragdarm et al., 0215, PNAS, E7110-E7111; Allerson et al., J. Med. Chem. 2005, 48:901-904; Bramsen et al., Front. Genet., 2012, 3:154; Deng et al., PNAS, 2015, 112:11870-11875; Sharma et al., MedChemComm., 2014, 5:1454-1471; Hendel et al., Nat. Biotechnol. (2015) 33(9): 985-989; Li et al., Nature Biomedical Engineering, 2017, 1, 0066 DOI:10.1038/s41551-017-0066). In some embodiments, the 5′ and/or 3′ end of a guide RNA is modified by a variety of functional moieties including fluorescent dyes, polyethylene glycol, cholesterol, proteins, or detection tags. (See Kelly et al., 2016, J. Biotech. 233:74-83). In certain embodiments, a guide comprises ribonucleotides in a region that binds to a target RNA and one or more deoxyribonucleotides and/or nucleotide analogs in a region that binds to a Cas effector. In an embodiment of the invention, deoxyribonucleotides and/or nucleotide analogs are incorporated in engineered guide structures, such as, without limitation, stem-loop regions, and the seed region. In certain embodiments, at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or 75 nucleotides of a guide is chemically modified. In some embodiments, 3-5 nucleotides at either the 3′ or the 5′ end of a guide is chemically modified. In some embodiments, only minor modifications are introduced in the seed region, such as 2′-F modifications. In some embodiments, 2′-F modification is introduced at the 3′ end of a guide. In certain embodiments, three to five nucleotides at the 5′ and/or the 3′ end of the guide are chemically modified with 2′-O-methyl (M), 2′-O-methyl 3′ phosphorothioate (MS), S-constrained ethyl(cEt), or 2′-O-methyl 3′ thioPACE (MSP). Such modification can enhance genome editing efficiency (see e.g., Hendel et al., Nat. Biotechnol. (2015) 33(9): 985-989). In certain embodiments, all of the phosphodiester bonds of a guide are substituted with phosphorothioates (PS) for enhancing levels of gene disruption. In certain embodiments, more than five nucleotides at the 5′ and/or the 3′ end of the guide are chemically modified with 2′-O-Me, 2′-F or S-constrained ethyl(cEt). Such chemically modified guide can mediate enhanced levels of gene disruption (see e.g., Ragdarm et al., 0215, PNAS, E7110-E7111). In an embodiment of the invention, a guide is modified to comprise a chemical moiety at its 3′ and/or 5′ end. Such moieties include, but are not limited to amine, azide, alkyne, thio, dibenzocyclooctyne (DBCO), or Rhodamine, peptides, nuclear localization sequence (NLS), peptide nucleic acid (PNA), polyethylene glycol (PEG), triethylene glycol, or tetraethyleneglycol (TEG). In certain embodiment, the chemical moiety is conjugated to the guide by a linker, such as an alkyl chain. In certain embodiment, the chemical moiety is conjugated to the guide by a linker, such as an alkyl chain. In certain embodiments, the chemical moiety of the modified guide can be used to attach the guide to another molecule, such as DNA, RNA, protein, or nanoparticles. Such chemically modified guide can be used to identify or enrich cells generically edited by a CRISPR system (see e.g., Lee et al., eLife, 2017, 6:e25312, DOI:10.7554).

In some embodiments, 3 nucleotides at each of the 3′ and 5′ ends are chemically modified. In a specific embodiment, the modifications comprise 2′-O-methyl or phosphorothioate analogs. In a specific embodiment, 12 nucleotides in the tetraloop and 16 nucleotides in the stem-loop region are replaced with 2′-O-methyl analogs. Such chemical modifications improve in vivo editing and stability (see e.g., Finn et al., Cell Reports (2018), 22: 2227-2235). In some embodiments, more than 60 or 70 nucleotides of the guide are chemically modified. In some embodiments, this modification comprises replacement of nucleotides with 2′-O-methyl or 2′-fluoro nucleotide analogs or phosphorothioate (PS) modification of phosphodiester bonds. In some embodiments, the chemical modification comprises 2′-O-methyl or 2′-fluoro modification of guide nucleotides extending outside of the nuclease protein when the CRISPR complex is formed or PS modification of 20 to 30 or more nucleotides of the 3′-terminus of the guide. In a particular embodiment, the chemical modification further comprises 2′-O-methyl analogs at the 5′ end of the guide or 2′-fluoro analogs in the seed and tail regions. Such chemical modifications improve stability to nuclease degradation and maintain or enhance genome-editing activity or efficiency, but modification of all nucleotides may abolish the function of the guide (see e.g., Yin et al., Nat. Biotech. (2018), 35(12): 1179-1187). Such chemical modifications may be guided by knowledge of the structure of the CRISPR complex, including knowledge of the limited number of nuclease and RNA 2′-OH interactions (see e.g., Yin et al., Nat. Biotech. (2018), 35(12): 1179-1187). In some embodiments, one or more guide RNA nucleotides may be replaced with DNA nucleotides. In some embodiments, up to 2, 4, 6, 8, 10, or 12 RNA nucleotides of the 5′-end tail/seed guide region are replaced with DNA nucleotides. In certain embodiments, the majority of guide RNA nucleotides at the 3′ end are replaced with DNA nucleotides. In particular embodiments, 16 guide RNA nucleotides at the 3′ end are replaced with DNA nucleotides. In particular embodiments, 8 guide RNA nucleotides of the 5′-end tail/seed region and 16 RNA nucleotides at the 3′ end are replaced with DNA nucleotides. In particular embodiments, guide RNA nucleotides that extend outside of the nuclease protein when the CRISPR complex is formed are replaced with DNA nucleotides. Such replacement of multiple RNA nucleotides with DNA nucleotides leads to decreased off-target activity but similar on-target activity compared to an unmodified guide; however, replacement of all RNA nucleotides at the 3′ end may abolish the function of the guide (see e.g., Yin et al., Nat. Chem. Biol. (2018) 14, 311-316). Such modifications may be guided by knowledge of the structure of the CRISPR complex, including knowledge of the limited number of nuclease and RNA 2′-OH interactions (see Yin et al., Nat. Chem. Biol. (2018) 14, 311-316).

In some embodiments, the guide molecule forms a stemloop with a separate non-covalently linked sequence, which can be DNA or RNA. In particular embodiments, the sequences forming the guide are first synthesized using the standard phosphoramidite synthetic protocol (see e.g., Herdewijn, P., ed., Methods in Molecular Biology Col 288, Oligonucleotide Synthesis: Methods and Applications, Humana Press, New Jersey (2012)). In some embodiments, these sequences can be functionalized to contain an appropriate functional group for ligation using the standard protocol known in the art (see e.g., Hermanson, G. T., Bioconjugate Techniques, Academic Press (2013)). Examples of functional groups include, but are not limited to, hydroxyl, amine, carboxylic acid, carboxylic acid halide, carboxylic acid active ester, aldehyde, carbonyl, chlorocarbonyl, imidazolylcarbonyl, hydrozide, semicarbazide, thio semicarbazide, thiol, maleimide, haloalkyl, sufonyl, ally, propargyl, diene, alkyne, and azide. Once this sequence is functionalized, a covalent chemical bond or linkage can be formed between this sequence and the direct repeat sequence. Examples of chemical bonds include, but are not limited to, those based on carbamates, ethers, esters, amides, imines, amidines, aminotrizines, hydrozone, disulfides, thioethers, thioesters, phosphorothioates, phosphorodithioates, sulfonamides, sulfonates, fulfones, sulfoxides, ureas, thioureas, hydrazide, oxime, triazole, photolabile linkages, C—C bond forming groups such as Diels-Alder cyclo-addition pairs or ring-closing metathesis pairs, and Michael reaction pairs.

In some embodiments, these stem-loop forming sequences can be chemically synthesized. In some embodiments, the chemical synthesis uses automated, solid-phase oligonucleotide synthesis machines with 2′-acetoxyethyl orthoester (2′-ACE) (see e.g., Scaringe et al., J. Am. Chem. Soc. (1998) 120: 11820-11821; Scaringe, Methods Enzymol. (2000) 317: 3-18) or 2′-thionocarbamate (2′-TC) chemistry (see e.g., Dellinger et al., J. Am. Chem. Soc. (2011) 133: 11540-11546; Hendel et al., Nat. Biotechnol. (2015) 33:985-989).

In certain embodiments, the guide molecule comprises (1) a guide sequence capable of hybridizing to a target locus and (2) a tracr mate or direct repeat sequence whereby the direct repeat sequence is located upstream (i.e., 5′) or downstream (i.e., 3′) from the guide sequence. In a particular embodiment the seed sequence (i.e., the sequence essential critical for recognition and/or hybridization to the sequence at the target locus) of the guide sequence is approximately within the first 10 nucleotides of the guide sequence.

In a particular embodiment the guide molecule comprises a guide sequence linked to a direct repeat sequence, wherein the direct repeat sequence comprises one or more stem loops or optimized secondary structures. In particular embodiments, the direct repeat has a minimum length of 16 nts and a single stem loop. In further embodiments the direct repeat has a length longer than 16 nts, preferably more than 17 nts, and has more than one stem loops or optimized secondary structures. In particular embodiments the guide molecule comprises or consists of the guide sequence linked to all or part of the natural direct repeat sequence. A CRISPR-cas guide molecule comprises (in 3′ to 5′ direction or in 5′ to 3′ direction): a guide sequence a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain embodiments, the direct repeat sequence retains its natural architecture and forms a single stem loop. In particular embodiments, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In particular embodiments, the stem comprises at least about 4 bp comprising complementary X and Y sequences, although stems of more, e.g., 5, 6, 7, 8, 9, 10, 11 or 12 or fewer, e.g., 3, 2, base pairs are also contemplated. Thus, for example X2-10 and Y2-10 (wherein X and Y represent any complementary set of nucleotides) may be contemplated. In one aspect, the stem made of the X and Y nucleotides, together with the loop will form a complete hairpin in the overall secondary structure; and, this may be advantageous and the amount of base pairs can be any amount that forms a complete hairpin. In one aspect, any complementary X:Y basepairing sequence (e.g., as to length) is tolerated, so long as the secondary structure of the entire guide molecule is preserved. In one aspect, the loop that connects the stem made of X:Y basepairs can be any sequence of the same length (e.g., 4 or 5 nucleotides) or longer that does not interrupt the overall secondary structure of the guide molecule. In one aspect, the stemloop can further comprise, e.g., an MS2 aptamer. In one aspect, the stem comprises about 5-7 bp comprising complementary X and Y sequences, although stems of more or fewer basepairs are also contemplated. In one aspect, non-Watson Crick basepairing is contemplated, where such pairing otherwise generally preserves the architecture of the stemloop at that position.

In particular embodiments the natural hairpin or stemloop structure of the guide molecule is extended or replaced by an extended stemloop. It has been demonstrated that extension of the stem can enhance the assembly of the guide molecule with the CRISPR-Cas protein (Chen et al. Cell. (2013); 155(7): 1479-1491). In particular embodiments the stem of the stemloop is extended by at least 1, 2, 3, 4, 5 or more complementary basepairs (i.e., corresponding to the addition of 2, 4, 6, 8, 10 or more nucleotides in the guide molecule). In particular embodiments these are located at the end of the stem, adjacent to the loop of the stemloop.

In particular embodiments, the susceptibility of the guide molecule to RNases or to decreased expression can be reduced by slight modifications of the sequence of the guide molecule which do not affect its function. For instance, in particular embodiments, premature termination of transcription, such as premature transcription of U6 Pol-III, can be removed by modifying a putative Pol-III terminator (4 consecutive U's) in the guide molecules sequence. Where such sequence modification is required in the stemloop of the guide molecule, it is preferably ensured by a base pair flip.

In a particular embodiment, the direct repeat may be modified to comprise one or more protein-binding RNA aptamers. In a particular embodiment, one or more aptamers may be included such as part of optimized secondary structure. Such aptamers may be capable of binding a bacteriophage coat protein as detailed further herein.

In some embodiments, the guide molecule forms a duplex with a target RNA comprising at least one target cytosine residue to be edited. Upon hybridization of the guide RNA molecule to the target RNA, the cytidine deaminase binds to the single strand RNA in the duplex made accessible by the mismatch in the guide sequence and catalyzes deamination of one or more target cytosine residues comprised within the stretch of mismatching nucleotides.

A guide sequence, and hence a nucleic acid-targeting guide RNA may be selected to target any target nucleic acid sequence. The target sequence may be mRNA.

In certain embodiments, the target sequence should be associated with a PAM (protospacer adjacent motif) or PFS (protospacer flanking sequence or site); that is, a short sequence recognized by the CRISPR complex. Depending on the nature of the CRISPR-Cas protein, the target sequence should be selected such that its complementary sequence in the DNA duplex (also referred to herein as the non-target sequence) is upstream or downstream of the PAM.

Further, engineering of the PAM Interacting (PI) domain may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592.

In particular embodiments, the guide is an escorted guide. By “escorted” is meant that the CRISPR-Cas system or complex or guide is delivered to a selected time or place within a cell, so that activity of the CRISPR-Cas system or complex or guide is spatially or temporally controlled. For example, the activity and destination of the 3 CRISPR-Cas system or complex or guide may be controlled by an escort RNA aptamer sequence that has binding affinity for an aptamer ligand, such as a cell surface protein or other localized cellular component. Alternatively, the escort aptamer may for example be responsive to an aptamer effector on or in the cell, such as a transient effector, such as an external energy source that is applied to the cell at a particular time.

The escorted CRISPR-Cas systems or complexes have a guide molecule with a functional structure designed to improve guide molecule structure, architecture, stability, genetic expression, or any combination thereof. Such a structure can include an aptamer.

Aptamers are biomolecules that can be designed or selected to bind tightly to other ligands, for example using a technique called systematic evolution of ligands by exponential enrichment (SELEX; see e.g., Tuerk C, Gold L: “Systematic evolution of ligands by exponential enrichment: RNA ligands to bacteriophage T4 DNA polymerase.” Science 1990, 249:505-510). Nucleic acid aptamers can for example be selected from pools of random-sequence oligonucleotides, with high binding affinities and specificities for a wide range of biomedically relevant targets, suggesting a wide range of therapeutic utilities for aptamers (see e.g., Keefe, Anthony D., Supriya Pai, and Andrew Ellington. “Aptamers as therapeutics.” Nature Reviews Drug Discovery 9.7 (2010): 537-550). These characteristics also suggest a wide range of uses for aptamers as drug delivery vehicles (see e.g., Levy-Nissenbaum, Etgar, et al. “Nanotechnology and aptamers: applications in drug delivery.” Trends in biotechnology 26.8 (2008): 442-449; and Hicke B J, Stephens A W. “Escort aptamers: a delivery service for diagnosis and therapy.” J Clin Invest 2000, 106:923-928.). Aptamers may also be constructed that function as molecular switches, responding to a que by changing properties, such as RNA aptamers that bind fluorophores to mimic the activity of green fluorescent protein (see e.g., Paige, Jeremy S., Karen Y. Wu, and Samie R. Jaffrey. “RNA mimics of green fluorescent protein.” Science 333.6042 (2011): 642-646). It has also been suggested that aptamers may be used as components of targeted siRNA therapeutic delivery systems, for example targeting cell surface proteins (see e.g., Zhou, Jiehua, and John J. Rossi. “Aptamer-targeted cell-specific RNA interference.” Silence 1.1 (2010): 4).

Accordingly, in particular embodiments, the guide molecule is modified, e.g., by one or more aptamer(s) designed to improve guide molecule delivery, including delivery across the cellular membrane, to intracellular compartments, or into the nucleus. Such a structure can include, either in addition to the one or more aptamer(s) or without such one or more aptamer(s), moiety(ies) so as to render the guide molecule deliverable, inducible or responsive to a selected effector. In some embodiments, the guide molecule is responsive to normal or pathological physiological conditions, including without limitation pH, hypoxia, 02 concentration, temperature, protein concentration, enzymatic concentration, lipid structure, light exposure, mechanical disruption (e.g., ultrasound waves), magnetic fields, electric fields, or electromagnetic radiation.

In some embodiments, the inducible system is light responsive. Light responsiveness of an inducible system may be achieved via the activation and binding of cryptochrome-2 and CIB1. Blue light stimulation induces an activating conformational change in cryptochrome-2, resulting in recruitment of its binding partner CIB1. This binding is fast and reversible, achieving saturation in <15 sec following pulsed stimulation and returning to baseline <15 min after the end of stimulation. These rapid binding kinetics result in a system temporally bound only by the speed of transcription/translation and transcript/protein degradation, rather than uptake and clearance of inducing agents. Crytochrome-2 activation is also highly sensitive, allowing for the use of low light intensity stimulation and mitigating the risks of phototoxicity. Further, in a context such as the intact mammalian brain, variable light intensity may be used to control the size of a stimulated region, allowing for greater precision than vector delivery alone may offer.

In some embodiments, the inducible system is responsive to one or more energy sources, such as electromagnetic radiation, sound energy or thermal energy to induce the guide. Advantageously, the electromagnetic radiation is a component of visible light. In a preferred embodiment, the light is a blue light with a wavelength of about 450 to about 495 nm. In an especially preferred embodiment, the wavelength is about 488 nm. In another preferred embodiment, the light stimulation is via pulses. The light power may range from about 0-9 mW/cm2. In a preferred embodiment, a stimulation paradigm of as low as 0.25 sec every 15 sec should result in maximal activation.

The chemical or energy responsive guide may undergo a conformational change upon induction by the binding of a chemical source or by the energy allowing it act as a guide and have the CRISPR-Cas system or complex function. The invention can involve applying the chemical source or energy so as to have the guide function and the CRISPR-Cas system or complex function; and optionally further determining that the expression of the genomic locus is altered.

Exemplary designs of this chemical inducible system are ABI-PYL based system inducible by Abscisic Acid (ABA) an FKBP-FRB based system inducible by rapamycin (or related chemicals based on rapamycin), GID1-GAI based system inducible by Gibberellin (GA), and those set forth in Kallunki et al. (Cells. 2019. “How to Choose the Right Inducible Gene Expression System, for Mammalian Studies?” 8:796 doi:10.3390/cells8080796

A chemical inducible system can be an estrogen receptor (ER) based system inducible by 4-hydroxytamoxifen (4OHT) (see e.g., www.pnas.org/content/104/3/1027.abstract). A mutated ligand-binding domain of the estrogen receptor called ERT2 translocates into the nucleus of cells upon binding of 4-hydroxytamoxifen. In further embodiments of the invention any naturally occurring or engineered derivative of any nuclear receptor, thyroid hormone receptor, retinoic acid receptor, estrogen receptor, estrogen-related receptor, glucocorticoid receptor, progesterone receptor, androgen receptor may be used in inducible systems analogous to the ER based inducible system.

Another inducible system is based on the design using Transient receptor potential (TRP) ion channel based system inducible by energy, heat or radio-wave (see, e.g., www.sciencemag.org/content/336/6081/604). These TRP family proteins respond to different stimuli, including light and heat. When this protein is activated by light or heat, the ion channel will open and allow the entering of ions such as calcium into the plasma membrane. This influx of ions will bind to intracellular ion interacting partners linked to a polypeptide including the guide and the other components of the CRISPR-Cas complex or system, and the binding will induce the change of sub-cellular localization of the polypeptide, leading to the entire polypeptide entering the nucleus of cells. Once inside the nucleus, the guide protein and the other components of the CRISPR-Cas complex will be active and modulating target gene expression in cells.

While light activation may be an advantageous embodiment, sometimes it may be disadvantageous especially for in vivo applications in which the light may not penetrate the skin or other organs. In this instance, other methods of energy activation are contemplated, in particular, electric field energy and/or ultrasound which have a similar effect.

Electric field energy is preferably administered substantially as described in the art, using one or more electric pulses of from about 1 Volt/cm to about 10 kVolts/cm under in vivo conditions. Instead of or in addition to the pulses, the electric field may be delivered in a continuous manner. The electric pulse may be applied for between 1 μs and 500 milliseconds, preferably between 1 μs and 100 milliseconds. The electric field may be applied continuously or in a pulsed manner for 5 about minutes.

As used herein, ‘electric field energy’ is the electrical energy to which a cell is exposed. Preferably the electric field has a strength of from about 1 Volt/cm to about 10 kVolts/cm or more under in vivo conditions (see WO97/49450).

As used herein, the term “electric field” includes one or more pulses at variable capacitance and voltage and including exponential and/or square wave and/or modulated wave and/or modulated square wave forms. References to electric fields and electricity should be taken to include reference the presence of an electric potential difference in the environment of a cell. Such an environment may be set up by way of static electricity, alternating current (AC), direct current (DC), etc., as known in the art. The electric field may be uniform, non-uniform or otherwise, and may vary in strength and/or direction in a time dependent manner.

Single or multiple applications of electric field, as well as single or multiple applications of ultrasound are also possible, in any order and in any combination. The ultrasound and/or the electric field may be delivered as single or multiple continuous applications, or as pulses (pulsatile delivery).

Electroporation has been used in both in vitro and in vivo procedures to introduce foreign material into living cells. With in vitro applications, a sample of live cells is first mixed with the agent of interest and placed between electrodes such as parallel plates. Then, the electrodes apply an electrical field to the cell/implant mixture. Examples of systems that perform in vitro electroporation include the Electro Cell Manipulator ECM600 product, and the Electro Square Porator T820, both made by the BTX Division of Genetronics, Inc (see e.g., U.S. Pat. No. 5,869,326).

The known electroporation techniques (both in vitro and in vivo) function by applying a brief high voltage pulse to electrodes positioned around the treatment region. The electric field generated between the electrodes causes the cell membranes to temporarily become porous, whereupon molecules of the agent of interest enter the cells. In known electroporation applications, this electric field comprises a single square wave pulse on the order of 1000 V/cm, of about 100 .mu.s duration. Such a pulse may be generated, for example, in known applications of the Electro Square Porator T820.

Preferably, the electric field has a strength of from about 1 V/cm to about 10 kV/cm under in vitro conditions. Thus, the electric field may have a strength of 1 V/cm, 2 V/cm, 3 V/cm, 4 V/cm, 5 V/cm, 6 V/cm, 7 V/cm, 8 V/cm, 9 V/cm, 10 V/cm, 20 V/cm, 50 V/cm, 100 V/cm, 200 V/cm, 300 V/cm, 400 V/cm, 500 V/cm, 600 V/cm, 700 V/cm, 800 V/cm, 900 V/cm, 1 kV/cm, 2 kV/cm, 5 kV/cm, 10 kV/cm, 20 kV/cm, 50 kV/cm or more. More preferably from about 0.5 kV/cm to about 4.0 kV/cm under in vitro conditions. Preferably the electric field has a strength of from about 1 V/cm to about 10 kV/cm under in vivo conditions. However, the electric field strengths may be lowered where the number of pulses delivered to the target site are increased. Thus, pulsatile delivery of electric fields at lower field strengths is envisaged.

Preferably the application of the electric field is in the form of multiple pulses such as double pulses of the same strength and capacitance or sequential pulses of varying strength and/or capacitance. As used herein, the term “pulse” includes one or more electric pulses at variable capacitance and voltage and including exponential and/or square wave and/or modulated wave/square wave forms.

Preferably the electric pulse is delivered as a waveform selected from an exponential wave form, a square wave form, a modulated wave form and a modulated square wave form.

A preferred embodiment employs direct current at low voltage. Thus, Applicants disclose the use of an electric field which is applied to the cell, tissue or tissue mass at a field strength of between 1V/cm and 20V/cm, for a period of 100 milliseconds or more, preferably 15 minutes or more.

Ultrasound is advantageously administered at a power level of from about 0.05 W/cm2 to about 100 W/cm2. Diagnostic or therapeutic ultrasound may be used, or combinations thereof.

As used herein, the term “ultrasound” refers to a form of energy which consists of mechanical vibrations the frequencies of which are so high they are above the range of human hearing. Lower frequency limit of the ultrasonic spectrum may generally be taken as about 20 kHz. Most diagnostic applications of ultrasound employ frequencies in the range 1 and 15 MHz’ (From Ultrasonics in Clinical Diagnosis, P. N. T. Wells, ed., 2nd. Edition, Publ. Churchill Livingstone [Edinburgh, London & NY, 1977]).

Ultrasound has been used in both diagnostic and therapeutic applications. When used as a diagnostic tool (“diagnostic ultrasound”), ultrasound is typically used in an energy density range of up to about 100 mW/cm2 (FDA recommendation), although energy densities of up to 750 mW/cm2 have been used. In physiotherapy, ultrasound is typically used as an energy source in a range up to about 3 to 4 W/cm2 (WHO recommendation). In other therapeutic applications, higher intensities of ultrasound may be employed, for example, HIFU at 100 W/cm up to 1 kW/cm2 (or even higher) for short periods of time. The term “ultrasound” as used in this specification is intended to encompass diagnostic, therapeutic and focused ultrasound.

Focused ultrasound (FUS) allows thermal energy to be delivered without an invasive probe (see e.g., Morocz et al. 1998 Journal of Magnetic Resonance Imaging Vol. 8, No. 1, pp. 136-142. Another form of focused ultrasound is high intensity focused ultrasound (HIFU) which is reviewed by Moussatov et al. in Ultrasonics (1998) Vol. 36, No. 8, pp. 893-900 and TranHuuHue et al in Acustica (1997) Vol. 83, No. 6, pp. 1103-1106.

Preferably, a combination of diagnostic ultrasound and a therapeutic ultrasound is employed. This combination is not intended to be limiting, however, and the skilled reader will appreciate that any variety of combinations of ultrasound may be used. Additionally, the energy density, frequency of ultrasound, and period of exposure may be varied.

Preferably the exposure to an ultrasound energy source is at a power density of from about 0.05 to about 100 Wcm-2. Even more preferably, the exposure to an ultrasound energy source is at a power density of from about 1 to about 15 Wcm-2.

Preferably the exposure to an ultrasound energy source is at a frequency of from about 0.015 to about 10.0 MHz. More preferably the exposure to an ultrasound energy source is at a frequency of from about 0.02 to about 5.0 MHz or about 6.0 MHz. Most preferably, the ultrasound is applied at a frequency of 3 MHz.

Preferably the exposure is for periods of from about 10 milliseconds to about 60 minutes. Preferably the exposure is for periods of from about 1 second to about 5 minutes. More preferably, the ultrasound is applied for about 2 minutes. Depending on the particular target cell to be disrupted, however, the exposure may be for a longer duration, for example, for 15 minutes.

Advantageously, the target tissue is exposed to an ultrasound energy source at an acoustic power density of from about 0.05 Wcm-2 to about 10 Wcm-2 with a frequency ranging from about 0.015 to about 10 MHz (see WO 98/52609). However, alternatives are also possible, for example, exposure to an ultrasound energy source at an acoustic power density of above 100 Wcm-2, but for reduced periods of time, for example, 1000 Wcm-2 for periods in the millisecond range or less.

Preferably the application of the ultrasound is in the form of multiple pulses; thus, both continuous wave and pulsed wave (pulsatile delivery of ultrasound) may be employed in any combination. For example, continuous wave ultrasound may be applied, followed by pulsed wave ultrasound, or vice versa. This may be repeated any number of times, in any order and combination. The pulsed wave ultrasound may be applied against a background of continuous wave ultrasound, and any number of pulses may be used in any number of groups.

Preferably, the ultrasound may comprise pulsed wave ultrasound. In a highly preferred embodiment, the ultrasound is applied at a power density of 0.7 Wcm-2 or 1.25 Wcm-2 as a continuous wave. Higher power densities may be employed if pulsed wave ultrasound is used.

Use of ultrasound is advantageous as, like light, it may be focused accurately on a target. Moreover, ultrasound is advantageous as it may be focused more deeply into tissues unlike light. It is therefore better suited to whole-tissue penetration (such as but not limited to a lobe of the liver) or whole organ (such as but not limited to the entire liver or an entire muscle, such as the heart) therapy. Another important advantage is that ultrasound is a non-invasive stimulus which is used in a wide variety of diagnostic and therapeutic applications. By way of example, ultrasound is well known in medical imaging techniques and, additionally, in orthopedic therapy. Furthermore, instruments suitable for the application of ultrasound to a subject vertebrate are widely available and their use is well known in the art.

In particular embodiments, the guide molecule is modified by a secondary structure to increase the specificity of the CRISPR-Cas system and the secondary structure can protect against exonuclease activity and allow for 5′ additions to the guide sequence also referred to herein as a protected guide molecule.

In one aspect, the invention provides for hybridizing a “protector RNA” to a sequence of the guide molecule, wherein the “protector RNA” is an RNA strand complementary to the 3′ end of the guide molecule to thereby generate a partially double-stranded guide RNA. In an embodiment of the invention, protecting mismatched bases (i.e. the bases of the guide molecule which do not form part of the guide sequence) with a perfectly complementary protector sequence decreases the likelihood of target RNA binding to the mismatched basepairs at the 3′ end. In particular embodiments of the invention, additional sequences comprising an extended length may also be present within the guide molecule such that the guide comprises a protector sequence within the guide molecule. This “protector sequence” ensures that the guide molecule comprises a “protected sequence” in addition to an “exposed sequence” (comprising the part of the guide sequence hybridizing to the target sequence). In particular embodiments, the guide molecule is modified by the presence of the protector guide to comprise a secondary structure such as a hairpin. Advantageously there are three or four to thirty or more, e.g., about 10 or more, contiguous base pairs having complementarity to the protected sequence, the guide sequence or both. It is advantageous that the protected portion does not impede thermodynamics of the CRISPR-Cas system interacting with its target. By providing such an extension including a partially double stranded guide molecule, the guide molecule is considered protected and results in improved specific binding of the CRISPR-Cas complex, while maintaining specific activity.

In particular embodiments, use is made of a truncated guide (tru-guide), i.e., a guide molecule which comprises a guide sequence which is truncated in length with respect to the canonical guide sequence length. As described by Nowak et al. (Nucleic Acids Res (2016) 44 (20): 9555-9564), such guides may allow catalytically active CRISPR-Cas enzyme to bind its target without cleaving the target RNA. In particular embodiments, a truncated guide is used which allows the binding of the target but retains only nickase activity of the CRISPR-Cas enzyme.

The methods and tools provided herein are exemplified for certain Cas effectors. Further nucleases with similar properties can be identified using methods described in the art (see e.g., Shmakov et al. 2015, 60:385-397; Abudayeh et al. 2016, Science, 5; 353(6299)). In particular embodiments, such methods for identifying novel CRISPR effector proteins may comprise the steps of selecting sequences from the database encoding a seed which identifies the presence of a CRISPR Cas locus, identifying loci located within 10 kb of the seed comprising Open Reading Frames (ORFs) in the selected sequences, selecting therefrom loci comprising ORFs of which only a single ORF encodes a novel CRISPR effector having greater than 700 amino acids and no more than 90% homology to a known CRISPR effector. In particular embodiments, the seed is a protein that is common to the CRISPR-Cas system, such as Cas1. In further embodiments, the CRISPR array is used as a seed to identify new effector proteins.

Also, “Dimeric CRISPR RNA-guided FokI nucleases for highly specific genome editing”, Shengdar Q. Tsai, Nicolas Wyvekens, Cyd Khayter, Jennifer A. Foden, Vishal Thapar, Deepak Reyon, Mathew J. Goodwin, Martin J. Aryee, J. Keith Joung Nature Biotechnology 32(6): 569-77 (2014), relates to dimeric RNA-guided FokI Nucleases that recognize extended sequences and can edit endogenous genes with high efficiencies in human cells, the teachings of which can be adapted for use with the present invention.

With respect to general information on CRISPR-Cas Systems, components thereof, and delivery of such components, including methods, materials, delivery vehicles, vectors, particles, AAV, and making and using thereof, including as to amounts and formulations, all useful in the practice of the instant invention, reference is made to: U.S. Pat. Nos. 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014.

Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS.

Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, 62/285,349, 22 Oct. 2015, 62/296,522, 17 Feb. 2016, and 62/320,231, 8 Apr. 2016, NOVEL CRISPR ENZYMES AND SYSTEMS, U.S. application 62/232,067, 24 Sep. 2015, U.S. application Ser. No. 14/975,085, 18 Dec. 2015, European application No. 16150428.7, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES.

In addition, mention is made of PCT application PCT/US14/70057, Attorney Reference 47627.99.2060 and BI-2013/107 entitled “DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS (claiming priority from one or more or all of US provisional patent applications: 62/054,490, filed Sep. 24, 2014; 62/010,441, filed Jun. 10, 2014; and 61/915,118, 61/915,215 and 61/915,148, each filed on Dec. 12, 2013) (“the Particle Delivery PCT”), incorporated herein by reference, and of PCT application PCT/US14/70127, Attorney Reference 47627.99.2091 and BI-2013/101 entitled “DELIVERY, USE AND THERAPEUTIC APPLICATIONS OF THE CRISPR-CAS SYSTEMS AND COMPOSITIONS FOR GENOME EDITING” (claiming priority from one or more or all of US provisional patent applications: 61/915,176; 61/915,192; 61/915,215; 61/915,107, 61/915,145; 61/915,148; and 61/915,153 each filed Dec. 12, 2013) (“the Eye PCT”), incorporated herein by reference, with respect to a method of preparing an sgRNA-and-Cas protein containing particle comprising admixing a mixture comprising an sgRNA and Cas effector protein (and optionally HDR template) with a mixture comprising or consisting essentially of or consisting of surfactant, phospholipid, biodegradable polymer, lipoprotein and alcohol; and particles from such a process. For example, wherein the Cas protein and sgRNA were mixed together at a suitable, e.g., 3:1 to 1:3 or 2:1 to 1:2 or 1:1 molar ratio, at a suitable temperature, e.g., 15-30 C, e.g., 20-25 C, e.g., room temperature, for a suitable time, e.g., 15-45, such as 30 minutes, advantageously in sterile, nuclease free buffer, e.g., 1×PBS. Separately, particle components such as or comprising: a surfactant, e.g., cationic lipid, e.g., 1,2-dioleoyl-3-trimethylammonium-propane (DOTAP); phospholipid, e.g., dimyristoylphosphatidylcholine (DMPC); biodegradable polymer, such as an ethylene-glycol polymer or PEG, and a lipoprotein, such as a low-density lipoprotein, e.g., cholesterol were dissolved in an alcohol, advantageously a C1-6 alkyl alcohol, such as methanol, ethanol, isopropanol, e.g., 100% ethanol. The two solutions were mixed together to form particles containing the Cas9-sgRNA complexes. Accordingly, sgRNA may be pre-complexed with the Cas protein, before formulating the entire complex in a particle. Formulations may be made with a different molar ratio of different components known to promote delivery of nucleic acids into cells (e.g., 1,2-dioleoyl-3-trimethylammonium-propane (DOTAP), 1,2-ditetradecanoyl-sn-glycero-3-phosphocholine (DMPC), polyethylene glycol (PEG), and cholesterol) For example DOTAP:DMPC:PEG:Cholesterol Molar Ratios may be DOTAP 100, DMPC 0, PEG 0, Cholesterol 0; or DOTAP 90, DMPC 0, PEG 10, Cholesterol 0; or DOTAP 90, DMPC 0, PEG 5, Cholesterol 5. DOTAP 100, DMPC 0, PEG 0, Cholesterol 0. Other example nucleotide-binding systems and proteins.

### Other Exemplary Site-Specific Nuclease or Nucleic Acid Binding Enzymes

In certain example embodiments, the retrotransposons may be used with other nucleotide-binding molecule that are not CRISPR-Cas system. Examples of the other nucleotide-binding molecules may be components of transcription activator-like effector nuclease (TALEN), Zn finger nucleases, meganucleases, a functional fragment thereof, a variant thereof, of any combination thereof. In certain example embodiments, the site-specific nuclease is an IscB. In certain other example embodiments, the site-specific nuclease is a TnpA.

### TALE Systems

In some embodiment, the nucleotide-binding molecule in the systems may be a transcription activator-like effector nuclease, a functional fragment thereof, or a variant thereof. The present disclosure also includes nucleotide sequences that are or encode one or more components of a TALE system. As disclosed herein editing can be made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference.

In some embodiments, provided herein include isolated, non-naturally occurring, recombinant or engineered DNA binding proteins that comprise TALE monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such polypeptide monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)z, where in an advantageous embodiment, z is at least 5 to 40. In a further advantageous embodiment, z is at least 10 to 26.

The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

The TALE polypeptides used in methods of the invention are isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In a preferred embodiment of the invention, polypeptide monomers having RVDs RN, NN, NK, SN, NH, KN, HN, NQ, HH, RG, KH, RH and SS preferentially bind to guanine. In a much more advantageous embodiment of the invention, polypeptide monomers having RVDs RN, NK, NQ, HH, KH, RH, SS and SN preferentially bind to guanine and thereby allow the generation of TALE polypeptides with high binding specificity for guanine containing target nucleic acid sequences. In an even more advantageous embodiment of the invention, polypeptide monomers having RVDs HH, KH, NH, NK, NQ, RH, RN and SS preferentially bind to guanine and thereby allow the generation of TALE polypeptides with high binding specificity for guanine containing target nucleic acid sequences. In a further advantageous embodiment, the RVDs that have high binding specificity for guanine are RN, NH RH and KH. Furthermore, polypeptide monomers having an RVD of NV preferentially bind to adenine and guanine. In more preferred embodiments of the invention, polypeptide monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the TALE polypeptides will bind. As used herein the polypeptide monomers and at least one or more half polypeptide monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases this region may be referred to as repeat 0. In animal genomes, TALE binding sites do not necessarily have to begin with a thymine (T) and TALE polypeptides may target DNA sequences that begin with T, A, G or C. The tandem repeat of TALE monomers always ends with a half-length repeat or a stretch of sequence that may share identity with only the first 20 amino acids of a repetitive full length TALE monomer and this half repeat may be referred to as a half-monomer (FIG. 8), which is included in the term “TALE monomer”. Therefore, it follows that the length of the nucleic acid or DNA being targeted is equal to the number of full polypeptide monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 21)
M D P I R S R T P S P A R E L L S G P Q P D G V Q P T A D R G V S P
P A G G P L D G L P A R R T M S R T R L P S P P A P S P A F S A D S
F S D L L R Q F D P S L F N T S L F D S L P P F G A H H T E A A T G
E W D E V Q S G L R A A D A P P P T M R V A V T A A R P P R A K P A
P R R R A A Q P S D A S P A A Q V D L R T L G Y S Q Q Q Q E K I K P
K V R S T V A Q H H E A L V G H G F T H A H I V A L S Q H P A A L G
T V A V K Y Q D M I A A L P E A T H E A I V G V G K Q W S G A R A L
E A L L T V A G E L R G P P L Q L D T G Q L L K I A K R G G V T A V
E A V H A W R N A L T G A P L N.

An exemplary amino acid sequence of a C-terminal capping region is:

(SEQ ID NO: 22)
R P A L E S I V A Q L S R P D P A L A A L T N D H L V A L A C L G
G R P A L D A V K K G L P H A P A L I K R T N R R I P E R T S H R
V A D H A Q V V R V L G F F Q C H S H P A Q A F D D A M T Q F G M
S R H G L L Q L F R R V G V T E L E A R S G T L P P A S Q R W D R
I L Q A S G M K R A K P S P T S T Q T P D Q A S L H A F A D S L E
R D L D A P S P M H E G D Q T R A S.

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full length capping region, while fragments that include the C-terminal 20 amino acids retain greater than 50% of the efficacy of the full length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies may be generated by any of a number of computer programs known in the art, which include but are not limited to BLAST or FASTA. Suitable computer program for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination the activities described herein.

### Zn-Finger Nucleases

In some embodiment, the nucleotide-binding molecule of the systems may be a Zn-finger nuclease, a functional fragment thereof, or a variant thereof. The composition may comprise one or more Zn-finger nucleases or nucleic acids encoding thereof. In some cases, the nucleotide sequences may comprise coding sequences for Zn-Finger nucleases. Other preferred tools for genome editing for use in the context of this invention include zinc finger systems and TALE systems. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Meganucleases

In some embodiment, the nucleotide-binding domain may be a meganuclease, a functional fragment thereof, or a variant thereof. The composition may comprise one or more meganucleases or nucleic acids encoding thereof. As disclosed herein editing can be made by way of meganucleases, which are endodeoxyribonucleases characterized by a large recognition site (double-stranded DNA sequences of 12 to 40 base pairs). In some cases, the nucleotide sequences may comprise coding sequences for meganucleases. Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, any of the nucleases, including the modified nucleases as described herein, may be used in the methods, compositions, and kits according to the invention. In particular embodiments, nuclease activity of an unmodified nuclease may be compared with nuclease activity of any of the modified nucleases as described herein, e.g. to compare for instance off-target or on-target effects. Alternatively, nuclease activity (or a modified activity as described herein) of different modified nucleases may be compared, e.g. to compare for instance off-target or on-target effects.

### RNase Domains

The compositions and systems herein may further comprise one or more RNase domains. The RNase domain may be connected to the Cas polypeptide and/or the non-LTR retrotransposon polypeptide. Ribonucleases (RNases) are a type of nuclease that catalyzes the degradation of RNA into smaller components. RNases can be divided into endoribonucleases and exoribonucleases and play key roles in the maturation of all RNA molecules, both messenger RNAs that carry genetic material for making proteins, and non-coding RNAs that function in varied cellular processes. In addition, active RNA degradation systems are a first defense against RNA viruses and provide the underlying machinery for more advanced cellular immune strategies such as RNAi. Examples of RNase domain include RNase A, RNaseH, RNaseIII, RNase L, and RNase P. In a particular example, the RNase domain is RNaseH.

RNase A is an RNase that is one of the hardiest enzymes in common laboratory usage; one method of isolating it is to boil a crude cellular extract until all enzymes other than RNase A are denatured. It is specific for single-stranded RNAs, where it cleaves the 3′-end of unpaired C and U residues, ultimately forming a 3′-phosphorylated product via a 2′,3′-cyclic monophosphate intermediate. It does not require any cofactors for its activity.

RNaseH is a non-sequence-specific endonuclease that cleaves the RNA in a DNA/RNA duplex to via a hydrolytic mechanism to produce ssDNA. Members of the RNase H family can be found in nearly all organisms, from bacteria to archaea to eukaryotes. Ribonuclease H enzymes cleave the phosphodiester bonds of RNA in a double-stranded RNA:DNA hybrid, leaving a 3′ hydroxyl and a 5′ phosphate group on either end of the cut site. RNase H1 and H2 have distinct substrate preferences and distinct but overlapping functions in the cell. In prokaryotes and lower eukaryotes, neither enzyme is essential, whereas both are believed to be essential in higher eukaryotes. The combined activity of both H1 and H2 enzymes is associated with maintenance of genome stability due to the enzymes' degradation of the RNA component of R loops.

RNase III is a type of ribonuclease that cleaves rRNA (16s rRNA and 23s rRNA) from transcribed polycistronic RNA operon in prokaryotes. It also digests double stranded RNA (dsRNA)-Dicer family of RNAse, cutting pre-miRNA (60-70 bp long) at a specific site and transforming it in miRNA (22-30 bp), that is actively involved in the regulation of transcription and mRNA life-time.

RNase L is an interferon-induced nuclease that, upon activation, destroys all RNA within the cell.

RNase P is a type of ribonuclease that is unique in that it is a ribozyme—a ribonucleic acid that acts as a catalyst in the same way as an enzyme. One of its functions is to cleave off a leader sequence from the 5′ end of one stranded pre-tRNA. RNase P is one of two known multiple turnover ribozymes in nature (the other being the ribosome). In bacteria RNase P is also responsible for the catalytic activity of holoenzymes, which consist of an apoenzyme that forms an active enzyme system by combination with a coenzyme and determines the specificity of this system for a substrate.

In some embodiments, the engineered systems described herein, further comprise an RNase domain. In specific embodiments, the RNase domain may comprise, but is not necessarily limited to, an RNase H domain.

### Nuclear Localization Sequences

In some embodiments, the polypeptides herein (e.g., site-specific nuclease polypeptides, the VirD2 polypeptide, VirD1 polypeptide, DNA polymerase, or fusion protein thereof) may further comprise (e.g., fused to) one or more nuclear localization sequences (NLSs), such as about or more than about 1, 2, 3, 4, 5, 6, 7, 8, 9, 10, or more NLSs. In some embodiments, the polypeptides and proteins comprise about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). The NLS(s) may be at an internal location of the protein, i.e., not at the C-terminus or N-terminus. When more than one NLS is present, each may be selected independently of the others, such that a single NLS may be present in more than one copy and/or in combination with one or more other NLSs present in one or more copies. In a preferred embodiment of the invention, the polypeptides comprise at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus.

In the cases of fusion protein comprising a site-specific nuclease polypeptide and a retrotransposon polypeptide, the one or more NLSs may be on any part of the fusion protein. In some examples, the NLS(s) is at the N-terminus of the fusion protein. In some examples, the NLS(s) is at the C-terminus of the fusion protein. In some example, the NLS(s) is at an internal location of the fusion protein, e.g., between the site-specific nuclease polypeptide and the retrotransposon polypeptide. Non-limiting examples of NLSs include an NLS sequence derived from: the NLS of the SV40 virus large T-antigen, having the amino acid sequence PKKKRKV (SEQ ID NO: 23); the NLS from nucleoplasmin (e.g., the nucleoplasmin bipartite NLS with the sequence KRPAATKKAGQAKKKK (SEQ ID NO: 24); the c-myc NLS having the amino acid sequence PAAKRVKLD (SEQ ID NO: 25) or RQRRNELKRSP (SEQ ID NO: 26); the hRNPA1 M9 NLS having the sequence NQSSNFGPMKGGNFGGRSSGPYGGGGQYFAKPRNQGGY (SEQ ID NO: 27); the sequence RMRIZFKNKGKDTAELRRRRVEVSVELRKAKKDEQILKRRNV (SEQ ID NO: 28) of the IBB domain from importin-alpha; the sequences VSRKRPRP (SEQ ID NO: 29) and PPKKARED (SEQ ID NO: 30) of the myoma T protein; the sequence PQPKKKPL (SEQ ID NO: 31) of human p53; the sequence SALIKKKKKMAP (SEQ ID NO: 32) of mouse c-abl IV; the sequences DRLRR (SEQ ID NO: 33) and PKQKKRK (SEQ ID NO: 34) of the influenza virus NS1; the sequence RKLKKKIKKL (SEQ ID NO: 35) of the Hepatitis virus delta antigen; the sequence REKKKFLKRR (SEQ ID NO: 36) of the mouse Mx1 protein; the sequence KRKGDEVDGVDEVAKKKSKK (SEQ ID NO: 37) of the human poly(ADP-ribose) polymerase; and the sequence RKCLQAGMNLEARKTKK (SEQ ID NO: 38) of the steroid hormone receptors (human) glucocorticoid. In general, the one or more NLSs are of sufficient strength to drive accumulation of the polypeptides in a detectable amount in the nucleus of a eukaryotic cell. In general, strength of nuclear localization activity may derive from the number of NLSs in the polypeptides, the particular NLS(s) used, or a combination of these factors. Detection of accumulation in the nucleus may be performed by any suitable technique. For example, a detectable marker may be fused to the polypeptides, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of complex formation (e.g., assay for DNA cleavage or mutation at the target sequence, or assay for altered gene expression activity affected by complex formation and/or enzyme activity), as compared to a control no exposed to the polypeptides or complex or exposed to a polypeptides lacking the one or more NLSs. In certain embodiments of the herein described polypeptides or complexes and systems, the codon optimized polypeptides comprise an NLS attached to the C-terminal of the protein. In certain embodiments, other localization tags may be fused to the polypeptides, such as without limitation for localizing the polypeptides to particular sites in a cell, such as organelles, such as mitochondria, plastids, chloroplast, vesicles, golgi, (nuclear or cellular) membranes, ribosomes, nucleoluse, ER, cytoskeleton, vacuoles, centrosome, nucleosome, granules, centrioles, etc.

In certain embodiments of the invention, at least one nuclear localization signal (NLS) is attached to the nucleic acid sequences encoding the polypeptides. In preferred embodiments at least one or more C-terminal or N-terminal NLSs are attached (and hence nucleic acid molecule(s) coding for the Cas protein can include coding for NLS(s) so that the expressed product has the NLS(s) attached or connected). In a preferred embodiment a C-terminal NLS is attached for optimal expression and nuclear targeting in eukaryotic cells, preferably human cells. The invention also encompasses methods for delivering multiple nucleic acid components, wherein each nucleic acid component is specific for a different target locus of interest thereby modifying multiple target loci of interest. The nucleic acid component of the complex may comprise one or more protein-binding RNA aptamers. The one or more aptamers may be capable of binding a bacteriophage coat protein.

### Exemplary Systems and Compositions

In some embodiments, the non-naturally occurring or engineered nucleic acid modification systems or compositions comprise a Cas nickase fused with one or more VirD2 polypeptides, one or more DNA polymerases, optionally a guide molecule (such as a guide RNA for Cas targeting insertion site on genome of a cell). The systems and compositions herein can further include a donor construct that may comprise a donor polynucleotide flanked by one or 2 boundary sequences on the 5′ end, the 3′ end, or both the 5′ and 3′ end of the donor polynucleotide sequence. The donor construct can be double-stranded or single stranded.

In some examples, the non-naturally occurring or engineered systems or compositions described herein comprise a wild type Cas9 or a Cas9 nickase (e.g., with D10A and/or H840A mutations) fused with DNA polymerase, VirD2 polypeptide, and optionally a donor polynucleotide sequence. In some embodiments, the non-naturally occurring or engineered systems or compositions comprise a wild type Cas9 or a Cas9 nickase (e.g., with D10A and/or H840A mutations) fused with DNA polymerase and/or VirD2 polypeptide and a guide RNA for Cas9 targeting insertion site on genome. the non-naturally occurring or engineered systems or compositions comprise one or more donor constructs as described elsewhere herein. In some embodiments, the systems and composition further include a guide molecule for Cas targeting of a target polynucleotide.

In some examples, the non-naturally occurring or nucleic acid modification engineered systems or compositions comprise a Type II Cas polypeptide or a Type V Cas polypeptide fused with DNA polymerase and/or VirD2 polypeptide. In some embodiments, the non-naturally occurring or engineered nucleic acid modification systems or compositions comprise a Type II Cas polypeptide or a Type V Cas polypeptide fused with DNA polymerase and/or VirD2 polypeptide and a suitable guide RNA for Cas targeting insertion site on genome. In some embodiments, the non-naturally occurring or engineered nucleic acid modification systems or compositions comprise one or more donor constructs as described elsewhere herein. In some examples, the non-naturally occurring or engineered nucleic acid modification systems or compositions comprise a Cpf1 or a Cas12b polypeptide fused with DNA polymerase and/or VirD2 polypeptide. In some embodiments, the systems and composition further include a guide molecule for Cas targeting of a target polynucleotide.

In some embodiments the systems and compositions comprise a nickase capable of nicking the non-targeted strand (see e.g., FIG. 4). In some embodiments, this nickase can be a Cas nickase. In some embodiments, this nickase is connected to or can otherwise complex with the site-specific nuclease, DNA polymerase, VirD2, and/or donor polynucleotide sequence. In some embodiments, the systems and composition further include a guide molecule for Cas targeting of a target polynucleotide.

In some embodiments, the non-naturally occurring or engineered nucleic acid modification systems or compositions comprise a Cas polypeptide comprising a transposon or domain thereof (e.g., an IscB or TpnA domain) fused with one or more VirD2 polypeptides, DNA polymerase, and donor polynucleotide, and optionally a guide RNA for Cas targeting insertion site on a polynucleotide (such as on a genome in a cell), and optionally a donor construct. In some examples, the non-naturally occurring or engineered nucleic acid modification systems or compositions comprise a Cas9 nickase fused with the transposon domain. In some examples, the non-naturally occurring or engineered nucleic acid modification systems or compositions comprise a Cas12b or Cpf1 fused with the transposon domain. In some examples, the non-naturally occurring or engineered nucleic acid modification systems or compositions comprise a Type II or a Type V fused with the transposon domain. In some embodiments, the systems and composition further include a guide molecule for Cas targeting of a target polynucleotide.

In some examples, the non-naturally occurring or engineered nucleic acid modification systems or compositions comprise a wildtype Cas fused with DNA polymerase, VirD2 polypeptide, and optionally a donor polynucleotide sequence. In some embodiments, the systems and compositions comprise a donor construct as described in greater detail elsewhere herein. In some examples, the non-naturally occurring or engineered nucleic acid modification systems or compositions comprise a wildtype Cas9 fused with DNA polymerase, VirD2 polypeptide, and optionally a donor polynucleotide sequence. In some embodiments, the systems and compositions comprise a donor construct as described in greater detail elsewhere herein. In some examples, the non-naturally occurring or engineered nucleic acid modification systems or compositions comprise a wildtype Type II Cas polypeptide, a wild-type Type V Cas polypeptide, fused with DNA polymerase, VirD2 polypeptide, and optionally a donor polynucleotide sequence. In some embodiments, the systems and compositions comprise a donor construct as described in greater detail elsewhere herein. In some examples, the non-naturally occurring or engineered nucleic acid modification systems or compositions comprise a wildtype Cas 12b polypeptide or wild-type Cpf1 polypeptide fused with DNA polymerase, VirD2 polypeptide, and optionally a donor polynucleotide sequence. In some embodiments, the systems and compositions comprise a donor construct as described in greater detail elsewhere herein. In some embodiments, the systems and composition further include a guide molecule for Cas targeting of a target polynucleotide.

In some embodiments, the Cas polypeptide contains an inactivated HNH domain. In some embodiments, the Cas polypeptide contains an inactivated RuvC domain. In some embodiments, the Cas polypeptide is a Cas9 and contains an inactivated HNH domain. In some embodiments, the Cas polypeptide is a Cas9 and contains an inactivated RuvC domain. In some embodiments, the Cas polypeptide lacks nuclease activity. In some embodiments, the Cas polypeptide is a nickase.

In some embodiments, the engineered systems and compositions may comprise two Cas proteins, each is associated with (e.g., fused to) a VirD2 polypeptide and DNA polymerase. Each of the VirD2 polypeptides can be fused to a donor polynucleotide sequence. Each of the Cas proteins can be optionally complexed with a guide molecule. Directed by their guide RNA, the Cas proteins bind to different target sites on a target polynucleotide. Each Cas protein may make a break (double-stranded or single-stranded) on its target site. In some embodiments, each Cas is selected from a wild-type Cas or an engineered Cas or Cas variant. In some embodiments, each Cas is selected from a Type V Cas polypeptide or a Type II Cas polypeptide.

In some embodiments one or both of the Cas polypeptides lacks nuclease activity. In some embodiments, one or both of the Cas polypeptides is a nickase. In some embodiments, one or both Cas polypeptides is a dCas. In some embodiments, one or both Cas polypeptides is a Cas9 or Cas9 variant. In some embodiments, one or both Cas polypeptides is a Cas12b or Cas12b variant. In some embodiments, one or both Cas polypeptides is a Cpf1 or Cpf1 variant. In some embodiments, one of the Cas polypeptides contains an inactivated HNH domain. In some embodiments, one of the Cas polypeptides contains an inactivated RuvC domain. In some embodiments, one of the Cas polypeptides is a Cas9 and contains an inactivated HNH domain. In some embodiments, one of the Cas polypeptides is a Cas9 and contains an inactivated RuvC domain.

### Polynucleotides and Vectors

The engineered systems and/or components thereof herein may comprise one or more polynucleotides and/or be encoded by said polynucleotides and/or vectors. The polynucleotide(s) may comprise coding sequences of a site-specific nuclease (such as a Cas protein(s)), VirD1, VirD2, DNA polymerase, guide sequences, or any combination thereof. The present disclosure further provides vectors or vector systems comprising one or more polynucleotides herein. The vectors or vector systems include those described in the delivery sections herein.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones, see, e.g., Eckstein, 1991; Baserga et al., 1992; Milligan, 1993; WO 97/03211; WO 96/39154; Mata, 1997; Strauss-Soukup, 1997; and Samstag, 1996. A polynucleotide may comprise one or more modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. As used herein the term “wild type” is a term of the art understood by skilled persons and means the typical form of an organism, strain, gene or characteristic as it occurs in nature as distinguished from mutant or variant forms. A “wild type” can be a base line. As used herein the term “variant” should be taken to mean the exhibition of qualities that have a pattern that deviates from what occurs in nature. The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature. “Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions. As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tm). The Tm is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tm. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this invention, it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. As used herein, “expression of a genomic locus” or “gene expression” is the process by which information from a gene is used in the synthesis of a functional gene product. The products of gene expression are often proteins, but in non-protein coding genes such as rRNA genes or tRNA genes, the product is functional RNA. The process of gene expression is used by all known life—eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell. The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics. As used herein, the term “domain” or “protein domain” refers to a part of a protein sequence that may exist and function independently of the rest of the protein chain. As described in aspects of the invention, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

In certain embodiments, the polynucleotide sequence is recombinant DNA. In further embodiments, the polynucleotide sequence further comprises additional sequences as described elsewhere herein. In certain embodiments, the nucleic acid sequence is synthesized in vitro.

Aspects of the invention relate to polynucleotide molecules that encode one or more components of the site specific-nuclease system or protein (e.g., aCRISPR-Cas system or Cas protein) as referred to in any embodiment herein. In certain embodiments, the polynucleotide molecules may comprise further regulatory sequences. By means of guidance and not limitation, the polynucleotide sequence can be part of an expression plasmid, a minicircle, a lentiviral vector, a retroviral vector, an adenoviral or adeno-associated viral vector, a piggyback vector, or a tol2 vector. In certain embodiments, the polynucleotide sequence may be a bicistronic expression construct. In further embodiments, the isolated polynucleotide sequence may be incorporated in a cellular genome. In yet further embodiments, the isolated polynucleotide sequence may be part of a cellular genome. In further embodiments, the isolated polynucleotide sequence may be comprised in an artificial chromosome. In certain embodiments, the 5′ and/or 3′ end of the isolated polynucleotide sequence may be modified to improve the stability of the sequence of actively avoid degradation. In certain embodiments, the isolated polynucleotide sequence may be comprised in a bacteriophage. In other embodiments, the isolated polynucleotide sequence may be contained in agrobacterium species. In certain embodiments, the isolated polynucleotide sequence is lyophilized. mRNA

In some embodiments, the composition comprises mRNA molecules comprising coding sequences of (i) the site-specific nuclease polypeptide(s) and/or (ii) the non-LTR retrotransposon polypeptide(s). In certain examples, a single mRNA molecule comprises coding sequences of (i) the site-specific nuclease polypeptide(s) and (ii) the non-LTR retrotransposon polypeptide(s), e.g., a fusion protein comprising (i) and (ii).

In some embodiments, the mRNA molecules comprise a poly-A tail (e.g., at its 3′ end). A poly-A tail refers to a sequence a sequence of adenyl (A) residues located on the end (e.g., 3′ end) of the RNA molecule. In some examples, an mRNA molecule comprising one or more coding sequences of the site-specific nuclease polypeptide(s) comprises a poly-A tail. In some examples, an mRNA molecule comprising one or more coding sequences of the non-LTR retrotransposon polypeptide(s) comprises a poly-A tail. In some examples, an mRNA molecule comprising one or more coding sequences of both (i) the site-specific nuclease polypeptide(s) and (ii) the non-LTR retrotransposon polypeptide(s) (e.g., a fusion protein comprising (i) and (ii)) comprises a poly-A tail.

For example, the poly-A tail may comprise from 1 to 500, from 50 to 400, from 50 to 350, from 50 to 300, from 100 to 250, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350 adenyl (A) residues.

### Codon Optimization

Aspects of the invention relate to polynucleotide molecules that encode one or more components of one or more CRISPR-Cas systems as described in any of the embodiments herein, wherein at least one or more regions of the polynucleotide molecule may be codon optimized for expression in a eukaryotic cell. In certain embodiments, the polynucleotide molecules that encode one or more components of one or more CRISPR-Cas systems as described in any of the embodiments herein are optimized for expression in a mammalian cell or a plant cell.

An example of a codon optimized sequence, is in this instance a sequence optimized for expression in a eukaryote, e.g., humans (i.e. being optimized for expression in humans), or for another eukaryote, animal or mammal as herein discussed; see, e.g., SaCas9 human codon optimized sequence in International Patent Publication No. WO 2014/093622 (PCT/US2013/074667) as an example of a codon optimized sequence (from knowledge in the art and this disclosure, codon optimizing coding nucleic acid molecule(s), especially as to effector protein is within the ambit of the skilled artisan). Whilst this is preferred, it will be appreciated that other examples are possible and codon optimization for a host species other than human, or for codon optimization for specific organs is known. In some embodiments, an enzyme coding sequence encoding a DNA/RNA-targeting Cas protein is codon optimized for expression in particular cells, such as eukaryotic cells. The eukaryotic cells may be those of or derived from a particular organism, such as a plant or a mammal, including but not limited to human, or non-human eukaryote or animal or mammal as herein discussed, e.g., mouse, rat, rabbit, dog, livestock, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence.

Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein corresponds to the most frequently used codon for a particular amino acid.

### Vector Systems

The present disclosure provides vector systems one or more vectors, the one or more vectors comprising one or more polynucleotides encoding components in retrotransposon herein, or combination thereof. The one or more polynucleotides in the vector systems may comprise one or more regulatory elements operably configures to express the polypeptide(s) and/or the nucleic acid component(s), optionally wherein the one or more regulatory elements comprise inducible promoters. In some polynucleotide molecule encoding the Cas polypeptide, VirD2 polypeptide, VirD1 polypeptide, DNA polymerase, or any combination thereof is/are codon optimized for expression in a eukaryotic cell. Suitable vectors and systems are further described elsewhere herein—see e.g., section titled “Delivery”.

### Method of Inserting Polynucleotides

The present disclosure further provides methods of inserting a polynucleotide into a target nucleic acid. Examples of the methods comprise introducing the engineered or non-naturally occurring systems or compositions herein to a cell or population of cells, wherein the site-specific nuclease or complex (such as a CRISPR-Cas complex) directs the DNA polymerase and the donor polynucleotide sequence to a target sequence, and wherein the DNA polymerase facilitates insertion of the donor polynucleotide encoded by the retrotransposon RNA at or adjacent to the target sequence.

### Immune Orthogonal Orthologs

In some embodiments, when components of the systems and compositions need to be expressed or administered in a subject, immunogenicity of components of the systems and compositions may be reduced by sequentially expressing or administering immune orthogonal orthologs of the components of the systems and compositions to the subject. As used herein, the term “immune orthogonal orthologs” refer to orthologous proteins that have similar or substantially the same function or activity but have no or low cross-reactivity with the immune response generated by one another. In some embodiments, sequential expression or administration of such orthologs elicits low or no secondary immune response. The immune orthogonal orthologs can avoid being neutralized by antibodies (e.g., existing antibodies in the host before the orthologs are expressed or administered). Cells expressing the orthologs can avoid being cleared by the host's immune system (e.g., by activated CTLs). In some examples, CRISPR enzyme orthologs from different species may be immune orthogonal orthologs.

Immune orthogonal orthologs may be identified by analyzing the sequences, structures, and/or immunogenicity of a set of candidates orthologs. In an example method, a set of immune orthogonal orthologs may be identified by a) comparing the sequences of a set of candidate orthologs (e.g., orthologs from different species) to identify a subset of candidates that have low or no sequence similarity; b) assessing immune overlap among the members of the subset of candidates to identify candidates that have no or low immune overlap. In some cases, immune overlap among candidates may be assessed by determining the binding (e.g., affinity) between a candidate ortholog and MHC (e.g., MHC type I and/or MHC II) of the host. Alternatively, or additionally, immune overlap among candidates may be assessed by determining B-cell epitopes for the candidate orthologs. In one example, immune orthogonal orthologs may be identified using the method described in Moreno A M et al., BioRxiv, published online Jan. 10, 2018, doi: doi.org/10.1101/245985.

### Delivery

The present disclosure also provides delivery systems for introducing components of the systems and compositions herein to cells, tissues, organs, or organisms. A delivery system may comprise one or more delivery vehicles and/or cargos. Exemplary delivery systems and methods include those described in paragraphs [00117] to [00278] of Feng Zhang et al., (WO2016106236A1), and pages 1241-1251 and Table 1 of Lino C A et al., Delivering CRISPR: a review of the challenges and approaches, DRUG DELIVERY, 2018, VOL. 25, NO. 1, 1234-1257, which are incorporated by reference herein in their entireties.

In some embodiments, the delivery systems may be used to introduce the components of the systems and compositions to plant cells. For example, the components may be delivered to plant using electroporation, microinjection, aerosol beam injection of plant cell protoplasts, biolistic methods, DNA particle bombardment, and/or Agrobacterium-mediated transformation. Examples of methods and delivery systems for plants include those described in Fu et al., Transgenic Res. 2000 February; 9(1):11-9; Klein R M, et al., Biotechnology. 1992; 24:384-6; Casas A M et al., Proc Natl Acad Sci USA. 1993 Dec. 1; 90(23): 11212-11216; and U.S. Pat. No. 5,563,055, Davey M R et al., Plant Mol Biol. 1989 September; 13(3):273-85, which are incorporated by reference herein in their entireties.

### Cargos

The delivery systems may comprise one or more cargos. The cargos may comprise one or more components of the CRISPR-Cas systems and compositions herein. A cargo may comprise one or more of the following: i) a vector or vector system (viral or non-viral) encoding one or more Cas proteins; ii) a vector or vector system (viral or non-viral) encoding one or more guide RNAs described herein, iii) mRNA of one or more Cas proteins; iv) one or more guide RNAs; v) one or more Cas proteins; vi) one or more polynucleotides encoding one or more Cas proteins; vii) one or more polynucleotides encoding one or more guide RNAs, or viii) any combination thereof. In some examples, a cargo may comprise a plasmid encoding one or more Cas protein and one or more (e.g., a plurality of) guide RNAs. In some embodiments, a cargo may comprise mRNA encoding one or more Cas proteins and one or more guide RNA.

In some embodiments, a cargo may comprise one or more Cas proteins described herein and one or more guide RNAs, e.g., in the form of ribonucleoprotein complexes (RNP). The ribonucleoprotein complexes may be delivered by methods and systems herein. In some cases, the ribonucleoprotein may be delivered by way of a polypeptide-based shuttle agent. In one example, the ribonucleoprotein may be delivered using synthetic peptides comprising an endosome leakage domain (ELD) operably linked to a cell penetrating domain (CPD), to a histidine-rich domain and a CPD, e.g., as describe in WO2016161516. RNP may also be used for delivering the compositions and systems to plant cells, e.g., as described in Wu J W, et al., Nat Biotechnol. 2015 November; 33(11):1162-4.

In some embodiments, the cargo(s) can be any of the polynucleotide(s), e.g., CRISPR-Cas System polynucleotides described herein.

### Physical Delivery

In some embodiments, the cargos may be introduced to cells by physical delivery methods. Examples of physical methods include microinjection, electroporation, and hydrodynamic delivery. Both nucleic acid and proteins may be delivered using such methods. For example, Cas protein may be prepared in vitro, isolated, (refolded, purified if needed), and introduced to cells.

### Microinjection

Microinjection of the cargo directly to cells can achieve high efficiency, e.g., above 90% or about 100%. In some embodiments, microinjection may be performed using a microscope and a needle (e.g., with 0.5-5.0 m in diameter) to pierce a cell membrane and deliver the cargo directly to a target site within the cell. Microinjection may be used for in vitro and ex vivo delivery.

Plasmids comprising coding sequences for Cas proteins and/or guide RNAs, mRNAs, and/or guide RNAs, may be microinjected. In some cases, microinjection may be used i) to deliver DNA directly to a cell nucleus, and/or ii) to deliver mRNA (e.g., in vitro transcribed) to a cell nucleus or cytoplasm. In certain examples, microinjection may be used to delivery sgRNA directly to the nucleus and Cas-encoding mRNA to the cytoplasm, e.g., facilitating translation and shuttling of Cas to the nucleus.

Microinjection may be used to generate genetically modified animals. For example, gene editing cargos may be injected into zygotes to allow for efficient germline modification. Such approach can yield normal embryos and full-term mouse pups harboring the desired modification(s). Microinjection can also be used to provide transiently up- or down-regulate a specific gene within the genome of a cell, e.g., using CRISPRa and CRISPRi.

### Electroporation

In some embodiments, the cargos and/or delivery vehicles may be delivered by electroporation. Electroporation may use pulsed high-voltage electrical currents to transiently open nanometer-sized pores within the cellular membrane of cells suspended in buffer, allowing for components with hydrodynamic diameters of tens of nanometers to flow into the cell. In some cases, electroporation may be used on various cell types and efficiently transfer cargo into cells. Electroporation may be used for in vitro and ex vivo delivery.

Electroporation may also be used to deliver the cargo to into the nuclei of mammalian cells by applying specific voltage and reagents, e.g., by nucleofection. Such approaches include those described in Wu Y, et al. (2015). Cell Res 25:67-79; Ye L, et al. (2014). Proc Natl Acad Sci USA 111:9591-6; Choi P S, Meyerson M. (2014). Nat Commun 5:3728; Wang J, Quake S R. (2014). Proc Natl Acad Sci 111:13157-62. Electroporation may also be used to deliver the cargo in vivo, e.g., with methods described in Zuckermann M, et al. (2015). Nat Commun 6:7391.

### Hydrodynamic Delivery

Hydrodynamic delivery may also be used for delivering the cargos, e.g., for in vivo delivery. In some examples, hydrodynamic delivery may be performed by rapidly pushing a large volume (8-10% body weight) solution containing the gene editing cargo into the bloodstream of a subject (e.g., an animal or human), e.g., for mice, via the tail vein. As blood is incompressible, the large bolus of liquid may result in an increase in hydrodynamic pressure that temporarily enhances permeability into endothelial and parenchymal cells, allowing for cargo not normally capable of crossing a cellular membrane to pass into cells. This approach may be used for delivering naked DNA plasmids and proteins. The delivered cargos may be enriched in liver, kidney, lung, muscle, and/or heart.

### Transfection

The cargos, e.g., nucleic acids and/or polypeptides, may be introduced to cells by transfection methods for introducing nucleic acids into cells. Examples of transfection methods include calcium phosphate-mediated transfection, cationic transfection, liposome transfection, dendrimer transfection, heat shock transfection, magnetofection, lipofection, impalefection, optical transfection, proprietary agent-enhanced uptake of nucleic acid.

### Transduction

The cargos, e.g., nucleic acids and/or polypeptides, can be introduced to cells by transduction by a viral or pseudoviral particle. Methods of packaging the cargos in viral particles can be accomplished using any suitable viral vector or vector systems. Such viral vector and vector systems are described in greater detail elsewhere herein. As used in this context herein “transduction” refers to the process by which foreign nucleic acids and/or proteins are introduced to a cell (prokaryote or eukaryote) by a viral or pseudo viral particle. After packaging in a viral particle or pseudo viral particle, the viral particles can be exposed to cells (e.g., in vitro, ex vivo, or in vivo) where the viral or pseudoviral particle infects the cell and delivers the cargo to the cell via transduction. Viral and pseudoviral particles can be optionally concentrated prior to exposure to target cells. In some embodiments, the virus titer of a composition containing viral and/or pseudoviral particles can be obtained and a specific titer be used to transduce cells.

### Biolistics

The cargos, e.g., nucleic acids and/or polypeptides, can be introduced to cells using a biolistic method or technique. The term of art “biolistic”, as used herein refers to the delivery of nucleic acids to cells by high-speed particle bombardment. In some embodiments, the cargo(s) can be attached, associated with, or otherwise coupled to particles, which than can be delivered to the cell via a gene-gun (see e.g., Liang et al. 2018. Nat. Protocol. 13:413-430; Svitashev et al. 2016. Nat. Comm. 7:13274; Ortega-Escalante et al., 2019. Plant. J. 97:661-672). In some embodiments, the particles can be gold, tungsten, palladium, rhodium, platinum, or iridium particles.

### Implantable Devices

In some embodiments, the delivery system can include an implantable device that incorporates or is coated with a CRISPR-Cas system or component thereof described herein. Various implantable devices are described in the art, and include any device, graft, or other composition that can be implanted into a subject.

### Delivery Vehicles

The delivery systems may comprise one or more delivery vehicles. The delivery vehicles may deliver the cargo into cells, tissues, organs, or organisms (e.g., animals or plants). The cargos may be packaged, carried, or otherwise associated with the delivery vehicles. The delivery vehicles may be selected based on the types of cargo to be delivered, and/or the delivery is in vitro and/or in vivo. Examples of delivery vehicles include vectors, viruses (e.g., virus particles), non-viral vehicles, and other delivery reagents described herein.

The delivery vehicles in accordance with the present invention may have an average greatest dimension (e.g., diameter, length, width, etc.) of less than 100 microns (μm). In some embodiments, the delivery vehicles have average greatest dimension of less than 10 μm. In some embodiments, the delivery vehicles may have average greatest dimension of less than 2000 nanometers (nm). In some embodiments, the delivery vehicles may have average greatest dimension of less than 1000 nanometers (nm). In some embodiments, the delivery vehicles may have average greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have average greatest dimension ranging between 25 nm and 200 nm.

The delivery vehicles in accordance with the present invention may have a greatest dimension (e.g., diameter, length, width, etc.)) of less than 100 microns (μm). In some embodiments, the delivery vehicles have a greatest dimension of less than 10 μm. In some embodiments, the delivery vehicles may have a greatest dimension of less than 2000 nanometers (nm). In some embodiments, the delivery vehicles may have a greatest dimension of less than 1000 nanometers (nm). In some embodiments, the delivery vehicles may have a greatest dimension (e.g., diameter) of less than 900 nm, less than 800 nm, less than 700 nm, less than 600 nm, less than 500 nm, less than 400 nm, less than 300 nm, less than 200 nm, less than 150 nm, or less than 100 nm, less than 50 nm. In some embodiments, the delivery vehicles may have a greatest dimension ranging between 25 nm and 200 nm.

In some embodiments, the delivery vehicles may be or comprise particles. For example, the delivery vehicle may be or comprise nanoparticles (e.g., particles with a greatest dimension (e.g., diameter) no greater than 1000 nm. The particles may be provided in different forms, e.g., as solid particles (e.g., metal such as silver, gold, iron, titanium), non-metal, lipid-based solids, polymers), suspensions of particles, or combinations thereof. Metal, dielectric, and semiconductor particles may be prepared, as well as hybrid structures (e.g., core-shell particles).

Nanoparticles may also be used to deliver the compositions and systems to plant cells, e.g., as described in WO 2008042156, US 20130185823, and WO2015089419. In general, a “nanoparticle” refers to any particle having a diameter of less than 1000 nm. In certain preferred embodiments, nanoparticles of the invention have a greatest dimension (e.g., diameter) of 500 nm or less. In other preferred embodiments, nanoparticles of the invention have a greatest dimension ranging between 25 nm and 200 nm. In other preferred embodiments, nanoparticles of the invention have a greatest dimension of 100 nm or less. In other preferred embodiments, nanoparticles of the invention have a greatest dimension ranging between 35 nm and 60 nm. It will be appreciated that reference made herein to particles or nanoparticles can be interchangeable, where appropriate. Nanoparticles made of semiconducting material may also be labeled quantum dots if they are small enough (typically sub 10 nm) that quantization of electronic energy levels occurs. Such nanoscale particles are used in biomedical applications as drug carriers or imaging agents and may be adapted for similar purposes in the present invention. Semi-solid and soft nanoparticles have been manufactured and are within the scope of the present invention. Nanoparticles with one half hydrophilic and the other half hydrophobic are termed Janus particles and are particularly effective for stabilizing emulsions. They can self-assemble at water/oil interfaces and act as solid surfactants.

Particle characterization (including e.g., characterizing morphology, dimension, etc.) is done using a variety of different techniques. Common techniques are electron microscopy (TEM, SEM), atomic force microscopy (AFM), dynamic light scattering (DLS), X-ray photoelectron spectroscopy (XPS), powder X-ray diffraction (XRD), Fourier transform infrared spectroscopy (FTIR), matrix-assisted laser desorption/ionization time-of-flight mass spectrometry (MALDI-TOF), ultraviolet-visible spectroscopy, dual polarization interferometry and nuclear magnetic resonance (NMR). Characterization (dimension measurements) may be made as to native particles (i.e., preloading) or after loading of the cargo (herein cargo refers to e.g., one or more components of CRISPR-Cas system e.g., CRISPR enzyme or mRNA or guide RNA, or any combination thereof, and may include additional carriers and/or excipients) to provide particles of an optimal size for delivery for any in vitro, ex vivo and/or in vivo application of the present invention. In certain preferred embodiments, particle dimension (e.g., diameter) characterization is based on measurements using dynamic laser scattering (DLS). Mention is made of U.S. Pat. Nos. 8,709,843; 6,007,845; 5,855,913; 5,985,309; 5,543,158; and the publication by James E. Dahlman and Carmen Barnes et al. Nature Nanotechnology (2014) published online 11 May 2014, doi:10.1038/nnano.2014.84, describing particles, methods of making and using them and measurements thereof.

### Vectors and Vector Systems

Also provided herein are vectors that can contain one or more of the engineered nucleic acid modification system polynucleotides described herein. In certain embodiments, the vector can contain one or more polynucleotides encoding one or more elements of a engineered nucleic acid modification system described herein. The vectors can be useful in producing bacterial, fungal, yeast, plant cells, animal cells, and transgenic animals that can express one or more components of the engineered nucleic acid modification system described herein. Within the scope of this disclosure are vectors containing one or more of the polynucleotide sequences described herein. One or more of the polynucleotides that are part of the engineered nucleic acid modification system described herein can be included in a vector or vector system. The vectors and/or vector systems can be used, for example, to express one or more of the polynucleotides in a cell, such as a producer cell, to produce engineered nucleic acid modification system containing virus particles described elsewhere herein. Other uses for the vectors and vector systems described herein are also within the scope of this disclosure. In general, and throughout this specification, the term “vector” refers to a tool that allows or facilitates the transfer of an entity from one environment to another. In some contexts which will be appreciated by those of ordinary skill in the art, “vector” can be a term of art to refer to a nucleic acid molecule capable of transporting another nucleic acid to which it has been linked. A vector can be a replicon, such as a plasmid, phage, or cosmid, into which another DNA segment may be inserted so as to bring about the replication of the inserted segment. Generally, a vector is capable of replication when associated with the proper control elements.

Vectors include, but are not limited to, nucleic acid molecules that are single-stranded, double-stranded, or partially double-stranded; nucleic acid molecules that comprise one or more free ends, no free ends (e.g., circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g., retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can be composed of a nucleic acid (e.g., a polynucleotide) of the invention in a form suitable for expression of the nucleic acid in a host cell, which means that the recombinant expression vectors include one or more regulatory elements, which can be selected on the basis of the host cells to be used for expression, that is operatively-linked to the nucleic acid sequence to be expressed. Within a recombinant expression vector, “operably linked” and “operatively-linked” are used interchangeably herein and further defined elsewhere herein. In the context of a vector, the term “operably linked” is intended to mean that the nucleotide sequence of interest is linked to the regulatory element(s) in a manner that allows for expression of the nucleotide sequence (e.g., in an in vitro transcription/translation system or in a host cell when the vector is introduced into the host cell). Advantageous vectors include lentiviruses and adeno-associated viruses, and types of such vectors can also be selected for targeting particular types of cells. These and other embodiments of the vectors and vector systems are described elsewhere herein.

In some embodiments, the vector can be a bicistronic vector. In some embodiments, a bicistronic vector can be used for one or more elements of the engineered nucleic acid modification system described herein. In some embodiments, expression of elements of the engineered nucleic acid modification system described herein can be driven by the CBh promoter or other ubiquitous promoter. Where the element of the engineered nucleic acid modification system is an RNA, its expression can be driven by a Pol III promoter, such as a U6 promoter. In some embodiments, the two are combined.

In some embodiments, a vector capable of delivering one or more proteins of the engineered nucleic acid modification system and optionally at least one CRISPR-Cas system guide RNA to a cell can be composed of or contain a minimal promoter operably linked to a polynucleotide sequence encoding the effector protein and a second minimal promoter operably linked to a polynucleotide sequence encoding at least one guide RNA, wherein the length of the vector sequence comprising the minimal promoters and polynucleotide sequences is less than 4.4 Kb. In an embodiment, the vector can be a viral vector. In certain embodiments, the viral vector is an is an adeno-associated virus (AAV) or an adenovirus vector. In another embodiment, the engineered nucleic acid modification system protein(s) or other components delivered by the vector is a site-specific nuclease (including but not limited to a Cas polypeptide), DNA polymerase, a Vir polypeptide (e.g., a VirD2 and/or VirD1 polypeptide), guide molecule, or any combination thereof. In a further embodiment, the Cas is Cas9.

In some embodiments, the vector is a lentiviral vector for delivery of one or more components of the engineered nucleic acid modification system described herein (e.g., a site-specific nuclease (including but not limited to a Cas polypeptide), DNA polymerase, a Vir polypeptide (e.g., a VirD2 and/or VirD1 polypeptide), guide molecule, or any combination thereof) to a cell can be composed of or contain a promoter operably linked to a polynucleotide sequence encoding one or more of the proteins of the engineered nucleic acid modification system described herein (including but not limited to a site-specific nuclease polypeptide (e.g., a Cas), Vir polypeptide (e.g., a VirD2 and/or VirD1 polypeptide), DNA polymerase, and any combination thereof) and a second promoter operably linked to a polynucleotide sequence encoding at least one guide molecule, wherein the polynucleotide sequences are in reverse orientation.

In one embodiment, the invention provides a vector system comprising one or more vectors. In some embodiments, the system comprises: (a) a first regulatory element operably linked to a direct repeat sequence and one or more insertion sites for inserting one or more guide sequences up- or downstream (whichever applicable) of the direct repeat sequence, wherein when expressed, the one or more guide sequence(s) direct(s) sequence-specific binding of the CRISPR complex to the one or more target sequence(s) in a eukaryotic cell, wherein the CRISPR complex comprises a Cas enzyme complexed with the one or more guide sequence(s) that is hybridized to the one or more target sequence(s); and (b) a second regulatory element operably linked to an enzyme-coding sequence encoding said Cas enzyme, preferably comprising at least one nuclear localization sequence and/or at least one NES; wherein components (a) and (b) are located on the same or different vectors of the system. Where applicable, a tracr sequence may also be provided. In some embodiments, component (a) further comprises two or more guide sequences operably linked to the first regulatory element, wherein when expressed, each of the two or more guide sequences direct sequence specific binding of a Cas CRISPR complex to a different target sequence in a eukaryotic cell. In some embodiments, the CRISPR complex comprises one or more nuclear localization sequences and/or one or more NES of sufficient strength to drive accumulation of said Cas CRISPR complex in a detectable amount in or out of the nucleus of a eukaryotic cell. In some embodiments, the first regulatory element is a polymerase III promoter. In some embodiments, the second regulatory element is a polymerase II promoter. In some embodiments, each of the guide sequences is at least 16, 17, 18, 19, 20, 25 nucleotides, or between 16-30, or between 16-25, or between 16-20 nucleotides in length.

These and others are further detailed and described elsewhere herein.

### Cell-Based Vector Amplification and Expression

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g., amplifying a plasmid as part of a viral vector packaging system). The vectors can be viral-based or non-viral based. In some embodiments, a prokaryote is used to amplify copies of a vector and express one or more nucleic acids, such as to provide a source of one or more proteins for delivery to a host cell or host organism.

Vectors can be designed for expression of one or more elements of the engineered nucleic acid modification system of the present invention described herein (e.g., nucleic acid transcripts, proteins, enzymes, and combinations thereof) in a suitable host cell. In some embodiments, the suitable host cell is a prokaryotic cell. Suitable host cells include, but are not limited to, bacterial cells, yeast cells, insect cells, and mammalian cells. In some embodiments, the suitable host cell is a eukaryotic cell.

In some embodiments, the suitable host cell is a suitable bacterial cell. Suitable bacterial cells include, but are not limited to, bacterial cells from the bacteria of the species Escherichia coli. Many suitable strains of E. coli are known in the art for expression of vectors. These include, but are not limited to Pir1, Stbl2, Stbl3, Stbl4, TOP10, XL1 Blue, and XL10 Gold. In some embodiments, the host cell is a suitable insect cell. Suitable insect cells include those from Spodoptera frugiperda. Suitable strains of S. frugiperda cells include, but are not limited to, Sf9 and Sf21. In some embodiments, the host cell is a suitable yeast cell. In some embodiments, the yeast cell can be from Saccharomyces cerevisiae. In some embodiments, the host cell is a suitable mammalian cell. Many types of mammalian cells have been developed to express vectors. Suitable mammalian cells include, but are not limited to, HEK293, Chinese Hamster Ovary Cells (CHOs), mouse myeloma cells, HeLa, U20S, A549, HT1080, CAD, P19, NIH 3T3, L929, N2a, MCF-7, Y79, SO-Rb50, HepG G2, DIKX-X11, J558L, Baby hamster kidney cells (BHK), and chicken embryo fibroblasts (CEFs). Suitable host cells are discussed further in Goeddel, GENE EXPRESSION TECHNOLOGY: METHODS IN ENZYMOLOGY 185, Academic Press, San Diego, Calif (1990).

In some embodiments, the vector can be a yeast expression vector. Examples of vectors for expression in yeast Saccharomyces cerevisiae include pYepSec1 (Baldari, et al., 1987. EMBO J. 6: 229-234), pMFa (Kuijan and Herskowitz, 1982. Cell 30: 933-943), pJRY88 (Schultz et al., 1987. Gene 54: 113-123), pYES2 (Invitrogen Corporation, San Diego, Calif), and picZ (InVitrogen Corp, San Diego, Calif). As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors can contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

In some embodiments, the vector is a baculovirus vector or expression vector and can be suitable for expression of polynucleotides and/or proteins in insect cells. In some embodiments, the suitable host cell is an insect cell. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39). rAAV (recombinant Adeno-associated viral) vectors are preferably produced in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, the vector is a mammalian expression vector. In some embodiments, the mammalian expression vector is capable of expressing one or more polynucleotides and/or polypeptides in a mammalian cell. Examples of mammalian expression vectors include, but are not limited to, pCDM8 (Seed, 1987. Nature 329: 840) and pMT2PC (Kaufman, et al., 1987. EMBO J. 6: 187-195). The mammalian expression vector can include one or more suitable regulatory elements capable of controlling expression of the one or more polynucleotides and/or proteins in the mammalian cell. For example, commonly used promoters are derived from polyoma, adenovirus 2, cytomegalovirus, simian virus 40, and others disclosed herein and known in the art. More detail on suitable regulatory elements are described elsewhere herein.

For other suitable expression vectors and vector systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; see e.g., Pinkert, et al., 1987. Genes Dev. 1: 268-277), lymphoid-specific promoters (see e.g., Calame and Eaton, 1988. Adv. Immunol. 43: 235-275), in particular promoters of T cell receptors (see e.g., Winoto and Baltimore, 1989. EMBO J. 8: 729-733) and immunoglobulins (see e.g., Baneiji, et al., 1983. Cell 33: 729-740; Queen and Baltimore, 1983. Cell 33: 741-748), neuron-specific promoters (see e.g., the neurofilament promoter; Byrne and Ruddle, 1989. Proc. Natl. Acad. Sci. USA 86: 5473-5477), pancreas-specific promoters (see e.g., Edlund, et al., 1985. Science 230: 912-916), and mammary gland-specific promoters (e.g., milk whey promoter; see e.g., U.S. Pat. No. 4,873,316 and European Application Publication No. 264,166). Developmentally-regulated promoters are also encompassed, e.g., the murine hox promoters (see e.g., Kessel and Gruss, 1990. Science 249: 374-379) and the α-fetoprotein promoter (see e.g., Campes and Tilghman, 1989. Genes Dev. 3: 537-546). With regards to these prokaryotic and eukaryotic vectors, mention is made of U.S. Pat. No. 6,750,059, the contents of which are incorporated by reference herein in their entirety. Other embodiments can utilize viral vectors, with regards to which mention is made of U.S. patent application Ser. No. 13/092,085, the contents of which are incorporated by reference herein in their entirety. Tissue-specific regulatory elements are known in the art and in this regard, mention is made of U.S. Pat. No. 7,776,321, the contents of which are incorporated by reference herein in their entirety. In some embodiments, a regulatory element can be operably linked to one or more elements of the engineered nucleic acid modification system so as to drive expression of the one or more elements of the engineered nucleic acid modification system of the present invention described herein.

In some embodiments, the vector can be a fusion vector or fusion expression vector. In some embodiments, fusion vectors add a number of amino acids to a protein encoded therein, such as to the amino terminus, carboxy terminus, or both of a recombinant protein. Such fusion vectors can serve one or more purposes, such as: (i) to increase expression of recombinant protein; (ii) to increase the solubility of the recombinant protein; and (iii) to aid in the purification of the recombinant protein by acting as a ligand in affinity purification. In some embodiments, expression of polynucleotides (such as non-coding polynucleotides) and proteins in prokaryotes can be carried out in Escherichia coli with vectors containing constitutive or inducible promoters directing the expression of either fusion or non-fusion polynucleotides and/or proteins. In some embodiments, the fusion expression vector can include a proteolytic cleavage site, which can be introduced at the junction of the fusion vector backbone or other fusion moiety and the recombinant polynucleotide or protein to enable separation of the recombinant polynucleotide or protein from the fusion vector backbone or other fusion moiety subsequent to purification of the fusion polynucleotide or protein. Such enzymes, and their cognate recognition sequences, include Factor Xa, thrombin and enterokinase. Example fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, one or more vectors driving expression of one or more elements of an engineered nucleic acid modification system of the present invention described herein are introduced into a host cell such that expression of the elements of the engineered nucleic acid modification system of the present invention described herein system described herein direct formation a CRISPR-Cas complex at one or more target sites. For example, a CRISPR-Cas effector protein (and other proteins of the engineered nucleic acid modification system of the present invention (e.g., VirD2 and DNA polymerase) described herein and a nucleic acid component (e.g., a guide polynucleotide) can each be operably linked to separate regulatory elements on separate vectors. RNA(s) of different elements of CRISPR-engineered nucleic acid modification system of the present invention described herein can be delivered to an animal, plant, microorganism or cell thereof to produce an animal (e.g., a mammal, reptile, avian, etc.), plant, microorganism or cell thereof that constitutively, inducibly, or conditionally expresses different elements of the engineered nucleic acid modification system of the present invention described herein that incorporates one or more elements of the engineered nucleic acid modification system of the present invention described herein or contains one or more cells that incorporates and/or expresses one or more elements of the engineered nucleic acid modification system of the present invention described herein.

In some embodiments, two or more of the elements expressed from the same or different regulatory element(s), can be combined in a single vector, with one or more additional vectors providing any components of the system not included in the first vector. Engineered nucleic acid modification system polynucleotides that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding one or more engineered nucleic acid modification system of the present invention described herein proteins and/or polynucleotides, embedded within one or more intron sequences (e.g., each in a different intron, two or more in at least one intron, or all in a single intron). In some embodiments, the C engineered nucleic acid modification system polynucleotides can be operably linked to and expressed from the same promoter.

### Cell-Free Vector and Polvnucleotide Expression

In some embodiments, the polynucleotide encoding one or more features of the engineered nucleic acid modification system of the present invention can be expressed from a vector or suitable polynucleotide in a cell-free in vitro system. In other words, the polynucleotide can be transcribed and optionally translated in vitro. In vitro transcription/translation systems and appropriate vectors are generally known in the art and commercially available. Generally, in vitro transcription and in vitro translation systems replicate the processes of RNA and protein synthesis, respectively, outside of the cellular environment. Vectors and suitable polynucleotides for in vitro transcription can include T7, SP6, T3, promoter regulatory sequences that can be recognized and acted upon by an appropriate polymerase to transcribe the polynucleotide or vector.

In vitro translation can be stand-alone (e.g., translation of a purified polyribonucleotide) or linked/coupled to transcription. In some embodiments, the cell-free (or in vitro) translation system can include extracts from rabbit reticulocytes, wheat germ, and/or E. coli. The extracts can include various macromolecular components that are needed for translation of exogenous RNA (e.g., 70S or 80S ribosomes, tRNAs, aminoacyl-tRNA, synthetases, initiation, elongation factors, termination factors, etc.). Other components can be included or added during the translation reaction, including but not limited to, amino acids, energy sources (ATP, GTP), energy regenerating systems (creatine phosphate and creatine phosphokinase (eukaryotic systems)) (phosphoenol pyruvate and pyruvate kinase for bacterial systems), and other co-factors (Mg2+, K+, etc.). As previously mentioned, in vitro translation can be based on RNA or DNA starting material. Some translation systems can utilize an RNA template as starting material (e.g., reticulocyte lysates and wheat germ extracts). Some translation systems can utilize a DNA template as a starting material (e.g., E coli-based systems). In these systems transcription and translation are coupled and DNA is first transcribed into RNA, which is subsequently translated. Suitable standard and coupled cell-free translation systems are generally known in the art and are commercially available.

### Vector Features

The vectors can include additional features that can confer one or more functionalities to the vector, the polynucleotide to be delivered, a virus particle produced there from, or polypeptide expressed thereof. Such features include, but are not limited to, regulatory elements, selectable markers, molecular identifiers (e.g., molecular barcodes), stabilizing elements, and the like. It will be appreciated by those skilled in the art that the design of the expression vector and additional features included can depend on such factors as the choice of the host cell to be transformed, the level of expression desired, etc.

### Regulatory Elements

In certain embodiments, the polynucleotides and/or vectors thereof described herein (such as the engineered nucleic acid modification system polynucleotides of the present invention) can include one or more regulatory elements that can be operatively linked to the polynucleotide. The term “regulatory element” is intended to include promoters, enhancers, internal ribosomal entry sites (IRES), other expression control elements (e.g., transcription termination signals, such as polyadenylation signals and poly-U sequences) and cellular localization signals (e.g., nuclear localization signals). Such regulatory elements are described, for example, in Goeddel, GENE EXPRESSION TECHNOLOGY: METHODS IN ENZYMOLOGY 185, Academic Press, San Diego, Calif (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter can direct expression primarily in a desired tissue of interest, such as muscle, neuron, bone, skin, blood, specific organs (e.g., liver, pancreas), or particular cell types (e.g., lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) (see e.g., Boshart et al, Cell, 41:521-530 (1985)), the SV40 promoter, the dihydrofolate reductase promoter, the 3-actin promoter, the phosphoglycerol kinase (PGK) promoter, and the EF1a promoter. Also encompassed by the term “regulatory element” are enhancer elements, such as WPRE; CMV enhancers; the R-U5′ segment in LTR of HTLV-I (Mol. Cell. Biol., Vol. 8(1), p. 466-472, 1988); SV40 enhancer; and the intron sequence between exons 2 and 3 of rabbit β-globin (see e.g., Proc. Natl. Acad. Sci. USA., Vol. 78(3), p. 1527-31, 1981).

In some embodiments, the regulatory sequence can be a regulatory sequence described in U.S. Pat. No. 7,776,321, U.S. Pat. Pub. No. 2011/0027239, and International Patent Publication No. WO 2011/028929, the contents of which are incorporated by reference herein in their entirety. In some embodiments, the vector can contain a minimal promoter. In some embodiments, the minimal promoter is the Mecp2 promoter, tRNA promoter, or U6. In a further embodiment, the minimal promoter is tissue specific. In some embodiments, the length of the vector polynucleotide the minimal promoters and polynucleotide sequences is less than 4.4 Kb.

To express a polynucleotide, the vector can include one or more transcriptional and/or translational initiation regulatory sequences, e.g., promoters, that direct the transcription of the gene and/or translation of the encoded protein in a cell. In some embodiments a constitutive promoter may be employed. Suitable constitutive promoters for mammalian cells are generally known in the art and include, but are not limited to SV40, CAG, CMV, EF-1α, β-actin, RSV, and PGK. Suitable constitutive promoters for bacterial cells, yeast cells, and fungal cells are generally known in the art, such as a T-7 promoter for bacterial expression and an alcohol dehydrogenase promoter for expression in yeast.

In some embodiments, the regulatory element can be a regulated promoter. “Regulated promoter” refers to promoters that direct gene expression not constitutively, but in a temporally- and/or spatially-regulated manner, and includes tissue-specific, tissue-preferred and inducible promoters. Regulated promoters include conditional promoters and inducible promoters. In some embodiments, conditional promoters can be employed to direct expression of a polynucleotide in a specific cell type, under certain environmental conditions, and/or during a specific state of development. Suitable tissue specific promoters can include, but are not limited to, liver specific promoters (e.g., APOA2, SERPIN A1 (hAAT), CYP3A4, and MIR122), pancreatic cell promoters (e.g., INS, IRS2, Pdxl, Alx3, Ppy), cardiac specific promoters (e.g., Myh6 (alpha MHC), MYL2 (MLC-2v), TNI3 (cTnl), NPPA (ANF), Slc8a1 (Ncx1)), central nervous system cell promoters (e.g., SYN1, GFAP, INA, NES, MOBP, MBP, TH, FOXA2 (HNF3 beta)), skin cell specific promoters (e.g., FLG, K14, TGM3), immune cell specific promoters, (e.g., ITGAM, CD43 promoter, CD14 promoter, CD45 promoter, CD68 promoter), urogenital cell specific promoters (e.g., Pbsn, Upk2, Sbp, Ferl14), endothelial cell specific promoters (e.g. ENG), pluripotent and embryonic germ layer cell specific promoters (e.g., Oct4, NANOG, Synthetic Oct4, T brachyury, NES, SOX17, FOXA2, MIR122), and muscle cell specific promoter (e.g., Desmin). Other tissue and/or cell specific promoters are generally known in the art and are within the scope of this disclosure.

Inducible/conditional promoters can be positively inducible/conditional promoters (e.g., a promoter that activates transcription of the polynucleotide upon appropriate interaction with an activated activator, or an inducer (compound, environmental condition, or other stimulus) or a negative/conditional inducible promoter (e.g., a promoter that is repressed (e.g., bound by a repressor) until the repressor condition of the promotor is removed (e.g., inducer binds a repressor bound to the promoter stimulating release of the promoter by the repressor or removal of a chemical repressor from the promoter environment). The inducer can be a compound, environmental condition, or other stimulus. Thus, inducible/conditional promoters can be responsive to any suitable stimuli such as chemical, biological, or other molecular agents, temperature, light, and/or pH. Suitable inducible/conditional promoters include, but are not limited to, Tet-On, Tet-Off, Lac promoter, pBad, AlcA, LexA, Hsp70 promoter, Hsp90 promoter, pDawn, XVE/OlexA, GVG, and pOp/LhGR.

Where expression in a plant cell is desired, the components of the CRISPR-Cas system described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. Different promoters may direct the expression of a gene in different tissues or cell types, or at different stages of development, or in response to different environmental conditions. In particular embodiments, one or more of the engineered nucleic acid modification system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the engineered nucleic acid modification system of the present invention are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-691.

Examples of promoters that are inducible and that can allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (e.g., FKBP, ABA, etc.), or light inducible systems (Phytochrome, LOV domains, or cryptochrome), such as a Light Inducible Transcriptional Effector (LITE) that direct changes in transcriptional activity in a sequence-specific manner. Other inducible promoters can be found in e.g., Kallunki et al. Cells. 2019. 8:796 doi:10.3390/cells8080796. The components of a light inducible system may include one or more elements of the CRISPR-Cas system described herein, a light-responsive cytochrome heterodimer (e.g., from Arabidopsis thaliana), and a transcriptional activation/repression domain. In some embodiments, the vector can include one or more of the inducible DNA binding proteins provided in International Patent Publication No. WO 2014/018423 and U.S. Patent Publication Nos. 2015/0291966, 2017/0166903, 2019/0203212, which describe e.g., embodiments of inducible DNA binding proteins and methods of use and can be adapted for use with the present invention.

In some embodiments, transient or inducible expression can be achieved by including, for example, chemical-regulated promotors, i.e., whereby the application of an exogenous chemical induces gene expression. Modulation of gene expression can also be obtained by including a chemical-repressible promoter, where application of the chemical represses gene expression. Chemical-inducible promoters include, but are not limited to, the maize ln2-2 promoter, activated by benzene sulfonamide herbicide safeners (see e.g., De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (see e.g., Ono et al., (2004) Biosci Biotechnol Biochem 68:803-7) activated by salicylic acid. Promoters which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156) can also be used herein.

In some embodiments, the polynucleotide, vector or system thereof can include one or more elements capable of translocating and/or expressing an engineered nucleic acid modification system polynucleotide to/in a specific cell component or organelle. Such organelles can include, but are not limited to, nucleus, ribosome, endoplasmic reticulum, Golgi apparatus, chloroplast, mitochondria, vacuole, lysosome, cytoskeleton, plasma membrane, cell wall, peroxisome, centrioles, etc. Such regulatory elements can include, but are not limited to, nuclear localization signals (examples of which are described in greater detail elsewhere herein), any such as those that are annotated in the LocSigDB database (see e.g., http://genome.unmc.edu/LocSigDB/ and Negi et al., 2015. Database. 2015: bav003; doi: 10.1093/database/bav003), nuclear export signals (e.g., LXXXLXXLXL and others described elsewhere herein), endoplasmic reticulum localization/retention signals (e.g., KDEL, KDXX, KKXX, KXX, and others described elsewhere herein; and see e.g., Liu et al. 2007 Mol. Biol. Cell. 18(3):1073-1082 and Gorleku et al., 2011. J. Biol. Chem. 286:39573-39584), mitochondria (see e.g., Cell Reports. 22:2818-2826, particularly at FIG. 2; Doyle et al. 2013. PLoS ONE 8, e67938; Funes et al. 2002. J. Biol. Chem. 277:6051-6058; Matouschek et al. 1997. PNAS USA 85:2091-2095; Oca-Cossio et al., 2003. 165:707-720; Waltner et al., 1996. J. Biol. Chem. 271:21226-21230; Wilcox et al., 2005. PNAS USA 102:15435-15440; Galanis et al., 1991. FEBS Lett 282:425-430, peroxisome (e.g. (S/A/C)-(K/R/H)-(L/A), SLK, (R/K)-(L/V/I)-XXXXX-(H/Q)-(L/A/F) (SEQ ID NO: 41). Suitable protein targeting motifs can also be designed or identified using any suitable database or prediction tool, including but not limited to Minimotif Miner (http:minimotifminer.org, http://mitominer.mrc-mbu.cam.ac.uk/release-4.0/embodiment.do?name=Protein %20MTS), LocDB (see above), PTSs predictor ( ), TargetP-2.0 (http://www.cbs.dtu.dk/services/TargetP/), ChloroP (http://www.cbs.dtu.dk/services/ChloroP/); NetNES (http://www.cbs.dtu.dk/services/NetNES/), Predotar (https://urgi.versailles.inra.fr/predotar/), and SignalP (http://www.cbs.dtu.dk/services/SignalP/).

### Selectable Markers and Tags

One or more of the engineered nucleic acid modification system polynucleotides can be operably linked, fused to, or otherwise modified to include a polynucleotide that encodes or is a selectable marker or tag, which can be a polynucleotide or polypeptide. In some embodiments, the polypeptide encoding a polypeptide selectable marker can be incorporated in the engineered nucleic acid modification polynucleotide such that the selectable marker polypeptide, when translated, is inserted between two amino acids between the N- and C-terminus of the engineered nucleic acid modification polypeptide or at the N- and/or C-terminus of the engineered nucleic acid modification polypeptide. In some embodiments, the selectable marker or tag is a polynucleotide barcode or unique molecular identifier (UMI).

It will be appreciated that the polynucleotide encoding such selectable markers or tags can be incorporated into a polynucleotide encoding one or more components of the engineered nucleic acid modification described herein in an appropriate manner to allow expression of the selectable marker or tag. Such techniques and methods are described elsewhere herein and will be instantly appreciated by one of ordinary skill in the art in view of this disclosure. Many such selectable markers and tags are generally known in the art and are intended to be within the scope of this disclosure.

Suitable selectable markers and tags include, but are not limited to, affinity tags, such as chitin binding protein (CBP), maltose binding protein (MBP), glutathione-S-transferase (GST), poly(His) tag; solubilization tags such as thioredoxin (TRX) and poly(NANP), MBP, and GST; chromatography tags such as those consisting of polyanionic amino acids, such as FLAG-tag; epitope tags such as V5-tag, Myc-tag, HA-tag and NE-tag; protein tags that can allow specific enzymatic modification (such as biotinylation by biotin ligase) or chemical modification (such as reaction with FlAsH-EDT2 for fluorescence imaging), DNA and/or RNA segments that contain restriction enzyme or other enzyme cleavage sites; DNA segments that encode products that provide resistance against otherwise toxic compounds including antibiotics, such as, spectinomycin, ampicillin, kanamycin, tetracycline, Basta, neomycin phosphotransferase II (NEO), hygromycin phosphotransferase (HPT)) and the like; DNA and/or RNA segments that encode products that are otherwise lacking in the recipient cell (e.g., tRNA genes, auxotrophic markers); DNA and/or RNA segments that encode products which can be readily identified (e.g., phenotypic markers such as β-galactosidase, GUS; fluorescent proteins such as green fluorescent protein (GFP), cyan (CFP), yellow (YFP), red (RFP), luciferase, and cell surface proteins); polynucleotides that can generate one or more new primer sites for PCR (e.g., the juxtaposition of two DNA sequences not previously juxtaposed), DNA sequences not acted upon or acted upon by a restriction endonuclease or other DNA modifying enzyme, chemical, etc.; epitope tags (e.g. GFP, FLAG- and His-tags), and, DNA sequences that make a molecular barcode or unique molecular identifier (UMI), DNA sequences required for a specific modification (e.g., methylation) that allows its identification. Other suitable markers will be appreciated by those of skill in the art.

Selectable markers and tags can be operably linked to one or more components of the engineered nucleic acid modification system described herein via suitable linker, such as a glycine or glycine serine linkers as short as GS or GG up to (GGGGG)₃ (SEQ ID NO: 39) or (GGGGS)₃ (SEQ ID NO: 5). Other suitable linkers are described elsewhere herein.

The vector or vector system can include one or more polynucleotides encoding one or more targeting moieties. In some embodiments, the targeting moiety encoding polynucleotides can be included in the vector or vector system, such as a viral vector system, such that they are expressed within and/or on the virus particle(s) produced such that the virus particles can be targeted to specific cells, tissues, organs, etc. In some embodiments, the targeting moiety encoding polynucleotides can be included in the vector or vector system such that the engineered nucleic acid modification system polynucleotide(s) and/or products expressed therefrom include the targeting moiety and can be targeted to specific cells, tissues, organs, etc. In some embodiments, such as non-viral carriers, the targeting moiety can be attached to the carrier (e.g., polymer, lipid, inorganic molecule etc.) and can be capable of targeting the carrier and any attached or associated engineered nucleic acid modification system polynucleotide(s) to specific cells, tissues, organs, etc.

### Codon Optimization of Vector Polynucleotides

As described elsewhere herein, the polynucleotide encoding one or more embodiments of the engineered nucleic acid modification system described herein can be codon optimized. In some embodiments, one or more polynucleotides contained in a vector (“vector polynucleotides”) described herein that are in addition to an optionally codon optimized polynucleotide encoding embodiments of the engineered nucleic acid modification system described herein can be codon optimized. In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a DNA/RNA-targeting Cas protein or other component of the engineered nucleic acid modification system of the present invention described herein corresponds to the most frequently used codon for a particular amino acid. As to codon usage in yeast, reference is made to the online Yeast Genome database available at http://www.yeastgenome.org/community/codon_usage.shtml, or Codon selection in yeast, Bennetzen and Hall, J Biol Chem. 1982 Mar. 25; 257(6):3026-31. As to codon usage in plants including algae, reference is made to Codon usage in higher plants, green algae, and cyanobacteria, Campbell and Gowri, Plant Physiol. 1990 January; 92(1): 1-11.; as well as Codon usage in plant genes, Murray et al, Nucleic Acids Res. 1989 Jan. 25; 17(2):477-98; or Selection on the codon bias of chloroplast and cyanelle genes in different plant and algal lineages, Morton B R, J Mol Evol. 1998 April; 46(4):449-59.

The vector polynucleotide can be codon optimized for expression in a specific cell-type, tissue type, organ type, and/or subject type. In some embodiments, a codon optimized sequence is a sequence optimized for expression in a eukaryote, e.g., humans (i.e., being optimized for expression in a human or human cell), or for another eukaryote, such as another animal (e.g., a mammal or avian) as is described elsewhere herein. Such codon optimized sequences are within the ambit of the ordinary skilled artisan in view of the description herein. In some embodiments, the polynucleotide is codon optimized for a specific cell type. Such cell types can include, but are not limited to, epithelial cells (including skin cells, cells lining the gastrointestinal tract, cells lining other hollow organs), nerve cells (nerves, brain cells, spinal column cells, nerve support cells (e.g., astrocytes, glial cells, Schwann cells etc.), muscle cells (e.g., cardiac muscle, smooth muscle cells, and skeletal muscle cells), connective tissue cells (e.g., fat and other soft tissue padding cells, bone cells, tendon cells, cartilage cells), blood cells, stem cells and other progenitor cells, immune system cells, germ cells, and combinations thereof. Such codon optimized sequences are within the ambit of the ordinary skilled artisan in view of the description herein. In some embodiments, the polynucleotide is codon optimized for a specific tissue type. Such tissue types can include, but are not limited to, muscle tissue, connective tissue, connective tissue, nervous tissue, and epithelial tissue. Such codon optimized sequences are within the ambit of the ordinary skilled artisan in view of the description herein. In some embodiments, the polynucleotide is codon optimized for a specific organ. Such organs include, but are not limited to, muscles, skin, intestines, liver, spleen, brain, lungs, stomach, heart, kidneys, gallbladder, pancreas, bladder, thyroid, bone, blood vessels, blood, and combinations thereof. Such codon optimized sequences are within the ambit of the ordinary skilled artisan in view of the description herein.

In some embodiments, a vector polynucleotide is codon optimized for expression in particular cells, such as prokaryotic or eukaryotic cells. The eukaryotic cells may be those of or derived from a particular organism, such as a plant or a mammal, including but not limited to human, or non-human eukaryote or animal or mammal as discussed herein, e.g., mouse, rat, rabbit, dog, livestock, or non-human mammal or primate.

### Vector Construction

The vectors described herein can be constructed using any suitable process or technique. In some embodiments, one or more suitable recombination and/or cloning methods or techniques can be used to the vector(s) described herein. Suitable recombination and/or cloning techniques and/or methods can include, but not limited to, those described in U.S. Patent Publication No. US 2004/0171156 A1. Other suitable methods and techniques are described elsewhere herein.

Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989). Any of the techniques and/or methods can be used and/or adapted for constructing an AAV or other vector described herein. AAV vectors are discussed in greater detail elsewhere herein.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. When multiple different guide polynucleotides are used, a single expression construct may be used to target nucleic acid-targeting activity to multiple different, corresponding target sequences within a cell. For example, a single vector may comprise about or more than about 1, 2, 3, 4, 5, 6, 7, 8, 9, 10, 15, 20, or more guide s polynucleotides. In some embodiments, about or more than about 1, 2, 3, 4, 5, 6, 7, 8, 9, 10, or more such guide-polynucleotide-containing vectors may be provided, and optionally delivered to a cell.

Delivery vehicles, vectors, particles, nanoparticles, formulations and components thereof for expression of one or more elements of a CRISPR-Cas system described herein are as used in the foregoing documents, such as International Patent Publication No. WO 2014/093622 (PCT/US2013/074667) and are discussed in greater detail herein.

### Viral Vectors

In some embodiments, the vector is a viral vector. The term of art “viral vector” and as used herein in this context refers to polynucleotide based vectors that contain one or more elements from or based upon one or more elements of a virus that can be capable of expressing and packaging a polynucleotide, such as a engineered nucleic acid modification system polynucleotide of the present invention, into a virus particle and producing said virus particle when used alone or with one or more other viral vectors (such as in a viral vector system). Viral vectors and systems thereof can be used for producing viral particles for delivery of and/or expression of one or more components of the engineered nucleic acid modification system described herein. The viral vector can be part of a viral vector system involving multiple vectors. In some embodiments, systems incorporating multiple viral vectors can increase the safety of these systems. Suitable viral vectors can include retroviral-based vectors, lentiviral-based vectors, adenoviral-based vectors, adeno associated vectors, helper-dependent adenoviral (HdAd) vectors, hybrid adenoviral vectors, herpes simplex virus-based vectors, poxvirus-based vectors, and Epstein-Barr virus-based vectors. Other embodiments of viral vectors and viral particles produce therefrom are described elsewhere herein. In some embodiments, the viral vectors are configured to produce replication incompetent viral particles for improved safety of these systems.

In certain embodiments, the virus structural component, which can be encoded by one or more polynucleotides in a viral vector or vector system, comprises one or more capsid proteins including an entire capsid. In certain embodiments, such as wherein a viral capsid comprises multiple copies of different proteins, the delivery system can provide one or more of the same protein or a mixture of such proteins. For example, AAV comprises 3 capsid proteins, VP1, VP2, and VP3, thus delivery systems of the invention can comprise one or more of VP1, and/or one or more of VP2, and/or one or more of VP3. Accordingly, the present invention is applicable to a virus within the family Adenoviridae, such as Atadenovirus, e.g., Ovine atadenovirus D, Aviadenovirus, e.g., Fowl aviadenovirus A, Ichtadenovirus, e.g., Sturgeon ichtadenovirus A, Mastadenovirus (which includes adenoviruses such as all human adenoviruses), e.g., Human mastadenovirus C, and Siadenovirus, e.g., Frog siadenovirus A. Thus, a virus of within the family Adenoviridae is contemplated as within the invention with discussion herein as to adenovirus applicable to other family members. Target-specific AAV capsid variants can be used or selected. Non-limiting examples include capsid variants selected to bind to chronic myelogenous leukemia cells, human CD34 PBPC cells, breast cancer cells, cells of lung, heart, dermal fibroblasts, melanoma cells, stem cell, glioblastoma cells, coronary artery endothelial cells and keratinocytes. See, e.g., Buning et al, 2015, Current Opinion in Pharmacology 24, 94-104. From teachings herein and knowledge in the art as to modifications of adenovirus (see, e.g., U.S. Pat. Nos. 9,410,129, 7,344,872, 7,256,036, 6,911,199, 6,740,525; Matthews, “Capsid-Incorporation of Antigens into Adenovirus Capsid Proteins for a Vaccine Approach,” Mol Pharm, 8(1): 3-11 (2011)), as well as regarding modifications of AAV, the skilled person can readily obtain a modified adenovirus that has a large payload protein or a CRISPR-protein, despite that heretofore it was not expected that such a large protein could be provided on an adenovirus. And as to the viruses related to adenovirus mentioned herein, as well as to the viruses related to AAV mentioned elsewhere herein, the teachings herein as to modifying adenovirus and AAV, respectively, can be applied to those viruses without undue experimentation from this disclosure and the knowledge in the art.

In some embodiments, the viral vector is configured such that when the cargo is packaged the cargo(s) (e.g., one or more components of the CRISPR-Cas system, including but not limited to a Cas effector, is external to the capsid or virus particle. In the sense that it is not inside the capsid (enveloped or encompassed with the capsid) but is externally exposed so that it can contact the target genomic DNA. In some embodiments, the viral vector is configured such that all the carog(s) are contained within the capsid after packaging.

### Split Viral Vector Systems

When the engineered nucleic acid modification system viral vector or vector system (be it a retroviral (e.g., AAV) or lentiviral vector) is designed so as to position the cargo(s) (e.g., one or more CRISPR-Cas system components) at the internal surface of the capsid once formed, the cargo(s) will fill most or all of internal volume of the capsid. In other embodiments, an engineered nucleic acid modification system polypeptide may be modified or divided so as to occupy a less of the capsid internal volume. Accordingly, in certain embodiments, the engineered nucleic acid modification system or a component thereof (e.g., a DNA polymerase, a site-specific nuclease polypeptide (e.g., a Cas polypeptide), a Vir polypeptide (e.g., a VirD1 or VirD2) or any combination thereof) can be divided in two or more portions, one portion included in one viral particle or capsid and the second (or more) portion included in a second or more viral particles or capsids. In certain embodiments, by splitting the engineered nucleic acid modification system or a component thereof thereof in two portions, space is made available to link one or more heterologous domains to one or both the engineered nucleic acid modification system component (e.g., a DNA polymerase, a site-specific nuclease polypeptide (e.g., a Cas polypeptide), a Vir polypeptide (e.g., a VirD1 or VirD2) or any combination thereof) portions. Such systems can be referred to as “split vector systems” or in the context of the present disclosure a “engineered nucleic acid modification system” a “split CRISPR protein”, a “split Cas protein”, a “split VirD2”, a “split DNA polymerase”, and the like. This split protein approach is also described elsewhere herein. When the concept is applied to a vector system, it thus describes putting pieces of the split proteins on different vectors thus reducing the payload of any one vector. This approach can facilitate delivery of systems where the total system size is close to or exceeds the packaging capacity of the vector. This is independent of any regulation of the engineered nucleic acid modification system that can be achieved with a split system or split protein design.

Split engineered nucleic acid modification system proteins that can be incorporated into the AAV or other vectors described herein are set forth elsewhere herein and in documents incorporated herein by reference in further detail herein. In certain embodiments, each part of a split engineered nucleic acid modification system proteins are attached to a member of a specific binding pair, and when bound with each other, the members of the specific binding pair maintain the parts of the engineered nucleic acid modification system protein in proximity. In certain embodiments, each part of a split engineered nucleic acid modification system protein is associated with an inducible binding pair. An inducible binding pair is one which is capable of being switched “on” or “off” by a protein or small molecule that binds to both members of the inducible binding pair. In general, according to the invention, engineered nucleic acid modification system proteins may preferably split between domains, leaving domains intact. Preferred, non-limiting examples of split CRISPR proteins that can be used in the engineered nucleic acid modification system include, without limitation, Cas protein, and orthologues. Preferred, non-limiting examples of split points include, with reference to SpCas9: a split position between 202A/2035; a split position between 255F/256D; a split position between 310E/311I; a split position between 534R/535K; a split position between 572E/573C; a split position between 7135/714G; a split position between 1003L/104E; a split position between 1054G/1055E; a split position between 1114N/1115S; a split position between 1152K/1153S; a split position between 1245K/1246G; or a split between 1098 and 1099. Corresponding positions in other Cas proteins can be appreciated in view of these positions made with reference to SpCas9.

In some embodiments, any AAV serotype is preferred. In some embodiments, the VP2 domain associated with the engineered nucleic acid modification system protein(s) is an AAV serotype 2 VP2 domain. In some embodiments, the VP2 domain associated with the engineered nucleic acid modification system protein (s) is an AAV serotype 8 VP2 domain. The serotype can be a mixed serotype as is known in the art.

### Retroviral and Lentiviral Vectors

Retroviral vectors can be composed of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Suitable retroviral vectors for the engineered nucleic acid modification system can include those based upon murine leukemia virus (MuLV), gibbon ape leukemia virus (GaLV), Simian immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Selection of a retroviral gene transfer system may therefore depend on the target tissue.

The tropism of a retrovirus can be altered by incorporating foreign envelope proteins, expanding the potential target population of target cells. Lentiviral vectors are retroviral vectors that are able to transduce or infect non-dividing cells and are described in greater detail elsewhere herein. A retrovirus can also be engineered to allow for conditional expression of the inserted transgene, such that only certain cell types are infected by the lentivirus.

Lentiviruses are complex retroviruses that have the ability to infect and express their genes in both mitotic and post-mitotic cells. Advantages of using a lentiviral approach can include the ability to transduce or infect non-dividing cells and their ability to typically produce high viral titers, which can increase efficiency or efficacy of production and delivery. Suitable lentiviral vectors include, but are not limited to, human immunodeficiency virus (HIV)-based lentiviral vectors, feline immunodeficiency virus (FIV)-based lentiviral vectors, simian immunodeficiency virus (SIV)-based lentiviral vectors, Moloney Murine Leukaemia Virus (Mo-MLV), Visna.maedi virus (VMV)-based lentiviral vector, carpine arthritis-encephalitis virus (CAEV)-based lentiviral vector, bovine immune deficiency virus (BIV)-based lentiviral vector, and Equine infectious anemia (EIAV)-based lentiviral vector. In some embodiments, an HIV-based lentiviral vector system can be used. In some embodiments, a FIV-based lentiviral vector system can be used.

In some embodiments, the lentiviral vector is an EIAV-based lentiviral vector or vector system. EIAV vectors have been used to mediate expression, packaging, and/or delivery in other contexts, such as for ocular gene therapy (see, e.g., Balagaan, J Gene Med 2006; 8: 275-285). In another embodiment, RetinoStat®, (see, e.g., Binley et al., HUMAN GENE THERAPY 23:980-991 (September 2012)), which describes RetinoStat®, an equine infectious anemia virus-based lentiviral gene therapy vector that expresses angiostatic proteins endostatin and angiostatin that is delivered via a subretinal injection for the treatment of the wet form of age-related macular degeneration. Any of these vectors described in these publications can be modified for the elements of the engineered nucleic acid modification system system described herein.

In some embodiments, the lentiviral vector or vector system thereof can be a first-generation lentiviral vector or vector system thereof. First-generation lentiviral vectors can contain a large portion of the lentivirus genome, including the gag and pol genes, other additional viral proteins (e.g., VSV-G) and other accessory genes (e.g., vif, vprm vpu, nef, and combinations thereof), regulatory genes (e.g., tat and/or rev) as well as the gene of interest between the LTRs. First generation lentiviral vectors can result in the production of virus particles that can be capable of replication in vivo, which may not be appropriate for some instances or applications.

In some embodiments, the lentiviral vector or vector system thereof can be a second-generation lentiviral vector or vector system thereof. Second-generation lentiviral vectors do not contain one or more accessory virulence factors and do not contain all components necessary for virus particle production on the same lentiviral vector. This can result in the production of a replication-incompetent virus particle and thus increase the safety of these systems over first-generation lentiviral vectors. In some embodiments, the second-generation vector lacks one or more accessory virulence factors (e.g., vif, vprm, vpu, nef, and combinations thereof). Unlike the first-generation lentiviral vectors, no single second generation lentiviral vector includes all features necessary to express and package a polynucleotide into a virus particle. In some embodiments, the envelope and packaging components are split between two different vectors with the gag, pol, rev, and tat genes being contained on one vector and the envelope protein (e.g., VSV-G) are contained on a second vector. The gene of interest, its promoter, and LTRs can be included on a third vector that can be used in conjunction with the other two vectors (packaging and envelope vectors) to generate a replication-incompetent virus particle.

In some embodiments, the lentiviral vector or vector system thereof can be a third-generation lentiviral vector or vector system thereof. Third-generation lentiviral vectors and vector systems thereof have increased safety over first- and second-generation lentiviral vectors and systems thereof because, for example, the various components of the viral genome are split between two or more different vectors but used together in vitro to make virus particles, they can lack the tat gene (when a constitutively active promoter is included up-stream of the LTRs), and they can include one or more deletions in the 3′LTR to create self-inactivating (SIN) vectors having disrupted promoter/enhancer activity of the LTR. In some embodiments, a third-generation lentiviral vector system can include (i) a vector plasmid that contains the polynucleotide of interest and upstream promoter that are flanked by the 5′ and 3′ LTRs, which can optionally include one or more deletions present in one or both of the LTRs to render the vector self-inactivating; (ii) a “packaging vector(s)” that can contain one or more genes involved in packaging a polynucleotide into a virus particle that is produced by the system (e.g., gag, pol, and rev) and upstream regulatory sequences (e.g., promoter(s)) to drive expression of the features present on the packaging vector, and (iii) an “envelope vector” that contains one or more envelope protein genes and upstream promoters. In certain embodiments, the third-generation lentiviral vector system can include at least two packaging vectors, with the gag-pol being present on a different vector than the rev gene.

In some embodiments, self-inactivating lentiviral vectors with an siRNA targeting a common exon shared by HIV tat/rev, a nucleolar-localizing TAR decoy, and an anti-CCR5-specific hammerhead ribozyme (see, e.g., DiGiusto et al. (2010) Sci Transl Med 2:36ra43) can be used/and or adapted to the engineered nucleic acid modification system of the present invention.

In some embodiments, the pseudotype and infectivity or tropisim of a lentivirus particle can be tuned by altering the type of envelope protein(s) included in the lentiviral vector or system thereof. As used herein, an “envelope protein” or “outer protein” means a protein exposed at the surface of a viral particle that is not a capsid protein. For example, envelope or outer proteins typically comprise proteins embedded in the envelope of the virus. In some embodiments, a lentiviral vector or vector system thereof can include a VSV-G envelope protein. VSV-G mediates viral attachment to an LDL receptor (LDLR) or an LDLR family member present on a host cell, which triggers endocytosis of the viral particle by the host cell. Because LDLR is expressed by a wide variety of cells, viral particles expressing the VSV-G envelope protein can infect or transduce a wide variety of cell types. Other suitable envelope proteins can be incorporated based on the host cell that a user desires to be infected by a virus particle produced from a lentiviral vector or system thereof described herein and can include, but are not limited to, feline endogenous virus envelope protein (RD 114) (see e.g., Hanawa et al. Molec. Ther. 2002 5(3) 242-251), modified Sindbis virus envelope proteins (see e.g., Morizono et al. 2010. J. Virol. 84(14) 6923-6934; Morizono et al. 2001. J. Virol. 75:8016-8020; Morizono et al. 2009. J. Gene Med. 11:549-558; Morizono et al. 2006 Virology 355:71-81; Morizono et al J. Gene Med. 11:655-663, Morizono et al. 2005 Nat. Med. 11:346-352), baboon retroviral envelope protein (see e.g., Girard-Gagnepain et al. 2014. Blood. 124: 1221-1231); Tupaia paramyxovirus glycoproteins (see e.g., Enkirch T. et al., 2013. Gene Ther. 20:16-23); measles virus glycoproteins (see e.g., Funke et al. 2008. Molec. Ther. 16(8): 1427-1436), rabies virus envelope proteins, MLV envelope proteins, Ebola envelope proteins, baculovirus envelope proteins, filovirus envelope proteins, hepatitis E1 and E2 envelope proteins, gp41 and gp120 of HIV, hemagglutinin, neuraminidase, M2 proteins of influenza virus, and combinations thereof.

In some embodiments, the tropism of the resulting lentiviral particle can be tuned by incorporating cell targeting peptides into a lentiviral vector such that the cell targeting peptides are expressed on the surface of the resulting lentiviral particle. In some embodiments, a lentiviral vector can contain an envelope protein that is fused to a cell targeting protein (see e.g., Buchholz et al. 2015. Trends Biotechnol. 33:777-790; Bender et al. 2016. PLoS Pathog. 12(e1005461); and Friedrich et al. 2013. Mol. Ther. 2013. 21: 849-859.

In some embodiments, a split-intein-mediated approach to target lentiviral particles to a specific cell type can be used (see e.g., Chamoun-Emaneulli et al. 2015. Biotechnol. Bioeng. 112:2611-2617, Ramirez et al. 2013. Protein. Eng. Des. Sel. 26:215-233. In these embodiments, a lentiviral vector can contain one half of a splicing-deficient variant of the naturally split intein from Nostoc punctiforme fused to a cell targeting peptide and the same or different lentiviral vector can contain the other half of the split intein fused to an envelope protein, such as a binding-deficient, fusion-competent virus envelope protein. This can result in production of a virus particle from the lentiviral vector or vector system that includes a split intein that can function as a molecular Velcro linker to link the cell-binding protein to the pseudotyped lentivirus particle. This approach can be advantageous for use where surface-incompatibilities can restrict the use of, e.g., cell targeting peptides.

In some embodiments, a covalent-bond-forming protein-peptide pair can be incorporated into one or more of the lentiviral vectors described herein to conjugate a cell targeting peptide to the virus particle (see e.g., Kasaraneni et al. 2018. Sci. Reports (8) No. 10990). In some embodiments, a lentiviral vector can include an N-terminal PDZ domain of InaD protein (PDZ1) and its pentapeptide ligand (TEFCA) from NorpA, which can conjugate the cell targeting peptide to the virus particle via a covalent bond (e.g., a disulfide bond). In some embodiments, the PDZ1 protein can be fused to an envelope protein, which can optionally be binding deficient and/or fusion competent virus envelope protein and included in a lentiviral vector. In some embodiments, the TEFCA can be fused to a cell targeting peptide and the TEFCA-CPT fusion construct can be incorporated into the same or a different lentiviral vector as the PDZ1-envenlope protein construct. During virus production, specific interaction between the PDZ1 and TEFCA facilitates producing virus particles covalently functionalized with the cell targeting peptide and thus capable of targeting a specific cell-type based upon a specific interaction between the cell targeting peptide and cells expressing its binding partner. This approach can be advantageous for use where surface-incompatibilities can restrict the use of, e.g., cell targeting peptides.

Lentiviral vectors have been disclosed as in the treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors have also been disclosed for delivery to the brain, see, e.g., US Patent Publication Nos. US20110293571; US20110293571, US20040013648, US20070025970, US20090111106 and U.S. Pat. No. 7,259,015. Any of these systems or a variant thereof can be used to deliver an engineered nucleic acid modification system polynucleotide described herein to a cell.

In some embodiments, a lentiviral vector system can include one or more transfer plasmids. Transfer plasmids can be generated from various other vector backbones and can include one or more features that can work with other retroviral and/or lentiviral vectors in the system that can, for example, improve safety of the vector and/or vector system, increase virial titers, and/or increase or otherwise enhance expression of the desired insert to be expressed and/or packaged into the viral particle. Suitable features that can be included in a transfer plasmid can include, but are not limited to, 5′LTR, 3′LTR, SIN/LTR, origin of replication (Ori), selectable marker genes (e.g., antibiotic resistance genes), Psi (Ψ), RRE (rev response element), cPPT (central polypurine tract), promoters, WPRE (woodchuck hepatitis post-transcriptional regulatory element), SV40 polyadenylation signal, pUC origin, SV40 origin, F1 origin, and combinations thereof.

In another embodiment, Cocal vesiculovirus envelope pseudotyped retroviral or lentiviral vector particles are contemplated (see, e.g., US Patent Publication No. 20120164118 assigned to the Fred Hutchinson Cancer Research Center). Cocal virus is in the Vesiculovirus genus and is a causative agent of vesicular stomatitis in mammals. Cocal virus was originally isolated from mites in Trinidad (see e.g., Jonkers et al., Am. J. Vet. Res. 25:236-242 (1964)), and infections have been identified in Trinidad, Brazil, and Argentina from insects, cattle, and horses. Many of the vesiculoviruses that infect mammals have been isolated from naturally infected arthropods, suggesting that they are vector-borne. Antibodies to vesiculoviruses are common among people living in rural areas where the viruses are endemic and laboratory-acquired; infections in humans usually result in influenza-like symptoms. The Cocal virus envelope glycoprotein shares 71.5% identity at the amino acid level with VSV-G Indiana, and phylogenetic comparison of the envelope gene of vesiculoviruses shows that Cocal virus is serologically distinct from, but most closely related to, VSV-G Indiana strains among the vesiculoviruses (see e.g., Jonkers et al., Am. J. Vet. Res. 25:236-242 (1964) and Travassos da Rosa et al., Am. J. Tropical Med. & Hygiene 33:999-1006 (1984)). The Cocal vesiculovirus envelope pseudotyped retroviral vector particles may include for example, lentiviral, alpharetroviral, betaretroviral, gammaretroviral, deltaretroviral, and epsilonretroviral vector particles that may comprise retroviral Gag, Pol, and/or one or more accessory protein(s) and a Cocal vesiculovirus envelope protein. In certain embodiments of these embodiments, the Gag, Pol, and accessory proteins are lentiviral and/or gammaretroviral. In some embodiments, a retroviral vector can contain encoding polypeptides for one or more Cocal vesiculovirus envelope proteins such that the resulting viral or pseudoviral particles are Cocal vesiculovirus envelope pseudotyped.

### Adenoviral Vectors, Helper-Dependent Adenoviral Vectors, and Hybrid Adenoviral Vectors

In some embodiments, the vector is an adenoviral vector. In some embodiments, the adenoviral vector includes elements such that the virus particle produced using the vector or system thereof can be serotype 2 or serotype 5. In some embodiments, the polynucleotide to be delivered via the adenoviral particle can be up to about 8 kb in size. Thus, in some embodiments, an adenoviral vector can include a DNA polynucleotide to be delivered that can range in size from about 0.001 kb to about 8 kb. Adenoviral vectors have been used successfully in several contexts (see e.g., Teramato et al. 2000. Lancet. 355:1911-1912; Lai et al. 2002. DNA Cell. Biol. 21:895-913; Flotte et al., 1996. Hum. Gene. Ther. 7:1145-1159; and Kay et al. 2000. Nat. Genet. 24:257-261).

In some embodiments the vector can be a helper-dependent adenoviral vector or system thereof. These are also referred to in the art as “gutless” or “gutted” vectors and are a modified generation of adenoviral vectors (see e.g., Thrasher et al. 2006. Nature. 443:E5-7). In certain embodiments of the helper-dependent adenoviral vector system one vector (the helper) can contain all the viral genes required for replication but contains a conditional gene defect in the packaging domain. The second vector of the system can contain only the ends of the viral genome, one or more engineered nucleic acid modification system polynucleotides, and the native packaging recognition signal, which can allow selective packaged release from the cells (see e.g., Cideciyan et al. 2009. N Engl J Med. 361:725-727). Helper-dependent adenoviral vector systems have been successful for gene delivery in several contexts (see e.g., Simonelli et al. 2010. J Am Soc Gene Ther. 18:643-650; Cideciyan et al. 2009. N Engl J Med. 361:725-727; Crane et al. 2012. Gene Ther. 19(4):443-452; Alba et al. 2005. Gene Ther. 12:18-S27; Croyle et al. 2005. Gene Ther. 12:579-587; Amalfitano et al. 1998. J. Virol. 72:926-933; and Morral et al. 1999. PNAS. 96:12816-12821). The techniques and vectors described in these publications can be adapted for inclusion and delivery of the engineered nucleic acid modification system polynucleotides described herein. In some embodiments, the polynucleotide to be delivered via the viral particle produced from a helper-dependent adenoviral vector or system thereof can be up to about 37 kb. Thus, in some embodiments, an adenoviral vector can include a DNA polynucleotide to be delivered that can range in size from about 0.001 kb to about 37 kb (see e.g., Rosewell et al. 2011. J. Genet. Syndr. Gene Ther. Suppl. 5:001).

In some embodiments, the vector is a hybrid-adenoviral vector or system thereof. Hybrid adenoviral vectors are composed of the high transduction efficiency of a gene-deleted adenoviral vector and the long-term genome-integrating potential of adeno-associated, retroviruses, lentivirus, and transposon based-gene transfer. In some embodiments, such hybrid vector systems can result in stable transduction and limited integration site. See e.g., Balague et al. 2000. Blood. 95:820-828; Morral et al. 1998. Hum. Gene Ther. 9:2709-2716; Kubo and Mitani. 2003. J. Virol. 77(5): 2964-2971; Zhang et al. 2013. PloS One. 8(10) e76771; and Cooney et al. 2015. Mol. Ther. 23(4):667-674), whose techniques and vectors described therein can be modified and adapted for use in the engineered nucleic acid modification system of the present invention. In some embodiments, a hybrid-adenoviral vector can include one or more features of a retrovirus and/or an adeno-associated virus. In some embodiments the hybrid-adenoviral vector can include one or more features of a spuma retrovirus or foamy virus (FV). See e.g., Ehrhardt et al. 2007. Mol. Ther. 15:146-156 and Liu et al. 2007. Mol. Ther. 15:1834-1841, whose techniques and vectors described therein can be modified and adapted for use in the engineered nucleic acid modification system of the present invention. Advantages of using one or more features from the FVs in the hybrid-adenoviral vector or system thereof can include the ability of the viral particles produced therefrom to infect a broad range of cells, a large packaging capacity as compared to other retroviruses, and the ability to persist in quiescent (non-dividing) cells. See also e.g., Ehrhardt et al. 2007. Mol. Ther. 156:146-156 and Shuji et al. 2011. Mol. Ther. 19:76-82, whose techniques and vectors described therein can be modified and adapted for use in the engineered nucleic acid modification system of the present invention.

### Adeno Associated Viral (AAV) Vectors

In an embodiment, the vector can be an adeno-associated virus (AAV) vector. See, e.g., West et al., Virology 160:38-47 (1987); U.S. Pat. No. 4,797,368; WO 93/24641; Kotin, Human Gene Therapy 5:793-801 (1994); and Muzyczka, J. Clin. Invest. 94:1351 (1994). Although similar to adenoviral vectors in some of their features, AAVs have some deficiency in their replication and/or pathogenicity and thus can be safer that adenoviral vectors. In some embodiments the AAV can integrate into a specific site on chromosome 19 of a human cell with no observable side effects. In some embodiments, the capacity of the AAV vector, system thereof, and/or AAV particles can be up to about 4.7 kb. In some embodiments, utilizing homologs of the Cas effector protein that are shorter can be utilized, such for example those in

TABLE 1
Exemplary shorter Cas effector homologs.

| Species | Cas9 Size (nt) |
|---|---|
| Corynebacter diphtheriae | 3252 |
| Eubacterium ventriosum | 3321 |
| Streptococcus pasteurianus | 3390 |
| Lactobacillus farciminis | 3378 |
| Sphaerochaeta globus | 3537 |
| Azospirillum B510 | 3504 |
| Gluconacetobacter diazotrophicus | 3150 |
| Neisseria cinerea | 3246 |
| Roseburia intestinalis | 3420 |
| Parvibaculum lavamentivorans | 3111 |
| Staphylococcus aureus | 3159 |
| Nitratifractor salsuginis DSM 16511 | 3396 |
| Campylobacter lari CF89-12 | 3009 |
| Campylobacter jejuni | 2952 |
| Streptococcus thermophilus LMD-9 | 3396 |

The AAV vector or system thereof can include one or more regulatory molecules. In some embodiments the regulatory molecules can be promoters, enhancers, repressors and the like, which are described in greater detail elsewhere herein. In some embodiments, the AAV vector or system thereof can include one or more polynucleotides that can encode one or more regulatory proteins. In some embodiments, the one or more regulatory proteins can be selected from Rep78, Rep68, Rep52, Rep40, variants thereof, and combinations thereof.

The AAV vector or system thereof can include one or more polynucleotides that can encode one or more capsid proteins. The capsid proteins can be selected from VP1, VP2, VP3, and combinations thereof. The capsid proteins can be capable of assembling into a protein shell of the AAV virus particle. In some embodiments, the AAV capsid can contain 60 capsid proteins. In some embodiments, the ratio of VP1:VP2:VP3 in a capsid can be about 1:1:10.

In some embodiments, the AAV vector or system thereof can include one or more adenovirus helper factors or polynucleotides that can encode one or more adenovirus helper factors. Such adenovirus helper factors can include, but are not limited, E1A, E1B, E2A, E40RF6, and VA RNAs. In some embodiments, a producing host cell line expresses one or more of the adenovirus helper factors.

The AAV vector or system thereof can be configured to produce AAV particles having a specific serotype. In some embodiments, the serotype can be AAV-1, AAV-2, AAV-3, AAV-4, AAV-5, AAV-6, AAV-8, AAV-9 or any combinations thereof. In some embodiments, the AAV can be AAV1, AAV-2, AAV-5 or any combination thereof. One can select the AAV of the AAV with regard to the cells to be targeted; e.g., one can select AAV serotypes 1, 2, 5 or a hybrid capsid AAV-1, AAV-2, AAV-5 or any combination thereof for targeting brain and/or neuronal cells; and one can select AAV-4 for targeting cardiac tissue; and one can select AAV8 for delivery to the liver. Thus, in some embodiments, an AAV vector or system thereof capable of producing AAV particles capable of targeting the brain and/or neuronal cells can be configured to generate AAV particles having serotypes 1, 2, 5 or a hybrid capsid AAV-1, AAV-2, AAV-5 or any combination thereof. In some embodiments, an AAV vector or system thereof capable of producing AAV particles capable of targeting cardiac tissue can be configured to generate an AAV particle having an AAV-4 serotype. In some embodiments, an AAV vector or system thereof capable of producing AAV particles capable of targeting the liver can be configured to generate an AAV having an AAV-8 serotype. In some embodiments, the AAV vector is a hybrid AAV vector or system thereof. Hybrid AAVs are AAVs that include genomes with elements from one serotype that are packaged into a capsid derived from at least one different serotype. For example, if it is the rAAV2/5 that is to be produced, and if the production method is based on the helper-free, transient transfection method discussed above, the 1st plasmid and the 3rd plasmid (the adeno helper plasmid) will be the same as discussed for rAAV2 production. However, the second plasmid, the pRepCap will be different. In this plasmid, called pRep2/Cap5, the Rep gene is still derived from AAV2, while the Cap gene is derived from AAV5. The production scheme is the same as the above-mentioned approach for AAV2 production. The resulting rAAV is called rAAV2/5, in which the genome is based on recombinant AAV2, while the capsid is based on AAV5. It is assumed the cell or tissue-tropism displayed by this AAV2/5 hybrid virus should be the same as that of AAV5.

A tabulation of certain AAV serotypes as to these cells can be found in Grimm, D. et al, J. Virol. 82: 5887-5911 (2008), which is recapitulated in Table 2 below.

TABLE 2

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

In some embodiments, the AAV vector or system thereof is configured as a “gutless” vector, similar to that described in connection with a retroviral vector. In some embodiments, the “gutless” AAV vector or system thereof can have the cis-acting viral DNA elements involved in genome amplification and packaging in linkage with the heterologous sequences of interest (e.g., the engineered nucleic acid modification system polynucleotide(s) of the present invention).

In some embodiments, the AAV vectors are produced in in insect cells, e.g., Spodoptera frugiperda Sf9 insect cells, grown in serum-free suspension culture. Serum-free insect cells can be purchased from commercial vendors, e.g., Sigma Aldrich (EX-CELL 405).

In some embodiments, an AAV vector or vector system can contain or consists essentially of one or more polynucleotides encoding one or more components of a engineered nucleic acid modification system of the present invention. In some embodiments, the AAV vector or vector system can contain a plurality of cassettes comprising or consisting a first cassette comprising or consisting essentially of a promoter, a nucleic acid molecule encoding a engineered nucleic acid modification system polypeptide (e.g., a site-specific nuclease, a VirD1 or VirD2, a DNA polymerase, or other system polypeptide described herein and any combination thereof) and a terminator, and a two, or more, advantageously up to the packaging size limit of the vector, e.g., in total (including the first cassette) five, cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator . . . Promoter-gRNA(N)-terminator (where N is a number that can be inserted that is at an upper limit of the packaging size limit of the vector), or two or more individual rAAVs, each containing one or more than one cassette of a engineered nucleic acid modification system, e.g., a first rAAV containing the first cassette comprising or consisting essentially of a promoter, a nucleic acid molecule encoding one or more polypeptides of the engineered nucleic acid modification system of the present invention and a terminator, and a second rAAV containing a plurality, four, cassettes comprising or consisting essentially of a promoter, nucleic acid molecule encoding guide RNA (gRNA) and a terminator (e.g., each cassette schematically represented as Promoter-gRNA1-terminator, Promoter-gRNA2-terminator . . . Promoter-gRNA(N)-terminator (where N is a number that can be inserted that is at an upper limit of the packaging size limit of the vector). As rAAV is a DNA virus, the nucleic acid molecules in the herein discussion concerning AAV or rAAV are advantageously DNA. In some embodiments, the promoter is a tissue specific promoter or another tissue specific regulatory element. Suitable tissue specific regulatory elements, including promoters, are described in greater detail elsewhere herein.

In another embodiment, the invention provides an engineered nucleic acid modification system protein (e.g., a site-specific nuclease, DNA polymerase, a Vir protein (e.g., VirD1 or VirD2)) associated with Adeno Associated Virus (AAV), e.g., an AAV comprising an engineered nucleic acid modification system protein as a fusion, with or without a linker, to or with an AAV capsid protein such as VP1, VP2, and/or VP3. See e.g., Rybniker et al., “Incorporation of Antigens into Viral Capsids Augments Immunogenicity of Adeno-Associated Virus Vector-Based Vaccines,” J Virol. December 2012; 86(24): 13800-13804, Lux K, et al. 2005. Green fluorescent protein-tagged adeno-associated virus particles allow the study of cytosolic and nuclear trafficking. J. Virol. 79:11776-11787, Munch R C, et al. 2012. “Displaying high-affinity ligands on adeno-associated viral vectors enables tumor cell-specific and safe gene transfer.” Mol. Ther. doi:10.1038/mt.2012.186 and Warrington K H, Jr, et al. 2004. Adeno-associated virus type 2 VP2 capsid protein is nonessential and can tolerate large peptide insertions at its N terminus. J. Virol. 78:6595-6609), which are each incorporated herein by reference, and can be modified in view of the description herein to obtain an AAV capsid of the invention. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3). One can modify the cap gene to have expressed at a desired location a non-capsid protein advantageously a large payload protein, such as one or more engineered nucleic acid modification system proteins. Likewise, these can be fusions, with the protein, e.g., large payload protein such as one or more engineered nucleic acid modification system proteins fused in a manner analogous to prior art fusions. See e.g., US Patent Publication 20090215879; Nance et al., “Perspective on Adeno-Associated Virus Capsid Modification for Duchenne Muscular Dystrophy Gene Therapy,” Hum Gene Ther. 26(12):786-800 (2015) and documents cited therein, incorporated herein by reference. The skilled person, from this disclosure and the knowledge in the art can make and use modified AAV or AAV capsid as in the herein invention, and through this disclosure one knows now that large payload proteins can be fused to the AAV capsid. In some embodiments, one or more of the engineered nucleic acid modification system proteins are fused to a viral capsid (such as an AAV capsid) and those fusions can be a recombinant AAV that contains nucleic acid molecule(s) encoding or providing the one or more engineered nucleic acid modification system proteins or complexes and/or guide molecules, whereby the one or more engineered nucleic acid modification system protein is provided by the fusion to the capsid and the guide molecule is provided by coding of the recombinant virus, whereby in vivo, in a cell, the one or more engineered nucleic acid modification system proteins system is assembled from the nucleic acid molecule(s) of the recombinant virus providing the guide RNA and the outer surface of the virus providing the one or more engineered nucleic acid modification system protein.

The instant invention is also applicable to a virus in the genus Dependoparvovirus or in the family Parvoviridae, for instance, AAV, or a virus of Amdoparvovirus, e.g., Carnivore (enveloped or encompassed with the capsid) but is externally exposed so that it can contact the target genomic DNA). In some embodiments, the one or more engineered nucleic acid modification system proteins is/are associated with the AAV VP2 domain by way of a fusion protein. In some embodiments, the association may be considered to be a modification of the VP2 domain. Where reference is made herein to a modified VP2 domain, then this will be understood to include any association discussed herein of the VP2 domain and the one or more engineered nucleic acid modification system proteins. In some embodiments, the AAV VP2 domain may be associated (or tethered) to the one or more engineered nucleic acid modification system proteins via a connector protein, for example using a system such as the streptavidin-biotin system. In an embodiment, the present invention provides a polynucleotide encoding one or more engineered nucleic acid modification system proteins and associated AAV VP2 domain. In one embodiment, the invention provides a non-naturally occurring modified AAV having a VP2-engineered nucleic acid modification system protein capsid protein, wherein the one or more engineered nucleic acid modification system proteins is/are part of or tethered to the VP2 domain. In some preferred embodiments, the one or more engineered nucleic acid modification system proteins is/are fused to the VP2 domain so that, in another embodiment, the invention provides a non-naturally occurring modified AAV having a VP2-engineered nucleic acid modification system protein fusion capsid protein. Thus, a VP2-engineered nucleic acid modification system protein capsid protein may also include a VP2-engineered nucleic acid modification system protein fusion capsid protein. In some embodiments, the VP2-engineered nucleic acid modification system protein capsid protein further comprises a linker, whereby the VP2-engineered nucleic acid modification system protein is distanced from the remainder of the AAV. In some embodiments, the VP2-engineered nucleic acid modification system protein capsid protein further comprises at least one protein complex, e.g., engineered nucleic acid modification system complex, such as an engineered nucleic acid modification system complex guide RNA that targets a particular DNA, TALE, etc. An engineered nucleic acid modification system complex, such as engineered nucleic acid modification system comprising the VP2-engineered nucleic acid modification system protein capsid protein and at least one engineered nucleic acid modification system complex, such as a DNA polymerase, VirD2, CRISPR-Cas complex guide RNA that targets a particular DNA, is also provided in one embodiment.

In one embodiment, the invention provides a non-naturally occurring or engineered composition comprising an engineered nucleic acid modification system protein (e.g., a Cas, a DNA polymerase, a VirD protein (e.g., VirD1 or VirD2), which is part of or tethered to an AAV capsid domain, i.e., VP1, VP2, or VP3 domain of Adeno-Associated Virus (AAV) capsid. In some embodiments, part of or tethered to an AAV capsid domain includes associated with associated with a AAV capsid domain. In some embodiments, the engineered nucleic acid modification system protein may be fused to the AAV capsid domain. In some embodiments, the fusion may be to the N-terminal end of the AAV capsid domain. As such, in some embodiments, the C-terminal end of the engineered nucleic acid modification system protein is fused to the N-terminal end of the AAV capsid domain. In some embodiments, an NLS and/or a linker (such as a GlySer linker) may be positioned between the C-terminal end of the engineered nucleic acid modification system protein and the N-terminal end of the AAV capsid domain. In some embodiments, the fusion may be to the C-terminal end of the AAV capsid domain. In some embodiments, this is not preferred due to the fact that the VP1, VP2 and VP3 domains of AAV are alternative splices of the same RNA and so a C-terminal fusion may affect all three domains. In some embodiments, the AAV capsid domain is truncated. In some embodiments, some or all of the AAV capsid domain is removed. In some embodiments, some of the AAV capsid domain is removed and replaced with a linker (such as a GlySer linker), typically leaving the N-terminal and C-terminal ends of the AAV capsid domain intact, such as the first 2, 5 or 10 amino acids. In this way, the internal (non-terminal) portion of the VP3 domain may be replaced with a linker. It is particularly preferred that the linker is fused to the engineered nucleic acid modification system protein. A branched linker may be used, with the engineered nucleic acid modification system protein fused to the end of one of the branches. This allows for some degree of spatial separation between the capsid and the engineered nucleic acid modification system protein. In this way, the engineered nucleic acid modification system protein is part of (or fused to) the AAV capsid domain.

In other embodiments, the engineered nucleic acid modification system protein may be fused in frame within, i.e., internal to, the AAV capsid domain. Thus, in some embodiments, the AAV capsid domain again preferably retains its N-terminal and C-terminal ends. In this case, a linker is preferred, in some embodiments, either at one or both ends of the engineered nucleic acid modification system protein. In this way, the engineered nucleic acid modification system protein is again part of (or fused to) the AAV capsid domain. In certain embodiments, the positioning of the engineered nucleic acid modification system protein is such that the engineered nucleic acid modification system protein is at the external surface of the viral capsid once formed. In one embodiment, the invention provides a non-naturally occurring or engineered composition comprising an engineered nucleic acid modification system protein associated with a AAV capsid domain of Adeno-Associated Virus (AAV) capsid. Here, associated may mean in some embodiments fused, or in some embodiments bound to, or in some embodiments tethered to. The engineered nucleic acid modification system protein may, in some embodiments, be tethered to the VP1, VP2, or VP3 domain. This may be via a connector protein or tethering system such as the biotin-streptavidin system. In one example, a biotinylation sequence (15 amino acids) could therefore be fused to the engineered nucleic acid modification system protein. When a fusion of the AAV capsid domain, especially the N-terminus of the AAV AAV capsid domain, with streptavidin is also provided, the two will therefore associate with very high affinity. Thus, in some embodiments, provided is a composition or system comprising an engineered nucleic acid modification system protein-biotin fusion and a streptavidin-AAV capsid domain arrangement, such as a fusion. The engineered nucleic acid modification system protein-biotin and streptavidin-AAV capsid domain forms a single complex when the two parts are brought together. NLSs may also be incorporated between the engineered nucleic acid modification system protein and the biotin; and/or between the streptavidin and the AAV capsid domain.

As such, provided is a fusion of an engineered nucleic acid modification system protein with a connector protein specific for a high affinity ligand for that connector, whereas the AAV VP2 domain is bound to said high affinity ligand. For example, streptavidin may be the connector fused to the engineered nucleic acid modification system protein, while biotin may be bound to the AAV VP2 domain. Upon co-localization, the streptavidin will bind to the biotin, thus connecting the engineered nucleic acid modification system protein to the AAV VP2 domain. The reverse arrangement is also possible. In some embodiments, a biotinylation sequence (15 amino acids) could therefore be fused to the AAV VP2 domain, especially the N-terminus of the AAV VP2 domain. A fusion of the engineered nucleic acid modification system protein with streptavidin is also preferred, in some embodiments. In some embodiments, the biotinylated AAV capsids with streptavidin-CRISPR enzyme are assembled in vitro. This way the AAV capsids should assemble in a straightforward manner and the engineered nucleic acid modification system protein-streptavidin fusion can be added after assembly of the capsid. In other embodiments, a biotinylation sequence (15 amino acids) could therefore be fused to the engineered nucleic acid modification system protein, together with a fusion of the AAV VP2 domain, especially the N-terminus of the AAV VP2 domain, with streptavidin. For simplicity, a fusion of the engineered nucleic acid modification system protein and the AAV VP2 domain is preferred in some embodiments. In some embodiments, the fusion may be to the N-terminal end of the engineered nucleic acid modification system protein. In other words, in some embodiments, the AAV and engineered nucleic acid modification system protein are associated via fusion. In some embodiments, the AAV and engineered nucleic acid modification system protein are associated via fusion including a linker. Suitable linkers are discussed herein and include, without limitation, Gly Ser linkers. Fusion to the N-term of AAV VP2 domain is preferred, in some embodiments. In some embodiments, the CRISPR enzyme comprises at least one Nuclear Localization Signal (NLS). In a further embodiment, the present invention provides compositions comprising the CRISPR enzyme and associated AAV VP2 domain or the polynucleotides or vectors described herein. Such compositions and formulations are discussed elsewhere herein.

An alternative tether may be to fuse or otherwise associate the AAV capsid domain to an adaptor protein which binds to or recognizes to a corresponding RNA sequence or motif. In some embodiments, the adaptor is or comprises a binding protein which recognizes and binds (or is bound by) an RNA sequence specific for said binding protein. In some embodiments, a preferred example is the MS2 (see e.g., Konermann et al. December 2014, cited infra, incorporated herein by reference) binding protein which recognizes and binds (or is bound by) an RNA sequence specific for the MS2 protein.

With the AAV capsid domain associated with the adaptor protein, the engineered nucleic acid modification system protein may, in some embodiments, be tethered to the adaptor protein of the AAV capsid domain. The engineered nucleic acid modification system protein may, in some embodiments, be tethered to the adaptor protein of the AAV capsid domain via the engineered nucleic acid modification system protein being in a complex with a modified guide, see Konermann et al. The modified guide is, in some embodiments, a sgRNA. In some embodiments, the modified guide comprises a distinct RNA sequence; see, e.g., International Patent Application No. PCT/US14/70175, incorporated herein by reference.

In some embodiments, distinct RNA sequence is an aptamer. Thus, corresponding aptamer-adaptor protein systems are preferred. One or more functional domains may also be associated with the adaptor protein. An example of a preferred arrangement would be: [AAV AAV capsid domain—adaptor protein]—[modified guide—engineered nucleic acid modification system protein].

In certain embodiments, the positioning of the engineered nucleic acid modification system protein is such that the engineered nucleic acid modification system protein is at the internal surface of the viral capsid once formed. In one embodiment, the invention provides a non-naturally occurring or engineered composition comprising an engineered nucleic acid modification system protein associated with an internal surface of an AAV capsid domain. Here again, associated may mean in some embodiments fused, or in some embodiments bound to, or in some embodiments tethered to. The engineered nucleic acid modification system protein may, in some embodiments, be tethered to the VP1, VP2, or VP3 domain such that it locates to the internal surface of the viral capsid once formed. This may be via a connector protein or tethering system such as the biotin-streptavidin system as described above and/or elsewhere herein.

In one embodiment, the invention provides an engineered, non-naturally occurring engineered nucleic acid modification system comprising a AAV-engineered nucleic acid modification system protein (such as a Cas, VirD1 or VirD2, DNA polymerase, and any combination thereof), a donor construct, and a guide RNA that targets a DNA molecule encoding a gene product in a cell, whereby the guide RNA targets the DNA molecule encoding the gene product and the Cas protein optionally cleaves or nicks (or has no nuclease activity) and/or the VirD2 and/or DNA polymerase act to insert a donor polynucleotide from the donor construct into the DNA molecule encoding the gene product, whereby expression of the gene product is altered; and, wherein the Cas protein and the guide RNA do not naturally occur together. The invention comprehends the guide RNA comprising a guide sequence fused to a tracr sequence. In a preferred embodiment the Cas protein is a Cas9 protein. In some embodiments, the polynucleotide encoding the engineered nucleic acid modification system protein is codon optimized for expression in a eukaryotic cell. In some embodiments, the eukaryotic cell is a mammalian cell and in a more preferred embodiment the mammalian cell is a human cell. In a further embodiment, the expression of the gene product is decreased.

In another embodiment, the invention provides an engineered, non-naturally occurring vector system comprising one or more vectors comprising a first regulatory element operably linked to an engineered nucleic acid modification system protein system guide RNA that targets a DNA molecule encoding a gene product, and an AAV-engineered nucleic acid modification system protein, and donor construct. The components may be located on same or different vectors of the system or may be on the same vector whereby the AAV-engineered nucleic acid modification system protein also delivers the guide RNA of the engineered nucleic acid modification system. The guide RNA can targets the DNA molecule encoding the gene product in a cell and an AAV-engineered nucleic acid modification system protein may cleave or nick the DNA molecule encoding the gene product (it may cleave one or both strands or have substantially no nuclease activity) (e.g., a Cas), and the VirD1 and/or VirD2 and DNA polymerase act to insert a donor polynucleotide into the gene such that the gene or expression of the gene product is altered; and, wherein the AAV-engineered nucleic acid modification system protein and the guide RNA do not naturally occur together. The invention comprehends the guide RNA comprising a guide sequence fused to a tracr sequence. In an embodiment of the invention the AAV-engineered nucleic acid modification system protein is a type II AAV-engineered nucleic acid modification system protein. In an exemplary embodiment the AAV-engineered nucleic acid modification system protein is an AAV-Cas protein AAV-VirD2 polypeptide. In some embodiments the AAV-engineered nucleic acid modification system protein(s) is/are optimized for expression in a eukaryotic cell. In a preferred embodiment, the eukaryotic cell is a mammalian cell and in a more preferred embodiment the mammalian cell is a human cell. In a further embodiment of the invention, the expression of the gene product is decreased. In a further embodiment of the invention, the gene is modified such that a donor polynucleotide is inserted into the gene,

In one embodiment, the invention provides a vector system comprising one or more vectors. In some embodiments, the vector system comprises: (a) a first regulatory element operably linked to a tracr mate sequence and one or more insertion sites for inserting one or more guide sequences upstream of the tracr mate sequence, wherein when expressed, the guide sequence directs sequence-specific binding of a AAV-site specific nuclease of an engineered nucleic acid modification system to a target sequence optionally in a eukaryotic cell, wherein the engineered nucleic acid modification complex comprises a AAV-site specific nuclease that is complexed with (1) the guide sequence that is hybridized to the target sequence, and (2) the tracr mate sequence that is hybridized to the tracr sequence; and (b) said AAV-site specific nuclease comprising at least one nuclear localization sequence and/or at least one NES; wherein components (a) and (b) are located on or in the same or different vectors of the system. In some embodiments, component (a) further comprises the tracr sequence downstream of the tracr mate sequence under the control of the first regulatory element. In some embodiments, component (a) further comprises two or more guide sequences operably linked to the first regulatory element, wherein when expressed, each of the two or more guide sequences direct sequence specific binding of an AAV-site specific nuclease and thus the engineered nucleic acid modification system complex to a different target sequence in a eukaryotic cell. In some embodiments, the system comprises the tracr sequence under the control of a third regulatory element, such as a polymerase III promoter. In some embodiments, the tracr sequence exhibits at least 50%, 60%, 70%, 80%, 90%, 95%, or 99% of sequence complementarity along the length of the tracr mate sequence when optimally aligned. Determining optimal alignment is within the purview of one of skill in the art. For example, there are publicly and commercially available alignment algorithms and programs such as, but not limited to, ClustalW, Smith-Waterman in matlab, Bowtie, Geneious, Biopython and SeqMan. In some embodiments, the AAV-engineered nucleic acid modification system complex comprises one or more nuclear localization sequences of sufficient strength to drive accumulation of said engineered nucleic acid modification system complex in a detectable amount in the nucleus of a eukaryotic cell. Without wishing to be bound by theory, it is believed that a nuclear localization sequence is not necessary for AAV-engineered nucleic acid modification system complex activity in eukaryotes, but that including such sequences enhances activity of the system, especially as to targeting nucleic acid molecules in the nucleus and/or having molecules exit the nucleus. In some embodiments, the AAV-engineered nucleic acid modification system protein comprises an AAV-Cas enzyme. In some embodiments, the AAV-Cas enzyme is derived from S. pneumoniae, S. pyogenes, S. thermophiles, F. novicida or S. aureus Cas9 (e.g., a Cas protein of one of these organisms modified to have or be associated with at least one AAV) and may include further mutations or alterations or be a chimeric Cas9. The Cas may be an Cas9 homolog or ortholog. In some embodiments, the AAV-engineered nucleic acid modification system protein is codon-optimized for expression in a eukaryotic cell. In some embodiments, the AAV-engineered nucleic acid modification system protein directs cleavage or nicking of one or two strands at the location of the target sequence, cleavage of a donor polynucleotide from a donor construct, and/or facilitates insertion or incorporation of a donor polynucleotide into a target polynucleotide. In some embodiments, the AAV-site specific nuclease of the engineered nucleic acid modification system lacks DNA strand cleavage activity. In some embodiments, the first regulatory element is a polymerase III promoter. In some embodiments, the second regulatory element is a polymerase II promoter. In some embodiments, the guide sequence is at least 15, 16, 17, 18, 19, 20, 25 nucleotides, or between 10-30, or between 15-25, or between 15-20 nucleotides in length.

In general, in some embodiments, the AAV further comprises a repair template. It will be appreciated that comprises here may mean encompassed within the viral capsid or that the virus encodes the comprised protein. In some embodiments, one or more, preferably two or more guide RNAs, may be comprised/encompassed within the AAV vector. Two may be preferred, in some embodiments, as it allows for multiplexing or dual nickase approaches. Particularly for multiplexing, two or more guides may be used. In fact, in some embodiments, three or more, four or more, five or more, or even six or more guide RNAs may be comprised/encompassed within the AAV. More space has been freed up within the AAV by virtue of the fact that the AAV no longer needs to comprise/encompass the CRISPR enzyme. In each of these instances, a repair template may also be provided comprised/encompassed within the AAV. In some embodiments, the repair template corresponds to or includes the DNA target.

### Herpes Simplex Viral Vectors

In some embodiments, the vector can be a Herpes Simplex Viral (HSV)-based vector or system thereof. HSV systems can include the disabled infections single copy (DISC) viruses, which are composed of a glycoprotein H defective mutant HSV genome. When the defective HSV is propagated in complementing cells, virus particles can be generated that are capable of infecting subsequent cells permanently replicating their own genome but are not capable of producing more infectious particles. See e.g., 2009. Trobridge. Exp. Opin. Biol. Ther. 9:1427-1436, whose techniques and vectors described therein can be modified and adapted for use in the engineered nucleic acid modification system of the present invention. In some embodiments where an HSV vector or system thereof is utilized, the host cell can be a complementing cell. In some embodiments, HSV vector or system thereof can be capable of producing virus particles capable of delivering a polynucleotide cargo of up to 150 kb. Thus, in some embodiments, the engineered nucleic acid modification system polynucleotide(s) included in the HSV-based viral vector or system thereof can sum from about 0.001 to about 150 kb. HSV-based vectors and systems thereof have been successfully used in several contexts including various models of neurologic disorders. See e.g., Cockrell et al. 2007. Mol. Biotechnol. 36:184-204; Kafri T. 2004. Mol. Biol. 246:367-390; Balaggan and Ali. 2012. Gene Ther. 19:145-153; Wong et al. 2006. Hum. Gen. Ther. 2002. 17:1-9; Azzouz et al. J. Neruosci. 22L10302-10312; and Betchen and Kaplitt. 2003. Curr. Opin. Neurol. 16:487-493, whose techniques and vectors described therein can be modified and adapted for use in the engineered nucleic acid modification system of the present invention.

### Poxvirus Vectors

In some embodiments, the vector can be a poxvirus vector or system thereof. In some embodiments, the poxvirus vector can result in cytoplasmic expression of one or more engineered nucleic acid modification system polynucleotides of the present invention. In some embodiments the capacity of a poxvirus vector or system thereof can be about 25 kb or more. In some embodiments, a poxvirus vector or system thereof can include one or more engineered nucleic acid modification system polynucleotides described herein.

### Viral Vectors for Delivery to Plants

The systems and compositions may be delivered to plant cells using viral vehicles. In particular embodiments, the compositions and systems may be introduced in the plant cells using a plant viral vector (e.g., Scholthof et al. 1996, Annu Rev Phytopathol. 1996; 34:299-323). Such viral vector may be a vector from a DNA virus, e.g., geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). The viral vector may be a vector from an RNA virus, e.g., tobravirus (e.g., tobacco rattle virus, tobacco mosaic virus), potexvirus (e.g., potato virus X), or hordeivirus (e.g., barley stripe mosaic virus). The replicating genomes of plant viruses may be non-integrative vectors.

Virus Particle Production from Viral Vectors

### Retroviral Production

In some embodiments, one or more viral vectors and/or system thereof can be delivered to a suitable cell line for production of virus particles containing the polynucleotide or other payload to be delivered to a host cell. Suitable host cells for virus production from viral vectors and systems thereof described herein are known in the art and are commercially available. For example, suitable host cells include HEK 293 cells and its variants (HEK 293T and HEK 293TN cells). In some embodiments, the suitable host cell for virus production from viral vectors and systems thereof described herein can stably express one or more genes involved in packaging (e.g., pol, gag, and/or VSV-G) and/or other supporting genes.

In some embodiments, after delivery of one or more viral vectors to the suitable host cells for or virus production from viral vectors and systems thereof, the cells are incubated for an appropriate length of time to allow for viral gene expression from the vectors, packaging of the polynucleotide to be delivered (e.g., an engineered nucleic acid modification polynucleotide), and virus particle assembly, and secretion of mature virus particles into the culture media. Various other methods and techniques are generally known to those of ordinary skill in the art.

Mature virus particles can be collected from the culture media by a suitable method. In some embodiments, this can involve centrifugation to concentrate the virus. The titer of the composition containing the collected virus particles can be obtained using a suitable method. Such methods can include transducing a suitable cell line (e.g., NIH 3T3 cells) and determining transduction efficiency, infectivity in that cell line by a suitable method. Suitable methods include PCR-based methods, flow cytometry, and antibiotic selection-based methods. Various other methods and techniques are generally known to those of ordinary skill in the art. The concentration of virus particle can be adjusted as needed. In some embodiments, the resulting composition containing virus particles can contain 1×10¹-1×10²⁰ particles/mL.

Lentiviruses may be prepared from any lentiviral vector or vector system described herein. In one example embodiment, after cloning pCasES10 (which contains a lentiviral transfer plasmid backbone), HEK293FT at low passage (p=5) can be seeded in a T-75 flask to 50% confluence the day before transfection in DMEM with 10% fetal bovine serum and without antibiotics. After 20 hours, the media can be changed to OptiMEM (serum-free) media and transfection of the lentiviral vectors can done 4 hours later. Cells can be transfected with 10 μg of lentiviral transfer plasmid (pCasES10) and the appropriate packaging plasmids (e.g., 5 μg of pMD2.G (VSV-g pseudotype), and 7.5 ug of psPAX2 (gag/pol/rev/tat)). Transfection can be carried out in 4 mL OptiMEM with a cationic lipid delivery agent (50 uL Lipofectamine 2000 and 100 ul Plus reagent). After 6 hours, the media can be changed to antibiotic-free DMEM with 10% fetal bovine serum. These methods can use serum during cell culture, but serum-free methods are preferred.

Following transfection and allowing the producing cells (also referred to as packaging cells) to package and produce virus particles with packaged cargo, the lentiviral particles can be purified. In an exemplary embodiment, virus-containing supernatants can be harvested after 48 hours. Collected virus-containing supernatants can first be cleared of debris and filtered through a 0.45 um low protein binding (PVDF) filter. They can then be spun in an ultracentrifuge for 2 hours at 24,000 rpm. The resulting virus-containing pellets can be resuspended in 50 ul of DMEM overnight at 4 degrees C. They can be then aliquoted and used immediately or immediately frozen at −80 degrees C. for storage.

### AAV Particle Production

There are two main strategies for producing AAV particles from AAV vectors and systems thereof, such as those described herein, which depend on how the adenovirus helper factors are provided (helper v. helper free). In some embodiments, a method of producing AAV particles from AAV vectors and systems thereof can include adenovirus infection into cell lines that stably harbor AAV replication and capsid encoding polynucleotides along with AAV vector containing the polynucleotide to be packaged and delivered by the resulting AAV particle (e.g., the engineered nucleic acid modification system polynucleotide(s)). In some embodiments, a method of producing AAV particles from AAV vectors and systems thereof can be a “helper free” method, which includes co-transfection of an appropriate producing cell line with three vectors (e.g., plasmid vectors): (1) an AAV vector that contains a polynucleotide of interest (e.g., the engineered nucleic acid modification system polynucleotide(s)) between 2 ITRs; (2) a vector that carries the AAV Rep-Cap encoding polynucleotides; and (helper polynucleotides. One of skill in the art will appreciate various methods and variations thereof that are both helper and -helper free and as well as the different advantages of each system.

### Non-Viral Vectors

In some embodiments, the vector is a non-viral vector or vector system. The term of art “Non-viral vector” and as used herein in this context refers to molecules and/or compositions that are vectors but that are not based on one or more component of a virus or virus genome (excluding any nucleotide to be delivered and/or expressed by the non-viral vector) that can be capable of incorporating engineered nucleic acid modification polynucleotide(s) of the present invention and delivering said engineered nucleic acid modification polynucleotide(s) to a cell and/or expressing the polynucleotide in the cell. It will be appreciated that this does not exclude vectors containing a polynucleotide designed to target a virus-based polynucleotide that is to be delivered. For example, if a gRNA to be delivered is directed against a virus component and it is inserted or otherwise coupled to an otherwise non-viral vector or carrier, this would not make said vector a “viral vector”. Non-viral vectors can include, without limitation, naked polynucleotides and polynucleotide (non-viral) based vector and vector systems.

### Naked Polynucleotides

In some embodiments one or more engineered nucleic acid modification system polynucleotides described elsewhere herein can be included in a naked polynucleotide. The term of art “naked polynucleotide” as used herein refers to polynucleotides that are not associated with another molecule (e.g., proteins, lipids, and/or other molecules) that can often help protect it from environmental factors and/or degradation. As used herein, associated with includes, but is not limited to, linked to, adhered to, adsorbed to, enclosed in, enclosed in or within, mixed with, and the like. Naked polynucleotides that include one or more of the engineered nucleic acid modification system polynucleotides described herein can be delivered directly to a host cell and optionally expressed therein. The naked polynucleotides can have any suitable two- and three-dimensional configurations. By way of non-limiting examples, naked polynucleotides can be single-stranded molecules, double stranded molecules, circular molecules (e.g., plasmids and artificial chromosomes), molecules that contain portions that are single stranded and portions that are double stranded (e.g., ribozymes), and the like. In some embodiments, the naked polynucleotide contains only the engineered nucleic acid modification system polynucleotide(s) of the present invention. In some embodiments, the naked polynucleotide can contain other nucleic acids and/or polynucleotides in addition to the engineered nucleic acid modification system polynucleotide(s) of the present invention. The naked polynucleotides can include one or more elements of a transposon system. Transposons and system thereof are described in greater detail elsewhere herein.

### Non-Viral Polynucleotide Vectors

In some embodiments, one or more of the engineered nucleic acid modification system polynucleotides can be included in a non-viral polynucleotide vector. Suitable non-viral polynucleotide vectors include, but are not limited to, transposon vectors and vector systems, plasmids, bacterial artificial chromosomes, yeast artificial chromosomes, AR(antibiotic resistance)-free plasmids and miniplasmids, circular covalently closed vectors (e.g. minicircles, minivectors, miniknots,), linear covalently closed vectors (“dumbbell shaped”), MIDGE (minimalistic immunologically defined gene expression) vectors, MiLV (micro-linear vector) vectors, Ministrings, mini-intronic plasmids, PSK systems (post-segregationally killing systems), ORT (operator repressor titration) plasmids, and the like. See e.g., Hardee et al. 2017. Genes. 8(2):65.

In some embodiments, the non-viral polynucleotide vector can have a conditional origin of replication. In some embodiments, the non-viral polynucleotide vector can be an ORT plasmid. In some embodiments, the non-viral polynucleotide vector can have a minimalistic immunologically defined gene expression. In some embodiments, the non-viral polynucleotide vector can have one or more post-segregationally killing system genes. In some embodiments, the non-viral polynucleotide vector is AR-free. In some embodiments, the non-viral polynucleotide vector is a minivector. In some embodiments, the non-viral polynucleotide vector includes a nuclear localization signal. In some embodiments, the non-viral polynucleotide vector can include one or more CpG motifs. In some embodiments, the non-viral polynucleotide vectors can include one or more scaffold/matrix attachment regions (S/MARs). See e.g., Mirkovitch et al. 1984. Cell. 39:223-232, Wong et al. 2015. Adv. Genet. 89:113-152, whose techniques and vectors can be adapted for use in the present invention. S/MARs are AT-rich sequences that play a role in the spatial organization of chromosomes through DNA loop base attachment to the nuclear matrix. S/MARs are often found close to regulatory elements such as promoters, enhancers, and origins of DNA replication. Inclusion of one or S/MARs can facilitate a once-per-cell-cycle replication to maintain the non-viral polynucleotide vector as an episome in daughter cells. In certain embodiments, the S/MAR sequence is located downstream of an actively transcribed polynucleotide (e.g., one or more engineered nucleic acid modification system polynucleotides of the present invention) included in the non-viral polynucleotide vector. In some embodiments, the S/MAR can be a S/MAR from the beta-interferon gene cluster. See e.g., Verghese et al. 2014. Nucleic Acid Res. 42:e53; Xu et al. 2016. Sci. China Life Sci. 59:1024-1033; Jin et al. 2016. 8:702-711; Koirala et al. 2014. Adv. Exp. Med. Biol. 801:703-709; and Nehlsen et al. 2006. Gene Ther. Mol. Biol. 10:233-244, whose techniques and vectors can be adapted for use in the present invention.

In some embodiments, the non-viral vector is a transposon vector or system thereof. As used herein, “transposon” (also referred to as transposable element) refers to a polynucleotide sequence that is capable of moving form location in a genome to another. There are several classes of transposons. Transposons include retrotransposons and DNA transposons. Retrotransposons require the transcription of the polynucleotide that is moved (or transposed) in order to transpose the polynucleotide to a new genome or polynucleotide. DNA transposons are those that do not require reverse transcription of the polynucleotide that is moved (or transposed) in order to transpose the polynucleotide to a new genome or polynucleotide. In some embodiments, the non-viral polynucleotide vector can be a retrotransposon vector. In some embodiments, the retrotransposon vector includes long terminal repeats. In some embodiments, the retrotransposon vector does not include long terminal repeats. In some embodiments, the non-viral polynucleotide vector can be a DNA transposon vector. DNA transposon vectors can include a polynucleotide sequence encoding a transposase. In some embodiments, the transposon vector is configured as a non-autonomous transposon vector, meaning that the transposition does not occur spontaneously on its own. In some of these embodiments, the transposon vector lacks one or more polynucleotide sequences encoding proteins required for transposition. In some embodiments, the non-autonomous transposon vectors lack one or more Ac elements.

In some embodiments a non-viral polynucleotide transposon vector system can include a first polynucleotide vector that contains the engineered nucleic acid modification system polynucleotide(s) of the present invention flanked on the 5′ and 3′ ends by transposon terminal inverted repeats (TIRs) and a second polynucleotide vector that includes a polynucleotide capable of encoding a transposase coupled to a promoter to drive expression of the transposase. When both are expressed in the same cell the transposase can be expressed from the second vector and can transpose the material between the TIRs on the first vector (e.g., the engineered nucleic acid modification system polynucleotide(s) of the present invention) and integrate it into one or more positions in the host cell's genome. In some embodiments the transposon vector or system thereof can be configured as a gene trap. In some embodiments, the TIRs can be configured to flank a strong splice acceptor site followed by a reporter and/or other gene (e.g., one or more of the engineered nucleic acid modification system polynucleotide(s) of the present invention) and a strong poly A tail. When transposition occurs while using this vector or system thereof, the transposon can insert into an intron of a gene and the inserted reporter or other gene can provoke a mis-splicing process and as a result it in activates the trapped gene.

Any suitable transposon system can be used. Suitable transposon and systems thereof can include, without limitation, Sleeping Beauty transposon system (Tc1/mariner superfamily) (see e.g., Ivics et al. 1997. Cell. 91(4): 501-510), piggyBac (piggyBac superfamily) (see e.g., Li et al. 2013 110(25): E2279-E2287 and Yusa et al. 2011. PNAS. 108(4): 1531-1536), Tol2 (superfamily hAT), Frog Prince (Tc1/mariner superfamily) (see e.g., Miskey et al. 2003 Nucleic Acid Res. 31(23):6873-6881) and variants thereof.

### Non-Vector Delivery Vehicles

The delivery vehicles may comprise non-viral vehicles. In general, methods and vehicles capable of delivering nucleic acids and/or proteins may be used for delivering the systems compositions herein. Examples of non-viral vehicles include lipid nanoparticles, cell-penetrating peptides (CPPs), DNA nanoclews, metal nanoparticles, streptolysin O, multifunctional envelope-type nanodevices (MENDs), lipid-coated mesoporous silica particles, and other inorganic nanoparticles.

### Lipid Particles

The delivery vehicles may comprise lipid particles, e.g., lipid nanoparticles (LNPs) and liposomes. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, International Patent Publication Nos. WO 91/17424 and WO 91/16024. The preparation of lipid:nucleic acid complexes, including targeted liposomes such as immunolipid complexes, is well known to one of skill in the art (see e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

### Lipid Nanoparticles (LNPs)

LNPs may encapsulate nucleic acids within cationic lipid particles (e.g., liposomes), and may be delivered to cells with relative ease. In some examples, lipid nanoparticles do not contain any viral components, which helps minimize safety and immunogenicity concerns. Lipid particles may be used for in vitro, ex vivo, and in vivo deliveries. Lipid particles may be used for various scales of cell populations.

In some examples. LNPs may be used for delivering DNA molecules (e.g., those comprising coding sequences of engineered nucleic acid modification system polypeptides (e.g., site-specific nuclease, DNA polymerase, Vir polypeptides (e.g., VirD1, VirD2, etc.) and/or RNA molecules (e.g., mRNA of engineered nucleic acid modification system proteins, gRNAs). In certain cases, LNPs may be use for delivering RNP complexes of engineered nucleic acid modification polypeptides (e.g., site specific nuclease) and gRNA.

Components in LNPs may comprise cationic lipids 1,2-dilineoyl-3-dimethylammonium-propane (DLinDAP), 1,2-dilinoleyloxy-3-N,N-dimethylaminopropane (DLinDMA), 1,2-dilinoleyloxyketo-N,N-dimethyl-3-aminopropane (DLinK-DMA), 1,2-dilinoleyl-4-(2-dimethylaminoethyl)-[1,3]-dioxolane (DLinKC2-DMA), (3-o-[2″-(methoxypolyethyleneglycol 2000) succinoyl]-1,2-dimyristoyl-sn-glycol (PEG-S-DMG), R-3-[(ro-methoxy-poly(ethylene glycol)2000) carbamoyl]-1,2-dimyristyloxlpropyl-3-amine (PEG-C-DOMG, and any combination thereof. Preparation of LNPs and encapsulation may be adapted from Rosin et al, Molecular Therapy, vol. 19, no. 12, pages 1286-2200, December 2011).

In some embodiments, an LNP delivery vehicle can be used to deliver a virus particle containing an engineered nucleic acid modification system and/or component(s) thereof. In some embodiments, the virus particle(s) can be adsorbed to the lipid particle, such as through electrostatic interactions, and/or can be attached to the liposomes via a linker.

In some embodiments, the LNP contains a nucleic acid, wherein the charge ratio of nucleic acid backbone phosphates to cationic lipid nitrogen atoms is about 1: 1.5-7 or about 1:4.

In some embodiments, the LNP also includes a shielding compound, which is removable from the lipid composition under in vivo conditions. In some embodiments, the shielding compound is a biologically inert compound. In some embodiments, the shielding compound does not carry any charge on its surface or on the molecule as such. In some embodiments, the shielding compounds are polyethylenglycoles (PEGs), hydroxyethylglucose (HEG) based polymers, polyhydroxyethyl starch (polyHES) and polypropylene. In some embodiments, the PEG, HEG, polyHES, and a polypropylene weight between about 500 to 10,000 Da or between about 2000 to 5000 Da. In some embodiments, the shielding compound is PEG2000 or PEG5000.

In some embodiments, the LNP can include one or more helper lipids. In some embodiments, the helper lipid can be a phosphor lipid or a steroid. In some embodiments, the helper lipid is between about 20 mol % to 80 mol % of the total lipid content of the composition. In some embodiments, the helper lipid component is between about 35 mol % to 65 mol % of the total lipid content of the LNP. In some embodiments, the LNP includes lipids at 50 mol % and the helper lipid at 50 mol % of the total lipid content of the LNP.

Other non-limiting, exemplary LNP delivery vehicles are described in U.S. Patent Publication Nos. US 20160174546, US 20140301951, US 20150105538, US 20150250725, Wang et al., J. Control Release, 2017 Jan. 31. pii: S0168-3659(17)30038-X. doi: 10.1016/j.jconrel.2017.01.037. [Epub ahead of print]; Altmoglu et al., Biomater Sci., 4(12):1773-80, Nov. 15, 2016; Wang et al., PNAS, 113(11):2868-73 Mar. 15, 2016; Wang et al., PloS One, 10(11): e0141860. doi: 10.1371/journal.pone.0141860. eCollection 2015, Nov. 3, 2015; Takeda et al., Neural Regen Res. 10(5):689-90, May 2015; Wang et al., Adv. Healthc Mater., 3(9):1398-403, September 2014; and Wang et al., Agnew Chem Int Ed Engl., 53(11):2893-8, Mar. 10, 2014; James E. Dahlman and Carmen Barnes et al. Nature Nanotechnology (2014) published online 11 May 2014, doi:10.1038/nnano.2014.84; Coelho et al., N Engl J Med 2013; 369:819-29; Aleku et al., Cancer Res., 68(23): 9788-98 (Dec. 1, 2008), Strumberg et al., Int. J. Clin. Pharmacol. Ther., 50(1): 76-8 (January 2012), Schultheis et al., J. Clin. Oncol., 32(36): 4141-48 (Dec. 20, 2014), and Fehring et al., Mol. Ther., 22(4): 811-20 (Apr. 22, 2014); Novobrantseva, Molecular Therapy-Nucleic Acids (2012) 1, e4; doi:10.1038/mtna.2011.3; WO2012135025; US 20140348900; US 20140328759; US 20140308304; WO 2005/105152; WO 2006/069782; WO 2007/121947; US 2015/082080; US 20120251618; 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos 1766035; 1519714; 1781593 and 1664316.

### Liposomes

In some embodiments, a lipid particle may be liposome. Liposomes are spherical vesicle structures composed of a uni- or multilamellar lipid bilayer surrounding internal aqueous compartments and a relatively impermeable outer lipophilic phospholipid bilayer. In some embodiments, liposomes are biocompatible, nontoxic, can deliver both hydrophilic and lipophilic drug molecules, protect their cargo from degradation by plasma enzymes, and transport their load across biological membranes and the blood brain barrier (BBB).

Liposomes can be made from several different types of lipids, e.g., phospholipids. A liposome may comprise natural phospholipids and lipids such as 1,2-distearoryl-sn-glycero-3-phosphatidyl choline (DSPC), sphingomyelin, egg phosphatidylcholines, monosialoganglioside, or any combination thereof.

Several other additives may be added to liposomes in order to modify their structure and properties. For instance, liposomes may further comprise cholesterol, sphingomyelin, and/or 1,2-dioleoyl-sn-glycero-3-phosphoethanolamine (DOPE), e.g., to increase stability and/or to prevent the leakage of the liposomal inner cargo.

In some embodiments, a liposome delivery vehicle can be used to deliver a virus particle containing an engineered nucleic acid modification system and/or component(s) thereof. In some embodiments, the virus particle(s) can be adsorbed to the liposome, such as through electrostatic interactions, and/or can be attached to the liposomes via a linker.

In some embodiments, the liposome can be a Trojan Horse liposome (also known in the art as Molecular Trojan Horses), see e.g., http://cshprotocols.cshlp.org/content/2010/4/pdb.prot5407.long, the teachings of which can be applied and/or adapted to generated and/or deliver the CRISPR-Cas systems described herein.

Other non-limiting, exemplary liposomes can be those as set forth in Wang et al., ACS Synthetic Biology, 1, 403-07 (2012); Wang et al., PNAS, 113(11) 2868-2873 (2016); Spuch and Navarro, Journal of Drug Delivery, vol. 2011, Article ID 469679, 12 pages, 2011. doi:10.1155/2011/469679; WO 2008/042973; U.S. Pat. No. 8,071,082; WO 2014/186366; 20160257951; US20160129120; US 20160244761; 20120251618; WO2013/093648; Lipofectin (a combination of DOTMA and DOPE), Lipofectase, LIPOFECTAMINE® (e.g., LIPOFECTAMINE® 2000, LIPOFECTAMINE® 3000, LIPOFECTAMINE® RNAiMAX, LIPOFECTAMINE® LTX), SAINT-RED (Synvolux Therapeutics, Groningen Netherlands), DOPE, Cytofectin (Gilead Sciences, Foster City, Calif.), and Eufectins (JBL, San Luis Obispo, Calif.).

### Stable Nucleic-Acid-Lipid Particles (SNALPs)

In some embodiments, the lipid particles may be stable nucleic acid lipid particles (SNALPs). SNALPs may comprise an ionizable lipid (DLinDMA) (e.g., cationic at low pH), a neutral helper lipid, cholesterol, a diffusible polyethylene glycol (PEG)-lipid, or any combination thereof. In some examples, SNALPs may comprise synthetic cholesterol, dipalmitoylphosphatidylcholine, 3-N-[(w-methoxy polyethylene glycol)2000)carbamoyl]-1,2-dimyrestyloxypropylamine, and cationic 1,2-dilinoleyloxy-3-N,Ndimethylaminopropane. In some examples, SNALPs may comprise synthetic cholesterol, 1,2-distearoyl-sn-glycero-3-phosphocholine, PEG-cDMA, and 1,2-dilinoleyloxy-3-(N;N-dimethyl)aminopropane (DLinDMAo).

Other non-limiting, exemplary SNALPs that can be used to deliver the engineered nucleic acid modification systems described herein can be any such SNALPs as described in Morrissey et al., Nature Biotechnology, Vol. 23, No. 8, August 2005, Zimmerman et al., Nature Letters, Vol. 441, 4 May 2006; Geisbert et al., Lancet 2010; 375: 1896-905; Judge, J. Clin. Invest. 119:661-673 (2009); and Semple et al., Nature Niotechnology, Volume 28 Number 2 Feb. 2010, pp. 172-177.

### Other Lipids

The lipid particles may also comprise one or more other types of lipids, e.g., cationic lipids, such as amino lipid 2,2-dilinoleyl-4-dimethylaminoethyl-[1,3]-dioxolane (DLin-KC2-DMA), DLin-KC2-DMA4, C12-200 and colipids disteroylphosphatidyl choline, cholesterol, and PEG-DMG.

In some embodiments, the delivery vehicle can be or include a lipidoid, such as any of those set forth in, for example, US 20110293703.

In some embodiments, the delivery vehicle can be or include an amino lipid, such as any of those set forth in, for example, Jayaraman, Angew. Chem. Int. Ed. 2012, 51, 8529-8533.

In some embodiments, the delivery vehicle can be or include a lipid envelope, such as any of those set forth in, for example, Korman et al., 2011. Nat. Biotech. 29:154-157.

### Lipoplexes/Polyplexes

In some embodiments, the delivery vehicles comprise lipoplexes and/or polyplexes. Lipoplexes may bind to negatively charged cell membrane and induce endocytosis into the cells. Examples of lipoplexes may be complexes comprising lipid(s) and non-lipid components. Examples of lipoplexes and polyplexes include FuGENE-6 reagent, a non-liposomal solution containing lipids and other components, zwitterionic amino lipids (ZALs), Ca2 [symbol] (e.g., forming DNA/Ca²⁺ microcomplexes), polyethenimine (PEI) (e.g., branched PEI), and poly(L-lysine) (PLL).

### Sugar-Based Particles

In some embodiments, the delivery vehicle can be a sugar-based particle. In some embodiments, the sugar-based particles can be or include GalNAc, such as any of those described in WO2014118272; US 20020150626; Nair, J K et al., 2014, Journal of the American Chemical Society 136 (49), 16958-16961; Ostergaard et al., Bioconjugate Chem., 2015, 26 (8), pp 1451-1455.

### Cell Penetrating Peptides

In some embodiments, the delivery vehicles comprise cell penetrating peptides (CPPs). CPPs are short peptides that facilitate cellular uptake of various molecular cargo (e.g., from nanosized particles to small chemical molecules and large fragments of DNA).

CPPs may be of different sizes, amino acid sequences, and charges. In some examples, CPPs can translocate the plasma membrane and facilitate the delivery of various molecular cargoes to the cytoplasm or an organelle. CPPs may be introduced into cells via different mechanisms, e.g., direct penetration in the membrane, endocytosis-mediated entry, and translocation through the formation of a transitory structure.

CPPs may have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Another type of CPPs is the trans-activating transcriptional activator (Tat) from Human Immunodeficiency Virus 1 (HIV-1). Examples of CPPs include to Penetratin, Tat (48-60), Transportan, and (R-AhX-R4) (Ahx refers to aminohexanoyl), Kaposi fibroblast growth factor (FGF) signal peptide sequence, integrin 33 signal peptide sequence, polyarginine peptide Args sequence, Guanine rich-molecular transporters, and sweet arrow peptide. Examples of CPPs and related applications also include those described in U.S. Pat. No. 8,372,951.

CPPs can be used for in vitro and ex vivo work quite readily, and extensive optimization for each cargo and cell type is usually required. In some examples, CPPs may be covalently attached to an engineered nucleic acid modification system protein directly, which is then complexed with the gRNA and/or donor construct and/or donor polynucleotide and delivered to cells. In some examples, separate delivery of CPP-engineered nucleic acid modification system protein(s) and/or CPP-gRNA and/or CPP-donor construct to multiple cells may be performed. CPP may also be used to delivery RNPs.

CPPs may be used to deliver the engineered nucleic acid modification system or component(s) thereof to plants. In some examples, CPPs may be used to deliver the engineered nucleic acid modification system and/or components to plant protoplasts, which are then regenerated to plant cells and further to plants.

### DNA Nanoclews

In some embodiments, the delivery vehicles comprise DNA nanoclews. A DNA nanoclew refers to a sphere-like structure of DNA (e.g., with a shape of a ball of yarn). The nanoclew may be synthesized by rolling circle amplification with palindromic sequences that aide in the self-assembly of the structure. The sphere may then be loaded with a payload. An example of DNA nanoclew is described in Sun W et al, J Am Chem Soc. 2014 Oct. 22; 136(42):14722-5; and Sun W et al, Angew Chem Int Ed Engl. 2015 Oct. 5; 54(41):12029-33. DNA nanoclew may have a palindromic sequences to be partially complementary to the gRNA within a site specific nuclease (e.g., Cas):gRNA ribonucleoprotein complex. A DNA nanoclew may be coated, e.g., coated with PEI to induce endosomal escape.

### Metal Nanoparticles

In some embodiments, the delivery vehicles comprise gold nanoparticles (also referred to AuNPs or colloidal gold). Gold nanoparticles may form complex with cargos, e.g., engineered nucleic acid modification systems and/or components thereof site specific nuclease (e.g., Cas):gRNA RNP. Gold nanoparticles may be coated, e.g., coated in a silicate and an endosomal disruptive polymer, PAsp(DET). Examples of gold nanoparticles include AuraSense Therapeutics' Spherical Nucleic Acid (SNA™) constructs, and those described in Mout R, et al. (2017). ACS Nano 11:2452-8; Lee K, et al. (2017). Nat Biomed Eng 1:889-901. Other metal nanoparticles can also be complexed with cargo(s). Such metal particles include tungsten, palladium, rhodium, platinum, and iridium particles. Other non-limiting, exemplary metal nanoparticles are described in US 20100129793. iTOP

In some embodiments, the delivery vehicles comprise iTOP. iTOP refers to a combination of small molecules drives the highly efficient intracellular delivery of native proteins, independent of any transduction peptide. iTOP may be used for induced transduction by osmocytosis and propanebetaine, using NaCl-mediated hyperosmolality together with a transduction compound (propanebetaine) to trigger macropinocytotic uptake into cells of extracellular macromolecules. Examples of iTOP methods and reagents include those described in D'Astolfo D S, Pagliero R J, Pras A, et al. (2015). Cell 161:674-690.

### Polymer-Based Particles

In some embodiments, the delivery vehicles may comprise polymer-based particles (e.g., nanoparticles). In some embodiments, the polymer-based particles may mimic a viral mechanism of membrane fusion. The polymer-based particles may be a synthetic copy of Influenza virus machinery and form transfection complexes with various types of nucleic acids ((siRNA, miRNA, plasmid DNA or shRNA, mRNA) that cells take up via the endocytosis pathway, a process that involves the formation of an acidic compartment. The low pH in late endosomes acts as a chemical switch that renders the particle surface hydrophobic and facilitates membrane crossing. Once in the cytosol, the particle releases its payload for cellular action. This Active Endosome Escape technology is safe and maximizes transfection efficiency as it is using a natural uptake pathway. In some embodiments, the polymer-based particles may comprise alkylated and carboxyalkylated branched polyethylenimine. In some examples, the polymer-based particles are VIROMER, e.g., VIROMER RNAi, VIROMER RED, VIROMER mRNA, VIROMER CRISPR. Example methods of delivering the systems and compositions of the present invention herein include those described in Bawage S S et al., Synthetic mRNA expressed Cas13a mitigates RNA virus infections, www.biorxiv.org/content/10.1101/370460v1.full doi: doi.org/10.1101/370460, Viromer® RED, a powerful tool for transfection of keratinocytes. doi: 10.13140/RG.2.2.16993.61281, Viromer® Transfection—Factbook 2018: technology, product overview, users' data, doi:10.13140/RG.2.2.23912.16642. Other exemplary and non-limiting polymeric particles are described in US 20170079916, US 20160367686, US 20110212179, US 20130302401, 6,007,845, 5,855,913, 5,985,309, 5,543,158, WO2012135025, US 20130252281, US 20130245107, US 20130244279; US 20050019923, 20080267903.

### Streptolysin O (SLO)

The delivery vehicles may be streptolysin O (SLO). SLO is a toxin produced by Group A streptococci that works by creating pores in mammalian cell membranes. SLO may act in a reversible manner, which allows for the delivery of proteins (e.g., up to 100 kDa) to the cytosol of cells without compromising overall viability. Examples of SLO include those described in Sierig G, et al. (2003). Infect Immun 71:446-55; Walev I, et al. (2001). Proc Natl Acad Sci USA 98:3185-90; Teng K W, et al. (2017). Elife 6:e25460.

### Multifunctional Envelope-Type Nanodevice (MEND)

The delivery vehicles may comprise multifunctional envelope-type nanodevice (MENDs). MENDs may comprise condensed plasmid DNA, a PLL core, and a lipid film shell. A MEND may further comprise cell-penetrating peptide (e.g., stearyl octaarginine). The cell penetrating peptide may be in the lipid shell. The lipid envelope may be modified with one or more functional components, e.g., one or more of: polyethylene glycol (e.g., to increase vascular circulation time), ligands for targeting of specific tissues/cells, additional cell-penetrating peptides (e.g., for greater cellular delivery), lipids to enhance endosomal escape, and nuclear delivery tags. In some examples, the MEND may be a tetra-lamellar MEND (T-MEND), which may target the cellular nucleus and mitochondria. In certain examples, a MEND may be a PEG-peptide-DOPE-conjugated MEND (PPD-MEND), which may target bladder cancer cells. Examples of MENDs include those described in Kogure K, et al. (2004). J Control Release 98:317-23; Nakamura T, et al. (2012). Acc Chem Res 45:1113-21.

### Lipid-Coated Mesoporous Silica Particles

The delivery vehicles may comprise lipid-coated mesoporous silica particles. Lipid-coated mesoporous silica particles may comprise a mesoporous silica nanoparticle core and a lipid membrane shell. The silica core may have a large internal surface area, leading to high cargo (e.g., engineered nucleic acid modification systems or components thereof) loading capacities. In some embodiments, pore sizes, pore chemistry, and overall particle sizes may be modified for loading different types of cargos. The lipid coating of the particle may also be modified to maximize cargo loading, increase circulation times, and provide precise targeting and cargo release. Examples of lipid-coated mesoporous silica particles include those described in Du X, et al. (2014). Biomaterials 35:5580-90; Durfee P N, et al. (2016). ACS Nano 10:8325-45.

### Inorganic Nanoparticles

The delivery vehicles may comprise inorganic nanoparticles. Examples of inorganic nanoparticles include carbon nanotubes (CNTs) (e.g., as described in Bates K and Kostarelos K. (2013). Adv Drug Deliv Rev 65:2023-33.), bare mesoporous silica nanoparticles (MSNPs) (e.g., as described in Luo G F, et al. (2014). Sci Rep 4:6064), and dense silica nanoparticles (SiNPs) (as described in Luo D and Saltzman W M. (2000). Nat Biotechnol 18:893-5).

### Exosomes

The delivery vehicles may comprise exosomes. Exosomes include membrane bound extracellular vesicles, which can be used to contain and delivery various types of biomolecules, such as proteins, carbohydrates, lipids, and nucleic acids, and complexes thereof (e.g., RNPs). Examples of exosomes include those described in Schroeder A, et al., J Intern Med. 2010 January; 267(1):9-21; E1-Andaloussi S, et al., Nat Protoc. 2012 December; 7(12):2112-26; Uno Y, et al., Hum Gene Ther. 2011 June; 22(6):711-9; Zou W, et al., Hum Gene Ther. 2011 April; 22(4):465-75.

In some examples, the exosome may form a complex (e.g., by binding directly or indirectly) to one or more components of the cargo (e.g., engineered nucleic acid modification systems or components thereof). In certain examples, a molecule of an exosome may be fused with first adapter protein and a component of the cargo may be fused with a second adapter protein. The first and the second adapter protein may specifically bind each other, thus associating the cargo with the exosome. Examples of such exosomes include those described in Ye Y, et al., Biomater Sci. 2020 Apr. 28. doi: 10.1039/d0bm00427h.

Other non-limiting, exemplary exosomes include any of those set forth in Alvarez-Erviti et al. 2011, Nat Biotechnol 29: 341; [1401] E1-Andaloussi et al. (Nature Protocols 7:2112-2126(2012); and Wahlgren et al. (Nucleic Acids Research, 2012, Vol. 40, No. 17 e130).

### Spherical Nucleic Acids (SNAs)

In some embodiments, the delivery vehicle can be a SNA. SNAs are three dimensional nanostructures that can be composed of densely functionalized and highly oriented nucleic acids that can be covalently attached to the surface of spherical nanoparticle cores. The core of the spherical nucleic acid can impart the conjugate with specific chemical and physical properties, and it can act as a scaffold for assembling and orienting the oligonucleotides into a dense spherical arrangement that gives rise to many of their functional properties, distinguishing them from all other forms of matter. In some embodiments, the core is a crosslinked polymer. Non-limiting, exemplary SNAs can be any of those set forth in Cutler et al., J. Am. Chem. Soc. 2011 133:9254-9257, Hao et al., Small. 2011 7:3158-3162, Zhang et al., ACS Nano. 2011 5:6962-6970, Cutler et al., J. Am. Chem. Soc. 2012 134:1376-1391, Young et al., Nano Lett. 2012 12:3867-71, Zheng et al., Proc. Natl. Acad. Sci. USA. 2012 109:11975-80, Mirkin, Nanomedicine 2012 7:635-638 Zhang et al., J. Am. Chem. Soc. 2012 134:16488-1691, Weintraub, Nature 2013 495:S14-S16, Choi et al., Proc. Natl. Acad. Sci. USA. 2013 110(19):7625-7630, Jensen et al., Sci. Transl. Med. 5, 209ra152 (2013) and Mirkin, et al., and Small, 10:186-192.

### Self-Assembling Nanoparticles

In some embodiments, the delivery vehicle is a self-assembling nanoparticle. The self-assembling nanoparticles can contain one or more polymers. The self-assembling nanoparticles can be PEGylated. Self-assembling nanoparticles are known in the art. Non-limiting, exemplary self-assembling nanoparticles are set forth in Schiffelers et al., Nucleic Acids Research, 2004, Vol. 32, No. 19, Bartlett et al. (PNAS, Sep. 25, 2007, vol. 104, no. 39; Davis et al., Nature, Vol 464, 15 Apr. 2010.

### Supercharged Proteins

In some embodiments, the delivery vehicle can be a supercharged protein. As used herein “Supercharged proteins” are a class of engineered or naturally occurring proteins with unusually high positive or negative net theoretical charge. Non-limiting, exemplary supercharged proteins can be any of those set forth in Lawrence et al., 2007, Journal of the American Chemical Society 129, 10110-10112.

### Targeted Delivery

In some embodiments, the delivery vehicle can allow for targeted delivery to a specific cell, tissue, organ, or system. In such embodiments, the delivery vehicle can include one or more targeting moieties that can direct targeted delivery of the cargo(s) (e.g., the engineered nucleic acid modification systems or components thereof of the present invention). In an embodiment, the delivery vehicle comprises a targeting moiety, such as active targeting of a lipid entity of the invention, e.g., lipid particle or nanoparticle or liposome or lipid bilayer of the invention comprising a targeting moiety for active targeting.

With regard to targeting moieties, mention is made of Deshpande et al, “Current trends in the use of liposomes for tumor targeting,” Nanomedicine (Lond). 8(9), doi:10.2217/nnm.13.118 (2013), and the documents it cites, all of which are incorporated herein by reference and the teachings of which can be applied and/or adapted for targeted delivery of one or more engineered nucleic acid modification systems or components thereof described herein. Mention is also made of International Patent Publication No. WO 2016/027264, and the documents it cites, all of which are incorporated herein by reference, the teachings of which can be applied and/or adapted for targeted delivery of one or more engineered nucleic acid modification systems or components thereof described herein. And mention is made of Lorenzer et al, “Going beyond the liver: Progress and challenges of targeted delivery of siRNA therapeutics,” Journal of Controlled Release, 203: 1-15 (2015), and the documents it cites, all of which are incorporated herein by reference, the teachings of which can be applied and/or adapted for targeted delivery of one or more engineered nucleic acid modification systems or components thereof described herein.

An actively targeting lipid particle or nanoparticle or liposome or lipid bilayer delivery system (generally as to embodiments of the invention, “lipid entity of the invention” delivery systems) are prepared by conjugating targeting moieties, including small molecule ligands, peptides and monoclonal antibodies, on the lipid or liposomal surface; for example, certain receptors, such as folate and transferrin (Tf) receptors (TfR), are overexpressed on many cancer cells and have been used to make liposomes tumor cell specific. Liposomes that accumulate in the tumor microenvironment can be subsequently endocytosed into the cells by interacting with specific cell surface receptors. To efficiently target liposomes to cells, such as cancer cells, it is useful that the targeting moiety have an affinity for a cell surface receptor and to link the targeting moiety in sufficient quantities to have optimum affinity for the cell surface receptors; and determining these embodiments are within the ambit of the skilled artisan. In the field of active targeting, there are a number of cell-, e.g., tumor-, specific targeting ligands.

Also, as to active targeting, with regard to targeting cell surface receptors such as cancer cell surface receptors, targeting ligands on liposomes can provide attachment of liposomes to cells, e.g., vascular cells, via a noninternalizing epitope; and this can increase the extracellular concentration of that which is being delivered, thereby increasing the amount delivered to the target cells. A strategy to target cell surface receptors, such as cell surface receptors on cancer cells, such as overexpressed cell surface receptors on cancer cells, is to use receptor-specific ligands or antibodies. Many cancer cell types display upregulation of tumor-specific receptors. For example, TfRs and folate receptors (FRs) are greatly overexpressed by many tumor cell types in response to their increased metabolic demand. Folic acid can be used as a targeting ligand for specialized delivery owing to its ease of conjugation to nanocarriers, its high affinity for FRs and the relatively low frequency of FRs, in normal tissues as compared with their overexpression in activated macrophages and cancer cells, e.g., certain ovarian, breast, lung, colon, kidney and brain tumors. Overexpression of FR on macrophages is an indication of inflammatory diseases, such as psoriasis, Crohn's disease, rheumatoid arthritis and atherosclerosis; accordingly, folate-mediated targeting of the invention can also be used for studying, addressing or treating inflammatory disorders, as well as cancers. Folate-linked lipid particles or nanoparticles or liposomes or lipid bylayers of the invention (“lipid entity of the invention”) deliver their cargo intracellularly through receptor-mediated endocytosis. Intracellular trafficking can be directed to acidic compartments that facilitate cargo release, and, most importantly, release of the cargo can be altered or delayed until it reaches the cytoplasm or vicinity of target organelles. Delivery of cargo (e.g., the engineered nucleic acid modification systems or components thereof) using a lipid entity of the invention having a targeting moiety, such as a folate-linked lipid entity of the invention, can be superior to nontargeted lipid entity of the invention. The attachment of folate directly to the lipid head groups may not be favorable for intracellular delivery of folate-conjugated lipid entity of the invention, since they may not bind as efficiently to cells as folate attached to the lipid entity of the invention surface by a spacer, which may can enter cancer cells more efficiently. A lipid entity of the invention coupled to folate can be used for the delivery of complexes of lipid, e.g., liposome, e.g., anionic liposome and virus or capsid or envelope or virus outer protein, such as those herein discussed such as adenovirous or AAV. Tf is a monomeric serum glycoprotein of approximately 80 KDa involved in the transport of iron throughout the body. Tf binds to the TfR and translocates into cells via receptor-mediated endocytosis. The expression of TfR is higher in certain cells, such as tumor cells (as compared with normal cells and is associated with the increased iron demand in rapidly proliferating cancer cells. Accordingly, the invention comprehends a TfR-targeted lipid entity of the invention, e.g., as to liver cells, liver cancer, breast cells such as breast cancer cells, colon such as colon cancer cells, ovarian cells such as ovarian cancer cells, head, neck and lung cells, such as head, neck and non-small-cell lung cancer cells, cells of the mouth such as oral tumor cells.

Also, as to active targeting, a lipid entity of the invention can be multifunctional, i.e., employ more than one targeting moiety such as CPP, along with Tf; a bifunctional system; e.g., a combination of Tf and poly-L-arginine which can provide transport across the endothelium of the blood-brain barrier. EGFR is a tyrosine kinase receptor belonging to the ErbB family of receptors that mediates cell growth, differentiation and repair in cells, especially non-cancerous cells, but EGF is overexpressed in certain cells such as many solid tumors, including colorectal, non-small-cell lung cancer, squamous cell carcinoma of the ovary, kidney, head, pancreas, neck and prostate, and especially breast cancer. The invention comprehends EGFR-targeted monoclonal antibody(ies) linked to a lipid entity of the invention. HER-2 is often overexpressed in patients with breast cancer, and is also associated with lung, bladder, prostate, brain and stomach cancers. HER-2, encoded by the ERBB2 gene. The invention comprehends a HER-2-targeting lipid entity of the invention, e.g., an anti-HER-2-antibody (or binding fragment thereof)-lipid entity of the invention, a HER-2-targeting-PEGylated lipid entity of the invention (e.g., having an anti-HER-2-antibody or binding fragment thereof), a HER-2-targeting-maleimide-PEG polymer-lipid entity of the invention (e.g., having an anti-HER-2-antibody or binding fragment thereof). Upon cellular association, the receptor-antibody complex can be internalized by formation of an endosome for delivery to the cytoplasm.

With respect to receptor-mediated targeting, the skilled artisan takes into consideration ligand/target affinity and the quantity of receptors on the cell surface, and that PEGylation can act as a barrier against interaction with receptors. The use of antibody-lipid entity of the invention targeting can be advantageous. Multivalent presentation of targeting moieties can also increase the uptake and signaling properties of antibody fragments. In practice of the invention, the skilled person takes into account ligand density (e.g., high ligand densities on a lipid entity of the invention may be advantageous for increased binding to target cells). Preventing early by macrophages can be addressed with a sterically stabilized lipid entity of the invention and linking ligands to the terminus of molecules such as PEG, which is anchored in the lipid entity of the invention (e.g., lipid particle or nanoparticle or liposome or lipid bilayer). The microenvironment of a cell mass such as a tumor microenvironment can be targeted; for instance, it may be advantageous to target cell mass vasculature, such as the tumor vasculature microenvironment. Thus, the invention comprehends targeting VEGF. VEGF and its receptors are well-known proangiogenic molecules and are well-characterized targets for antiangiogenic therapy. Many small-molecule inhibitors of receptor tyrosine kinases, such as VEGFRs or basic FGFRs, have been developed as anticancer agents and the invention comprehends coupling any one or more of these peptides to a lipid entity of the invention, e.g., phage IVO peptide(s) (e.g., via or with a PEG terminus), tumor-homing peptide APRPG such as APRPG-PEG-modified. VCAM, the vascular endothelium plays a key role in the pathogenesis of inflammation, thrombosis and atherosclerosis. CAMs are involved in inflammatory disorders, including cancer, and are a logical target, E- and P-selectins, VCAM-1 and ICAMs. Can be used to target a lipid entity of the invention, e.g., with PEGylation.

Matrix metalloproteases (MMPs) belong to the family of zinc-dependent endopeptidases. They are involved in tissue remodeling, tumor invasiveness, resistance to apoptosis and metastasis. There are four MMP inhibitors called TIMP1-4, which determine the balance between tumor growth inhibition and metastasis; a protein involved in the angiogenesis of tumor vessels is MT1-MMP, expressed on newly formed vessels and tumor tissues. The proteolytic activity of MT1-MMP cleaves proteins, such as fibronectin, elastin, collagen and laminin, at the plasma membrane and activates soluble MMPs, such as MMP-2, which degrades the matrix. An antibody or fragment thereof such as a Fab′ fragment can be used in the practice of the invention such as for an antihuman MT1-MMP monoclonal antibody linked to a lipid entity of the invention, e.g., via a spacer such as a PEG spacer. αβ-integrins or integrins are a group of transmembrane glycoprotein receptors that mediate attachment between a cell and its surrounding tissues or extracellular matrix.

Integrins contain two distinct chains (heterodimers) called α- and β-subunits. The tumor tissue-specific expression of integrin receptors can be utilized for targeted delivery in the invention, e.g., whereby the targeting moiety can be an RGD peptide such as a cyclic RGD.

Aptamers are ssDNA or RNA oligonucleotides that impart high affinity and specific recognition of the target molecules by electrostatic interactions, hydrogen bonding and hydrophobic interactions as opposed to the Watson-Crick base pairing, which is typical for the bonding interactions of oligonucleotides. Aptamers as a targeting moiety can have advantages over antibodies: aptamers can demonstrate higher target antigen recognition as compared with antibodies; aptamers can be more stable and smaller in size as compared with antibodies; aptamers can be easily synthesized and chemically modified for molecular conjugation; and aptamers can be changed in sequence for improved selectivity and can be developed to recognize poorly immunogenic targets. Such moieties as a sgc8 aptamer can be used as a targeting moiety (e.g., via covalent linking to the lipid entity of the invention, e.g., via a spacer, such as a PEG spacer).

Also, as to active targeting, the invention also comprehends intracellular delivery. Since liposomes follow the endocytic pathway, they are entrapped in the endosomes (pH 6.5-6) and subsequently fuse with lysosomes (pH<5), where they undergo degradation that results in a lower therapeutic potential. The low endosomal pH can be taken advantage of to escape degradation. Fusogenic lipids or peptides, which destabilize the endosomal membrane after the conformational transition/activation at a lowered pH. Amines are protonated at an acidic pH and cause endosomal swelling and rupture by a buffer effect Unsaturated dioleoylphosphatidylethanolamine (DOPE) readily adopts an inverted hexagonal shape at a low pH, which causes fusion of liposomes to the endosomal membrane. This process destabilizes a lipid entity containing DOPE and releases the cargo into the cytoplasm; fusogenic lipid GALA, cholesteryl-GALA and PEG-GALA may show a highly efficient endosomal release; a pore-forming protein listeriolysin O may provide an endosomal escape mechanism; and histidine-rich peptides have the ability to fuse with the endosomal membrane, resulting in pore formation, and can buffer the proton pump causing membrane lysis.

The invention comprehends a lipid entity of the invention modified with CPP(s), for intracellular delivery that may proceed via energy dependent macropinocytosis followed by endosomal escape. The invention further comprehends organelle-specific targeting. A lipid entity of the invention surface-functionalized with the triphenylphosphonium (TPP) moiety or a lipid entity of the invention with a lipophilic cation, rhodamine 123 can be effective in delivery of cargo to mitochondria. DOPE/sphingomyelin/stearyl-octa-arginine can delivers cargos to the mitochondrial interior via membrane fusion. A lipid entity of the invention surface modified with a lysosomotropic ligand, octadecyl rhodamine B can delivercargoto lysosomes. Ceramides are useful in inducing lysosomal membrane permeabilization; the invention comprehends intracellular delivery of a lipid entity of the invention having a ceramide. The invention further comprehends a lipid entity of the invention targeting the nucleus, e.g., via a DNA-intercalating moiety. The invention also comprehends multifunctional liposomes for targeting, i.e., attaching more than one functional group to the surface of the lipid entity of the invention, for instance to enhances accumulation in a desired site and/or promotes organelle-specific delivery and/or target a particular type of cell and/or respond to the local stimuli such as temperature (e.g., elevated), pH (e.g., decreased), respond to externally applied stimuli such as a magnetic field, light, energy, heat or ultrasound and/or promote intracellular delivery of the cargo. All of these are considered actively targeting moieties.

It should be understood that as to each possible targeting or active targeting moiety herein-discussed, there is an embodiment of the invention wherein the delivery system comprises such a targeting or active targeting moiety. Likewise, Table 3 provides exemplary targeting moieties that can be used in the practice of the invention an as to each an embodiment of the invention provides a delivery system that comprises such a targeting moiety.

TABLE 3

| Targeting Moiety | Target Molecule | Target Cell or Tissue |
|---|---|---|
| folate | folate receptor | cancer cells |
| transferrin | transferrin receptor | cancer cells |
| Antibody CC52 | rat CC531 | rat colon adenocarcinoma CC531 |
| anti- HER2 antibody | HER2 | HER2 -overexpressing tumors |
| anti-GD2 | GD2 | neuroblastoma, melanoma |
| anti-EGFR | EGFR | tumor cells overexpressing EGFR |
| pH-dependent fusogenic | | ovarian carcinoma |
| peptide diINF-7 | | |
| anti-VEGFR | VEGF Receptor | tumor vasculature |
| anti-CD19 | CD19 (B cell marker) | leukemia, lymphoma |
| cell-penetrating peptide | | blood-brain barrier |
| cyclic arginine-glycine-aspartic | avβ3 | glioblastoma cells, human |
| acid-tyrosine-cysteine peptide | | umbilical vein endothelial cells, |
| (c(RGDyC)-LP) | | tumor angiogenesis |
| ASSHN peptide | | endothelial progenitor cells; anti-cancer |
| PR_b peptide | α₅β₁ integrin | cancer cells |
| AG86 peptide | α₆β₄ integrin | cancer cells |
| KCCYSL (P6.1 | HER-2 receptor | cancer cells |
| peptide) | | |
| affinity peptide LN | Aminopeptidase N | APN-positive tumor |
| (YEVGHRC) | (APN/CD13) | |
| synthetic somatostatin | Somatostatin receptor 2 | breast cancer |
| analogue | (SSTR2) | |
| anti-CD20 monoclonal | B-lymphocytes | B cell lymphoma |
| antibody | | |

Thus, in an embodiment of the delivery system, the targeting moiety comprises a receptor ligand, such as, for example, hyaluronic acid for CD44 receptor, galactose for hepatocytes, or antibody or fragment thereof such as a binding antibody fragment against a desired surface receptor, and as to each of a targeting moiety comprising a receptor ligand, or an antibody or fragment thereof such as a binding fragment thereof, such as against a desired surface receptor, there is an embodiment of the invention wherein the delivery system comprises a targeting moiety comprising a receptor ligand, or an antibody or fragment thereof such as a binding fragment thereof, such as against a desired surface receptor, or hyaluronic acid for CD44 receptor, galactose for hepatocytes (see e.g., Surace et al, “Lipoplexes targeting the CD44 hyaluronic acid receptor for efficient transfection of breast cancer cells,” J. Mol Pharm 6(4):1062-73; doi: 10.1021/mp800215d (2009); Sonoke et al, “Galactose-modified cationic liposomes as a liver-targeting delivery system for small interfering RNA,” Biol Pharm Bull. 34(8):1338-42 (2011); Torchilin, “Antibody-modified liposomes for cancer chemotherapy,” Expert Opin. Drug Deliv. 5 (9), 1003-1025 (2008); Manjappa et al, “Antibody derivatization and conjugation strategies: application in preparation of stealth immunoliposome to target chemotherapeutics to tumor,” J. Control. Release 150 (1), 2-22 (2011); Sofou S “Antibody-targeted liposomes in cancer therapy and imaging,” Expert Opin. Drug Deliv. 5 (2): 189-204 (2008); Gao J et al, “Antibody-targeted immunoliposomes for cancer treatment,” Mini. Rev. Med. Chem. 13(14): 2026-2035 (2013); Molavi et al, “Anti-CD30 antibody conjugated liposomal doxorubicin with significantly improved therapeutic efficacy against anaplastic large cell lymphoma,” Biomaterials 34(34):8718-25 (2013), each of which and the documents cited therein are hereby incorporated herein by reference), the teachings of which can be applied and/or adapted for targeted delivery of one or more engineered nucleic acid modification systems or components thereof described herein.

Other exemplary targeting moieties are described elsewhere herein, such as epitope tags and the like.

### Responsive Delivery

In some embodiments, the delivery vehicle can allow for responsive delivery of the cargo(s). Responsive delivery, as used in this context herein, refers to delivery of cargo(s) by the delivery vehicle in response to an external stimuli. Examples of suitable stimuli include, without limitation, an energy (light, heat, cold, and the like), a chemical stimuli (e.g., chemical composition, etc.), and a biologic or physiologic stimuli (e.g., environmental pH, osmolarity, salinity, biologic molecule, etc.). In some embodiments, the targeting moiety can be responsive to an external stimuli and facilitate responsive delivery. In other embodiments, responsiveness is determined by a non-targeting moiety component of the delivery vehicle.

The delivery vehicle can be stimuli-sensitive, e.g., sensitive to an externally applied stimuli, such as magnetic fields, ultrasound or light; and pH-triggering can also be used, e.g., a labile linkage can be used between a hydrophilic moiety such as PEG and a hydrophobic moiety such as a lipid entity of the invention, which is cleaved only upon exposure to the relatively acidic conditions characteristic of the a particular environment or microenvironment such as an endocytic vacuole or the acidotic tumor mass. pH-sensitive copolymers can also be incorporated in embodiments of the invention can provide shielding; diortho esters, vinyl esters, cysteine-cleavable lipopolymers, double esters and hydrazones are a few examples of pH-sensitive bonds that are quite stable at pH 7.5, but are hydrolyzed relatively rapidly at pH 6 and below, e.g., a terminally alkylated copolymer of N-isopropylacrylamide and methacrylic acid that copolymer facilitates destabilization of a lipid entity of the invention and release in compartments with decreased pH value; or, the invention comprehends ionic polymers for generation of a pH-responsive lipid entity of the invention (e.g., poly(methacrylic acid), poly(diethylaminoethyl methacrylate), poly(acrylamide) and poly(acrylic acid)).

Temperature-triggered delivery is also within the ambit of the invention. Many pathological areas, such as inflamed tissues and tumors, show a distinctive hyperthermia compared with normal tissues. Utilizing this hyperthermia is an attractive strategy in cancer therapy since hyperthermia is associated with increased tumor permeability and enhanced uptake. This technique involves local heating of the site to increase microvascular pore size and blood flow, which, in turn, can result in an increased extravasation of embodiments of the invention. Temperature-sensitive lipid entity of the invention can be prepared from thermosensitive lipids or polymers with a low critical solution temperature. Above the low critical solution temperature (e.g., at site such as tumor site or inflamed tissue site), the polymer precipitates, disrupting the liposomes to release. Lipids with a specific gel-to-liquid phase transition temperature are used to prepare these lipid entities of the invention; and a lipid for a thermosensitive embodiment can be dipalmitoylphosphatidylcholine. Thermosensitive polymers can also facilitate destabilization followed by release, and a useful thermosensitive polymer is poly (N-isopropylacrylamide). Another temperature triggered system can employ lysolipid temperature-sensitive liposomes.

The invention also comprehends redox-triggered delivery. The difference in redox potential between normal and inflamed or tumor tissues, and between the intra- and extra-cellular environments has been exploited for delivery, e.g., GSH is a reducing agent abundant in cells, especially in the cytosol, mitochondria and nucleus. The GSH concentrations in blood and extracellular matrix are just one out of 100 to one out of 1000 of the intracellular concentration, respectively. This high redox potential difference caused by GSH, cysteine and other reducing agents can break the reducible bonds, destabilize a lipid entity of the invention and result in release of payload. The disulfide bond can be used as the cleavable/reversible linker in a lipid entity of the invention, because it causes sensitivity to redox owing to the disulfideto-thiol reduction reaction; a lipid entity of the invention can be made reduction sensitive by using two (e.g., two forms of a disulfide-conjugated multifunctional lipid as cleavage of the disulfide bond (e.g., via tris(2-carboxyethyl)phosphine, dithiothreitol, L-cysteine or GSH), can cause removal of the hydrophilic head group of the conjugate and alter the membrane organization leading to release of payload. Calcein release from reduction-sensitive lipid entity of the invention containing a disulfide conjugate can be more useful than a reduction-insensitive embodiment.

Enzymes can also be used as a trigger to release payload. Enzymes, including MMPs (e.g., MMP2), phospholipase A2, alkaline phosphatase, transglutaminase or phosphatidylinositol-specific phospholipase C, have been found to be overexpressed in certain tissues, e.g., tumor tissues. In the presence of these enzymes, specially engineered enzyme-sensitive lipid entity of the invention can be disrupted and release the payload. an MMP2-cleavable octapeptide (Gly-Pro-Leu-Gly-Ile-Ala-Gly-Gln) (SEQ ID NO: 40) can be incorporated into a linker, and can have antibody targeting, e.g., antibody 2C5.

The invention also comprehends light- or energy-triggered delivery, e.g., the lipid entity of the invention can be light-sensitive, such that light or energy can facilitate structural and conformational changes, which lead to direct interaction of the lipid entity of the invention with the target cells via membrane fusion, photo-isomerism, photofragmentation or photopolymerization; such a moiety therefor can be benzoporphyrin photosensitizer. Ultrasound can be a form of energy to trigger delivery; a lipid entity of the invention with a small quantity of particular gas, including air or perfluorated hydrocarbon can be triggered to release with ultrasound, e.g., low-frequency ultrasound (LFUS). Magnetic delivery: A lipid entity of the invention can be magnetized by incorporation of magnetites, such as Fe3O4 or γ-Fe₂O₃, e.g., those that are less than 10 nm in size. Targeted delivery can be then by exposure to a magnetic field.

### Pharmaceutical Formulations

Also described herein are pharmaceutical formulations that can contain an amount, effective amount, and/or least effective amount, and/or therapeutically effective amount of one or more compounds, molecules, compositions, vectors, vector systems, cells, or a combination thereof of the present invention (which are also referred to as the primary active agent or ingredient elsewhere herein) and a pharmaceutically acceptable carrier or excipient. As used herein, “pharmaceutical formulation” refers to the combination of an active agent, compound, or ingredient with a pharmaceutically acceptable carrier or excipient, making the composition suitable for diagnostic, therapeutic, or preventive use in vitro, in vivo, or ex vivo. As used herein, “pharmaceutically acceptable carrier or excipient” refers to a carrier or excipient that is useful in preparing a pharmaceutical formulation that is generally safe, non-toxic, and is neither biologically or otherwise undesirable, and includes a carrier or excipient that is acceptable for veterinary use as well as human pharmaceutical use. A “pharmaceutically acceptable carrier or excipient” as used in the specification and claims includes both one and more than one such carrier or excipient. When present, the compound can optionally be present in the pharmaceutical formulation as a pharmaceutically acceptable salt. In some embodiments, the pharmaceutical formulation can include, such as an active ingredient, a engineered nucleic acid modification systems or components thereof described in greater detail elsewhere herein. In some embodiments, the pharmaceutical formulation can include, such as an active ingredient, an engineered nucleic acid modification systems or components thereof in greater detail elsewhere herein. In some embodiments, the pharmaceutical formulation can include, such as an active ingredient one or more modified cells, such as one or more modified cells described in greater detail elsewhere herein.

In some embodiments, the active ingredient is present as a pharmaceutically acceptable salt of the active ingredient. As used herein, “pharmaceutically acceptable salt” refers to any acid or base addition salt whose counter-ions are non-toxic to the subject to which they are administered in pharmaceutical doses of the salts. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

The pharmaceutical formulations described herein can be administered to a subject in need thereof via any suitable method or route to a subject in need thereof. Suitable administration routes can include, but are not limited to auricular (otic), buccal, conjunctival, cutaneous, dental, electro-osmosis, endocervical, endosinusial, endotracheal, enteral, epidural, extra-amniotic, extracorporeal, hemodialysis, infiltration, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal (dental), intracoronary, intracorporus cavernosum, intradermal, intradiscal, intraductal, intraduodenal, intradural, intraepidermal, intraesophageal, intragastric, intragingival, intraileal, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravesical, intravitreal, iontophoresis, irrigation, laryngeal, nasal, nasogastric, occlusive dressing technique, ophthalmic, oral, oropharyngeal, other, parenteral, percutaneous, periarticular, peridural, perineural, periodontal, rectal, respiratory (inhalation), retrobulbar, soft tissue, subarachnoid, subconjunctival, subcutaneous, sublingual, submucosal, topical, transdermal, transmucosal, transplacental, transtracheal, transtympanic, ureteral, urethral, and/or vaginal administration, and/or any combination of the above administration routes, which typically depends on the disease to be treated and/or the active ingredient(s).

Where appropriate, compounds, molecules, compositions, vectors, vector systems, cells, or a combination thereof described in greater detail elsewhere herein can be provided to a subject in need thereof as an ingredient, such as an active ingredient or agent, in a pharmaceutical formulation. As such, also described are pharmaceutical formulations containing one or more of the compounds and salts thereof, or pharmaceutically acceptable salts thereof described herein. Suitable salts include, hydrobromide, iodide, nitrate, bisulfate, phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, camphorsulfonate, napthalenesulfonate, propionate, malonate, mandelate, malate, phthalate, and pamoate.

In some embodiments, the subject in need thereof has or is suspected of having a hematopoietic disease or a symptom thereof. In some embodiments, the subject in need thereof has or is suspected of having, a neurobiological disease or disorder, a psychiatric disease or disorder, a cancer, an autoimmune disease or disorder, a thrombosis disease, a heart disease, a kidney disease, a lung disease, or a blood vessel disease, or a combination thereof. As used herein, “agent” refers to any substance, compound, molecule, and the like, which can be biologically active or otherwise can induce a biological and/or physiological effect on a subject to which it is administered to. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed.

### Pharmaceutically Acceptable Carriers and Secondary Ingredients and Agents

The pharmaceutical formulation can include a pharmaceutically acceptable carrier. Suitable pharmaceutically acceptable carriers include, but are not limited to water, salt solutions, alcohols, gum arabic, vegetable oils, benzyl alcohols, polyethylene glycols, gelatin, carbohydrates such as lactose, amylose or starch, magnesium stearate, talc, silicic acid, viscous paraffin, perfume oil, fatty acid esters, hydroxy methylcellulose, and polyvinyl pyrrolidone, which do not deleteriously react with the active composition.

The pharmaceutical formulations can be sterilized, and if desired, mixed with agents, such as lubricants, preservatives, stabilizers, wetting agents, emulsifiers, salts for influencing osmotic pressure, buffers, coloring, flavoring and/or aromatic substances, and the like which do not deleteriously react with the active compound.

In some embodiments, the pharmaceutical formulation can also include an effective amount of secondary active agents, including but not limited to, biologic agents or molecules including, but not limited to, e.g., polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Effective Amounts

In some embodiments, the amount of the primary active agent and/or optional secondary agent can be an effective amount, least effective amount, and/or therapeutically effective amount. As used herein, “effective amount” refers to the amount of the primary and/or optional secondary agent included in the pharmaceutical formulation that achieve one or more therapeutic effects or desired effect. As used herein, “least effective” amount refers to the lowest amount of the primary and/or optional secondary agent that achieves the one or more therapeutic or other desired effects. As used herein, “therapeutically effective amount” refers to the amount of the primary and/or optional secondary agent included in the pharmaceutical formulation that achieves one or more therapeutic effects. In some embodiments, the therapeutic effect is polynucleotide modification.

The effective amount, least effective amount, and/or therapeutically effective amount of the primary and optional secondary active agent described elsewhere herein contained in the pharmaceutical formulation can range from about 0 to 10, 20, 30, 40, 50, 60, 70, 80, 90, 100, 110, 120,130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pg, ng, g, mg, or g or be any numerical value with any of these ranges.

In some embodiments, the effective amount, least effective amount, and/or therapeutically effective amount can be an effective concentration, least effective concentration, and/or therapeutically effective concentration, which can each range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 pM, nM, μM, mM, or M or be any numerical value with any of these ranges.

In other embodiments, the effective amount, least effective amount, and/or therapeutically effective amount of the primary and optional secondary active agent can range from about 0 to 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 IU or be any numerical value with any of these ranges.

In some embodiments, the primary and/or the optional secondary active agent present in the pharmaceutical formulation can range from about 0 to 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.11, 0.12, 0.13, 0.14, 0.15, 0.16, 0.17, 0.18, 0.19, 0.2, 0.21, 0.22, 0.23, 0.24, 0.25, 0.26, 0.27, 0.28, 0.29, 0.3, 0.31, 0.32, 0.33, 0.34, 0.35, 0.36, 0.37, 0.38, 0.39, 0.4, 0.41, 0.42, 0.43, 0.44, 0.45, 0.46, 0.47, 0.48, 0.49, 0.5, 0.51, 0.52, 0.53, 0.54, 0.55, 0.56, 0.57, 0.58, 0.59, 0.6, 0.61, 0.62, 0.63, 0.64, 0.65, 0.66, 0.67, 0.68, 0.69, 0.7, 0.71, 0.72, 0.73, 0.74, 0.75, 0.76, 0.77, 0.78, 0.79, 0.8, 0.81, 0.82, 0.83, 0.84, 0.85, 0.86, 0.87, 0.88, 0.89, 0.9, 0.91, 0.92, 0.93, 0.94, 0.95, 0.96, 0.97, 0.98, 0.9, to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the pharmaceutical formulation.

In some embodiments where a cell population is present in the pharmaceutical formulation (e.g., as a primary and/or or secondary active agent), the effective amount of cells can range from about 2 cells to 1×10¹/mL, 1×10²⁰/mL or more, such as about 1×10¹/mL, 1×10²/mL, 1×10³/mL, 1×10⁴/mL, 1×10⁵/mL, 1×10⁶/mL, 1×10⁷/mL, 1×10⁸/mL, 1×10⁹/mL, 1×10¹⁰/mL, 1×10¹¹/mL, 1×10¹²/mL, 1×10¹³/mL, 1×10¹⁴/mL, 1×10¹⁵/mL, 1×10¹⁶/mL, 1×10¹⁷/mL, 1×10¹⁸/mL, 1×10¹⁹/mL, to/or about 1×10²⁰/mL.

In some embodiments, the amount or effective amount, particularly where an infective particle is being delivered (e.g., a virus particle having the primary or secondary agent as a cargo), the effective amount of virus particles can be expressed as a titer (plaque forming units per unit of volume) or as a MOI (multiplicity of infection). In some embodiments, the effective amount can be 1×10¹ particles per pL, nL, μL, mL, or L to 1×10²⁰/particles per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³ 1×10¹⁴, 1×10¹⁵, 1×10¹⁶, 1×10¹⁷, 1×10¹⁸, 1×10¹⁹, to/or about 1×10²⁰ particles per pL, nL, μL, mL, or L. In some embodiments, the effective titer can be about 1×10¹ transforming units per pL, nL, μL, mL, or L to 1×10²⁰/transforming units per pL, nL, μL, mL, or L or more, such as about 1×10¹, 1×10², 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, 1×10¹⁶, 1×10¹⁷, 1×10¹⁸, 1×10¹⁹, to/or about 1×10²⁰ transforming units per pL, nL, μL, mL, or L. In some embodiments, the MOI of the pharmaceutical formulation can range from about 0.1 to 10 or more, such as 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9, 8, 8.1, 8.2, 8.3, 8.4, 8.5, 8.6, 8.7, 8.8, 8.9, 9, 9.1, 9.2, 9.3, 9.4, 9.5, 9.6, 9.7, 9.8, 9.9, 10 or more.

In some embodiments, the amount or effective amount of the one or more of the active agent(s) described herein contained in the pharmaceutical formulation can range from about 1 pg/kg to about 10 mg/kg based upon the bodyweight of the subject in need thereof or average bodyweight of the specific patient population to which the pharmaceutical formulation can be administered.

In embodiments where there is a secondary agent contained in the pharmaceutical formulation, the effective amount of the secondary active agent will vary depending on the secondary agent, the primary agent, the administration route, subject age, disease, stage of disease, among other things, which will be one of ordinary skill in the art.

When optionally present in the pharmaceutical formulation, the secondary active agent can be included in the pharmaceutical formulation or can exist as a stand-alone compound or pharmaceutical formulation that can be administered contemporaneously or sequentially with the compound, derivative thereof, or pharmaceutical formulation thereof.

In some embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total secondary active agent in the pharmaceutical formulation. In additional embodiments, the effective amount of the secondary active agent can range from about 0 to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, 99.9% w/w, v/v, or w/v of the total pharmaceutical formulation.

### Dosage Forms

In some embodiments, the pharmaceutical formulations described herein can be provided in a dosage form. The dosage form can be administered to a subject in need thereof. The dosage form can be effective generate specific concentration, such as an effective concentration, at a given site in the subject in need thereof. As used herein, “dose,” “unit dose,” or “dosage” can refer to physically discrete units suitable for use in a subject, each unit containing a predetermined quantity of the primary active agent, and optionally present secondary active ingredient, and/or a pharmaceutical formulation thereof calculated to produce the desired response or responses in association with its administration. In some embodiments, the given site is proximal to the administration site. In some embodiments, the given site is distal to the administration site. In some cases, the dosage form contains a greater amount of one or more of the active ingredients present in the pharmaceutical formulation than the final intended amount needed to reach a specific region or location within the subject to account for loss of the active components such as via first and second pass metabolism.

The dosage forms can be adapted for administration by any appropriate route. Appropriate routes include, but are not limited to, oral (including buccal or sublingual), rectal, intraocular, inhaled, intranasal, topical (including buccal, sublingual, or transdermal), vaginal, parenteral, subcutaneous, intramuscular, intravenous, internasal, and intradermal. Other appropriate routes are described elsewhere herein. Such formulations can be prepared by any method known in the art.

Dosage forms adapted for oral administration can discrete dosage units such as capsules, pellets or tablets, powders or granules, solutions, or suspensions in aqueous or non-aqueous liquids; edible foams or whips, or in oil-in-water liquid emulsions or water-in-oil liquid emulsions. In some embodiments, the pharmaceutical formulations adapted for oral administration also include one or more agents which flavor, preserve, color, or help disperse the pharmaceutical formulation. Dosage forms prepared for oral administration can also be in the form of a liquid solution that can be delivered as a foam, spray, or liquid solution. The oral dosage form can be administered to a subject in need thereof. Where appropriate, the dosage forms described herein can be microencapsulated.

The dosage form can also be prepared to prolong or sustain the release of any ingredient. In some embodiments, compounds, molecules, compositions, vectors, vector systems, cells, or a combination thereof described herein can be the ingredient whose release is delayed. In some embodiments the primary active agent is the ingredient whose release is delayed. In some embodiments, an optional secondary agent can be the ingredient whose release is delayed. Suitable methods for delaying the release of an ingredient include, but are not limited to, coating or embedding the ingredients in material in polymers, wax, gels, and the like. Delayed release dosage formulations can be prepared as described in standard references such as “Pharmaceutical dosage form tablets,” eds. Liberman et. al. (New York, Marcel Dekker, Inc., 1989), “Remington—The science and practice of pharmacy”, 20th ed., Lippincott Williams & Wlkins, Baltimore, M D, 2000, and “Pharmaceutical dosage forms and drug delivery systems”, 6th Edition, Ansel et al., (Media, PA: Wlliams and Wlkins, 1995). These references provide information on excipients, materials, equipment, and processes for preparing tablets and capsules and delayed release dosage forms of tablets and pellets, capsules, and granules. The delayed release can be anywhere from about an hour to about 3 months or more.

Examples of suitable coating materials include, but are not limited to, cellulose polymers such as cellulose acetate phthalate, hydroxypropyl cellulose, hydroxypropyl methylcellulose, hydroxypropyl methylcellulose phthalate, and hydroxypropyl methylcellulose acetate succinate; polyvinyl acetate phthalate, acrylic acid polymers and copolymers, and methacrylic resins that are commercially available under the trade name EUDRAGIT® (Roth Pharma, Westerstadt, Germany), zein, shellac, and polysaccharides.

Coatings may be formed with a different ratio of water-soluble polymer, water insoluble polymers, and/or pH dependent polymers, with or without water insoluble/water soluble non-polymeric excipient, to produce the desired release profile. The coating is either performed on the dosage form (matrix or simple) which includes, but is not limited to, tablets (compressed with or without coated beads), capsules (with or without coated beads), beads, particle compositions, “ingredient as is” formulated as, but not limited to, suspension form or as a sprinkle dosage form.

Where appropriate, the dosage forms described herein can be a liposome. In these embodiments, primary active ingredient(s), and/or optional secondary active ingredient(s), and/or pharmaceutically acceptable salt thereof where appropriate are incorporated into a liposome. In embodiments where the dosage form is a liposome, the pharmaceutical formulation is thus a liposomal formulation. The liposomal formulation can be administered to a subject in need thereof.

Dosage forms adapted for topical administration can be formulated as ointments, creams, suspensions, lotions, powders, solutions, pastes, gels, sprays, aerosols, or oils. In some embodiments for treatments of the eye or other external tissues, for example the mouth or the skin, the pharmaceutical formulations are applied as a topical ointment or cream. When formulated in an ointment, a primary active ingredient, optional secondary active ingredient, and/or pharmaceutically acceptable salt thereof where appropriate can be formulated with a paraffinic or water-miscible ointment base. In other embodiments, the primary and/or secondary active ingredient can be formulated in a cream with an oil-in-water cream base or a water-in-oil base. Dosage forms adapted for topical administration in the mouth include lozenges, pastilles, and mouth washes.

Dosage forms adapted for nasal or inhalation administration include aerosols, solutions, suspension drops, gels, or dry powders. In some embodiments, a primary active ingredient, optional secondary active ingredient, and/or pharmaceutically acceptable salt thereof where appropriate can be in a dosage form adapted for inhalation is in a particle-size-reduced form that is obtained or obtainable by micronization. In some embodiments, the particle size of the size reduced (e.g., micronized) compound or salt or solvate thereof, is defined by a D₅₀ value of about 0.5 to about 10 microns as measured by an appropriate method known in the art. Dosage forms adapted for administration by inhalation also include particle dusts or mists. Suitable dosage forms wherein the carrier or excipient is a liquid for administration as a nasal spray or drops include aqueous or oil solutions/suspensions of an active (primary and/or secondary) ingredient, which may be generated by various types of metered dose pressurized aerosols, nebulizers, or insufflators. The nasal/inhalation formulations can be administered to a subject in need thereof.

In some embodiments, the dosage forms are aerosol formulations suitable for administration by inhalation. In some of these embodiments, the aerosol formulation contains a solution or fine suspension of a primary active ingredient, secondary active ingredient, and/or pharmaceutically acceptable salt thereof where appropriate and a pharmaceutically acceptable aqueous or non-aqueous solvent. Aerosol formulations can be presented in single or multi-dose quantities in sterile form in a sealed container. For some of these embodiments, the sealed container is a single dose or multi-dose nasal or an aerosol dispenser fitted with a metering valve (e.g. metered dose inhaler), which is intended for disposal once the contents of the container have been exhausted.

Where the aerosol dosage form is contained in an aerosol dispenser, the dispenser contains a suitable propellant under pressure, such as compressed air, carbon dioxide, or an organic propellant, including but not limited to a hydrofluorocarbon. The aerosol formulation dosage forms in other embodiments are contained in a pump-atomizer. The pressurized aerosol formulation can also contain a solution or a suspension of a primary active ingredient, optional secondary active ingredient, and/or pharmaceutically acceptable salt thereof. In further embodiments, the aerosol formulation also contains co-solvents and/or modifiers incorporated to improve, for example, the stability and/or taste and/or fine particle mass characteristics (amount and/or profile) of the formulation. Administration of the aerosol formulation can be once daily or several times daily, for example 2, 3, 4, or 8 times daily, in which 1, 2, 3 or more doses are delivered each time. The aerosol formulations can be administered to a subject in need thereof.

For some dosage forms suitable and/or adapted for inhaled administration, the pharmaceutical formulation is a dry powder inhalable-formulations. In addition to a primary active agent, optional secondary active ingredient, and/or pharmaceutically acceptable salt thereof where appropriate, such a dosage form can contain a powder base such as lactose, glucose, trehalose, manitol, and/or starch. In some of these embodiments, a primary active agent, secondary active ingredient, and/or pharmaceutically acceptable salt thereof where appropriate is in a particle-size reduced form. In further embodiments, a performance modifier, such as L-leucine or another amino acid, cellobiose octaacetate, and/or metals salts of stearic acid, such as magnesium or calcium stearate. In some embodiments, the aerosol formulations are arranged so that each metered dose of aerosol contains a predetermined amount of an active ingredient, such as the one or more of the compositions, compounds, vector(s), molecules, cells, and combinations thereof described herein.

Dosage forms adapted for vaginal administration can be presented as pessaries, tampons, creams, gels, pastes, foams, or spray formulations. Dosage forms adapted for rectal administration include suppositories or enemas. The vaginal formulations can be administered to a subject in need thereof.

Dosage forms adapted for parenteral administration and/or adapted for injection can include aqueous and/or non-aqueous sterile injection solutions, which can contain antioxidants, buffers, bacteriostats, solutes that render the composition isotonic with the blood of the subject, and aqueous and non-aqueous sterile suspensions, which can include suspending agents and thickening agents. The dosage forms adapted for parenteral administration can be presented in a single-unit dose or multi-unit dose containers, including but not limited to sealed ampoules or vials. The doses can be lyophilized and re-suspended in a sterile carrier to reconstitute the dose prior to administration. Extemporaneous injection solutions and suspensions can be prepared in some embodiments, from sterile powders, granules, and tablets. The parenteral formulations can be administered to a subject in need thereof.

For some embodiments, the dosage form contains a predetermined amount of a primary active agent, secondary active ingredient, and/or pharmaceutically acceptable salt thereof where appropriate per unit dose. In an embodiment, the predetermined amount of primary active agent, secondary active ingredient, and/or pharmaceutically acceptable salt thereof where appropriate can be an effective amount, a least effect amount, and/or a therapeutically effective amount. In other embodiments, the predetermined amount of a primary active agent, secondary active agent, and/or pharmaceutically acceptable salt thereof where appropriate, can be an appropriate fraction of the effective amount of the active ingredient.

### Co-Therapies and Combination Therapies

In some embodiments, the pharmaceutical formulation(s) described herein can be part of a combination treatment or combination therapy. The combination treatment can include the pharmaceutical formulation described herein and an additional treatment modality. The additional treatment modality can be a chemotherapeutic, a biological therapeutic, surgery, radiation, diet modulation, environmental modulation, a physical activity modulation, and combinations thereof.

In some embodiments, the co-therapy or combination therapy can additionally include but not limited to, polynucleotides, amino acids, peptides, polypeptides, antibodies, aptamers, ribozymes, hormones, immunomodulators, antipyretics, anxiolytics, antipsychotics, analgesics, antispasmodics, anti-inflammatories, anti-histamines, anti-infectives, chemotherapeutics, and combinations thereof.

### Administration of the Pharmaceutical Formulations

The pharmaceutical formulations or dosage forms thereof described herein can be administered one or more times hourly, daily, monthly, or yearly (e.g., 1, 2, 3, 4, 5, 6, 7, 8, 9, 10, 11, 12, 13, 14, 15, 16, 17, 18, 19, 20, or more times hourly, daily, monthly, or yearly). In some embodiments, the pharmaceutical formulations or dosage forms thereof described herein can be administered continuously over a period of time ranging from minutes to hours to days. Devices and dosages forms are known in the art and described herein that are effective to provide continuous administration of the pharmaceutical formulations described herein. In some embodiments, the first one or a few initial amount(s) administered can be a higher dose than subsequent doses. This is typically referred to in the art as a loading dose or doses and a maintenance dose, respectively. In some embodiments, the pharmaceutical formulations can be administered such that the doses over time are tapered (increased or decreased) overtime so as to wean a subject gradually off of a pharmaceutical formulation or gradually introduce a subject to the pharmaceutical formulation.

As previously discussed, the pharmaceutical formulation can contain a predetermined amount of a primary active agent, secondary active agent, and/or pharmaceutically acceptable salt thereof where appropriate. In some of these embodiments, the predetermined amount can be an appropriate fraction of the effective amount of the active ingredient. Such unit doses may therefore be administered once or more than once a day, month, or year (e.g., 1, 2, 3, 4, 5, 6, or more times per day, month, or year). Such pharmaceutical formulations may be prepared by any of the methods well known in the art.

Where co-therapies or multiple pharmaceutical formulations are to be delivered to a subject, the different therapies or formulations can be administered sequentially or simultaneously. Sequential administration is administration where an appreciable amount of time occurs between administrations, such as more than about 15, 20, 30, 45, 60 minutes or more. The time between administrations in sequential administration can be on the order of hours, days, months, or even years, depending on the active agent present in each administration. Simultaneous administration refers to administration of two or more formulations at the same time or substantially at the same time (e.g., within seconds or just a few minutes apart), where the intent is that the formulations be administered together at the same time.

### Applications in Non-Animal Organisms

The systems and methods herein may be used in non-animal organisms, e.g., plants, fungi. The system(s) (e.g., single or multiplexed) can be used in conjunction with recent advances in crop genomics. The systems described herein can be used to perform efficient and cost effective plant gene or genome interrogation or editing or manipulation—for instance, for rapid investigation and/or selection and/or interrogations and/or comparison and/or manipulations and/or transformation of plant genes or genomes; e.g., to create, identify, develop, optimize, or confer trait(s) or characteristic(s) to plant(s) or to transform a plant genome. There can accordingly be improved production of plants, new plants with new combinations of traits or characteristics or new plants with enhanced traits. The engineered nucleic acid modification systems of the present invention can be used with regard to plants in Site-Directed Integration (SDI) or Gene Editing (GE) or any Near Reverse Breeding (NRB) or Reverse Breeding (RB) techniques. Aspects of utilizing the herein described engineered nucleic acid modification systems or components thereof may be analogous to the use of the CRISPR-Cas system in plants, and mention is made of the University of Arizona website “CRISPR-PLANT” (www.genome.arizona.edu/crispr/) (supported by Penn State and AGI). Embodiments of the invention can be used with haploid induction. For example, a corn line capable of making pollen able to trigger haploid induction is transformed with a systems programmed to target genes related to desirable traits. The pollen is used to transfer the systems to other corn varieties otherwise resistant to CRISPR transfer. In certain embodiments, the engineered nucleic acid modification system-carrying corn pollen can edit or otherwise modify the DNA of wheat. Embodiments of the invention can be used in genome editing in plants or where RNAi or similar genome editing techniques have been used previously; see, e.g., Nekrasov, “Plant genome editing made easy: targeted mutagenesis in model and crop plants using the CRISPR-Cas system,” Plant Methods 2013, 9:39 (doi:10.1186/1746-4811-9-39); Brooks, “Efficient gene editing in tomato in the first generation using the CRISPR-Cas9 system,” Plant Physiology September 2014 pp 114.247577; Shan, “Targeted genome modification of crop plants using a CRISPR-Cas system,” Nature Biotechnology 31, 686-688 (2013); Feng, “Efficient genome editing in plants using a CRISPR/Cas system,” Cell Research (2013) 23:1229-1232. doi:10.1038/cr.2013.114; published online 20 Aug. 2013; Xie, “RNA-guided genome editing in plants using a CRISPR-Cas system,” Mol Plant. 2013 November; 6(6):1975-83. doi: 10.1093/mp/sst119. Epub 2013 Aug. 17; Xu, “Gene targeting using the Agrobacterium tumefaciens-mediated CRISPR-Cas system in rice,” Rice 2014, 7:5 (2014), Zhou et al., “Exploiting SNPs for biallelic CRISPR mutations in the outcrossing woody perennial Populus reveals 4-coumarate: CoA ligase specificity and Redundancy,” New Phytologist (2015) (Forum) 1-4 (available online only at www.newphytologist.com); Caliando et al, “Targeted DNA degradation using a CRISPR device stably carried in the host genome, NATURE COMMUNICATIONS 6:6989, DOI: 10.1038/ncomms7989, www.nature.com/naturecommunications DOI: 10.1038/ncomms7989; U.S. Pat. No. 6,603,061 —Agrobacterium-Mediated Plant Transformation Method; U.S. Pat. No. 7,868,149 Plant Genome Sequences and Uses Thereof and US 2009/0100536—Transgenic Plants with Enhanced Agronomic Traits, all the contents and disclosure of each of which are herein incorporated by reference in their entirety. In the practice of the invention, the contents and disclosure of Morrell et al “Crop genomics: advances and applications,” Nat Rev Genet. 2011 Dec. 29; 13(2):85-96; each of which is incorporated by reference herein including as to how herein embodiments may be used as to plants. Accordingly, reference herein to animal cells may also apply, mutatis mutandis, to plant cells unless otherwise apparent; and the enzymes herein having reduced off-target effects and systems employing such enzymes can be used in plant applications, including those mentioned herein.

In general, the term “plant” relates to any various photosynthetic, eukaryotic, unicellular or multicellular organism of the kingdom Plantae characteristically growing by cell division, containing chloroplasts, and having cell walls comprised of cellulose. The term plant encompasses monocotyledonous and dicotyledonous plants. Specifically, the plants are intended to comprise without limitation angiosperm and gymnosperm plants such as acacia, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. The term plant also encompasses Algae, which are mainly photoautotrophs unified primarily by their lack of roots, leaves and other organs that characterize higher plants.

The methods for genome editing using the systems as described herein can be used to confer desired traits on essentially any plant. A wide variety of plants and plant cell systems may be engineered for the desired physiological and agronomic characteristics described herein using the nucleic acid constructs of the present disclosure and the various transformation methods mentioned above. In preferred embodiments, target plants and plant cells for engineering include, but are not limited to, those monocotyledonous and dicotyledonous plants, such as crops including grain crops (e.g., wheat, maize, rice, millet, barley), fruit crops (e.g., tomato, apple, pear, strawberry, orange), forage crops (e.g., alfalfa), root vegetable crops (e.g., carrot, potato, sugar beets, yam), leafy vegetable crops (e.g., lettuce, spinach); flowering plants (e.g., petunia, rose, chrysanthemum), conifers and pine trees (e.g., pine fir, spruce); plants used in phytoremediation (e.g., heavy metal accumulating plants); oil crops (e.g., sunflower, rape seed) and plants used for experimental purposes (e.g., Arabidopsis). Plant cells and tissues for engineering include, without limitation, roots, stems, leaves, flowers, and reproductive structures, undifferentiated meristematic cells, parenchyma, collenchyma, sclerenchyma, xylem, phloem, epidermis, and germplasm. Thus, the methods and engineered nucleic acid modification systems or components thereof of the present invention can be used over a broad range of plants, such as for example with dicotyledonous plants belonging to the orders Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales; the methods and CRISPR-Cas systems can be used with monocotyledonous plants such as those belonging to the orders Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales, or with plants belonging to Gymnospermae, e.g those belonging to the orders Pinales, Ginkgoales, Cycadales, Araucariales, Cupressales and Gnetales.

The systems and methods of use described herein can be used over a broad range of plant species, included in the non-limitative list of dicot, monocot or gymnosperm genera hereunder: Atropa, Alseodaphne, Anacardium, Arachis, Beilschmiedia, Brassica, Carthamus, Cocculus, Croton, Cucumis, Citrus, Citrullus, Capsicum, Catharanthus, Cocos, Coffea, Cucurbita, Daucus, Duguetia, Eschscholzia, Ficus, Fragaria, Glaucium, Glycine, Gossypium, Helianthus, Hevea, Hyoscyamus, Lactuca, Landolphia, Linum, Litsea, Lycopersicon, Lupinus, Manihot, Majorana, Malus, Medicago, Nicotiana, Olea, Parthenium, Papaver, Persea, Phaseolus, Pistacia, Pisum, Pyrus, Prunus, Raphanus, Ricinus, Senecio, Sinomenium, Stephania, Sinapis, Solanum, Theobroma, Trifolium, Trigonella, Vicia, Vinca, Vilis, and Vigna; and the genera Allium, Andropogon, Aragrostis, Asparagus, Avena, Cynodon, Elaeis, Festuca, Festulolium, Heterocallis, Hordeum, Lemna, Lolium, Musa, Oryza, Panicum, Pannesetum, Phleum, Poa, Secale, Sorghum, Triticum, Zea, Abies, Cunninghamia, Ephedra, Picea, Pinus, and Pseudotsuga.

The systems and methods of use can also be used over a broad range of “algae” or “algae cells”; including for example algae selected from several eukaryotic phyla, including the Rhodophyta (red algae), Chlorophyta (green algae), Phaeophyta (brown algae), Bacillariophyta (diatoms), Eustigmatophyta and dinoflagellates as well as the prokaryotic phylum Cyanobacteria (blue-green algae). The term “algae” includes for example algae selected from: Amphora, Anabaena, Anikstrodesmis, Botryococcus, Chaetoceros, Chlamydomonas, Chlorella, Chlorococcum, Cyclotella, Cylindrotheca, Dunaliella, Emiliana, Euglena, Hematococcus, Isochrysis, Monochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Synechocystis, Tetraselmis, Thalassiosira, and Trichodesmium.

A part of a plant, e.g., a “plant tissue” may be treated according to the methods of the present invention to produce an improved plant. Plant tissue also encompasses plant cells. The term “plant cell” as used herein refers to individual units of a living plant, either in an intact whole plant or in an isolated form grown in in vitro tissue cultures, on media or agar, in suspension in a growth media or buffer or as a part of higher organized unites, such as, for example, plant tissue, a plant organ, or a whole plant.

A “protoplast” refers to a plant cell that has had its protective cell wall completely or partially removed using, for example, mechanical or enzymatic means resulting in an intact biochemical competent unit of living plant that can reform their cell wall, proliferate and regenerate grow into a whole plant under proper growing conditions.

The term “transformation” broadly refers to the process by which a plant host is genetically modified by the introduction of DNA by means of Agrobacteria or one of a variety of chemical or physical methods. As used herein, the term “plant host” refers to plants, including any cells, tissues, organs, or progeny of the plants. Many suitable plant tissues or plant cells can be transformed and include, but are not limited to, protoplasts, somatic embryos, pollen, leaves, seedlings, stems, calli, stolons, microtubers, and shoots. A plant tissue also refers to any clone of such a plant, seed, progeny, propagule whether generated sexually or asexually, and descendants of any of these, such as cuttings or seed.

The term “transformed” as used herein, refers to a cell, tissue, organ, or organism into which a foreign DNA molecule, such as a construct, has been introduced. The introduced DNA molecule may be integrated into the genomic DNA of the recipient cell, tissue, organ, or organism such that the introduced DNA molecule is transmitted to the subsequent progeny. In these embodiments, the “transformed” or “transgenic” cell or plant may also include progeny of the cell or plant and progeny produced from a breeding program employing such a transformed plant as a parent in a cross and exhibiting an altered phenotype resulting from the presence of the introduced DNA molecule. Preferably, the transgenic plant is fertile and capable of transmitting the introduced DNA to progeny through sexual reproduction.

The term “progeny”, such as the progeny of a transgenic plant, is one that is born of, begotten by, or derived from a plant or the transgenic plant. The introduced DNA molecule may also be transiently introduced into the recipient cell such that the introduced DNA molecule is not inherited by subsequent progeny and thus not considered “transgenic”. Accordingly, as used herein, a “non-transgenic” plant or plant cell is a plant which does not contain a foreign DNA stably integrated into its genome.

The term “plant promoter” as used herein is a promoter capable of initiating transcription in plant cells, whether or not its origin is a plant cell. Exemplary suitable plant promoters include, but are not limited to, those that are obtained from plants, plant viruses, and bacteria such as Agrobacterium or Rhizobium which comprise genes expressed in plant cells.

As used herein, a “fungal cell” refers to any type of eukaryotic cell within the kingdom of fungi. Phyla within the kingdom of fungi include Ascomycota, Basidiomycota, Blastocladiomycota, Chytridiomycota, Glomeromycota, Microsporidia, and Neocallimastigomycota. Fungal cells may include yeasts, molds, and filamentous fungi. In some embodiments, the fungal cell is a yeast cell.

As used herein, the term “yeast cell” refers to any fungal cell within the phyla Ascomycota and Basidiomycota. Yeast cells may include budding yeast cells, fission yeast cells, and mold cells. Without being limited to these organisms, many types of yeast used in laboratory and industrial settings are part of the phylum Ascomycota. In some embodiments, the yeast cell is an S. cerevisiae, Kluyveromyces marxianus, or Issatchenkia orientalis cell. Other yeast cells may include without limitation Candida spp. (e.g., Candida albicans), Yarrowia spp. (e.g., Yarrowia lipolytica), Pichia spp. (e.g., Pichia pastoris), Kluyveromyces spp. (e.g., Kluyveromyces lactis and Kluyveromyces marxianus), Neurospora spp. (e.g., Neurospora crassa), Fusarium spp. (e.g., Fusarium oxysporum), and Issatchenkia spp. (e.g., Issatchenkia orientalis, a.k.a. Pichia kudriavzevii and Candida acidothermophilum). In some embodiments, the fungal cell is a filamentous fungal cell. As used herein, the term “filamentous fungal cell” refers to any type of fungal cell that grows in filaments, i.e., hyphae or mycelia. Examples of filamentous fungal cells may include without limitation Aspergillus spp. (e.g., Aspergillus niger), Trichoderma spp. (e.g., Trichoderma reesei), Rhizopus spp. (e.g., Rhizopus oryzae), and Mortierella spp. (e.g., Mortierella isabellina).

In some embodiments, the fungal cell is an industrial strain. As used herein, “industrial strain” refers to any strain of fungal cell used in or isolated from an industrial process, e.g., production of a product on a commercial or industrial scale. Industrial strain may refer to a fungal species that is typically used in an industrial process, or it may refer to an isolate of a fungal species that may be also used for non-industrial purposes (e.g., laboratory research). Examples of industrial processes may include fermentation (e.g., in production of food or beverage products), distillation, biofuel production, production of a compound, and production of a polypeptide. Examples of industrial strains may include, without limitation, JAY270 and ATCC4124.

In some embodiments, the fungal cell is a polyploid cell. As used herein, a “polyploid” cell may refer to any cell whose genome is present in more than one copy. A polyploid cell may refer to a type of cell that is naturally found in a polyploid state, or it may refer to a cell that has been induced to exist in a polyploid state (e.g., through specific regulation, alteration, inactivation, activation, or modification of meiosis, cytokinesis, or DNA replication). A polyploid cell may refer to a cell whose entire genome is polyploid, or it may refer to a cell that is polyploid in a particular genomic locus of interest. Without wishing to be bound to theory, it is thought that the abundance of guideRNA may more often be a rate-limiting component in genome engineering of polyploidy cells than in haploid cells, and thus the methods using the systems described herein may take advantage of using a certain fungal cell type.

In some embodiments, the fungal cell is a diploid cell. As used herein, a “diploid” cell may refer to any cell whose genome is present in two copies. A diploid cell may refer to a type of cell that is naturally found in a diploid state, or it may refer to a cell that has been induced to exist in a diploid state (e.g., through specific regulation, alteration, inactivation, activation, or modification of meiosis, cytokinesis, or DNA replication). For example, the S. cerevisiae strain S228C may be maintained in a haploid or diploid state. A diploid cell may refer to a cell whose entire genome is diploid, or it may refer to a cell that is diploid in a particular genomic locus of interest. In some embodiments, the fungal cell is a haploid cell. As used herein, a “haploid” cell may refer to any cell whose genome is present in one copy. A haploid cell may refer to a type of cell that is naturally found in a haploid state, or it may refer to a cell that has been induced to exist in a haploid state (e.g., through specific regulation, alteration, inactivation, activation, or modification of meiosis, cytokinesis, or DNA replication). For example, the S. cerevisiae strain S228C may be maintained in a haploid or diploid state. A haploid cell may refer to a cell whose entire genome is haploid, or it may refer to a cell that is haploid in a particular genomic locus of interest.

As used herein, a “yeast expression vector” refers to a nucleic acid that contains one or more sequences encoding an RNA and/or polypeptide and may further contain any desired elements that control the expression of the nucleic acid(s), as well as any elements that enable the replication and maintenance of the expression vector inside the yeast cell. Many suitable yeast expression vectors and features thereof are known in the art; for example, various vectors and techniques are illustrated in in Yeast Protocols, 2nd edition, Xiao, W., ed. (Humana Press, New York, 2007) and Buckholz, R. G. and Gleeson, M. A. (1991) Biotechnology (NY) 9(11): 1067-72. Yeast vectors may contain, without limitation, a centromeric (CEN) sequence, an autonomous replication sequence (ARS), a promoter, such as an RNA Polymerase III promoter, operably linked to a sequence or gene of interest, a terminator such as an RNA polymerase III terminator, an origin of replication, and a marker gene (e.g., auxotrophic, antibiotic, or other selectable markers). Examples of expression vectors for use in yeast may include plasmids, yeast artificial chromosomes, 2 plasmids, yeast integrative plasmids, yeast replicative plasmids, shuttle vectors, and episomal plasmids.

### Stable Integration in the Genome of Plants and Plant Cells

In particular embodiments, it is envisaged that the polynucleotides encoding the components of the systems are introduced for stable integration into the genome of a plant cell. In these embodiments, the design of the transformation vector or the expression system can be adjusted depending on for when, where and under what conditions the engineered nucleic acid modification system gene(s) are expressed.

In particular embodiments, it is envisaged to introduce the components of the engineered nucleic acid modification systems or components thereof stably into the genomic DNA of a plant cell. Additionally, or alternatively, it is envisaged to introduce the components of the systems for stable integration into the DNA of a plant organelle such as, but not limited to a plastid, e mitochondrion or a chloroplast.

The expression system for stable integration into the genome of a plant cell may contain one or more of the following elements: a promoter element that can be used to express the engineered nucleic acid modification system polypeptide(s) and/or polynucleotide(s) in a plant cell; a 5′ untranslated region to enhance expression; an intron element to further enhance expression in certain cells, such as monocot cells; a multiple-cloning site to provide convenient restriction sites for inserting the engineered nucleic acid modification system polypeptide(s) and/or polynucleotide(s) and other desired elements; and a 3′ untranslated region to provide for efficient termination of the expressed transcript.

The elements of the expression system may be on one or more expression constructs which are either circular such as a plasmid or transformation vector, or non-circular such as linear double stranded DNA.

DNA construct(s) containing the components of the systems, and, where applicable, template sequence may be introduced into the genome of a plant, plant part, or plant cell by a variety of conventional techniques. The process generally comprises the steps of selecting a suitable host cell or host tissue, introducing the construct(s) into the host cell or host tissue.

In particular embodiments, the DNA construct may be introduced into the plant cell using techniques such as but not limited to electroporation, microinjection, aerosol beam injection of plant cell protoplasts, or the DNA constructs can be introduced directly to plant tissue using biolistic methods, such as DNA particle bombardment (see also Fu et al., Transgenic Res. 2000 February; 9(1):11-9). The basis of particle bombardment is the acceleration of particles coated with gene/s of interest toward cells, resulting in the penetration of the protoplasm by the particles and typically stable integration into the genome (see e.g., Klein et al, Nature (1987), Klein et ah, Bio/Technology (1992), Casas et ah, Proc. Natl. Acad. Sci. USA (1993)).

In particular embodiments, the DNA constructs containing components of the systems may be introduced into the plant by Agrobacterium-mediated transformation. The DNA constructs may be combined with suitable T-DNA flanking regions and introduced into a conventional Agrobacterium tumefaciens host vector. The foreign DNA can be incorporated into the genome of plants by infecting the plants or by incubating plant protoplasts with Agrobacterium bacteria, containing one or more Ti (tumor-inducing) plasmids (see e.g., Fraley et al., (1985), Rogers et al., (1987) and U.S. Pat. No. 5,563,055).

### Plant Promoters

In order to ensure appropriate expression in a plant cell, the components of the engineered nucleic acid modification system(s) of the present invention described herein are typically placed under control of a plant promoter, i.e., a promoter operable in plant cells. The use of different types of promoters is envisaged.

A constitutive plant promoter is a promoter that is able to express the open reading frame (ORF) that it controls in all or nearly all of the plant tissues during all or nearly all developmental stages of the plant (referred to as “constitutive expression”). One non-limiting example of a constitutive promoter is the cauliflower mosaic virus 35S promoter. “Regulated promoter” refers to promoters that direct gene expression not constitutively, but in a temporally- and/or spatially-regulated manner, and includes tissue-specific, tissue-preferred and inducible promoters. Different promoters may direct the expression of a gene in different tissues or cell types, or at different stages of development, or in response to different environmental conditions. In particular embodiments, one or more of the engineered nucleic acid modification system polypeptide(s) and/or polynucleotide(s) system components are expressed under the control of a constitutive promoter, such as the cauliflower mosaic virus 35S promoter issue-preferred promoters can be utilized to target enhanced expression in certain cell types within a particular plant tissue, for instance vascular cells in leaves or roots or in specific cells of the seed. Examples of particular promoters for use in the system are found in Kawamata et al., (1997) Plant Cell Physiol 38:792-803; Yamamoto et al., (1997) Plant J 12:255-65; Hire et al, (1992) Plant Mol Biol 20:207-18, Kuster et al, (1995) Plant Mol Biol 29:759-72, and Capana et al., (1994) Plant Mol Biol 25:681-91.

Examples of promoters that are inducible and that allow for spatiotemporal control of gene editing or gene expression may use a form of energy. The form of energy may include but is not limited to sound energy, electromagnetic radiation, chemical energy and/or thermal energy. Examples of inducible systems include tetracycline inducible promoters (Tet-On or Tet-Off), small molecule two-hybrid transcription activations systems (FKBP, ABA, etc.), or light inducible systems (Phytochrome, LOV domains, or cryptochrome), such as a Light Inducible Transcriptional Effector (LITE) that direct changes in transcriptional activity in a sequence-specific manner. The components of a light inducible system may include a Cas CRISPR enzyme, a light-responsive cytochrome heterodimer (e.g., from Arabidopsis thaliana), and a transcriptional activation/repression domain. Further examples of inducible DNA binding proteins and methods for their use are provided in U.S. 61/736,465 and U.S. 61/721,283, which is hereby incorporated by reference in its entirety.

In particular embodiments, transient or inducible expression can be achieved by using, for example, chemical-regulated promotors, i.e., whereby the application of an exogenous chemical induces gene expression. Modulating of gene expression can also be obtained by a chemical-repressible promoter, where application of the chemical represses gene expression. Chemical-inducible promoters include, but are not limited to, the maize ln2-2 promoter, activated by benzene sulfonamide herbicide safeners (see e.g., De Veylder et al., (1997) Plant Cell Physiol 38:568-77), the maize GST promoter (GST-ll-27, WO93/01294), activated by hydrophobic electrophilic compounds used as pre-emergent herbicides, and the tobacco PR-1 a promoter (Ono et al., (2004) Biosci Biotechnol Biochem 68:803-7) activated by salicylic acid. Promoters, which are regulated by antibiotics, such as tetracycline-inducible and tetracycline-repressible promoters (Gatz et al., (1991) Mol Gen Genet 227:229-37; U.S. Pat. Nos. 5,814,618 and 5,789,156), can also be used herein.

Translocation to and/or Expression in Specific Plant Organelles

The system may comprise elements for translocation to and/or expression in a specific plant organelle.

### Chloroplast Targeting

In particular embodiments, it is envisaged that the system is used to specifically modify chloroplast genes or to ensure expression in the chloroplast. For this purpose, use is made of chloroplast transformation methods or compartmentalization of the system components to the chloroplast. For instance, the introduction of genetic modifications in the plastid genome can reduce biosafety issues such as gene flow through pollen.

Methods of chloroplast transformation are known in the art and include Particle bombardment, PEG treatment, and microinjection. Additionally, methods involving the translocation of transformation cassettes from the nuclear genome to the plastid can be used as described in WO2010061186.

Alternatively, it is envisaged to target one or more of the system components to the plant chloroplast. This is achieved by incorporating in the expression construct a sequence encoding a chloroplast transit peptide (CTP) or plastid transit peptide, operably linked to the 5′ region of the sequence encoding the engineered nucleic acid modification system polypeptide(s) and/or polynucleotide(s). The CTP is removed in a processing step during translocation into the chloroplast. Chloroplast targeting of expressed proteins is well known to the skilled artisan (see for instance Protein Transport into Chloroplasts, 2010, Annual Review of Plant Biology, Vol. 61: 157-180). In such embodiments it is also desired to target the guide RNA to the plant chloroplast. Methods and constructs which can be used for translocating guide RNA into the chloroplast by means of a chloroplast localization sequence are described, for instance, in US 20040142476, incorporated herein by reference. Such variations of constructs can be incorporated into the expression systems of the invention to efficiently translocate the engineered nucleic acid modification system polypeptide(s) and/or polynucleotide(s).

### Introduction of Polynucleotides in Algal Cells.

Transgenic algae (or other plants such as rape) may be particularly useful in the production of vegetable oils or biofuels such as alcohols (especially methanol and ethanol) or other products. These may be engineered to express or overexpress high levels of oil or alcohols for use in the oil or biofuel industries.

U.S. Pat. No. 8,945,839 describes a method for engineering Micro-Algae (Chlamydomonas reinhardtii cells) species) using Cas9. Using similar tools, the methods of the systems described herein can be applied on Chlamydomonas species and other algae. In particular embodiments, engineered nucleic acid modification system polypeptide(s) and/or polynucleotide(s) are introduced in algae expressed using a vector that expresses one or more engineered nucleic acid modification system polypeptide(s) and/or polynucleotide(s) under the control of a constitutive promoter such as Hsp70A-Rbc S2 or Beta2-tubulin. Guide RNA and/or donor construct(s) is/are optionally delivered using a vector containing T7 promoter. Alternatively, engineered nucleic acid modification system mRNA and in vitro transcribed guide RNA and/or a donor construct can be delivered to algal cells. Electroporation protocols are available to the skilled person such as the standard recommended protocol from the GeneArt Chlamydomonas Engineering kit.

In particular embodiments, the endonuclease used herein is a split engineered nucleic acid modification system polypeptide(s). Split Cas enzymes are preferentially used in algae for targeted genome modification as has been described for Cas9 in WO 2015086795, which can be adapted for using split engineered nucleic acid modification system polypeptide(s) and/or polynucleotide(s) of the present invention. Without being bound by theory, such an approach can avoid the potential toxic effect of the protein (such as Cas) overexpression within the algae cell. In particular embodiments, said engineered nucleic acid modification system polypeptide(s) split domains (RuvC and HNH domains in the case of Cas9) can be simultaneously or sequentially introduced into the cell such that said split engineered nucleic acid modification system polypeptide(s) domain(s) process the target nucleic acid sequence in the algae cell. The reduced size of the split engineered nucleic acid modification system polypeptide(s) compared to the wild type engineered nucleic acid modification system polypeptide(s) allows other methods of delivery of the systems to the cells, such as the use of cell penetrating peptides as described herein. This method is of particular interest for generating genetically modified algae.

### Introduction of Polynucleotides in Yeast Cells

In particular embodiments, the invention relates to the use of the engineered nucleic acid modification system of the present invention for genome editing and/or modification of yeast cells. Methods for transforming yeast cells which can be used to introduce polynucleotides encoding the systems components are well known to the artisan and are reviewed by Kawai et al., 2010, Bioeng Bugs. 2010 November-December; 1(6): 395-403). Non-limiting examples include transformation of yeast cells by lithium acetate treatment (which may further include carrier DNA and PEG treatment), bombardment or by electroporation.

### Transient Expression of in Plants and Plant Cells

In particular embodiments, it is envisaged that the engineered nucleic acid modification system polypeptide(s) and/or polynucleotide(s) are transiently expressed in the plant cell. In these embodiments, the system can ensure modification of a target gene only when both the engineered nucleic acid modification system polypeptide(s), guide molecule, and/or donor construct is present in a cell, such that genomic modification can further be controlled. As the expression of the engineered nucleic acid modification system polypeptide(s) and/or polynucleotides is transient, plants regenerated from such plant cells typically contain no foreign DNA. In particular embodiments the engineered nucleic acid modification system polypeptide(s) is/are stably expressed by the plant cell and the guide molecule and/or donor construct is transiently expressed.

In particular embodiments, the engineered nucleic acid modification system and/or components thereof can be introduced in the plant cells using a plant viral vector (Scholthof et al. 1996, Annu Rev Phytopathol. 1996; 34:299-323). In further particular embodiments, said viral vector is a vector from a DNA virus. For example, geminivirus (e.g., cabbage leaf curl virus, bean yellow dwarf virus, wheat dwarf virus, tomato leaf curl virus, maize streak virus, tobacco leaf curl virus, or tomato golden mosaic virus) or nanovirus (e.g., Faba bean necrotic yellow virus). In other particular embodiments, said viral vector is a vector from an RNA virus. For example, tobravirus (e.g., tobacco rattle virus, tobacco mosaic virus), potexvirus (e.g., potato virus X), or hordeivirus (e.g., barley stripe mosaic virus). The replicating genomes of plant viruses are non-integrative vectors.

In particular embodiments, the vector used for transient expression of constructs is for instance a pEAQ vector, which is tailored for Agrobacterium-mediated transient expression (Sainsbury F. et al., Plant Biotechnol J. 2009 September; 7(7):682-93) in the protoplast. Precise targeting of genomic locations was demonstrated using a modified Cabbage Leaf Curl virus (CaLCuV) vector to express gRNAs in stable transgenic plants expressing a CRISPR enzyme (Scientific Reports 5, Article number: 14926 (2015), doi:10.1038/srep14926). Such a vector can be used with the engineered nucleic acid modification system of the present invention.

In particular embodiments, double-stranded DNA fragments encoding the engineered nucleic acid modification system polypeptide(s) and/or polynucleotide(s) can be transiently introduced into the plant cell. In such embodiments, the introduced double-stranded DNA fragments are provided in sufficient quantity to modify the cell but do not persist after a contemplated period of time has passed or after one or more cell divisions. Methods for direct DNA transfer in plants are known by the skilled artisan (see for instance Davey et al. Plant Mol Biol. 1989 September; 13(3):273-85.)

In other embodiments, an RNA polynucleotide encoding one or more engineered nucleic acid modification system polypeptide(s) or a guide molecule and/or a donor construct is introduced into the plant cell, which is then translated and processed by the host cell generating the protein in sufficient quantity to modify the cell (in the presence of at least one guide RNA and/or donor construct) but which does not persist after a contemplated period of time has passed or after one or more cell divisions. Methods for introducing mRNA to plant protoplasts for transient expression are known by the skilled artisan (see for instance in Gallie, Plant Cell Reports (1993), 13; 119-122).

Combinations of the different methods described above are also envisaged.

### Delivery to the Plant Cell

In particular embodiments, it is of interest to deliver one or more components of the system directly to the plant cell. This is of interest, inter alia, for the generation of non-transgenic plants (see below). In particular embodiments, one or more components of the engineered nucleic acid modification system is prepared outside the plant or plant cell and delivered to the cell. For instance, in particular embodiments, engineered nucleic acid modification system polypeptide(s) and/or polynucleotide(s) is prepared in vitro prior to introduction to the plant cell. Such polypeptides and/or polynucleotides can be prepared by various methods known by one of skill in the art and include recombinant production. After expression, one or more engineered nucleic acid modification system proteins and/or polynucleotides is/are isolated, refolded if needed, purified and optionally treated to remove any purification tags, such as a His-tag. Once crude, partially purified, or more completely purified proteins and/or polynucleotides is/are obtained, the protein and/or polynucleotides may be introduced to the plant cell.

In particular embodiments, the site-specific nuclease protein and/or other protein of the system of the present invention is mixed with guide RNA targeting the gene of interest to form a pre-assembled ribonucleoprotein.

The individual components or pre-assembled ribonucleoprotein can be introduced into the plant cell via electroporation, by bombardment with engineered nucleic acid modification system polypeptide and/or polynucleotide coated particles, by chemical transfection or by some other means of transport across a cell membrane. For instance, transfection of a plant protoplast with a pre-assembled CRISPR ribonucleoprotein has been demonstrated to ensure targeted modification of the plant genome (as described by Woo et al. Nature Biotechnology, 2015; DOI: 10.1038/nbt.3389), which can be adapted for use with the present invention.

In particular embodiments, the engineered nucleic acid modification system components are introduced into the plant cells using nanoparticles. The components, either as protein or nucleic acid or in a combination thereof, can be uploaded onto or packaged in nanoparticles and applied to the plants (such as for instance described in WO 2008042156 and US 20130185823). In particular, embodiments of the invention comprise nanoparticles uploaded with or packed with DNA molecule(s) encoding one or more engineered nucleic acid modification system polypeptide(s) and/or polynucleotide(s) (e.g., guide and/or donor construct) DNA molecules as described in WO2015089419.

Further means of introducing one or more components of the system to the plant cell is by using cell penetrating peptides (CPP). CPP for delivery of polypeptides and polynucleotides are described in greater detail elsewhere herein (see e.g., the section captioned “Delivery” elsewhere herein).

Other methods of delivering polynucleotides and polypeptides are described elsewhere herein and can be applied to plant cells so as to modify a plant cell using the engineered nucleic acid modification systems of the present invention. See e.g., the section captioned “Delivery” elsewhere herein.

### Making Genetically Modified Non-Transgenic Plants

In particular embodiments, the systems and methods described herein are used to modify endogenous genes or to modify their expression without the permanent introduction into the genome of the plant of any foreign gene, including those encoding engineered nucleic acid modification system components of the present invention, so as to avoid the presence of foreign DNA in the genome of the plant. This can be of interest as the regulatory requirements for non-transgenic plants are less rigorous.

In particular embodiments, this is ensured by transient expression of the system components. In particular embodiments, one or more of the systems components are expressed on one or more viral vectors which produce sufficient components of the systems to consistently and steadily ensure modification of a gene of interest according to a method described herein.

In particular embodiments, transient expression of constructs is ensured in plant protoplasts and thus not integrated into the genome. The limited window of expression can be sufficient to allow the system to ensure modification of a target gene as described herein.

In particular embodiments, the different components of the system are introduced in the plant cell, protoplast or plant tissue either separately or in mixture, with the aid of particulate delivering molecules such as nanoparticles or CPP molecules, or others as described herein above.

The expression of the components of the systems herein can induce targeted modification of the genome, either by direct activity of the system and optionally introduction of template DNA or by modification of genes targeted using the system as described herein. The different strategies described herein above allow Cas-mediated targeted genome editing without requiring the introduction of the components into the plant genome. Components which are transiently introduced into the plant cell are typically removed upon crossing.

### Detecting Modifications in the Plant Genome-Selectable Markers

In particular embodiments, where the method involves modification of an endogenous target gene of the plant genome, any suitable method can be used to determine, after the plant, plant part or plant cell is infected or transfected with the system, whether gene targeting or targeted mutagenesis has occurred at the target site. Where the method involves introduction of a transgene, a transformed plant cell, callus, tissue or plant may be identified and isolated by selecting or screening the engineered plant material for the presence of the transgene or for traits encoded by the transgene. Physical and biochemical methods may be used to identify plant or plant cell transformants containing inserted gene constructs or an endogenous DNA modification. These methods include but are not limited to: 1) Southern analysis or PCR amplification for detecting and determining the structure of the recombinant DNA insert or modified endogenous genes; 2) Northern blot, S1 RNase protection, primer-extension or reverse transcriptase-PCR amplification for detecting and examining RNA transcripts of the gene constructs; 3) enzymatic assays for detecting enzyme or ribozyme activity, where such gene products are encoded by the gene construct or expression is affected by the genetic modification; and/or 4) protein gel electrophoresis, Western blot techniques, immunoprecipitation, or enzyme-linked immunoassays, where the gene construct or endogenous gene products are proteins. Additional techniques, such as in situ hybridization, enzyme staining, and immunostaining, also may be used to detect the presence or expression of the recombinant construct or detect a modification of endogenous gene in specific plant organs and tissues. The methods for doing all these assays are well known to those skilled in the art.

Additionally (or alternatively), the expression system encoding the systems and components thereof of the present invention can be designed to comprise one or more selectable or detectable markers that provide a means to isolate or efficiently select cells that contain and/or have been modified by the system at an early stage and on a large scale.

In the case of Agrobacterium-mediated transformation, the marker cassette may be adjacent to or between flanking T-DNA borders and contained within a binary vector. In another embodiment, the marker cassette may be outside of the T-DNA. A selectable marker cassette may also be within or adjacent to the same T-DNA borders as the expression cassette or may be somewhere else within a second T-DNA on the binary vector (e.g., a 2 T-DNA system).

For particle bombardment or with protoplast transformation, the expression system can comprise one or more isolated linear fragments or may be part of a larger construct that might contain bacterial replication elements, bacterial selectable markers or other detectable elements. The expression cassette(s) comprising the polynucleotides encoding the guide and/or Cas may be physically linked to a marker cassette or may be mixed with a second nucleic acid molecule encoding a marker cassette. The marker cassette is comprised of necessary elements to express a detectable or selectable marker that allows for efficient selection of transformed cells.

The selection procedure for the cells based on the selectable marker will depend on the nature of the marker gene. In particular embodiments, use is made of a selectable marker, i.e., a marker which allows a direct selection of the cells based on the expression of the marker. A selectable marker can confer positive or negative selection and is conditional or non-conditional on the presence of external substrates (Miki et al. 2004, 107(3): 193-232). Most commonly, antibiotic or herbicide resistance genes are used as a marker, whereby selection is be performed by growing the engineered plant material on media containing an inhibitory amount of the antibiotic or herbicide to which the marker gene confers resistance. Examples of such genes are genes that confer resistance to antibiotics, such as hygromycin (hpt) and kanamycin (nptII), and genes that confer resistance to herbicides, such as phosphinothricin (bar) and chlorosulfuron (als),

Transformed plants and plant cells may also be identified by screening for the activities of a visible marker, typically an enzyme capable of processing a colored substrate (e.g., the β-glucuronidase, luciferase, B or C1 genes). Such selection and screening methodologies are well known to those skilled in the art.

### Plant Cultures and Regeneration

In particular embodiments, plant cells which have a modified genome and that are produced or obtained by any of the methods described herein, can be cultured to regenerate a whole plant which possesses the transformed or modified genotype and thus the desired phenotype. Conventional regeneration techniques are well known to those skilled in the art. Particular examples of such regeneration techniques rely on manipulation of certain phytohormones in a tissue culture growth medium, and typically relying on a biocide and/or herbicide marker which has been introduced together with the desired nucleotide sequences. In further particular embodiments, plant regeneration is obtained from cultured protoplasts, plant callus, explants, organs, pollens, embryos or parts thereof (see e.g., Evans et al. (1983), Handbook of Plant Cell Culture, Klee et al (1987) Ann. Rev. of Plant Phys.).

In particular embodiments, transformed or improved plants as described herein can be self-pollinated to provide seed for homozygous improved plants of the invention (homozygous for the DNA modification) or crossed with non-transgenic plants or different improved plants to provide seed for heterozygous plants. Where a recombinant DNA was introduced into the plant cell, the resulting plant of such a crossing is a plant which is heterozygous for the recombinant DNA molecule. Both such homozygous and heterozygous plants obtained by crossing from the improved plants and comprising the genetic modification (which can be a recombinant DNA) are referred to herein as “progeny”. Progeny plants are plants descended from the original transgenic plant and containing the genome modification or recombinant DNA molecule introduced by the methods provided herein. Alternatively, genetically modified plants can be obtained by one of the methods described supra using the Cfp1 enzyme whereby no foreign DNA is incorporated into the genome. Progeny of such plants, obtained by further breeding may also contain the genetic modification. Breedings are performed by any breeding methods that are commonly used for different crops (e.g., Allard, Principles of Plant Breeding, John Wiley & Sons, NY, U. of CA, Davis, CA, 50-98 (1960).

Generation of Plants with Enhanced Agronomic Traits

The systems provided herein can be used to introduce targeted double-strand or single-strand breaks and/or to introduce gene activator and or repressor systems and without being limitative, can be used for gene targeting, gene replacement, targeted mutagenesis, targeted deletions or insertions, targeted inversions and/or targeted translocations. By co-expression of multiple targeting RNAs directed to achieve multiple modifications in a single cell, multiplexed genome modification can be ensured. This technology can be used to high-precision engineering of plants with improved characteristics, including enhanced nutritional quality, increased resistance to diseases and resistance to biotic and abiotic stress, and increased production of commercially valuable plant products or heterologous compounds.

In particular embodiments, the engineered nucleic acid modification system as described herein can be used to introduce targeted single and/or double-strand breaks (DSB) in an endogenous DNA sequence. In some embodiments, the DSB activates cellular DNA repair pathways, which can be harnessed to achieve desired DNA sequence modifications near the break site. This is of interest where the inactivation of endogenous genes can confer or contribute to a desired trait. In particular embodiments, homologous recombination with a template sequence is promoted at the site of the DSB, in order to introduce a gene of interest.

In particular embodiments, the systems may be used as a generic nucleic acid binding protein with fusion to or being operably linked to a functional domain for activation and/or repression of endogenous plant genes. Exemplary functional domains may include but are not limited to translational initiator, translational activator, translational repressor, nucleases, in particular ribonucleases, a spliceosome, beads, a light inducible/controllable domain or a chemically inducible/controllable domain. Typically, in these embodiments, the Cas protein comprises at least one mutation, such that it has no more than 5% of the activity of the Cas protein not having the at least one mutation; the guide RNA comprises a guide sequence capable of hybridizing to a target sequence.

The methods described herein generally result in the generation of “improved plants” in that they have one or more desirable traits compared to the wildtype plant. In particular embodiments, the plants, plant cells or plant parts obtained are transgenic plants, comprising an exogenous DNA sequence incorporated into the genome of all or part of the cells of the plant. In particular embodiments, non-transgenic genetically modified plants, plant parts or cells are obtained, in that no exogenous DNA sequence is incorporated into the genome of any of the plant cells of the plant. In such embodiments, the improved plants are non-transgenic. Where only the modification of an endogenous gene is ensured and no foreign genes are introduced or maintained in the plant genome, the resulting genetically modified crops contain no foreign genes and can thus be considered, such as by a regulatory authority, non-transgenic. The different applications of the systems for plant genome editing are described more in detail below:

### Introduction of One or More Foreign Genes to Confer an Agricultural Trait of Interest

The invention provides methods of genome editing or modifying sequences associated with or at a target locus or polynucleotide of interest wherein the method comprises introducing an engineered nucleic acid modification system of the present invention into a plant cell, whereby the effector protein complex of the system (e.g., the Cas, DNA polymerase, and/or VirD1 and/or VirD2) effectively functions to integrate a DNA insert, e.g., encoding a foreign gene of interest, into the genome of the plant cell. In some embodiments, the DNA insert is from a donor construct of the system. In preferred embodiments the integration of the DNA insert is facilitated by HR with an exogenously introduced DNA template or repair template. Typically, the exogenously introduced DNA template or repair template is delivered together with the effector protein complex of the engineered nucleic acid modification system of the present invention, a component thereof, or a polynucleotide vector for expression of one or more component of the engineered nucleic acid modification system of the present invention.

The systems provided herein allow for targeted gene delivery. It has become increasingly clear that the efficiency of expressing a gene of interest is to a great extent determined by the location of integration into the genome. The present methods allow for targeted integration of the foreign gene into a desired location in the genome. The location can be selected based on information of previously generated events or can be selected by methods disclosed elsewhere herein.

In some embodiments, a method of modifying a cell includes introducing into the cell an engineered nucleic acid modification system or one or more components thereof (e.g., a system including site-specific nuclease coupled to a Vir polypeptide (e.g., a Vir D1 and/or Vir D2), where the site-specific nuclease optionally complexes with an optionally included guide molecule; and a DNA polymerase and a donor construct containing a polynucleotide of interest to be integrated into a polynucleotide (e.g., genomic or extragenomic DNA and/or RNA); and where the guide molecule and/or site-specific nuclease hybridizes to a target sequence endogenous to the plant cell and guiding the effector complex to the target sequence, and wherein the effector complex is capable of working in concert to integrate a polynucleotide of interest from the donor construct into a target polynucleotide at or near the target sequence.

In some embodiments, the site specific nuclease (such as a Cas polypeptide) can complex with the guide molecule, which can hybridize to the target sequence and induces a single or double strand break at or near the sequence to which the guide sequence is hybridized. In some embodiments, the method includes introducing into the cell a nucleotide encoding an HDR repair template and/or donor construct which encodes the polynucleotide of interest and which is introduced into the location of the DSB and/or single strand break and which is introduced into the a the location of the DSB and/or single strand break by the effector complex of the engineered nucleotide modification system of the present invention and/or one or more endogenous DNA repair mechanisms (e.g., HDR). The system and/or components thereof of the present invention can be introduced as polypeptides, encoding polynucleotides, and/or complexes of polypeptides and/or polynucleotides.

In particular embodiments, the polynucleotides are delivered into the cell by a DNA virus (e.g., a geminivirus) or an RNA virus (e.g., a tobravirus). In particular embodiments, the introducing steps include delivering to the plant cell a T-DNA containing one or more polynucleotide sequences encoding one or more components of the engineered nucleic acid modification system of the present invention, where the delivering is via Agrobacterium. The nucleic acid sequence encoding one or more components of the engineered nucleic acid modification system of the present invention can be operably linked to a promoter, such as a constitutive promoter (e.g., a cauliflower mosaic virus 35S promoter), or a cell specific or inducible promoter. In particular embodiments, the polynucleotide is introduced by microprojectile bombardment. In particular embodiments, the method further includes screening the plant cell after the introducing steps to determine whether the repair template i.e., the gene of interest has been introduced. In particular embodiments, the methods include the step of regenerating a plant from the plant cell. In further embodiments, the methods include cross breeding the plant to obtain a genetically desired plant lineage. Examples of foreign genes encoding a trait of interest are listed below.

### Editing of Endogenous Genes to Confer an Agricultural Trait of Interest

Also provided herein are methods of genome editing or otherwise modifying sequences associated with, in proximity to, or at a target locus of interest wherein the method comprises introducing an engineered nucleic acid modification system and/or components thereof of the present invention into a plant cell, whereby the system modifies the polynucleotide and/or expression thereof of an endogenous polynucleotide (such as a gene or other genomic polynucleotide and/or extragenomic polynucleotide) of the plant. This can be achieved in different ways. In some exemplary embodiments, the elimination of expression of an endogenous gene is desirable and the engineered nucleic acid modification system is used to target and cleave an endogenous gene so as to modify gene expression. In these embodiments the system and/or components thereof of the present invention are introduced into a plant cell as described in greater detail elsewhere herein whereby the system modifies one or more polynucleotides in the plant cell so es to edit or otherwise modify a polynucleotide associated with conferring an agricultural trait of interest. Polynucleotides and genes associated with conferring an agricultural trait of interest are described in greater detail elsewhere herein.

Such modified cells can result in or be used to generate, for example, plants with desired agricultural traits of interest, including but not limited to disease resistance, pest resistance, herbicide resistance, heat and/or cold tolerance, frost tolerance, improved growth performance, drought resistance, salinity tolerance, desired ripening profiles, and the like.

In particular embodiments of the methods described above, disease resistant crops are obtained by targeted mutation of disease susceptibility genes or genes encoding negative regulators (e.g., Mlo gene) of plant defense genes. In a particular embodiment, herbicide-tolerant crops are generated by targeted substitution of specific nucleotides in plant genes such as those encoding acetolactate synthase (ALS) and protoporphyrinogen oxidase (PPO). In particular embodiments drought and salt tolerant crops by targeted mutation of genes encoding negative regulators of abiotic stress tolerance, low amylose grains by targeted mutation of Waxy gene, rice or other grains with reduced rancidity by targeted mutation of major lipase genes in aleurone layer, etc. In particular embodiments. A more extensive list of endogenous genes encoding a traits of interest are listed below.

### Modulating Endogenous Genes by the System to Confer an Agricultural Trait of Interest

Also provided herein are methods for modulating (activating or repressing) endogenous gene expression using the engineered nucleic acid modification systems of the present invention provided herein. Such methods can make use of distinct RNA sequence(s) which are targeted to the plant genome by the system. More particularly the distinct RNA sequence(s) bind to two or more adaptor proteins (e.g., aptamers) whereby each adaptor protein is associated with one or more functional domains associated, wherein at least one or more functional domains have one or more activities comprising methylase activity, demethylase activity, transcription activation activity, transcription repression activity, transcription release factor activity, histone modification activity, DNA integration activity RNA cleavage activity, DNA Cleavage Activity or Nucleic Acid Binding Activity. These Functional Domains and Adaptors can be incorporated with, associated with, or otherwise coupled to one or more proteins of the engineered nucleic acid modification system of the present invention (such as, but not limited to, the site-specific nuclease, the Vir polypeptide (e.g., Vir D1 and/or Vir D2), and/or the DNA polymerase). These additional functional domains are used to modulate expression of an endogenous plant gene so as to obtain the desired trait. Typically, in these embodiments, the site specific nuclease protein (such as a Cas polypeptide) has one or more mutations such that it has no more than 5% of the nuclease activity.

In particular embodiments, the methods provided herein include the steps of (a) introducing into the cell a system comprising a guide RNA, comprising a direct repeat and a guide sequence, wherein the guide sequence hybridizes to a target sequence that is endogenous to the plant cell; (b) introducing into the plant cell a system; and wherein either the guide RNA is modified to comprise a distinct RNA sequence (aptamer) binding to a functional domain and/or the Cas effector protein is modified in that it is linked to a functional domain. In particular embodiments, the step of introducing can include delivering to the plant cell one or more polynucleotides encoding the (modified) Cas effector protein and the (modified) guide RNA. The details the components of the systems for use in these methods are described elsewhere herein.

### Modification of Polyploid Plants

Many plants are polyploid, which means they carry duplicate copies of their genomes-sometimes as many as six, as in wheat. The methods according to the present invention, which make use of the systems can be “multiplexed” to affect all copies of a gene, or to target dozens of genes at once. For instance, in particular embodiments, the methods of the present invention are used to simultaneously ensure a loss of function mutation in different genes responsible for suppressing defenses against a disease. In particular embodiments, the methods of the present invention are used to simultaneously suppress the expression of the TaMLO-A1, TaMLO-Bl and TaMLO-Dl nucleic acid sequence in a wheat plant cell and regenerating a wheat plant therefrom, in order to ensure that the wheat plant is resistant to powdery mildew (see also WO2015109752).

### Exemplary Genes Conferring Agronomic Traits

As described herein above, in particular embodiments, the invention encompasses the use of the systems and components thereof as described herein for the insertion of a DNA of interest, including one or more plant expressible gene(s). In further particular embodiments, the invention encompasses methods and tools using the Cas system as described herein for partial or complete deletion of one or more plant expressed gene(s). In other further particular embodiments, the invention encompasses methods and tools using the system as described herein to ensure modification of one or more plant-expressed genes by mutation, substitution, insertion of one of more nucleotides. In other particular embodiments, the invention encompasses the use of systems as described herein to ensure modification of expression of one or more plant-expressed genes by specific modification of one or more of the regulatory elements directing expression of said genes.

In particular embodiments, the invention encompasses methods which involve the introduction of exogenous genes and/or the targeting of endogenous genes and their regulatory elements, such as listed below:

1. Genes that Confer Resistance to Pests or Diseases:

Plant disease resistance genes. A plant can be transformed with cloned resistance genes to engineer plants that are resistant to specific pathogen strains. See, e.g., Jones et al., Science 266:789 (1994) (cloning of the tomato Cf-9 gene for resistance to Cladosporium fulvum); Martin et al., Science 262:1432 (1993) (tomato Pto gene for resistance to Pseudomonas syringae pv. tomato encodes a protein kinase); Mindrinos et al., Cell 78:1089 (1994) (Arabidopsmay be RSP2 gene for resistance to Pseudomonas syringae). A plant gene that is upregulated or down regulated during pathogen infection can be engineered for pathogen resistance. See, e.g., Thomazella et al., bioRxiv 064824; doi: doi.org/10.1101/064824 Epub. Jul. 23, 2016 (tomato plants with deletions in the SlDMR6-1 which is normally upregulated during pathogen infection).

Genes conferring resistance to a pest, such as soybean cyst nematode. See e.g., PCT Application WO 96/30517; PCT Application WO 93/19181.

Bacillus thuringiensis proteins see, e.g., Geiser et al., Gene 48:109 (1986).

Lectins, see, for example, Van Damme et al., Plant Molec. Biol. 24:25 (1994.

Vitamin-binding protein, such as avidin, see PCT application US93/06487, teaching the use of avidin and avidin homologues as larvicides against insect pests.

Enzyme inhibitors such as protease or proteinase inhibitors or amylase inhibitors. See, e.g., Abe et al., J. Biol. Chem. 262:16793 (1987), Huub et al., Plant Molec. Biol. 21:985 (1993)), Sumitani et al., Biosci. Biotech. Biochem. 57:1243 (1993) and U.S. Pat. No. 5,494,813.

Insect-specific hormones or pheromones such as ecdysteroid or juvenile hormone, a variant thereof, a mimetic based thereon, or an antagonist or agonist thereof. See, for example Hammock et al., Nature 344:458 (1990).

Insect-specific peptides or neuropeptides which, upon expression, disrupts the physiology of the affected pest. For example, Regan, J. Biol. Chem. 269:9 (1994) and Pratt et al., Biochem. Biophys. Res. Comm. 163:1243 (1989). See also U.S. Pat. No. 5,266,317.

Insect-specific venom produced in nature by a snake, a wasp, or any other organism. For example, see Pang et al., Gene 116: 165 (1992).

Enzymes responsible for a hyperaccumulation of a monoterpene, a sesquiterpene, a steroid, hydroxamic acid, a phenylpropanoid derivative or another nonprotein molecule with insecticidal activity.

Enzymes involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO93/02197, Kramer et al., Insect Biochem. Molec. Biol. 23:691 (1993) and Kawalleck et al., Plant Molec. Biol. 21:673 (1993).

Molecules that stimulates signal transduction. For example, see Botella et al., Plant Molec. Biol. 24:757 (1994), and Griess et al., Plant Physiol. 104:1467 (1994).

Viral-invasive proteins or a complex toxin derived therefrom. See Beachy et al., Ann. rev. Phytopathol. 28:451 (1990).

Developmental-arrestive proteins produced in nature by a pathogen or a parasite. See Lamb et al., Bio/Technology 10:1436 (1992) and Toubart et al., Plant J. 2:367 (1992).

A developmental-arrestive protein produced in nature by a plant. For example, Logemann et al., Bio/Technology 10:305 (1992).

In plants, pathogens are often host-specific. For example, some Fusarium species will causes tomato wilt but attacks only tomato, and other Fusarium species attack only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible or there can be partial resistance against all races of a pathogen, typically controlled by many genes and/or also complete resistance to some races of a pathogen but not to other races. Such resistance is typically controlled by a few genes. Using methods and components of the system, a new tool now exists to induce specific mutations in anticipation hereon. Accordingly, one can analyze the genome of sources of resistance genes, and in plants having desired characteristics or traits, use the method and components of the systems to induce the rise of resistance genes. The present systems can do so with more precision than previous mutagenic agents and hence accelerate and improve plant breeding programs.

2. Genes Involved in Plant Diseases, Such as Those Listed in WO 2013046247:

Rice diseases: Magnaporthe grisea, Cochliobolus miyabeanus, Rhizoctonia solani, Gibberella fujikuroi; Wheat diseases: Erysiphe graminis, Fusarium graminearum, F. avenaceum, F. culmorum, Microdochium nivale, Puccinia striiformis, P. graminis, P. recondita, Micronectriella nivale, Typhula sp., Ustilago tritici, Tilletia caries, Pseudocercosporella herpotrichoides, Mycosphaerella graminicola, Stagonospora nodorum, Pyrenophora tritici-repentis; Barley diseases: Erysiphe graminis, Fusarium graminearum, F. avenaceum, F. culmorum, Microdochium nivale, Puccinia striiformis, P. graminis, P. hordei, Ustilago nuda, Rhynchosporium secalis, Pyrenophora teres, Cochliobolus sativus, Pyrenophora graminea, Rhizoctonia solani; Maize diseases: Ustilago maydis, Cochliobolus heterostrophus, Gloeocercospora sorghi, Puccinia polysora, Cercospora zeae-maydis, Rhizoctonia solani;

Citrus diseases: Diaporthe citri, Elsinoe fawcetti, Penicillium digitatum, P. italicum, Phytophthora parasitica, Phytophthora citrophthora; Apple diseases: Monilinia mali, Valsa ceratosperma, Podosphaera leucotricha, Alternaria alternata apple pathotype, Venturia inaequalis, Colletotrichum acutatum, Phytophtora cactorum;

Pear diseases: Venturia nashicola, V. pirina, Alternaria alternata Japanese pear pathotype, Gymnosporangium haraeanum, Phytophtora cactorum;

Peach diseases: Monilinia fructicola, Cladosporium carpophilum, Phomopsis sp.;

Grape diseases: Elsinoe ampelina, Glomerella cingulata, Uninula necator, Phakopsora ampelopsidis, Guignardia bidwellii, Plasmopara viticola;

Persimmon diseases: Gloesporium kaki, Cercospora kaki, Mycosphaerela nawae;

Gourd diseases: Colletotrichum lagenarium, Sphaerotheca fuliginea, Mycosphaerella melonis, Fusarium oxysporum, Pseudoperonospora cubensis, Phytophthora sp., Pythium sp.;

Tomato diseases: Alternaria solani, Cladosporium fulvum, Phytophthora infestans; Pseudomonas syringae pv. Tomato; Phytophthora capsici; Xanthomonas

Eggplant diseases: Phomopsis vexans, Erysiphe cichoracearum;

Brassicaceous vegetable diseases: Alternaria japonica, Cercosporella brassicae, Plasmodiophora brassicae, Peronospora parasitica;

Welsh onion diseases: Puccinia allii, Peronospora destructor;

Soybean diseases: Cercospora kikuchii, Elsinoe glycines, Diaporthe phaseolorum var. sojae, Septoria glycines, Cercospora sojina, Phakopsora pachyrhizi, Phytophthora sojae, Rhizoctonia solani, Corynespora casiicola, Sclerotinia sclerotiorum;

Kidney bean diseases: Colletrichum lindemthianum;

Peanut diseases: Cercospora personata, Cercospora arachidicola, Sclerotium rolfsii;

Pea diseases pea: Erysiphe pisi;

Potato diseases: Alternaria solani, Phytophthora infestans, Phytophthora erythroseptica, Spongospora subterranean, f. sp. Subterranean;

Strawberry diseases: Sphaerotheca humuli, Glomerella cingulata;

Tea diseases: Exobasidium reticulatum, Elsinoe leucospila, Pestalotiopsis sp., Colletotrichum theae-sinensis;

Tobacco diseases: Alternaria longipes, Erysiphe cichoracearum, Colletotrichum tabacum, Peronospora tabacina, Phytophthora nicotianae;

Rapeseed diseases: Sclerotinia sclerotiorum, Rhizoctonia solani;

Cotton diseases: Rhizoctonia solani;

Beet diseases: Cercospora beticola, Thanatephorus cucumeris, Thanatephorus cucumeris, Aphanomyces cochlioides;

Rose diseases: Diplocarpon rosae, Sphaerotheca pannosa, Peronospora sparsa;

Diseases of chrysanthemum and asteraceae: Bremia lactuca, Septoria chrysanthemi-indici, Puccinia horiana;

Diseases of various plants: Pythium aphanidermatum, Pythium debarianum, Pythium graminicola, Pythium irregulare, Pythium ultimum, Botrytis cinerea, Sclerotinia sclerotiorum;

Radish diseases: Alternaria brassicicola;

Zoysia diseases: Sclerotinia homeocarpa, Rhizoctonia solani;

Banana diseases: Mycosphaerella fijiensis, Mycosphaerella musicola;

Sunflower diseases: Plasmopara halstedii;

Seed diseases or diseases in the initial stage of growth of various plants caused by Aspergillus spp., Penicillium spp., Fusarium spp., Gibberella spp., Tricoderma spp., Thielaviopsis spp., Rhizopus spp., Mucor spp., Corticium spp., Rhoma spp., Rhizoctonia spp., Diplodia spp., or the like;

Virus diseases of various plants mediated by Polymixa spp., Olpidium spp., or the like.

3. Examples of Genes that Confer Resistance to Herbicides:

Resistance to herbicides that inhibit the growing point or meristem, such as an imidazolinone or a sulfonylurea, for example, by Lee et al., EMBO J. 7:1241 (1988), and Miki et al., Theor. Appl. Genet. 80:449 (1990), respectively.

Glyphosate tolerance (resistance conferred by, e.g., mutant 5-enolpyruvylshikimate-3-phosphate synthase (EPSPs) genes, aroA genes and glyphosate acetyl transferase (GAT) genes, respectively), or resistance to other phosphono compounds such as by glufosinate (phosphinothricin acetyl transferase (PAT) genes from Streptomyces species, including Streptomyces hygroscopicus and Streptomyces viridichromogenes), and to pyridinoxy or phenoxy proprionic acids and cyclohexones by ACCase inhibitor-encoding genes. See, for example, U.S. Pat. Nos. 4,940,835 and 6,248,876, 4,769,061, EP No. 0 333 033 and U.S. Pat. No. 4,975,374. See also EP No. 0242246, DeGreef et al., Bio/Technology 7:61 (1989), Marshall et al., Theor. Appl. Genet. 83:435 (1992), WO 2005012515 to Castle et. al. and WO 2005107437.

Resistance to herbicides that inhibit photosynthesis, such as a triazine (psbA and gs+ genes) or a benzonitrile (nitrilase gene), and glutathione S-transferase in Przibila et al., Plant Cell 3:169 (1991), U.S. Pat. No. 4,810,648, and Hayes et al., Biochem. J. 285: 173 (1992).

Genes encoding Enzymes detoxifying the herbicide or a mutant glutamine synthase enzyme that is resistant to inhibition, e.g., described in U.S. patent application Ser. No. 11/760,602. Or a detoxifying enzyme is an enzyme encoding a phosphinothricin acetyltransferase (such as the bar or pat protein from Streptomyces species). Phosphinothricin acetyltransferases are for example described in U.S. Pat. Nos. 5,561,236; 5,648,477; 5,646,024; 5,273,894; 5,637,489; 5,276,268; 5,739,082; 5,908,810 and 7,112,665.

Hydroxyphenylpyruvatedioxygenases (HPPD) inhibitors, i.e., naturally occurring HPPD resistant enzymes, or genes encoding a mutated or chimeric HPPD enzyme as described in WO 96/38567, WO 99/24585, and WO 99/24586, WO 2009/144079, WO 2002/046387, or U.S. Pat. No. 6,768,044.

Examples of genes involved in Abiotic stress tolerance:

Transgene capable of reducing the expression and/or the activity of poly(ADP-ribose) polymerase (PARP) gene in the plant cells or plants as described in WO 00/04173 or, WO/2006/045633.

Transgenes capable of reducing the expression and/or the activity of the PARG encoding genes of the plants or plants cells, as described e.g., in WO 2004/090140.

Transgenes coding for a plant-functional enzyme of the nicotineamide adenine dinucleotide salvage synthesis pathway including nicotinamidase, nicotinate phosphoribosyltransferase, nicotinic acid mononucleotide adenyl transferase, nicotinamide adenine dinucleotide synthetase or nicotine amide phosphorybosyltransferase as described e.g., in EP 04077624.7, WO 2006/133827, PCT/EP07/002,433, EP 1999263, or WO 2007/107326.

Enzymes involved in carbohydrate biosynthesis include those described in e.g. EP 0571427, WO 95/04826, EP 0719338, WO 96/15248, WO 96/19581, WO 96/27674, WO 97/11188, WO 97/26362, WO 97/32985, WO 97/42328, WO 97/44472, WO 97/45545, WO 98/27212, WO 98/40503, WO99/58688, WO 99/58690, WO 99/58654, WO 00/08184, WO 00/08185, WO 00/08175, WO 00/28052, WO 00/77229, WO 01/12782, WO 01/12826, WO 02/101059, WO 03/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 00/22140, WO 2006/063862, WO 2006/072603, WO 02/034923, EP 06090134.5, EP 06090228.5, EP 06090227.7, EP 07090007.1, EP 07090009.7, WO 01/14569, WO 02/79410, WO 03/33540, WO 2004/078983, WO 01/19975, WO 95/26407, WO 96/34968, WO 98/20145, WO 99/12950, WO 99/66050, WO 99/53072, U.S. Pat. No. 6,734,341, WO 00/11192, WO 98/22604, WO 98/32326, WO 01/98509, WO 01/98509, WO 2005/002359, U.S. Pat. Nos. 5,824,790, 6,013,861, WO 94/04693, WO 94/09144, WO 94/11520, WO 95/35026 or WO 97/20936 or enzymes involved in the production of polyfructose, especially of the inulin and levan-type, as disclosed in EP 0663956, WO 96/01904, WO 96/21023, WO 98/39460, and WO 99/24593, the production of alpha-1,4-glucans as disclosed in WO 95/31553, US 2002031826, U.S. Pat. Nos. 6,284,479, 5,712,107, WO 97/47806, WO 97/47807, WO 97/47808 and WO 00/14249, the production of alpha-1,6 branched alpha-1,4-glucans, as disclosed in WO 00/73422, the production of alternan, as disclosed in e.g. WO 00/47727, WO 00/73422, EP 06077301.7, U.S. Pat. No. 5,908,975 and EP 0728213, the production of hyaluronan, as for example disclosed in WO 2006/032538, WO 2007/039314, WO 2007/039315, WO 2007/039316, JP 2006304779, and WO 2005/012529.

Genes that improve drought resistance. For example, WO 2013122472 discloses that the absence or reduced level of functional Ubiquitin Protein Ligase protein (UPL) protein, more specifically, UPL3, leads to a decreased need for water or improved resistance to drought of said plant. Other examples of transgenic plants with increased drought tolerance are disclosed in, for example, US 2009/0144850, US 2007/0266453, and WO 2002/083911. US2009/0144850 describes a plant displaying a drought tolerance phenotype due to altered expression of a DR02 nucleic acid. US 2007/0266453 describes a plant displaying a drought tolerance phenotype due to altered expression of a DR03 nucleic acid and WO 2002/08391 1 describes a plant having an increased tolerance to drought stress due to a reduced activity of an ABC transporter which is expressed in guard cells. Another example is the work by Kasuga and co-authors (1999), who describe that overexpression of cDNA encoding DREB1 A in transgenic plants activated the expression of many stress tolerance genes under normal growing conditions and resulted in improved tolerance to drought, salt loading, and freezing. However, the expression of DREB1A also resulted in severe growth retardation under normal growing conditions (Kasuga (1999) Nat Biotechnol 17(3) 287-291).

In further particular embodiments, crop plants can be improved by influencing specific plant traits. For example, by developing pesticide-resistant plants, improving disease resistance in plants, improving plant insect and nematode resistance, improving plant resistance against parasitic weeds, improving plant drought tolerance, improving plant nutritional value, improving plant stress tolerance, avoiding self-pollination, plant forage digestibility biomass, grain yield etc. A few specific non-limiting examples are provided hereinbelow.

In addition to targeted mutation of single genes, systems can be designed to allow targeted mutation of multiple genes, deletion of chromosomal fragment, site-specific integration of transgene, site-directed mutagenesis in vivo, and precise gene replacement or allele swapping in plants. Therefore, the methods described herein have broad applications in gene discovery and validation, mutational and cisgenic breeding, and hybrid breeding. These applications facilitate the production of a new generation of genetically modified crops with various improved agronomic traits such as herbicide resistance, disease resistance, abiotic stress tolerance, high yield, and superior quality.

### Creating Male Sterile Plants

Hybrid plants typically have advantageous agronomic traits compared to inbred plants. However, for self-pollinating plants, the generation of hybrids can be challenging. In different plant types, genes have been identified which are important for plant fertility, more particularly male fertility. For instance, in maize, at least two genes have been identified which are important in fertility (Amitabh Mohanty International Conference on New Plant Breeding Molecular Technologies Technology Development and Regulation, Oct. 9-10, 2014, Jaipur, India; Svitashev et al. Plant Physiol. 2015 October; 169(2):931-45; Djukanovic et al. Plant J. 2013 December; 76(5):888-99). The methods provided herein can be used to target genes required for male fertility so as to generate male sterile plants which can easily be crossed to generate hybrids. In particular embodiments, the systems provided herein is used for targeted mutagenesis of the cytochrome P450-like gene (MS26) or the meganuclease gene (MS45) thereby conferring male sterility to the maize plant. Maize plants which are as such genetically altered can be used in hybrid breeding programs.

### Increasing the Fertility Stage in Plants

In particular embodiments, the systems and methods provided herein are used to prolong the fertility stage of a plant such as of a rice plant. For instance, a rice fertility stage gene such as Ehd3 can be targeted in order to generate a mutation in the gene and plantlets can be selected for a prolonged regeneration plant fertility stage (as described in CN 104004782)

### Generating Genetic Variation in a Crop of Interest

The availability of wild germplasm and genetic variations in crop plants is the key to crop improvement programs, but the available diversity in germplasms from crop plants is limited. The present invention envisages methods for generating a diversity of genetic variations in a germplasm of interest. In this application of the system a library of guide RNAs targeting different locations in the plant genome is provided and is introduced into plant cells together with the Cas effector protein. In this way a collection of genome-scale point mutations and gene knock-outs can be generated. In particular embodiments, the methods comprise generating a plant part or plant from the cells so obtained and screening the cells for a trait of interest. The target genes can include both coding and non-coding regions. In particular embodiments, the trait is stress tolerance and the method is a method for the generation of stress-tolerant crop varieties

### Fruit-Ripening

Ripening is a normal phase in the maturation process of fruits and vegetables. Only a few days after it starts it renders a fruit or vegetable inedible. This process brings significant losses to both farmers and consumers. In particular embodiments, the methods of the present invention are used to reduce ethylene production. This is ensured by ensuring one or more of the following: a. Suppression of ACC synthase gene expression. ACC (1-aminocyclopropane-1-carboxylic acid) synthase is the enzyme responsible for the conversion of S-adenosylmethionine (SAM) to ACC; the second to the last step in ethylene biosynthesis. Enzyme expression is hindered when an antisense (“mirror-image”) or truncated copy of the synthase gene is inserted into the plant's genome; b. Insertion of the ACC deaminase gene. The gene coding for the enzyme is obtained from Pseudomonas chlororaphis, a common nonpathogenic soil bacterium. It converts ACC to a different compound thereby reducing the amount of ACC available for ethylene production; c. Insertion of the SAM hydrolase gene. This approach is similar to ACC deaminase wherein ethylene production is hindered when the amount of its precursor metabolite is reduced; in this case SAM is converted to homoserine. The gene coding for the enzyme is obtained from E. coli T3 bacteriophage and d. Suppression of ACC oxidase gene expression. ACC oxidase is the enzyme which catalyzes the oxidation of ACC to ethylene, the last step in the ethylene biosynthetic pathway. Using the methods described herein, down regulation of the ACC oxidase gene results in the suppression of ethylene production, thereby delaying fruit ripening. In particular embodiments, additionally or alternatively to the modifications described above, the methods described herein are used to modify ethylene receptors, so as to interfere with ethylene signals obtained by the fruit. In particular embodiments, expression of the ETR1 gene, encoding an ethylene binding protein is modified, more particularly suppressed. In particular embodiments, additionally or alternatively to the modifications described above, the methods described herein are used to modify expression of the gene encoding Polygalacturonase (PG), which is the enzyme responsible for the breakdown of pectin, the substance that maintains the integrity of plant cell walls. Pectin breakdown occurs at the start of the ripening process resulting in the softening of the fruit. Accordingly, in particular embodiments, the methods described herein are used to introduce a mutation in the PG gene or to suppress activation of the PG gene in order to reduce the amount of PG enzyme produced thereby delaying pectin degradation.

Thus, in particular embodiments, the methods comprise the use of the system to ensure one or more modifications of the genome of a plant cell, such as described above, and regenerating a plant therefrom. In particular embodiments, the plant is a tomato plant.

### Increasing Storage Life of Plants

In particular embodiments, the methods of the present invention are used to modify genes involved in the production of compounds which affect storage life of the plant or plant part. More particularly, the modification is in a gene that prevents the accumulation of reducing sugars in potato tubers. Upon high-temperature processing, these reducing sugars react with free amino acids, resulting in brown, bitter-tasting products and elevated levels of acrylamide, which is a potential carcinogen. In particular embodiments, the methods provided herein are used to reduce or inhibit expression of the vacuolar invertase gene (VInv), which encodes a protein that breaks down sucrose to glucose and fructose (Clasen et al. DOI: 10.1111/pbi.12370).

### The Use of the System to Ensure a Value Added Trait

In particular embodiments the system is used to produce nutritionally improved agricultural crops. In particular embodiments, the methods provided herein are adapted to generate “functional foods”, i.e., a modified food or food ingredient that may provide a health benefit beyond the traditional nutrients it contains and or “nutraceutical”, i.e., substances that may be considered a food or part of a food and provides health benefits, including the prevention and treatment of disease. In particular embodiments, the nutraceutical is useful in the prevention and/or treatment of one or more of cancer, diabetes, cardiovascular disease, and hypertension.

Examples of nutritionally improved crops include (Newell-McGloughlin, Plant Physiology, July 2008, Vol. 147, pp. 939-953): modified protein quality, content and/or amino acid composition, such as have been described for Bahiagrass (Luciani et al. 2005, Florida Genetics Conference Poster), Canola (Roesler et al., 1997, Plant Physiol 113 75-81), Maize (Cromwell et al, 1967, 1969 J Anim Sci 26 1325-1331, O'Quin et al. 2000 J Anim Sci 78 2144-2149, Yang et al. 2002, Transgenic Res 11 11-20, Young et al. 2004, Plant J 38 910-922), Potato (Yu J and Ao, 1997 Acta Bot Sin 39 329-334; Chakraborty et al. 2000, Proc Natl Acad Sci USA 97 3724-3729; Li et al. 2001) Chin Sci Bull 46 482-484, Rice (Katsube et al. 1999, Plant Physiol 120 1063-1074), Soybean (Dinkins et al. 2001, Rapp 2002, In Vitro Cell Dev Biol Plant 37 742-747), Sweet Potato (Egnin and Prakash 1997, In Vitro Cell Dev Biol 33 52A).

Essential amino acid content, such as has been described for Canola (Falco et al. 1995, Bio/Technology 13 577-582), Lupin (White et al. 2001, J Sci Food Agric 81 147-154), Maize (Lai and Messing, 2002, Agbios 2008 GM crop database (Mar. 11, 2008)), Potato (Zeh et al. 2001, Plant Physiol 127 792-802), Sorghum (Zhao et al. 2003, Kluwer Academic Publishers, Dordrecht, The Netherlands, pp 413-416), Soybean (Falco et al. 1995 Bio/Technology 13 577-582; Galili et al. 2002 Crit Rev Plant Sci 21 167-204). Oils and Fatty acids such as for Canola (Dehesh et al. (1996) Plant J 9 167-172 [PubMed]; Del Vecchio (1996) INFORM International News on Fats, Oils and Related Materials 7 230-243; Roesler et al. (1997) Plant Physiol 113 75-81 [PMC free article][PubMed]; Froman and Ursin (2002, 2003) Abstracts of Papers of the American Chemical Society 223 U35; James et al. (2003) Am J Clin Nutr 77 1140-1145 [PubMed]; Agbios (2008, above); coton (Chapman et al. (2001). J Am Oil Chem Soc 78 941-947; Liu et al. (2002) J Am Coll Nutr 21 205S-211S; O'Neill (2007) Australian Life Scientist. www.biotechnews.com.au/index.php/id; 866694817; fp; 4; fpid; 2 (Jun. 17, 2008), Linseed (Abbadi et al., 2004, Plant Cell 16: 2734-2748), Maize (Young et al., 2004, Plant J 38 910-922), oil palm (Jalani et al. 1997, J Am Oil Chem Soc 74 1451-1455; Parveez, 2003, AgBiotechNet 113 1-8), Rice (Anai et al., 2003, Plant Cell Rep 21 988-992), Soybean (Reddy and Thomas, 1996, Nat Biotechnol 14 639-642; Kinney and Kwolton, 1998, Blackie Academic and Professional, London, pp 193-213), Sunflower (Arcadia, Biosciences 2008)

Carbohydrates, such as Fructans described for Chicory (Smeekens (1997) Trends Plant Sci 2 286-287, Sprenger et al. (1997) FEBS Lett 400 355-358, Sevenier et al. (1998) Nat Biotechnol 16 843-846), Maize (Caimi et al. (1996) Plant Physiol 110 355-363), Potato (Hellwege et al., 1997 Plant J 12 1057-1065), Sugar Beet (Smeekens et al. 1997, above), Inulin, such as described for Potato (Hellewege et al. 2000, Proc Natl Acad Sci USA 97 8699-8704), Starch, such as described for Rice (Schwall et al. (2000) Nat Biotechnol 18 551-554, Chiang et al. (2005) Mol Breed 15 125-143),

Vitamins and carotenoids, such as described for Canola (Shintani and DellaPenna (1998) Science 282 2098-2100), Maize (Rocheford et al. (2002). J Am Coll Nutr 21 191S-198S, Cahoon et al. (2003) Nat Biotechnol 21 1082-1087, Chen et al. (2003) Proc Natl Acad Sci USA 100 3525-3530), Mustardseed (Shewmaker et al. (1999) Plant J 20 401-412, Potato (Ducreux et al., 2005, J Exp Bot 56 81-89), Rice (Ye et al. (2000) Science 287 303-305, Strawberry (Agius et al. (2003), Nat Biotechnol 21 177-181), Tomato (Rosati et al. (2000) Plant J 24 413-419, Fraser et al. (2001) J Sci Food Agric 81 822-827, Mehta et al. (2002) Nat Biotechnol 20 613-618, Diaz de la Garza et al. (2004) Proc Natl Acad Sci USA 101 13720-13725, Enfissi et al. (2005) Plant Biotechnol J 3 17-27, DellaPenna (2007) Proc Natl Acad Sci USA 104 3675-3676.

Functional secondary metabolites, such as described for Apple (stilbenes, Szankowski et al. (2003) Plant Cell Rep 22: 141-149), Alfalfa (resveratrol, Hipskind and Paiva (2000) Mol Plant Microbe Interact 13 551-562), Kiwi (resveratrol, Kobayashi et al. (2000) Plant Cell Rep 19 904-910), Maize and Soybean (flavonoids, Yu et al. (2000) Plant Physiol 124 781-794), Potato (anthocyanin and alkaloid glycoside, Lukaszewicz et al. (2004) J Agric Food Chem 52 1526-1533), Rice (flavonoids & resveratrol, Stark-Lorenzen et al. (1997) Plant Cell Rep 16 668-673, Shin et al. (2006) Plant Biotechnol J 4 303-315), Tomato (+resveratrol, chlorogenic acid, flavonoids, stilbene; Rosati et al. (2000) above, Muir et al. (2001) Nature 19 470-474, Niggeweg et al. (2004) Nat Biotechnol 22 746-754, Giovinazzo et al. (2005) Plant Biotechnol J 3 57-69), wheat (caffeic and ferulic acids, resveratrol; United Press International (2002)); and

Mineral availabilities such as described for Alfalfa (phytase, Austin-Phillips et al. (1999) www.molecularfarming.com/nonmedical.html), Lettuse (iron, Goto et al. (2000) Theor Appl Genet 100 658-664), Rice (iron, Lucca et al. (2002) J Am Coll Nutr 21 184S-190S), Maize, Soybean and wheate (phytase, Drakakaki et al. (2005) Plant Mol Biol 59 869-880, Denbow et al. (1998) Poult Sci 77 878-881, Brinch-Pedersen et al. (2000) Mol Breed 6 195-206).

In particular embodiments, the value-added trait is related to the envisaged health benefits of the compounds present in the plant. For instance, in particular embodiments, the value-added crop is obtained by applying the methods of the invention to ensure the modification of or induce/increase the synthesis of one or more of the following compounds:

Carotenoids, such as α-Carotene present in carrots which Neutralizes free radicals that may cause damage to cells or β-Carotene present in various fruits and vegetables which neutralizes free radicals

Lutein present in green vegetables which contributes to maintenance of healthy vision

Lycopene present in tomato and tomato products, which is believed to reduce the risk of prostate cancer

Zeaxanthin, present in citrus and maize, which contributes to maintenance of healthy vision

Dietary fiber such as insoluble fiber present in wheat bran which may reduce the risk of breast and/or colon cancer and β-Glucan present in oat, soluble fiber present in Psylium and whole cereal grains which may reduce the risk of cardiovascular disease (CVD)

Fatty acids, such as ω-3 fatty acids which may reduce the risk of CVD and improve mental and visual functions, Conjugated linoleic acid, which may improve body composition, may decrease risk of certain cancers and GLA which may reduce inflammation risk of cancer and CVD, may improve body composition

Flavonoids such as hydroxycinnamates, present in wheat which have Antioxidant-like activities, may reduce risk of degenerative diseases, flavonols, catechins and tannins present in fruits and vegetables which neutralize free radicals and may reduce risk of cancer

Glucosinolates, indoles, isothiocyanates, such as Sulforaphane, present in Cruciferous vegetables (broccoli, kale), horseradish, which neutralize free radicals, may reduce risk of cancer

Phenolics, such as stilbenes present in grape which May reduce risk of degenerative diseases, heart disease, and cancer, may have longevity effect and caffeic acid and ferulic acid present in vegetables and citrus which have Antioxidant-like activities, may reduce risk of degenerative diseases, heart disease, and eye disease, and epicatechin present in cacao which has Antioxidant-like activities, may reduce risk of degenerative diseases and heart disease

Plant stanols/sterols present in maize, soy, wheat and wooden oils which May reduce risk of coronary heart disease by lowering blood cholesterol levels

Fructans, inulins, fructo-oligosaccharides present in Jerusalem artichoke, shallot, onion powder which may improve gastrointestinal health

Saponins present in soybean, which may lower LDL cholesterol

Soybean protein present in soybean which may reduce risk of heart disease

Phytoestrogens such as isoflavones present in soybean which May reduce menopause symptoms, such as hot flashes, may reduce osteoporosis and CVD and lignans present in flax, rye and vegetables, which May protect against heart disease and some cancers, may lower LDL cholesterol, total cholesterol.

Sulfides and thiols such as diallyl sulphide present in onion, garlic, olive, leek and scallon and Allyl methyl trisulfide, dithiolthiones present in cruciferous vegetables which may lower LDL cholesterol, helps to maintain healthy immune system

Tannins, such as proanthocyanidins, present in cranberry, cocoa, which may improve urinary tract health, may reduce risk of CVD and high blood pressure.

In addition, the methods of the present invention also envisage modifying protein/starch functionality, shelf life, taste/aesthetics, fiber quality, and allergen, antinutrient, and toxin reduction traits.

Accordingly, the invention encompasses methods for producing plants with nutritional added value, said methods comprising introducing into a plant cell a gene encoding an enzyme involved in the production of a component of added nutritional value using the systems as described herein and regenerating a plant from said plant cell, said plant characterized in an increase expression of said component of added nutritional value. In particular embodiments, the systems is used to modify the endogenous synthesis of these compounds indirectly, e.g., by modifying one or more transcription factors that controls the metabolism of this compound. Methods for introducing a gene of interest into a plant cell and/or modifying an endogenous gene using the systems are described herein above.

Some specific examples of modifications in plants that have been modified to confer value-added traits are: plants with modified fatty acid metabolism, for example, by transforming a plant with an antisense gene of stearyl-ACP desaturase to increase stearic acid content of the plant. See Knultzon et al., Proc. Natl. Acad. Sci. U.S.A. 89:2624 (1992). Another example involves decreasing phytate content, for example by cloning and then reintroducing DNA associated with the single allele which may be responsible for maize mutants characterized by low levels of phytic acid. See Raboy et al, Maydica 35:383 (1990).

Similarly, expression of the maize (Zea mays) Tfs C1 and R, which regulate the production of flavonoids in maize aleurone layers under the control of a strong promoter, resulted in a high accumulation rate of anthocyanins in Arabidopsis (Arabidopsis thaliana), presumably by activating the entire pathway (Bruce et al., 2000, Plant Cell 12:65-80). DellaPenna (Welsch et al., 2007 Annu Rev Plant Biol 57: 711-738) found that Tf RAP2.2 and its interacting partner SINAT2 increased carotenogenesis in Arabidopsis leaves. Expressing the Tf Dof1 induced the up-regulation of genes encoding enzymes for carbon skeleton production, a marked increase of amino acid content, and a reduction of the Glc level in transgenic Arabidopsis (Yanagisawa, 2004 Plant Cell Physiol 45: 386-391), and the DOF Tf AtDof1.1 (OBP2) up-regulated all steps in the glucosinolate biosynthetic pathway in Arabidopsis (Skirycz et al., 2006 Plant J 47: 10-24).

### Reducing Allergen in Plants

In particular embodiments the methods provided herein are used to generate plants with a reduced level of allergens, making them safer for the consumer. In particular embodiments, the methods comprise modifying expression of one or more genes responsible for the production of plant allergens. For instance, in particular embodiments, the methods comprise down-regulating expression of a Lol p5 gene in a plant cell, such as a ryegrass plant cell and regenerating a plant therefrom so as to reduce allergenicity of the pollen of said plant (Bhalla et al. 1999, Proc. Natl. Acad. Sci. USA Vol. 96: 11676-11680).

Peanut allergies and allergies to legumes generally are a real and serious health concern. The systems of the present invention can be used to identify and then edit or silence genes encoding allergenic proteins of such legumes. Without limitation as to such genes and proteins, Nicolaou et al. identifies allergenic proteins in peanuts, soybeans, lentils, peas, lupin, green beans, and mung beans. See, Nicolaou et al., Current Opinion in Allergy and Clinical Immunology 2011; 11(3):222).

### Screening Methods for Endogenous Genes of Interest

The methods provided herein further allow the identification of genes of value encoding enzymes involved in the production of a component of added nutritional value or generally genes affecting agronomic traits of interest across species, phyla, and plant kingdoms. By selectively targeting e.g., genes encoding enzymes of metabolic pathways in plants using the systems as described herein, the genes responsible for certain nutritional aspects of a plant can be identified. Similarly, by selectively targeting genes which may affect a desirable agronomic trait, the relevant genes can be identified. Accordingly, the present invention encompasses screening methods for genes encoding enzymes involved in the production of compounds with a particular nutritional value and/or agronomic traits.

### Further Applications of the System of the Present Invention in Plants and Yeasts

### Biofuel Production

The term “biofuel” as used herein is an alternative fuel made from plant and plant-derived resources. Renewable biofuels can be extracted from organic matter whose energy has been obtained through a process of carbon fixation or are made through the use or conversion of biomass. This biomass can be used directly for biofuels or can be converted to convenient energy containing substances by thermal conversion, chemical conversion, and biochemical conversion. This biomass conversion can result in fuel in solid, liquid, or gas form. There are two types of biofuels: bioethanol and biodiesel. Bioethanol is mainly produced by the sugar fermentation process of cellulose (starch), which is mostly derived from maize and sugar cane. Biodiesel on the other hand is mainly produced from oil crops such as rapeseed, palm, and soybean. Biofuels are used mainly for transportation.

### Enhancing Plant Properties for Biofuel Production

In particular embodiments, the methods using the system as described herein are used to alter the properties of the cell wall in order to facilitate access by key hydrolyzing agents for a more efficient release of sugars for fermentation. In particular embodiments, the biosynthesis of cellulose and/or lignin are modified. Cellulose is the major component of the cell wall. The biosynthesis of cellulose and lignin are co-regulated. By reducing the proportion of lignin in a plant the proportion of cellulose can be increased. In particular embodiments, the methods described herein are used to downregulate lignin biosynthesis in the plant so as to increase fermentable carbohydrates. More particularly, the methods described herein are used to downregulate at least a first lignin biosynthesis gene selected from the group consisting of 4-coumarate 3-hydroxylase (C3H), phenylalanine ammonia-lyase (PAL), cinnamate 4-hydroxylase (C4H), hydroxycinnamoyl transferase (HCT), caffeic acid O-methyltransferase (COMT), caffeoyl CoA 3-O-methyltransferase (CCoAOMT), ferulate 5-hydroxylase (F5H), cinnamyl alcohol dehydrogenase (CAD), cinnamoyl CoA-reductase (CCR), 4-coumarate-CoA ligase (4CL), monolignol-lignin-specific glycosyltransferase, and aldehyde dehydrogenase (ALDH) as disclosed in WO 2008064289 A2.

In particular embodiments, the methods described herein are used to produce plant mass that produces lower levels of acetic acid during fermentation (see also WO 2010096488). More particularly, the methods disclosed herein are used to generate mutations in homologs to CaslL to reduce polysaccharide acetylation.

### Modifying Yeast for Biofuel Production

In particular embodiments, the systems provided herein is used for bioethanol production by recombinant micro-organisms. For instance, the systems can be used to engineer micro-organisms, such as yeast, to generate biofuel or biopolymers from fermentable sugars and optionally to be able to degrade plant-derived lignocellulose derived from agricultural waste as a source of fermentable sugars. More particularly, the invention provides methods whereby the system is used to introduce foreign genes required for biofuel production into micro-organisms and/or to modify endogenous genes why may interfere with the biofuel synthesis. More particularly the methods involve introducing into a micro-organism such as a yeast one or more nucleotide sequence encoding enzymes involved in the conversion of pyruvate to ethanol or another product of interest. In particular embodiments the methods ensure the introduction of one or more enzymes which allows the micro-organism to degrade cellulose, such as a cellulase. In yet further embodiments, the systems is used to modify endogenous metabolic pathways which compete with the biofuel production pathway.

Accordingly, in more particular embodiments, the methods described herein are used to modify a micro-organism as follows: to introduce at least one heterologous nucleic acid or increase expression of at least one endogenous nucleic acid encoding a plant cell wall degrading enzyme, such that said micro-organism is capable of expressing said nucleic acid and of producing and secreting said plant cell wall degrading enzyme; to introduce at least one heterologous nucleic acid or increase expression of at least one endogenous nucleic acid encoding an enzyme that converts pyruvate to acetaldehyde optionally combined with at least one heterologous nucleic acid encoding an enzyme that converts acetaldehyde to ethanol such that said host cell is capable of expressing said nucleic acid; and/or to modify at least one nucleic acid encoding for an enzyme in a metabolic pathway in said host cell, wherein said pathway produces a metabolite other than acetaldehyde from pyruvate or ethanol from acetaldehyde, and wherein said modification results in a reduced production of said metabolite, or to introduce at least one nucleic acid encoding for an inhibitor of said enzyme.

### Modifying Algae and Plants for Production of Vegetable Oils or Biofuels

Transgenic algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries.

According to particular embodiments of the invention, the system is used to generate lipid-rich diatoms which are useful in biofuel production.

In particular embodiments it is envisaged to specifically modify genes that are involved in the modification of the quantity of lipids and/or the quality of the lipids produced by the algal cell. Examples of genes encoding enzymes involved in the pathways of fatty acid synthesis can encode proteins having for instance acetyl-CoA carboxylase, fatty acid synthase, 3-ketoacyl_acyl-carrier protein synthase III, glycerol-3-phospate dehydrogenase (G3PDH), Enoyl-acyl carrier protein reductase (Enoyl-ACP-reductase), glycerol-3-phosphate acyltransferase, lysophosphatidic acyl transferase or diacylglycerol acyltransferase, phospholipid:diacylglycerol acyltransferase, phoshatidate phosphatase, fatty acid thioesters such as palmitoyl protein thioesters, or malic enzyme activities. In further embodiments it is envisaged to generate diatoms that have increased lipid accumulation. This can be achieved by targeting genes that decrease lipid categorization. Of particular interest for use in the methods of the present invention are genes involved in the activation of both triacylglycerol and free fatty acids, as well as genes directly involved in β-oxidation of fatty acids, such as acyl-CoA synthetase, 3-ketoacyl-CoA thiolase, acyl-CoA oxidase activity and phosphoglucomutase. The system and methods described herein can be used to specifically activate such genes in diatoms as to increase their lipid content.

Organisms such as microalgae are widely used for synthetic biology. Stovicek et al. (Metab. Eng. Comm., 2015; 2:13 describes genome editing of industrial yeast, for example, Saccharomyces cerevisae, to efficiently produce robust strains for industrial production. Stovicek used a CRISPR-Cas9 system codon-optimized for yeast to simultaneously disrupt both alleles of an endogenous gene and knock in a heterologous gene. Cas9 and gRNA were expressed from genomic or episomal 2-based vector locations. The authors also showed that gene disruption efficiency could be improved by optimization of the levels of Cas9 and gRNA expression. Hlavovi et al. (Biotechnol. Adv. 2015) discusses development of species or strains of microalgae using techniques such as CRISPR to target nuclear and chloroplast genes for insertional mutagenesis and screening. The methods of Stovicek and Hlavovi may be applied to the Cas effector protein system of the present invention.

U.S. Pat. No. 8,945,839 describes a method for engineering Micro-Algae (Chlamydomonas reinhardtii cells) species) using Cas9. Using similar tools, the methods of the systems described herein can be applied on Chlamydomonas species and other algae. In particular embodiments, Cas and guide RNA are introduced in algae expressed using a vector that expresses Cas under the control of a constitutive promoter such as Hsp70A-Rbc S2 or Beta2-tubulin. Guide RNA will be delivered using a vector containing T7 promoter. Alternatively, Cas mRNA and in vitro transcribed guide RNA can be delivered to algal cells. Electroporation protocol follows standard recommended protocol from the GeneArt Chlamydomonas Engineering kit.

### Generation of Micro-Organisms Capable of Fatty Acid Production

In particular embodiments, the methods of the invention are used for the generation of genetically engineered micro-organisms capable of the production of fatty esters, such as fatty acid methyl esters (“FAME”) and fatty acid ethyl esters (“FAEE”),

Typically, host cells can be engineered to produce fatty esters from a carbon source, such as an alcohol, present in the medium, by expression or overexpression of a gene encoding a thioesters, a gene encoding an acyl-CoA synthase, and a gene encoding an ester synthase. Accordingly, the methods provided herein are used to modify a micro-organisms so as to overexpress or introduce a thioesters gene, a gene encoding an acyl-CoA synthase, and a gene encoding an ester synthase. In particular embodiments, the thioesters gene is selected from tesA, ′tesA, tesB, fatB, fatB2, fatB3, fatA1, or fatA. In particular embodiments, the gene encoding an acyl-CoA synthase is selected from fadDJadK, BH3103, pfl-4354, EAV15023, fadD1, fadD2, RPC_4074, fadDD35, fadDD22, faa39, or an identified gene encoding an enzyme having the same properties. In particular embodiments, the gene encoding an ester synthase is a gene encoding a synthase/acyl-CoA:diacylglycerl acyltransferase from Simmondsia chinensis, Acinetobacter sp. ADP, Alcanivorax borkumensis, Pseudomonas aeruginosa, Fundibacter jadensis, Arabidopsis thaliana, or Alkaligenes eutrophus, or a variant thereof.

Additionally, or alternatively, the methods provided herein are used to decrease expression in said micro-organism of at least one of a gene encoding an acyl-CoA dehydrogenase, a gene encoding an outer membrane protein receptor, and a gene encoding a transcriptional regulator of fatty acid biosynthesis. In particular embodiments one or more of these genes is inactivated, such as by introduction of a mutation. In particular embodiments, the gene encoding an acyl-CoA dehydrogenase is fadE. In particular embodiments, the gene encoding a transcriptional regulator of fatty acid biosynthesis encodes a DNA transcription repressor, for example, fabR.

Additionally, or alternatively, said micro-organism is modified to reduce expression of at least one of a gene encoding a pyruvate formate lyase, a gene encoding a lactate dehydrogenase, or both. In particular embodiments, the gene encoding a pyruvate formate lyase is pflB. In particular embodiments, the gene encoding a lactate dehydrogenase is IdhA. In particular embodiments one or more of these genes is inactivated, such as by introduction of a mutation therein.

In particular embodiments, the micro-organism is selected from the genus Escherichia, Bacillus, Lactobacillus, Rhodococcus, Synechococcus, Synechoystis, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces.

### Generation of Micro-Organisms Capable of Organic Acid Production

The methods provided herein are further used to engineer micro-organisms capable of organic acid production, more particularly from pentose or hexose sugars. In particular embodiments, the methods comprise introducing into a micro-organism an exogenous LDH gene. In particular embodiments, the organic acid production in said micro-organisms is additionally or alternatively increased by inactivating endogenous genes encoding proteins involved in an endogenous metabolic pathway which produces a metabolite other than the organic acid of interest and/or wherein the endogenous metabolic pathway consumes the organic acid. In particular embodiments, the modification ensures that the production of the metabolite other than the organic acid of interest is reduced. According to particular embodiments, the methods are used to introduce at least one engineered gene deletion and/or inactivation of an endogenous pathway in which the organic acid is consumed or a gene encoding a product involved in an endogenous pathway which produces a metabolite other than the organic acid of interest. In particular embodiments, the at least one engineered gene deletion or inactivation is in one or more gene encoding an enzyme selected from the group consisting of pyruvate decarboxylase (pdc), fumarate reductase, alcohol dehydrogenase (adh), acetaldehyde dehydrogenase, phosphoenolpyruvate carboxylase (ppc), D-lactate dehydrogenase (d-ldh), L-lactate dehydrogenase (1-ldh), lactate 2-monooxygenase. In further embodiments the at least one engineered gene deletion and/or inactivation is in an endogenous gene encoding pyruvate decarboxylase (pdc).

In further embodiments, the micro-organism is engineered to produce lactic acid and the at least one engineered gene deletion and/or inactivation is in an endogenous gene encoding lactate dehydrogenase. Additionally, or alternatively, the micro-organism comprises at least one engineered gene deletion or inactivation of an endogenous gene encoding a cytochrome-dependent lactate dehydrogenase, such as a cytochrome B2-dependent L-lactate dehydrogenase.

### Generation of Improved Xylose or Cellobiose Utilizing Yeasts Strains

In particular embodiments, the systems disclosed herein may be applied to select for improved xylose or cellobiose utilizing yeast strains. Error-prone PCR can be used to amplify one (or more) genes involved in the xylose utilization or cellobiose utilization pathways. Examples of genes involved in xylose utilization pathways and cellobiose utilization pathways may include, without limitation, those described in Ha, S. J., et al. (2011) Proc. Natl. Acad. Sci. USA 108(2):504-9 and Galazka, J. M., et al. (2010) Science 330(6000):84-6. Resulting libraries of double-stranded DNA molecules, each comprising a random mutation in such a selected gene could be co-transformed with the components of the system into a yeast strain (for instance S288C) and strains can be selected with enhanced xylose or cellobiose utilization capacity, as described in WO2015138855.

### Generation of Improved Yeasts Strains for Use in Isoprenoid Biosynthesis

Tadas Jakociunas et al. described the successful application of a multiplex CRISPR/Cas9 system for genome engineering of up to 5 different genomic loci in one transformation step in baker's yeast Saccharomyces cerevisiae (Metabolic Engineering Volume 28, March 2015, Pages 213-222) resulting in strains with high mevalonate production, a key intermediate for the industrially important isoprenoid biosynthesis pathway. In particular embodiments, the systems may be applied in a multiplex genome engineering method as described herein for identifying additional high producing yeast strains for use in isoprenoid synthesis.

### Generation of Lactic Acid Producing Yeasts Strains

In another embodiment, successful application of a multiplex system is encompassed. In analogy with Vratislav Stovicek et al. (Metabolic Engineering Communications, Volume 2, December 2015, Pages 13-22), improved lactic acid-producing strains can be designed and obtained in a single transformation event. In a particular embodiment, the systems is used for simultaneously inserting the heterologous lactate dehydrogenase gene and disruption of two endogenous genes PDC1 and PDC5 genes.

### Further Applications of the System in Plants

In particular embodiments, the system can be used for visualization of genetic element dynamics. For example, CRISPR imaging can visualize either repetitive or non-repetitive genomic sequences, report telomere length change and telomere movements and monitor the dynamics of gene loci throughout the cell cycle (Chen et al., Cell, 2013). These methods may also be applied to plants.

Other applications of the systems, and preferably the systems described herein, is the targeted gene disruption positive-selection screening in vitro and in vivo (Malina et al., Genes and Development, 2013). These methods may also be applied to plants.

In particular embodiments, fusion of inactive Cas endonucleases with histone-modifying enzymes can introduce custom changes in the complex epigenome (Rusk et al., Nature Methods, 2014). These methods may also be applied to plants.

In particular embodiments, the systems, and preferably the systems described herein, can be used to purify a specific portion of the chromatin and identify the associated proteins, thus elucidating their regulatory roles in transcription (Waldrip et al., Epigenetics, 2014). These methods may also be applied to plants.

In particular embodiments, present invention can be used as a therapy for virus removal in plant systems as it is able to cleave both viral DNA and RNA. Previous studies in human systems have demonstrated the success of utilizing CRISPR in targeting the single strand RNA virus, hepatitis C (A. Price, et al., Proc. Natl. Acad. Sci, 2015) as well as the double stranded DNA virus, hepatitis B (V. Ramanan, et al., Sci. Rep, 2015). These methods may also be adapted for using the systems in plants.

In particular embodiments, present invention could be used to alter genome complexity. In further particular embodiment, the systems, and preferably the systems described herein, can be used to disrupt or alter chromosome number and generate haploid plants, which only contain chromosomes from one parent. Such plants can be induced to undergo chromosome duplication and converted into diploid plants containing only homozygous alleles (Karimi-Ashtiyani et al., PNAS, 2015; Anton et al., Nucleus, 2014). These methods may also be applied to plants.

In particular embodiments, the systems described herein, can be used for self-cleavage. In these embodiments, the promotor of the Cas enzyme and gRNA can be a constitutive promotor and a second gRNA is introduced in the same transformation cassette but controlled by an inducible promoter. This second gRNA can be designated to induce site-specific cleavage in the Cas gene in order to create a non-functional Cas. In a further particular embodiment, the second gRNA induces cleavage on both ends of the transformation cassette, resulting in the removal of the cassette from the host genome. This system offers a controlled duration of cellular exposure to the Cas enzyme and further minimizes off-target editing. Furthermore, cleavage of both ends of a CRISPR/Cas cassette can be used to generate transgene-free TO plants with bi-allelic mutations (as described for Cas9 e.g. Moore et al., Nucleic Acids Research, 2014; Schaeffer et al., Plant Science, 2015). The methods of Moore et al. may be applied to the systems described herein.

Sugano et al. (Plant Cell Physiol. 2014 March; 55(3):475-81. doi: 10.1093/pcp/pcu014. Epub 2014 Jan. 18) reports the application of CRISPR-Cas9 to targeted mutagenesis in the liverwort Marchantia polymorpha L., which has emerged as a model species for studying land plant evolution. The U6 promoter of M. polymorpha was identified and cloned to express the gRNA. The target sequence of the gRNA was designed to disrupt the gene encoding auxin response factor 1 (ARF1) in M. polymorpha. Using Agrobacterium-mediated transformation, Sugano et al. isolated stable mutants in the gametophyte generation of M. polymorpha. CRISPR-Cas9-based site-directed mutagenesis in vivo was achieved using either the Cauliflower mosaic virus 35S or M. polymorpha EF1α promoter to express Cas9. Isolated mutant individuals showing an auxin-resistant phenotype were not chimeric. Moreover, stable mutants were produced by asexual reproduction of T1 plants. Multiple arf1 alleles were easily established using CRIPSR-Cas9-based targeted mutagenesis. The methods of Sugano et al. may be applied to the Cas effector protein system of the present invention.

Kabadi et al. (Nucleic Acids Res. 2014 Oct. 29; 42(19):e147. doi: 10.1093/nar/gku749. Epub 2014 Aug. 13) developed a single lentiviral system to express a Cas9 variant, a reporter gene and up to four sgRNAs from independent RNA polymerase III promoters that are incorporated into the vector by a convenient Golden Gate cloning method. Each sgRNA was efficiently expressed and can mediate multiplex gene editing and sustained transcriptional activation in immortalized and primary human cells. The methods of Kabadi et al. may be applied to the Cas effector protein system of the present invention.

Ling et al. (BMC Plant Biology 2014, 14:327) developed a CRISPR-Cas9 binary vector set based on the pGreen or pCAMBIA backbone, as well as a gRNA This toolkit requires no restriction enzymes besides BsaI to generate final constructs harboring maize-codon optimized Cas9 and one or more gRNAs with high efficiency in as little as one cloning step. The toolkit was validated using maize protoplasts, transgenic maize lines, and transgenic Arabidopsis lines and was shown to exhibit high efficiency and specificity. More importantly, using this toolkit, targeted mutations of three Arabidopsis genes were detected in transgenic seedlings of the T1 generation. Moreover, the multiple-gene mutations could be inherited by the next generation. The (guide RNA)module vector can be provided as a set, such as a toolkit for multiplex genome editing in plants. The toolbox of Lin et al. may be applied to the Cas effector protein system of the present invention.

Protocols for targeted plant genome editing via CRISPR-Cas are also available based on those disclosed for the CRISPR-Cas9 system in volume 1284 of the series Methods in Molecular Biology pp 239-255 10 Feb. 2015. A detailed procedure to design, construct, and evaluate dual gRNAs for plant codon optimized Cas9 (pcoCas9) mediated genome editing using Arabidopsis thaliana and Nicotiana benthamiana protoplasts s model cellular systems are described. Strategies to apply the CRISPR-Cas9 system to generating targeted genome modifications in whole plants are also discussed. The protocols described in the chapter may be applied to the Cas effector protein system of the present invention.

Ma et al. (Mol Plant. 2015 Aug. 3; 8(8):1274-84. doi: 10.1016/j.molp.2015.04.007) reports robust CRISPR-Cas9 vector system, utilizing a plant codon optimized Cas9 gene, for convenient and high-efficiency multiplex genome editing in monocot and dicot plants. Ma et al. designed PCR-based procedures to rapidly generate multiple sgRNA expression cassettes, which can be assembled into the binary CRISPR-Cas9 vectors in one round of cloning by Golden Gate ligation or Gibson Assembly. With this system, Ma et al. edited 46 target sites in rice with an average 85.4% rate of mutation, mostly in biallelic and homozygous status. Ma et al. provide examples of loss-of-function gene mutations in TO rice and T1Arabidopsis plants by simultaneous targeting of multiple (up to eight) members of a gene family, multiple genes in a biosynthetic pathway, or multiple sites in a single gene. The methods of Ma et al. may be applied to the Cas effector protein system of the present invention.

Lowder et al. (Plant Physiol. 2015 Aug. 21. pii: pp. 00636.2015) also developed a CRISPR-Cas9 toolbox enables multiplex genome editing and transcriptional regulation of expressed, silenced or non-coding genes in plants. This toolbox provides researchers with a protocol and reagents to quickly and efficiently assemble functional CRISPR-Cas9 T-DNA constructs for monocots and dicots using Golden Gate and Gateway cloning methods. It comes with a full suite of capabilities, including multiplexed gene editing and transcriptional activation or repression of plant endogenous genes. T-DNA based transformation technology is fundamental to modern plant biotechnology, genetics, molecular biology and physiology. As such, Applicants developed a method for the assembly of Cas (WT, nickase or dCas) and gRNA(s) into a T-DNA destination-vector of interest. The assembly method is based on both Golden Gate assembly and MultiSite Gateway recombination. Three modules are required for assembly. The first module is a Cas entry vector, which contains promoterless Cas or its derivative genes flanked by attL1 and attR5 sites. The second module is a gRNA entry vector which contains entry gRNA expression cassettes flanked by attL5 and attL2 sites. The third module includes attR1-attR2-containing destination T-DNA vectors that provide promoters of choice for Cas expression. The toolbox of Lowder et al. may be applied to the Cas effector protein system of the present invention.

Wang et al. (bioRxiv 051342; doi: doi.org/10.1101/051342; Epub. May 12, 2016) demonstrate editing of homoeologous copies of four genes affecting important agronomic traits in hexaploid wheat using a multiplexed gene editing construct with several gRNA-tRNA units under the control of a single promoter.

In an advantageous embodiment, the plant may be a tree. The present invention may also utilize the herein disclosed systems for herbaceous systems (see, e.g., Belhaj et al., Plant Methods 9: 39 and Harrison et al., Genes & Development 28: 1859-1872). In a particularly advantageous embodiment, the Systems of the present invention may target single nucleotide polymorphisms (SNPs) in trees (see, e.g., Zhou et al., New Phytologist, Volume 208, Issue 2, pages 298-301, October 2015). In the Zhou et al. study, the authors applied a systems in the woody perennial Populus using the 4-coumarate:CoA ligase (4CL) gene family as a case study and achieved 100% mutational efficiency for two 4CL genes targeted, with every transformant examined carrying biallelic modifications. In the Zhou et al., study, the CRISPR-Cas9 system was highly sensitive to single nucleotide polymorphisms (SNPs), as cleavage for a third 4CL gene was abolished due to SNPs in the target sequence. These methods may be applied to the Cas effector protein system of the present invention.

The methods of Zhou et al. (New Phytologist, Volume 208, Issue 2, pages 298-301, October 2015) may be applied to the present invention as follows. Two 4CL genes, 4CL1 and 4CL2, associated with lignin and flavonoid biosynthesis, respectively are targeted for CRISPR-Cas9 editing. The Populus tremula×alba clone 717-1B4 routinely used for transformation is divergent from the genome-sequenced Populus trichocarpa. Therefore, the 4CL1 and 4CL2 gRNAs designed from the reference genome are interrogated with in-house 717 RNA-Seq data to ensure the absence of SNPs which could limit Cas efficiency. A third gRNA designed for 4CL5, a genome duplicate of 4CL1, is also included. The corresponding 717 sequence harbors one SNP in each allele near/within the PAM, both of which are expected to abolish targeting by the 4CL5-gRNA. All three gRNA target sites are located within the first exon. For 717 transformation, the gRNA is expressed from the Medicago U6.6 promoter, along with a human codon-optimized Cas under control of the CaMV 35S promoter in a binary vector. Transformation with the Cas-only vector can serve as a control. Randomly selected 4CL1 and 4CL2 lines are subjected to amplicon-sequencing. The data is then processed and biallelic mutations are confirmed in all cases. These methods may be applied to the Cas effector protein system of the present invention.

In plants, pathogens are often host-specific. For example, Fusarium oxysporum f. sp. lycopersici causes tomato wilt but attacks only tomato, and F. oxysporum f. dianthii Puccinia graminis f. sp. tritici attacks only wheat. Plants have existing and induced defenses to resist most pathogens. Mutations and recombination events across plant generations lead to genetic variability that gives rise to susceptibility, especially as pathogens reproduce with more frequency than plants. In plants there can be non-host resistance, e.g., the host and pathogen are incompatible. There can also be Horizontal Resistance, e.g., partial resistance against all races of a pathogen, typically controlled by many genes and Vertical Resistance, e.g., complete resistance to some races of a pathogen but not to other races, typically controlled by a few genes. In a Gene-for-Gene level, plants and pathogens evolve together, and the genetic changes in one balance changes in other. Accordingly, using Natural Variability, breeders combine most useful genes for Yield, Quality, Uniformity, Hardiness, Resistance. The sources of resistance genes include native or foreign Varieties, Heirloom Varieties, Wild Plant Relatives, and Induced Mutations, e.g., treating plant material with mutagenic agents. Using the present invention, plant breeders are provided with a new tool to induce mutations. Accordingly, one skilled in the art can analyze the genome of sources of resistance genes, and in Varieties having desired characteristics or traits employ the present invention to induce the rise of resistance genes, with more precision than previous mutagenic agents and hence accelerate and improve plant breeding programs.

Table 4 below provides additional references and related fields for which the systems, complexes, modified effector proteins, systems, and methods of optimization may be used to improve bioproduction.

TABLE 4

| | | |
|---|---|---|
| Feb. 17, 2014 | PCT/US15/63434 | Compositions and methods for efficient gene editing |
| | (WO2016/099887) | in E. coli using guide RNA/Cas endonuclease |
| | | systems in combination with circular polynucleotide |
| | | modification templates. |
| Aug. 13, 2014 | PCT/US15/41256 | Genetic targeting in non-conventional yeast using |
| | (WO2016/025131) | an RNA-guided endonuclease. |
| Nov. 6, 2014 | PCT/US15/58760 | Peptide-mediated delivery of RNA-guided |
| | WO2016/073433) | endonuclease into cells. |
| Oct. 12, 2015 | PCT/US16/56404 | Protected DNA templates for gene modification and |
| | (WO2017/066175) | increased homologous recombination in cells and |
| | | methods of use. |
| Dec. 11, 2015 | PCT/US16/65070 | Methods and compositions for enhanced nuclease- |
| | (WO2017/100158) | mediated genome modification and reduced off- |
| | | target site effects. |
| Dec. 18, 2015 | PCT/US16/65537 | Methods and compositions for T-RNA based guide |
| | (WO2017/105991) | RNA expression. |
| Dec. 18, 2015 | PCT/US16/66772 | Methods and compositions for polymerase II (Pol-II) |
| | (WO2017/106414) | based guide RNA expression. |
| Dec. 16, 2014 | PCT/US15/65693 | Fungal genome modification systems and methods |
| | (WO2016/100272) | of use. |
| Dec. 16, 2014 | PCT/US15/66195 | Fungal genome modification systems and methods |
| | (WO2016/100571) | of use |
| Dec. 16, 2014 | PCT/US15/66192 | Fungal genome modification systems and methods |
| | WO 2016/100568) | of use. |
| Dec. 16, 2014 | PCT/US15/66178 | Use of a helper strain with silenced NHEJ to |
| | (WO 2016/100562) | improve homologous integration of targeted DNA |
| | | cassettes in Trichoderma reesei. |
| Jul. 28, 2015 | PCT/US16/44489 | Genome editing systems and methods of use. |
| | (WO 2017/019867) | |

### Improved Plants and Yeast Cells

The present invention also provides plants and yeast cells obtainable and obtained by the methods provided herein. The improved plants obtained by the methods described herein may be useful in food or feed production through expression of genes which, for instance ensure tolerance to plant pests, herbicides, drought, low or high temperatures, excessive water, etc.

The improved plants obtained by the methods described herein, especially crops and algae may be useful in food or feed production through expression of, for instance, higher protein, carbohydrate, nutrient or vitamin levels than would normally be seen in the wildtype. In this regard, improved plants, especially pulses and tubers are preferred.

Improved algae or other plants such as rape may be particularly useful in the production of vegetable oils or biofuels such as alcohols (especially methanol and ethanol), for instance. These may be engineered to express or overexpress high levels of oil or alcohols for use in the oil or biofuel industries.

The invention also provides for improved parts of a plant. Plant parts include, but are not limited to, leaves, stems, roots, tubers, seeds, endosperm, ovule, and pollen. Plant parts as envisaged herein may be viable, nonviable, regeneratable, and/or non-regeneratable.

In one embodiment, the method described in Soyk et al. (Nat Genet. 2017 January; 49(1):162-168), which used CRISPR-Cas9 mediated mutation targeting flowering repressor SP5G in tomatoes to produce early yield tomatoes may be modified for the system as disclosed in this invention.

It is also encompassed herein to provide plant cells and plants generated according to the methods of the invention. Gametes, seeds, germplasm, embryos, either zygotic or somatic, progeny or hybrids of plants comprising the genetic modification, which are produced by traditional breeding methods, are also included within the scope of the present invention. Such plants may contain a heterologous or foreign DNA sequence inserted at or instead of a target sequence. Alternatively, such plants may contain only an alteration (mutation, deletion, insertion, substitution) in one or more nucleotides. As such, such plants will only be different from their progenitor plants by the presence of the particular modification.

Thus, the invention provides a plant, animal or cell, produced by the present methods, or a progeny thereof. The progeny may be a clone of the produced plant or animal or may result from sexual reproduction by crossing with other individuals of the same species to introgress further desirable traits into their offspring. The cell may be in vivo or ex vivo in the cases of multicellular organisms, particularly animals or plants.

The methods for genome editing using the Cas system as described herein can be used to confer desired traits on essentially any plant, algae, fungus, yeast, etc. A wide variety of plants, algae, fungus, yeast, etc. and plant algae, fungus, yeast cell or tissue systems may be engineered for the desired physiological and agronomic characteristics described herein using the nucleic acid constructs of the present disclosure and the various transformation methods mentioned above.

In particular embodiments, the methods described herein are used to modify endogenous genes or to modify their expression without the permanent introduction into the genome of the plant, algae, fungus, yeast, etc. of any foreign gene, including those encoding CRISPR components, so as to avoid the presence of foreign DNA in the genome of the plant. This can be of interest as the regulatory requirements for non-transgenic plants are less rigorous.

The systems provided herein can be used to introduce targeted double-strand or single-strand breaks and/or to introduce gene activator and or repressor systems and without being limitative, can be used for gene targeting, gene replacement, targeted mutagenesis, targeted deletions or insertions, targeted inversions and/or targeted translocations. By co-expression of multiple targeting RNAs directed to achieve multiple modifications in a single cell, multiplexed genome modification can be ensured. This technology can be used to high-precision engineering of plants with improved characteristics, including enhanced nutritional quality, increased resistance to diseases and resistance to biotic and abiotic stress, and increased production of commercially valuable plant products or heterologous compounds.

The methods described herein generally result in the generation of “improved plants, algae, fungi, yeast, etc.” in that they have one or more desirable traits compared to the wildtype plant. In particular embodiments, the plants, algae, fungi, yeast, etc., cells or parts obtained are transgenic plants, comprising an exogenous DNA sequence incorporated into the genome of all or part of the cells. In particular embodiments, non-transgenic genetically modified plants, algae, fungi, yeast, etc., parts or cells are obtained, in that no exogenous DNA sequence is incorporated into the genome of any of the cells of the plant. In such embodiments, the improved plants, algae, fungi, yeast, etc. are non-transgenic. Where only the modification of an endogenous gene is ensured and no foreign genes are introduced or maintained in the plant, algae, fungi, yeast, etc. genome, the resulting genetically modified crops contain no foreign genes and can thus basically be considered non-transgenic. The different applications of the systems for plant, algae, fungi, yeast, etc. genome editing include, but are not limited to: introduction of one or more foreign genes to confer an agricultural trait of interest; editing of endogenous genes to confer an agricultural trait of interest; modulating of endogenous genes by the systems to confer an agricultural trait of interest. Exemplary genes conferring agronomic traits include, but are not limited to, genes that confer resistance to pests or diseases; genes involved in plant diseases, such as those listed in WO 2013046247; genes that confer resistance to herbicides, fungicides, or the like; genes involved in (abiotic) stress tolerance. Other aspects of the use of the systems include, but are not limited to: create (male) sterile plants; increasing the fertility stage in plants/algae etc.; generate genetic variation in a crop of interest; affect fruit-ripening; increasing storage life of plants/algae etc.; reducing allergen in plants/algae etc.; ensure a value added trait (e.g., nutritional improvement); Screening methods for endogenous genes of interest; biofuel, fatty acid, organic acid, etc. production.

### Applications in Non-Human Animals

The systems and methods of the present invention may be used in non-human animals and/or cells thereof to modify a polynucleotide in the non-human animal and/or cell(s) thereof. In an aspect, the invention provides a non-human eukaryotic organism; preferably a multicellular eukaryotic organism, comprising a eukaryotic host cell according to any of the described embodiments. In other aspects, the invention provides a eukaryotic organism; preferably a multicellular eukaryotic organism, comprising a eukaryotic host cell according to any of the described embodiments. The organism in some embodiments of these aspects may be an animal, for example a mammal. Also, the organism may be an arthropod such as an insect. The present invention may also be extended to other agricultural applications such as, for example, farm and production animals. For example, pigs have many features that make them attractive as biomedical models, especially in regenerative medicine. In particular, pigs with severe combined immunodeficiency (SCID) may provide useful models for regenerative medicine, xenotransplantation (discussed also elsewhere herein), and tumor development and will aid in developing therapies for human SCID patients. Lee et al., (Proc Natl Acad Sci USA. 2014 May 20; 111(20):7260-5) utilized a reporter-guided transcription activator-like effector nuclease (TALEN) system to generated targeted modifications of recombination activating gene (RAG) 2 in somatic cells at high efficiency, including some that affected both alleles. The Cas protein may be applied to a similar system.

The methods of Lee et al., (Proc Natl Acad Sci USA. 2014 May 20; 111(20):7260-5) may be applied to the present invention analogously as follows. Mutated pigs are produced by targeted insertion for example in RAG2 in fetal fibroblast cells followed by SCNT and embryo transfer. Constructs coding for CRISPR Cas and a reporter are electroporated into fetal-derived fibroblast cells. After 48 h, transfected cells expressing the green fluorescent protein are sorted into individual wells of a 96-well plate at an estimated dilution of a single cell per well. Targeted modification of RAG2 are screened by amplifying a genomic DNA fragment flanking any CRISPR Cas cutting sites followed by sequencing the PCR products. After screening and ensuring lack of off-site mutations, cells carrying targeted modification of RAG2 are used for SCNT. The polar body, along with a portion of the adjacent cytoplasm of oocyte, presumably containing the metaphase II plate, are removed, and a donor cell are placed in the perivitelline. The reconstructed embryos are then electrically porated to fuse the donor cell with the oocyte and then chemically activated. The activated embryos are incubated in Porcine Zygote Medium 3 (PZM3) with 0.5 μM Scriptaid (S7817; Sigma-Aldrich) for 14-16 h. Embryos are then washed to remove the Scriptaid and cultured in PZM3 until they were transferred into the oviducts of surrogate pigs.

The systems and components thereof of present invention is used to create a platform to model a disease or disorder of an animal, in some embodiments a mammal, in some embodiments a human. In certain embodiments, such models and platforms are rodent based, in non-limiting examples rat or mouse. Such models and platforms can take advantage of distinctions among and comparisons between inbred rodent strains. In certain embodiments, such models and platforms primate, horse, cattle, sheep, goat, swine, dog, cat or bird-based, for example to directly model diseases and disorders of such animals or to create modified and/or improved lines of such animals. Advantageously, in certain embodiments, an animal based platform or model is created to mimic a human disease or disorder. For example, the similarities of swine to humans make swine an ideal platform for modeling human diseases. Compared to rodent models, development of swine models has been costly and time intensive. On the other hand, swine and other animals are much more similar to humans genetically, anatomically, physiologically and pathophysiologically. The present invention provides a high efficiency platform for targeted gene and genome editing, gene and genome modification and gene and genome regulation to be used in such animal platforms and models. Though ethical standards block development of human models and in many cases models based on non-human primates, the present invention is used with in vitro systems, including but not limited to cell culture systems, three dimensional models and systems, and organoids to mimic, model, and investigate genetics, anatomy, physiology and pathophysiology of structures, organs, and systems of humans. The platforms and models provide manipulation of single or multiple targets.

In certain embodiments, the present invention is applicable to disease models like that of Schomberg et al. (FASEB Journal, April 2016; 30(1):Suppl 571.1). To model the inherited disease neurofibromatosis type 1 (NF-1) Schomberg used CRISPR-Cas9 to introduce mutations in the swine neurofibromin 1 gene by cytosolic microinjection of CRISPR/Cas9 components into swine embryos. CRISPR guide RNAs (gRNA) were created for regions targeting sites both upstream and downstream of an exon within the gene for targeted cleavage by Cas9 and repair was mediated by a specific single-stranded oligodeoxynucleotide (ssODN) template to introduce a 2500 bp deletion. The systems was also used to engineer swine with specific NF-1 mutations or clusters of mutations, and further can be used to engineer mutations that are specific to, or representative of a given human individual. The invention is similarly used to develop animal models, including but not limited to swine models, of human multigenic diseases. According to the invention, multiple genetic loci in one gene or in multiple genes are simultaneously targeted using multiplexed guides and optionally one or multiple templates.

The present invention is also applicable to modifying SNPs of other animals, such as cows. Tan et al. (Proc Natl Acad Sci USA. 2013 Oct. 8; 110(41): 16526-16531) expanded the livestock gene editing toolbox to include transcription activator-like (TAL) effector nuclease (TALEN)- and clustered regularly interspaced short palindromic repeats (CRISPR)/Cas9-stimulated homology-directed repair (HDR) using plasmid, rAAV, and oligonucleotide templates. Gene specific gRNA sequences were cloned into the Church lab gRNA vector (Addgene ID: 41824) according to their methods (Mali P, et al. (2013) RNA-Guided Human Genome Engineering via Cas9. Science 339(6121):823-826). The Cas9 nuclease was provided either by co-transfection of the hCas9 plasmid (Addgene ID: 41815) or mRNA synthesized from RCIScript-hCas9. This RCIScript-hCas9 was constructed by sub-cloning the XbaI-AgeI fragment from the hCas9 plasmid (encompassing the hCas9 cDNA) into the RCIScript plasmid.

Heo et al. (Stem Cells Dev. 2015 Feb. 1; 24(3):393-402. doi: 10.1089/scd.2014.0278. Epub 2014 Nov. 3) reported highly efficient gene targeting in the bovine genome using bovine pluripotent cells and clustered regularly interspaced short palindromic repeat (CRISPR)/Cas9 nuclease. First, Heo et al. generate induced pluripotent stem cells (iPSCs) from bovine somatic fibroblasts by the ectopic expression of yamanaka factors and GSK3β and MEK inhibitor (2i) treatment. Heo et al. observed that these bovine iPSCs are highly similar to naïve pluripotent stem cells with regard to gene expression and developmental potential in teratomas. Moreover, CRISPR-Cas9 nuclease, which was specific for the bovine NANOG locus, showed highly efficient editing of the bovine genome in bovine iPSCs and embryos.

Igenity® provides a profile analysis of animals, such as cows, to perform and transmit traits of economic traits of economic importance, such as carcass composition, carcass quality, maternal and reproductive traits and average daily gain. The analysis of a comprehensive Igenity® profile begins with the discovery of DNA markers (most often single nucleotide polymorphisms or SNPs). All the markers behind the Igenity® profile were discovered by independent scientists at research institutions, including universities, research organizations, and government entities such as USDA. Markers are then analyzed at Igenity® in validation populations. Igenity® uses multiple resource populations that represent various production environments and biological types, often working with industry partners from the seedstock, cow-calf, feedlot and/or packing segments of the beef industry to collect phenotypes that are not commonly available. Cattle genome databases are widely available, see, e.g., the NAGRP Cattle Genome Coordination Program (www.animalgenome.org/cattle/maps/db.html). Thus, the present invention maybe applied to target bovine SNPs. One of skill in the art may utilize the above protocols for targeting SNPs and apply them to bovine SNPs as described, for example, by Tan et al. or Heo et al, using the systems and components thereof of the present invention.

Qingjian Zou et al. (Journal of Molecular Cell Biology Advance Access published Oct. 12, 2015) demonstrated increased muscle mass in dogs by targeting the first exon of the dog Myostatin (MSTN) gene (a negative regulator of skeletal muscle mass). First, the efficiency of the sgRNA was validated, using cotransfection of the sgRNA targeting MSTN with a Cas9 vector into canine embryonic fibroblasts (CEFs). Thereafter, MSTN KO dogs were generated by micro-injecting embryos with normal morphology with a mixture of Cas9 mRNA and MSTN sgRNA and auto-transplantation of the zygotes into the oviduct of the same female dog. The knock-out puppies displayed an obvious muscular phenotype on thighs compared with its wild-type littermate sister. This can also be performed using the systems of the present invention and components thereof provided herein.

Viral targets in livestock may include, in some embodiments, porcine CD163, for example on porcine macrophages. CD163 is associated with infection (thought to be through viral cell entry) by PRRSv (Porcine Reproductive and Respiratory Syndrome virus, an arterivirus). Infection by PRRSv, especially of porcine alveolar macrophages (found in the lung), results in a previously incurable porcine syndrome (“Mystery swine disease” or “blue ear disease”) that causes suffering, including reproductive failure, weight loss and high mortality rates in domestic pigs. Opportunistic infections, such as enzootic pneumonia, meningitis and ear oedema, are often seen due to immune deficiency through loss of macrophage activity. It also has significant economic and environmental repercussions due to increased antibiotic use and financial loss (an estimated $660 m per year).

As reported by Kristin M Whitworth and Dr Randall Prather et al. (Nature Biotech 3434 published online 7 Dec. 2015) at the University of Missouri and in collaboration with Genus Plc, CD163 was targeted using CRISPR-Cas9 and the offspring of edited pigs were resistant when exposed to PRRSv. One founder male and one founder female, both of whom had mutations in exon 7 of CD163, were bred to produce offspring. The founder male possessed an 11-bp deletion in exon 7 on one allele, which results in a frameshift mutation and missense translation at amino acid 45 in domain 5 and a subsequent premature stop codon at amino acid 64. The other allele had a 2-bp addition in exon 7 and a 377-bp deletion in the preceding intron, which were predicted to result in the expression of the first 49 amino acids of domain 5, followed by a premature stop code at amino acid 85. The sow had a 7 bp addition in one allele that when translated was predicted to express the first 48 amino acids of domain 5, followed by a premature stop codon at amino acid 70. The sow's other allele was unamplifiable. Selected offspring were predicted to be a null animal (CD163−/−), i.e., a CD163 knock out.

Accordingly, in some embodiments, porcine alveolar macrophages may be targeted and/or modified using the engineered nucleic acid modification systems and components thereof of the present invention described herein. In some embodiments, porcine CD163 may be targeted by the engineered nucleic acid modification systems and components thereof of the present invention described herein. In some embodiments, porcine CD163 may be knocked out through insertion of a polynucleotide from, e.g., a donor instruction and/or induction of a DSB or through insertions or deletions, for example targeting deletion or modification of exon 7, including one or more of those described above, or in other regions of the gene, for example deletion or modification of exon 5.

An edited pig and its progeny are also envisaged, for example a CD163 knock out pig. This may be for livestock, breeding or modelling purposes (i.e., a porcine model). Semen comprising the gene knock out or other modification is also provided.

CD163 is a member of the scavenger receptor cysteine-rich (SRCR) superfamily. Based on in vitro studies SRCR domain 5 of the protein is the domain responsible for unpackaging and release of the viral genome. As such, other members of the SRCR superfamily may also be targeted by the systems and components thereof of the present invention in order to assess resistance to other viruses. PRRSV is also a member of the mammalian arterivirus group, which also includes murine lactate dehydrogenase-elevating virus, simian hemorrhagic fever virus and equine arteritis virus. The arteriviruses share important pathogenesis properties, including macrophage tropism and the capacity to cause both severe disease and persistent infection. Accordingly, arteriviruses, and in particular murine lactate dehydrogenase-elevating virus, simian hemorrhagic fever virus and equine arteritis virus, may be targeted, for example through porcine CD163 or homologues thereof in other species, and murine, simian and equine models and knockout also provided.

Indeed, this approach may be extended to viruses or bacteria that cause other livestock diseases that may be transmitted to humans, such as Swine Influenza Virus (SIV) strains which include influenza C and the subtypes of influenza A known as H1N1, H1N2, H2N1, H3N1, H3N2, and H2N3, as well as pneumonia, meningitis and edema mentioned above.

### Therapeutic Uses of the Engineered Systems and Methods of Treatment

Also provided herein are methods of diagnosing, prognosing, treating, and/or preventing a disease, state, or condition in or of a subject. Generally, the methods of diagnosing, prognosing, treating, and/or preventing a disease, state, or condition in or of a subject can include modifying a polynucleotide in a subject or cell thereof using a engineered nucleic acid modification system or component thereof of the present invention described herein and/or include detecting a diseased or healthy polynucleotide in a subject or cell thereof using a engineered nucleic acid modification system or component thereof of the present invention described herein. In some embodiments, the method of treatment or prevention can include using a engineered nucleic acid modification system or component thereof of the present invention described herein to modify a polynucleotide of an infectious organism (e.g., bacterial or virus) within a subject or cell thereof. In some embodiments, the method of treatment or prevention can include using a engineered nucleic acid modification system or component thereof of the present invention described herein to modify a polynucleotide of an infectious organism or symbiotic organism within a subject. The engineered nucleic acid modification system or component thereof of the present invention described herein can be used to develop models of diseases, states, or conditions. The engineered nucleic acid modification system or component thereof of the present invention described herein can be used to detect a disease state or correction thereof, such as by a method of treatment or prevention described herein. The engineered nucleic acid modification system or component thereof of the present invention described herein can be used to screen and select cells that can be used, for example, as treatments or preventions described herein. The engineered nucleic acid modification system or component thereof of the present invention described herein thereof can be used to develop biologically active agents that can be used to modify one or more biologic functions or activities in a subject or a cell thereof.

In general, the method can include delivering an engineered nucleic acid modification system or component thereof of the present invention described herein to a subject or cell(s) thereof, or to an infectious or symbiotic organism by a suitable delivery technique and/or composition. Exemplary delivery and administration techniques are described in greater detail elsewhere herein. Once administered the components can operate as described elsewhere herein to elicit a nucleic acid modification event. In some embodiments, the nucleic acid modification event can occur at the genomic, extragenomic, epigenomic, and/or transcriptomic level. DNA and/or RNA cleavage, gene activation, and/or gene deactivation can occur. Additional features, uses, and advantages are described in greater detail below. On the basis of this concept, several variations are appropriate to elicit a genomic locus event, including DNA cleavage, gene activation, or gene deactivation. Using the provided compositions, the person skilled in the art can advantageously and specifically target single or multiple loci with the same or different functional domains to elicit one or more genomic locus events. In addition to treating and/or preventing a disease in a subject, the compositions may be applied in a wide variety of methods for screening in libraries in cells and functional modeling in vivo (e.g., gene activation of lincRNA and identification of function; gain-of-function modeling; loss-of-function modeling; the use the compositions of the invention to establish cell lines and transgenic animals for optimization and screening purposes).

The engineered nucleic acid modification system or component thereof of the present invention described herein can be used to treat and/or prevent a disease, such as a genetic and/or epigenetic disease, in a subject. The engineered nucleic acid modification system or component thereof of the present invention described herein can be used to treat and/or prevent genetic infectious diseases in a subject, such as bacterial infections, viral infections, fungal infections, parasite infections, and combinations thereof. The engineered nucleic acid modification system or component thereof of the present invention described herein can be used to modify the composition or profile of a microbiome in a subject, which can in turn modify the health status of the subject. The engineered nucleic acid modification system or component thereof of the present invention described herein can be used to modify cells ex vivo, which can then be administered to the subject whereby the modified cells can treat or prevent a disease or symptom thereof. This is also referred to in some contexts as adoptive therapy. The engineered nucleic acid modification system or component thereof of the present invention described herein can be used to treat mitochondrial diseases, where the mitochondrial disease etiology involves a mutation in the mitochondrial DNA.

Also provided is a method of treating a subject, e.g., a subject in need thereof, comprising inducing gene editing by transforming the subject with the polynucleotide encoding one or more components of the engineered nucleic acid modification system or component thereof of the present invention described herein or any of polynucleotides or vectors described herein and administering them to the subject. A suitable repair template may also be provided, for example delivered by a vector comprising said repair template. Also provided is a method of treating a subject, e.g., a subject in need thereof, comprising inducing transcriptional activation or repression of multiple target gene loci by transforming the subject with the polynucleotides or vectors described herein, wherein said polynucleotide or vector encodes or comprises one or more components of engineered nucleic acid modification system or component thereof of the present invention described herein. In some embodiments, the engineered nucleic acid modification system or component thereof of the present invention described herein comprises multiple site specific nucleases (e.g., Cas polypeptides), Vir polypeptides, and/or DNA polymerases. Where any treatment is occurring ex vivo, for example in a cell culture, then it will be appreciated that the term ‘subject’ may be replaced by the phrase “cell or cell culture.”

Also provided is a method of treating a subject, e.g., a subject in need thereof, comprising inducing gene editing by transforming the subject with effector complex of the engineered nucleic acid modification system or component thereof (e.g., the site-specific nuclease, VirD1 and/or VirD2, DNA polymerase) of present invention described herein, advantageously encoding and expressing in vivo the remaining portions of the engineered nuclease modification system (e.g., RNA, guides, donor constructs, etc.). A suitable repair template may also be provided, for example delivered by a vector comprising said repair template. Also provided is a method of treating a subject, e.g., a subject in need thereof, comprising inducing transcriptional activation or repression by transforming the subject with an effector complex of the engineered nucleic acid modification system or component thereof (e.g., the site-specific nuclease, VirD1 and/or VirD2, DNA polymerase) and advantageously encoding and expressing in vivo the remaining portions of the engineered nuclease modification system (e.g., RNA guides, donor constructs, etc.); and advantageously in some embodiments the site-specific nuclease is a catalytically inactive Cas polypeptide and includes one or more associated functional domains. Where any treatment is occurring ex vivo, for example in a cell culture, then it will be appreciated that the term ‘subject’ may be replaced by the phrase “cell or cell culture.”

One or more components of the engineered nucleic modification system described herein can be included in a composition, such as a pharmaceutical composition, and administered to a host individually or collectively. Alternatively, these components may be provided in a single composition for administration to a host. Administration to a host may be performed via viral vectors known to the skilled person or described herein for delivery to a host (e.g., lentiviral vector, adenoviral vector, AAV vector). As explained herein, use of different selection markers (e.g., for lentiviral gRNA selection) and concentration of gRNA (e.g., dependent on whether multiple gRNAs are used) may be advantageous for eliciting an improved effect. Examples of such embodiments are described in greater detail elsewhere herein.

Thus, also described herein are methods of inducing one or more polynucleotide modifications in a eukaryotic or prokaryotic cell or component thereof (e.g., a mitochondria) of a subject, infectious organism, and/or organism of the microbiome of the subject. The modification can include the introduction, deletion, or substitution of one or more nucleotides at a target sequence of a polynucleotide of one or more cell(s). The modification can occur in vitro, ex vivo, in situ, or in vivo. The modification can be introduced using the engineered nucleic acid modification system or component thereof of the present invention described herein.

In some embodiments, the method of treating or inhibiting a condition or a disease caused by one or more mutations in a genomic locus in a eukaryotic organism or a non-human organism can include manipulation of a target sequence within a coding, non-coding or regulatory element of said genomic locus in a target sequence in a subject or a non-human subject in need thereof comprising modifying the subject or a non-human subject by manipulation of the target sequence and wherein the condition or disease is susceptible to treatment or inhibition by manipulation of the target sequence including providing treatment comprising delivering a composition comprising the particle delivery system or the delivery system or the virus particle of any one of the above embodiments or the cell of any one of the above embodiments. Such modification and/or manipulation of the target sequence can be mediated by an engineered nucleic acid modification system or component thereof of the present invention described herein.

Also provided herein is the use of the particle delivery system or the delivery system or the virus particle of any one of the above embodiment or the cell of any one of the above embodiment in ex vivo or in vivo gene or genome editing; or for use in in vitro, ex vivo or in vivo gene therapy. Also provided herein are particle delivery systems, non-viral delivery systems, and/or the virus particle of any one of the above embodiments or the cell of any one of the above embodiments used in the manufacture of a medicament for in vitro, ex vivo or in vivo gene or genome editing or for use in in vitro, ex vivo or in vivo gene therapy or for use in a method of modifying an organism or a non-human organism by manipulation of a target sequence in a genomic locus associated with a disease or in a method of treating or inhibiting a condition or disease caused by one or more mutations in a genomic locus in a eukaryotic organism or a non-human organism. Such delivery systems are described in greater detail elsewhere herein.

In some embodiments, polynucleotide modification can include the introduction, deletion, or substitution of 1-75 nucleotides at each target sequence of said polynucleotide of said cell(s). The modification can include the introduction, deletion, or substitution of 1, 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence. The modification can include the introduction, deletion, or substitution of 5, 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of 10, 11, 12, 13, 14, 15, 16, 17, 18, 19, 20, 21, 22, 23, 24, 25, 26, 27, 28, 29, 30, 35, 40, 45, 50, or 75 nucleotides at each target sequence of said cell(s). The modification can include the introduction, deletion, or substitution of 20, 21, 22, 23, 24, 25, 26, 27, 28, 29, 30, 35, 40, 45, 50, or 75 nucleotides at each target sequence of said cell(s). The modification can include the introduction, deletion, or substitution of 40, 45, 50, 75, 100, 200, 300, 400 or 500 nucleotides at each target sequence of said cell(s). The modification can include the introduction, deletion, or substitution of 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900 to/or 10000 nucleotides or more at each target sequence of said cell(s).

In some embodiments, the modifications can include the introduction, deletion, or substitution of nucleotides at each target sequence of said cell(s) via nucleic acid components (e.g., guide(s) RNA(s) or sgRNA(s)), such as those mediated by an engineered nucleic acid modification system or a component thereof of the present invention described elsewhere herein. In some embodiments, the modifications can include the introduction, deletion, or substitution of nucleotides at a target or random sequence of said cell(s) via a non an engineered nucleic acid modification system or technique.

The target sequences of polynucleotides to be modified to treat or prevent disease are described in greater detail below.

As is also discussed elsewhere herein, the engineered nucleic acid modification system can include a donor construct as previously described elsewhere herein.

As is also discussed elsewhere herein, the engineered nucleic acid modification system can include, in some embodiments, a template polynucleotide (also referred to herein as template nucleic acids or template sequence). In an embodiment, the template nucleic acid alters the structure of the target position by participating in homologous recombination. In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid can include sequence that corresponds to a site on the target sequence that is cleaved, nicked, or otherwise modified by one or more site-specific nuclease (e.g., a Cas polypeptide) mediated cleavage event(s). In an embodiment, the template nucleic acid can include sequence that corresponds to both, a first site on the target sequence that is cleaved, nicked, or otherwise modified in a first Cas effector mediated event, and a second site on the target sequence that is cleaved in a second Cas effector mediated event.

In certain embodiments, the donor construct and/or template nucleic acid can include a sequence (e.g., a donor polynucleotide of a donor construct) which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A donor polynucleotide and/or donor construct and/or template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The donor construct, template nucleic acid may include sequence (e.g., a donor polynucleotide in the context of a donor construct) which results in: a change in sequence of 1, 5, 10, 11, 12, 13, 14, 15, 16, 17, 18, 19, 20, 21, 22, 23, 24, 25, 26, 27, 28, 29, 30, 35, 40, 45, 50, or 75 or more nucleotides of the target sequence. In an embodiment, the template nucleic acid and/or donor polynucleotide of a donor construct may be 20+/−10, 30+/−10, 40+/−10, 50+/−10, 60+/−10, 70+/−10, 80+/−10, 9 0+/−10, 100+/−10, 110+/−10, 120+/−10, 130+/−10, 140+/−10, 150+/−10, 160+/−10, 170+/−10, 1 80+/−10, 190+/−10, 200+/−10, 210+/−10, of 220+/−10 nucleotides in length. In an embodiment, the template nucleic acid and/or donor polynucleotide of a donor construct may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid and/or donor polynucleotide of a donor construct is 10 to 1,000, 20 to 900, 30 to 800, 40 to 700, 50 to 600, 50 to 500, 50 to 400, 50 to 300, 50 to 200, or 50 to 100 nucleotides in length. In an embodiment, the template nucleic acid and/or donor polynucleotide of a donor construct is about 10, 20, 30, 40, 45, 50, 75, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900 to/or 10000 nucleotides or more.

A template nucleic acid and/or donor polynucleotide the following components: [5′ homology arm]-[replacement sequence]-[3′ homology arm]. The homology arms provide for recombination into the chromosome, thus replacing the undesired element, e.g., a mutation or signature, with the replacement sequence. In an embodiment, the homology arms flank the most distal cleavage sites. In an embodiment, the 3′ end of the 5′ homology arm is the position next to the 5′ end of the replacement sequence. In an embodiment, the 5′ homology arm can extend at least 10, 20, 30, 40, 50, 100, 200, 300, 400, 500, 600, 700, 800, 900, 1000, 1500, or 2000 nucleotides 5′ from the 5′ end of the replacement sequence. In an embodiment, the 5′ end of the 3′ homology arm is the position next to the 3′ end of the replacement sequence. In an embodiment, the 3′ homology arm can extend at least 10, 20, 30, 40, 50, 100, 200, 300, 400, 500, 600, 700, 800, 900, 1000, 1500, or 2000 nucleotides 3′ from the 3′ end of the replacement sequence.

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In certain embodiments, a donor polynucleotide and/or template nucleic acids for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

In some embodiments, the engineered nucleic acid modification system of the present invention or component thereof can promote Non-Homologous End-Joining (NHEJ). In some embodiments, modification of a polynucleotide by an engineered nucleic acid modification system of the present invention or component thereof of the present invention, such as a diseased polynucleotide, can include NHEJ. In some embodiments, promotion of this repair pathway by the engineered nucleic acid modification system of the present invention or component thereof of the present invention can be used to target gene or polynucleotide specific knock-outs and/or knock-ins. In some embodiments, promotion of this repair pathway by the engineered nucleic acid modification system of the present invention or component thereof of the present invention can be used to generate NHEJ-mediated indels. Nuclease-induced NHEJ can also be used to remove (e.g., delete) sequence in a gene of interest. Generally, NHEJ repairs a double-strand break in the DNA by joining together the two ends; however, generally, the original sequence is restored only if two compatible ends, exactly as they were formed by the double-strand break, are perfectly ligated. The DNA ends of the double-strand break are frequently the subject of enzymatic processing, resulting in the addition or removal of nucleotides, at one or both strands, prior to rejoining of the ends. This results in the presence of insertion and/or deletion (indel) mutations in the DNA sequence at the site of the NHEJ repair. The indel can range in size from 1-50 or more base pairs. In some embodiments thee indel can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5100, 5200, 5300, 5400, 5500, 5600, 5700, 5800, 5900, 6000, 6100, 6200, 6300, 6400, 6500, 6600, 6700, 6800, 6900, 7000, 7100, 7200, 7300, 7400, 7500, 7600, 7700, 7800, 7900, 8000, 8100, 8200, 8300, 8400, 8500, 8600, 8700, 8800, 8900, 9000, 9100, 9200, 9300, 9400, 9500, 9600, 9700, 9800, 9900 to/or 10000 base pairs or more. If a double-strand break is targeted near to a short target sequence, the deletion mutations caused by the NHEJ repair often span, and therefore remove, the unwanted nucleotides. For the deletion of larger DNA segments, introducing two double-strand breaks, one on each side of the sequence, can result in NHEJ between the ends with removal of the entire intervening sequence. Both of these approaches can be used to delete specific DNA sequences.

In some embodiments, engineered nucleic acid modification system mediated NHEJ can be used in the method to delete small sequence motifs. In some embodiments, engineered nucleic acid modification system mediated NHEJ can be used in the method to generate NHEJ-mediate indels that can be targeted to the gene, e.g., a coding region, e.g., an early coding region of a gene of interest can be used to knockout (i.e., eliminate expression of) a gene of interest. For example, early coding region of a gene of interest includes sequence immediately following a transcription start site, within a first exon of the coding sequence, or within 500 bp of the transcription start site (e.g., less than 500, 450, 400, 350, 300, 250, 200, 150, 100 or 50 bp). In an embodiment, in which a guide RNA and Cas effector generate a double strand break for the purpose of inducing NHEJ-mediated indels, a guide RNA may be configured to position one double-strand break in close proximity to a nucleotide of the target position. In an embodiment, the cleavage site may be between 0-500 bp away from the target position (e.g., less than 500, 400, 300, 200, 100, 50, 40, 30, 25, 20, 15, 10, 9, 8, 7, 6, 5, 4, 3, 2 or 1 bp from the target position). In an embodiment, in which two guide RNAs complexing with one or more Cas nickases induce two single strand breaks for the purpose of inducing NHEJ-mediated indels, two guide RNAs may be configured to position two single-strand breaks to provide for NHEJ repair a nucleotide of the target position.

For minimization of toxicity and off-target effect, it may be important to control the concentration of Cas mRNA and guide RNA delivered, such as in embodiments where the site-specific nuclease is a Cas polypeptide. Optimal concentrations of Cas mRNA and guide RNA can be determined by testing different concentrations in a cellular or non-human eukaryote animal model and using deep sequencing the analyze the extent of modification at potential off-target genomic loci. Alternatively, to minimize the level of toxicity and off-target effect, Cas nickase mRNA (for example S. pyogenes Cas9 with the D10A mutation) can be delivered with a pair of guide RNAs targeting a site of interest. Guide sequences and strategies to minimize toxicity and off-target effects can be as in WO 2014/093622 (PCT/US2013/074667); or, via mutation. Others are as described elsewhere herein.

In some embodiments, a method of modifying a target polynucleotide in a cell to treat or prevent a disease can include allowing an engineered nucleic acid modification system or component thereof to bind to the target polynucleotide, e.g., to effect cleavage, nicking, or other modification as the engineered nucleic acid modification system of the present invention is capable of said target polynucleotide, thereby modifying the target polynucleotide, wherein engineered nucleic acid modification system of the present invention or component thereof, complexes with a guide sequence, and hybridize said guide sequence to a target sequence within the target polynucleotide, wherein said guide sequence is optionally linked to a tracr mate sequence, which in turn can hybridize to a tracr sequence. In some of these embodiments, the engineered nucleic acid modification system of the present invention or component thereof can include a Cas polypeptide complexed with a guide sequence. In some embodiments, modification can include cleaving or nicking one or two strands at the location of the target sequence by one or more components of the engineered nucleic acid modification system or component thereof, such as one or more Cas polypeptides of the engineered system.

The cleavage, nicking, or other modification capable of being performed by the by one or more components of the engineered nucleic acid modification system or component thereof, such as one or more Cas polypeptides of the engineered system modify transcription of a target polynucleotide and/or facilitate introduction of a donor polynucleotide into a target polynucleotide. In some embodiments, modification of transcription can include decreasing transcription of a target polynucleotide. In some embodiments, modification can include increasing transcription of a target polynucleotide. In some embodiments, the method includes repairing said cleaved target polynucleotide by homologous recombination with an exogenous template polynucleotide, wherein said repair results in a modification such as, but not limited to, an insertion, deletion, or substitution of one or more nucleotides of said target polynucleotide. In some embodiments, said modification results in one or more amino acid changes in a protein expressed from a gene comprising the target sequence. In some embodiments, the modification imparted by the engineered nucleic acid modification system or component thereof provides a transcript and/or protein that can correct a disease or a symptom thereof, including but not limited to, any of those described in greater detail elsewhere herein.

In some embodiments, the method of treating or preventing a disease can include delivering one or more vectors or vector systems to a cell, such as a eukaryotic or prokaryotic cell, wherein one or more vectors or vector systems include the C engineered nucleic acid modification system or component thereof. In some embodiments, the vector(s) or vector system(s) can be a viral vector or vector system, such as an AAV or lentiviral vector system, which are described in greater detail elsewhere herein. In some embodiments, the method of treating or preventing a disease can include delivering one or more viral particles, such as an AAV or lentiviral particle, containing the engineered nucleic acid modification system or component thereof. In some embodiments, the viral particle has a tissue specific tropism. In some embodiments, the viral particle has a liver, muscle, eye, heart, pancreas, kidney, neuron, epithelial cell, endothelial cell, astrocyte, glial cell, immune cell, or red blood cell specific tropism.

It will be understood that the engineered nucleic acid modification systems or component(s) thereof according to the invention as described herein, may be suitably used for any type of application known for CRISPR-Cas systems and other nucleic acid modification systems generally known in the art, preferably in eukaryotes. In certain aspects, the application is therapeutic, preferably therapeutic in a eukaryote organism, such as including but not limited to animals (including human), plants, algae, fungi (including yeasts), etc. Alternatively, or in addition, in certain aspects, the application may involve accomplishing or inducing one or more particular traits or characteristics, such as genotypic and/or phenotypic traits or characteristics, as also described elsewhere herein.

### Treating Diseases of the Circulatory System

In some embodiments, the engineered nucleic acid modification system or component(s) thereof of the present invention can be used to treat and/or prevent a circulatory system disease. Exemplary diseases are provided, for example, in Tables 5 and 6, as well as a disease identified as being caused or attributed to a mtDNA mutation set forth at mitomap.org. In some embodiments the plasma exosomes of Wahlgren et al. (Nucleic Acids Research, 2012, Vol. 40, No. 17 e130) can be used to deliver the engineered nucleic acid modification system or component(s) thereof of the present invention to the blood, blood component, or cell therein. In some embodiments, the circulatory system disease can be treated by using a lentivirus to deliver the engineered nucleic acid modification system or component(s) thereof of the present invention to modify hematopoietic stem cells (HSCs) in vivo or ex vivo (see e.g., Drakopoulou, “Review Article, The Ongoing Challenge of Hematopoietic Stem Cell-Based Gene Therapy for β-Thalassemia,” Stem Cells International, Volume 2011, Article ID 987980, 10 pages, doi:10.4061/2011/987980, which can be adapted for use with the engineered nucleic acid modification system or component(s) thereof of the present invention herein in view of the description herein). In some embodiments, the circulatory system disorder can be treated by correcting HSCs as to the disease using a engineered nucleic acid modification system or component(s) thereof of the present invention, wherein the engineered nucleic acid modification system or component(s) thereof of the present invention optionally includes a suitable HDR repair template (see e.g. Cavazzana, “Outcomes of Gene Therapy for 0-Thalassemia Major via Transplantation of Autologous Hematopoietic Stem Cells Transduced Ex Vivo with a Lentiviral PA-T87Q-Globin Vector.”; Cavazzana-Calvo, “Transfusion independence and HMGA2 activation after gene therapy of human β-thalassaemia”, Nature 467, 318-322 (16 Sep. 2010) doi:10.1038/nature09328; Nienhuis, “Development of Gene Therapy for Thalassemia, Cold Spring Harbor Perspectives in Medicine, doi: 10.1101/cshperspect.a011833 (2012), LentiGlobin BB305, a lentiviral vector containing an engineered β-globin gene (PA-T87Q); and Xie et al., “Seamless gene correction of β-thalassaemia mutations in patient-specific iPSCs using CRISPR/Cas9 and piggyback” Genome Research gr.173427.114 (2014) http://www.genome.org/cgi/doi/10.1101/gr.173427.114 (Cold Spring Harbor Laboratory Press; [1599] Watts, “Hematopoietic Stem Cell Expansion and Gene Therapy” Cytotherapy 13(10):1164-1171. doi:10.3109/14653249.2011.620748 (2011), which can be adapted for use with the engineered nucleic acid modification system or component(s) thereof of the present invention in view of the description herein). In some embodiments, iPSCs can be modified using an engineered nucleic acid modification system or component(s) thereof of the present invention to correct a disease polynucleotide associated with a circulatory disease. In this regard, the teachings of Xu et al. (Sci Rep. 2015 Jul. 9; 5:12065. doi: 10.1038/srep12065) and Song et al. (Stem Cells Dev. 2015 May 1; 24(9):1053-65. doi: 10.1089/scd.2014.0347. Epub 2015 Feb. 5) with respect to modifying iPSCs can be adapted for use in view of the description herein with the engineered nucleic acid modification system or component(s) thereof of the present invention.

The term “Hematopoietic Stem Cell” or “HSC” refers broadly those cells considered to be an HSC, e.g., blood cells that give rise to all the other blood cells and are derived from mesoderm; located in the red bone marrow, which is contained in the core of most bones. HSCs of the invention include cells having a phenotype of hematopoietic stem cells, identified by small size, lack of lineage (lin) markers, and markers that belong to the cluster of differentiation series, like: CD34, CD38, CD90, CD133, CD105, CD45, and also c-kit,—the receptor for stem cell factor. Hematopoietic stem cells are negative for the markers that are used for detection of lineage commitment, and are, thus, called Lin−; and, during their purification by FACS, a number of up to 14 different mature blood-lineage markers, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter 19 for erythroid cells, Il7Ra, CD3, CD4, CD5, CD8 for T cells, etc. Mouse HSC markers: CD34lo/−, SCA-1+, Thy1.1+/lo, CD38+, C-kit+, lin−, and Human HSC markers: CD34+, CD59+, Thy1/CD90+, CD38lo/−, C-kit/CD117+, and lin−. HSCs are identified by markers. Hence in embodiments discussed herein, the HSCs can be CD34+ cells. HSCs can also be hematopoietic stem cells that are CD34−/CD38−. Stem cells that may lack c-kit on the cell surface that are considered in the art as HSCs are within the ambit of the invention, as well as CD133+ cells likewise considered HSCs in the art.

In some embodiments, the treatment or prevention for treating a circulatory system or blood disease can include modifying a human cord blood cell with any modification described herein. In some embodiments, the treatment or prevention for treating a circulatory system or blood disease can include modifying a granulocyte colony-stimulating factor-mobilized peripheral blood cell (mPB) with any modification described herein. In some embodiments, the human cord blood cell or mPB can be CD34+. In some embodiments, the cord blood cell(s) or mPB cell(s) modified can be autologous. In some embodiments, the cord blood cell(s) or mPB cell(s) can be allogenic. In addition to the modification of the disease gene(s), allogenic cells can be further modified using the composition, system, described herein to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the composition, system, herein. The modified cord blood cell(s) or mPB cell(s) can be optionally expanded in vitro. The modified cord blood cell(s) or mPB cell(s) can be derived to a subject in need thereof using any suitable delivery technique.

The engineered nucleic acid modification system or component(s) thereof of the present invention (system may be engineered to target genetic locus or loci in HSCs. In some embodiments, one or more components of the engineered nucleic acid modification system (e.g., a site-specific nuclease, DNA polymerase, and/or one or more Vir polypeptides (e.g., VirD1 and/or VirD2) can be codon-optimized for a eukaryotic cell and especially a mammalian cell, e.g., a human cell, for instance, HSC, or iPSC and sgRNA targeting a locus or loci in HSC, such as circulatory disease, can be prepared. These may be delivered via particles. The particles may be formed by mixing one or more polypeptides and/or effectors of the engineered nucleic acid modification system of the present invention and one or more guide molecules and/or donor constructs being admixed, such as with a mixture comprising or consisting essentially of or consisting of surfactant, phospholipid, biodegradable polymer, lipoprotein and alcohol, whereby particles containing the gRNA and effector polypeptide(s) may be formed. The invention comprehends so making particles and particles from such a method as well as uses thereof. Particles suitable delivery of the engineered nucleotide modification system of the present invention in the context of blood or circulatory system or HSC delivery to the blood or circulatory system are described in greater detail elsewhere herein.

In some embodiments, after ex vivo modification the HSCs or iPCS can be expanded prior to administration to the subject. Expansion of HSCs can be via any suitable method such as that described by, Lee, “Improved ex vivo expansion of adult hematopoietic stem cells by overcoming CUL4-mediated degradation of HOXB4.” Blood. 2013 May 16; 121(20):4082-9. doi: 10.1182/blood-2012-09-455204. Epub 2013 Mar. 21.

In some embodiments, the HSCs or iPSCs modified can be autologous. In some embodiments, the HSCs or iPSCs can be allogenic. In addition to the modification of the disease gene(s), allogenic cells can be further modified using the engineered nucleic acid modification system of the present invention to reduce the immunogenicity of the cells when delivered to the recipient. Such techniques are described elsewhere herein and e.g., Cartier, “MINI-SYMPOSIUM: X-Linked Adrenoleukodystrophypa, Hematopoietic Stem Cell Transplantation and Hematopoietic Stem Cell Gene Therapy in X-Linked Adrenoleukodystrophy,” Brain Pathology 20 (2010) 857-862, which can be adapted for use with the engineered nucleic acid modification system of the present invention.

### Treating Diseases of the Brain

In some embodiments, the engineered nucleic acid modification system of the present invention can be used to treat diseases of the brain and CNS. Delivery options for the brain include encapsulation of engineered nucleic acid modification system of the present invention and/or components thereof in the form of either DNA or RNA into liposomes and conjugating to molecular Trojan horses for trans-blood brain barrier (BBB) delivery. Molecular Trojan horses have been shown to be effective for delivery of B-gal expression vectors into the brain of non-human primates. The same approach can be used to delivery vectors containing and/or encoding the engineered nucleic acid modification system of the present invention. For instance, Xia C F and Boado R J, Pardridge W M (“Antibody-mediated targeting of siRNA via the human insulin receptor using avidin-biotin technology.” Mol Pharm. 2009 May-June; 6(3):747-51. doi: 10.1021/mp800194) describes how delivery of short interfering RNA (siRNA) to cells in culture, and in vivo, is possible with combined use of a receptor-specific monoclonal antibody (mAb) and avidin-biotin technology. The authors also report that because the bond between the targeting mAb and the siRNA is stable with avidin-biotin technology, and RNAi effects at distant sites such as brain are observed in vivo following an intravenous administration of the targeted siRNA, the teachings of which can be adapted for use with the engineered nucleic acid modification system of the present invention. In other embodiments, an artificial virus can be generated for CNS and/or brain delivery. See e.g., Zhang et al. (Mol Ther. 2003 January; 7(1):11-8.)), the teachings of which can be adapted for use with the CRISPR-Cas systems herein.

### Treating Hearing Diseases

In some embodiments the engineered nucleic acid modification system of the present invention can be used to treat a hearing disease or hearing loss in one or both ears. Deafness is often caused by lost or damaged hair cells that cannot relay signals to auditory neurons. In such cases, cochlear implants may be used to respond to sound and transmit electrical signals to the nerve cells. But these neurons often degenerate and retract from the cochlea as fewer growth factors are released by impaired hair cells.

In some embodiments, the engineered nucleic acid modification system of the present invention or modified cells can be delivered to one or both ears for treating or preventing hearing disease or loss by any suitable method or technique. Suitable methods and techniques include, but are not limited to, those set forth in e.g., U.S. Publication No. 2012/0328580, which describes injection of a pharmaceutical composition into the ear (e.g., auricular administration), such as into the luminae of the cochlea (e.g., the Scala media, Sc vestibulae, and Sc tympani), e.g., using a syringe, e.g., a single-dose syringe. For example, one or more of the engineered nucleic acid modification system of the present invention or components thereof described herein can be administered by intratympanic injection (e.g., into the middle ear), and/or injections into the outer, middle, and/or inner ear; administration in situ, via a catheter or pump (see e.g., McKenna et al., (U.S. Publication No. 2006/0030837) and Jacobsen et al., (U.S. Pat. No. 7,206,639); administration in combination with a mechanical device such as a cochlear implant or a hearing aid, which is worn in the outer ear (see e.g. U.S. Publication No. 2007/0093878, which provides an exemplary cochlear implant suitable for delivery of the engineered nucleic acid modification system of the present invention to the ear). Such methods are routinely used in the art, for example, for the administration of steroids and antibiotics into human ears. Injection can be, for example, through the round window of the ear or through the cochlear capsule. Other inner ear administration methods are known in the art (see, e.g., Salt and Plontke, Drug Discovery Today, 10:1299-1306, 2005). In some embodiments, a catheter or pump can be positioned, e.g., in the ear (e.g., the outer, middle, and/or inner ear) of a patient during a surgical procedure. In some embodiments, a catheter or pump can be positioned, e.g., in the ear (e.g., the outer, middle, and/or inner ear) of a patient without the need for a surgical procedure.

In general, the cell therapy methods described in U.S. patent application 20120328580 can be used to promote complete or partial differentiation of a cell to or towards a mature cell type of the inner ear (e.g., a hair cell) in vitro. Cells resulting from such methods can then be transplanted or implanted into a patient in need of such treatment. The cell culture methods required to practice these methods, including methods for identifying and selecting suitable cell types, methods for promoting complete or partial differentiation of selected cells, methods for identifying complete or partially differentiated cell types, and methods for implanting complete or partially differentiated cells are described below.

Cells suitable for use in the present invention include, but are not limited to, cells that are capable of differentiating completely or partially into a mature cell of the inner ear, e.g., a hair cell (e.g., an inner and/or outer hair cell), when contacted, e.g., in vitro, with one or more of the compounds described herein. Exemplary cells that are capable of differentiating into a hair cell include, but are not limited to stem cells (e.g., inner ear stem cells, adult stem cells, bone marrow derived stem cells, embryonic stem cells, mesenchymal stem cells, skin stem cells, iPS cells, and fat derived stem cells), progenitor cells (e.g., inner ear progenitor cells), support cells (e.g., Deiters' cells, pillar cells, inner phalangeal cells, tectal cells and Hensen's cells), and/or germ cells. The use of stem cells for the replacement of inner ear sensory cells is described in Li et al., (U.S. Publication No. 2005/0287127) and Li et al., (U.S. patent Ser. No. 11/953,797). The use of bone marrow derived stem cells for the replacement of inner ear sensory cells is described in Edge et al., PCT/US2007/084654. iPS cells are described, e.g., at Takahashi et al., Cell, Volume 131, Issue 5, Pages 861-872 (2007); Takahashi and Yamanaka, Cell 126, 663-76 (2006); Okita et al., Nature 448, 260-262 (2007); Yu, J. et al., Science 318(5858):1917-1920 (2007); Nakagawa et al., Nat. Biotechnol. 26:101-106 (2008); and Zaehres and Scholer, Cell 131(5):834-835 (2007). Such suitable cells can be identified by analyzing (e.g., qualitatively or quantitatively) the presence of one or more tissue specific genes. For example, gene expression can be detected by detecting the protein product of one or more tissue-specific genes. Protein detection techniques involve staining proteins (e.g., using cell extracts or whole cells) using antibodies against the appropriate antigen. In this case, the appropriate antigen is the protein product of the tissue-specific gene expression. Although, in principle, a first antibody (i.e., the antibody that binds the antigen) can be labeled, it is more common (and improves the visualization) to use a second antibody directed against the first (e.g., an anti-IgG). This second antibody is conjugated either with fluorochromes, or appropriate enzymes for colorimetric reactions, or gold beads (for electron microscopy), or with the biotin-avidin system, so that the location of the primary antibody, and thus the antigen, can be recognized.

The engineered nucleic acid modification system of the present invention or component(s)g thereof may be delivered to the ear by direct application of pharmaceutical composition to the outer ear, with compositions modified from U.S. Publication No. 2011/0142917. In some embodiments the pharmaceutical composition is applied to the ear canal. Delivery to the ear may also be referred to as aural or otic delivery.

In some embodiments, the engineered nucleic acid modification system of the present invention and/or vector(s) or vector systems encoding said systems can be delivered to ear via a transfection to the inner ear through the intact round window by a proteidic delivery technology which may be applied to the nucleic acid-targeting system of the present invention (see, e.g., Qi et al., Gene Therapy (2013), 1-9). About 40 μl of 10 mM RNA may be contemplated as the dosage for administration to the ear.

According to Rejali et al. (Hear Res. 2007 June; 228(1-2):180-7), cochlear implant function can be improved by good preservation of the spiral ganglion neurons, which are the target of electrical stimulation by the implant and brain derived neurotrophic factor (BDNF) has previously been shown to enhance spiral ganglion survival in experimentally deafened ears. Rejali et al. tested a modified design of the cochlear implant electrode that includes a coating of fibroblast cells transduced by a viral vector with a BDNF gene insert. To accomplish this type of ex vivo gene transfer, Rejali et al. transduced guinea pig fibroblasts with an adenovirus with a BDNF gene cassette insert and determined that these cells secreted BDNF and then attached BDNF-secreting cells to the cochlear implant electrode via an agarose gel, and implanted the electrode in the scala tympani. Rejali et al. determined that the BDNF expressing electrodes were able to preserve significantly more spiral ganglion neurons in the basal turns of the cochlea after 48 days of implantation when compared to control electrodes and demonstrated the feasibility of combining cochlear implant therapy with ex vivo gene transfer for enhancing spiral ganglion neuron survival. Such a system may be applied to the nucleic acid-targeting system of the present invention for delivery to the ear.

In some embodiments, the system set forth in Mukherjea et al. (Antioxidants & Redox Signaling, Volume 13, Number 5, 2010) can be adapted for transtympanic administration of the engineered nucleic acid modification system of the present invention or component(s) thereof to the ear. In some embodiments, a dosage of about 2 mg to about 4 mg of the engineered nucleic acid modification system of the present invention can be used for administration to a human.

In some embodiments, the system and dosages set forth in (Jung et al. (Molecular Therapy, vol. 21 no. 4, 834-841. 2013) can be adapted for vestibular epithelial delivery of the engineered nucleic acid modification system of the present invention or component(s) thereof to the ear. In some embodiments, a dosage of about 1 to about 30 mg of the engineered nucleic acid modification system of the present invention or component(s) thereof is used for administration to a human.

### Treating Diseases in Non-Dividing Cells

In some embodiments, the gene or transcript to be corrected is in a non-dividing cell. Exemplary non-dividing cells are muscle cells or neurons. Non-dividing (especially non-dividing, fully differentiated) cell types present issues for gene targeting or genome engineering, for example because homologous recombination (HR) is generally suppressed in the G1 cell-cycle phase. However, while studying the mechanisms by which cells control normal DNA repair systems, Durocher discovered a previously unknown switch that keeps HR “off” in non-dividing cells and devised a strategy to toggle this switch back on. Orthwein et al. (Daniel Durocher's lab at the Mount Sinai Hospital in Ottawa, Canada) recently reported (Nature 16142, published online 9 Dec. 2015) have shown that the suppression of HR can be lifted and gene targeting successfully concluded in both kidney (293T) and osteosarcoma (U20S) cells. Tumor suppressors, BRCA1, PALB2 and BRAC2 are known to promote DNA DSB repair by HR. They found that formation of a complex of BRCA1 with PALB2-BRAC2 is governed by a ubiquitin site on PALB2, such that action on the site by an E3 ubiquitin ligase. This E3 ubiquitin ligase is composed of KEAP1 (a PALB2-interacting protein) in complex with cullin-3 (CUL3)-RBX1. PALB2 ubiquitylation suppresses its interaction with BRCA1 and is counteracted by the deubiquitylase USP11, which is itself under cell cycle control. Restoration of the BRCA1-PALB2 interaction combined with the activation of DNA-end resection is sufficient to induce homologous recombination in G1, as measured by a number of methods including a CRISPR-Cas9-based gene-targeting assay directed at USP11 or KEAP1 (expressed from a pX459 vector). However, when the BRCA1-PALB2 interaction was restored in resection-competent G1 cells using either KEAP1 depletion or expression of the PALB2-KR mutant, a robust increase in gene-targeting events was detected. These teachings can be adapted for and/or applied to the engineered nucleic acid modification system of the present invention and/or component(s) thereof.

Thus, reactivation of HR in cells, especially non-dividing, fully differentiated cell types is preferred, in some embodiments. In some embodiments, promotion of the BRCA1-PALB2 interaction is preferred in some embodiments. In some embodiments, the target ell is a non-dividing cell. In some embodiments, the target cell is a neuron or muscle cell. In some embodiments, the target cell is targeted in vivo. In some embodiments, the cell is in G1 and HR is suppressed. In some embodiments, use of KEAP1 depletion, for example inhibition of expression of KEAP1 activity, is preferred. KEAP1 depletion may be achieved through siRNA, for example as shown in Orthwein et al. Alternatively, expression of the PALB2-KR mutant (lacking all eight Lys residues in the BRCA1-interaction domain is preferred, either in combination with KEAP1 depletion or alone. PALB2-KR interacts with BRCA1 irrespective of cell cycle position. Thus, promotion or restoration of the BRCA1-PALB2 interaction, especially in G1 cells, is preferred in some embodiments, especially where the target cells are non-dividing, or where removal and return (ex vivo gene targeting) is problematic, for example neuron or muscle cells. KEAP1 siRNA is available from ThermoFischer. In some embodiments, a BRCA1-PALB2 complex may be delivered to the G1 cell. In some embodiments, PALB2 deubiquitylation may be promoted for example by increased expression of the deubiquitylase USP11, so it is envisaged that a construct may be provided to promote or up-regulate expression or activity of the deubiquitylase USP11.

### Treating Diseases of the Eye

In some embodiments, the disease to be treated is a disease that affects the eyes. Thus, in some embodiments, the engineered nucleic acid modification system of the present invention or component(s) thereof is delivered to one or both eyes.

The engineered nucleic acid modification system of the present invention or component(s) thereof can be used to correct ocular defects that arise from several genetic mutations further described in Genetic Diseases of the Eye, Second Edition, edited by Elias I. Traboulsi, Oxford University Press, 2012.

In some embodiments, the condition to be treated or targeted is an eye disorder. In some embodiments, the eye disorder may include glaucoma. In some embodiments, the eye disorder includes a retinal degenerative disease. In some embodiments, the retinal degenerative disease is selected from Stargardt disease, Bardet-Biedl Syndrome, Best disease, Blue Cone Monochromacy, Choroidermia, Cone-rod dystrophy, Congenital Stationary Night Blindness, Enhanced S-Cone Syndrome, Juvenile X-Linked Retinoschisis, Leber Congenital Amaurosis, Malattia Leventinesse, Norrie Disease or X-linked Familial Exudative Vitreoretinopathy, Pattern Dystrophy, Sorsby Dystrophy, Usher Syndrome, Retinitis Pigmentosa, Achromatopsia or Macular dystrophies or degeneration, Retinitis Pigmentosa, Achromatopsia, and age related macular degeneration. In some embodiments, the retinal degenerative disease is Leber Congenital Amaurosis (LCA) or Retinitis Pigmentosa. Other exemplary eye diseases are described in greater detail elsewhere herein.

In some embodiments, the engineered nucleic acid modification system of the present invention or component(s) thereof is delivered to the eye, optionally via intravitreal injection or subretinal injection. Intraocular injections may be performed with the aid of an operating microscope. For subretinal and intravitreal injections, eyes may be prolapsed by gentle digital pressure and fundi visualized using a contact lens system consisting of a drop of a coupling medium solution on the cornea covered with a glass microscope slide coverslip. For subretinal injections, the tip of a 10-mm 34-gauge needle, mounted on a 5-μl Hamilton syringe may be advanced under direct visualization through the superior equatorial sclera tangentially towards the posterior pole until the aperture of the needle was visible in the subretinal space. Then, 2 μl of vector suspension may be injected to produce a superior bullous retinal detachment, thus confirming subretinal vector administration. This approach creates a self-sealing sclerotomy allowing the vector suspension to be retained in the subretinal space until it is absorbed by the RPE, usually within 48 h of the procedure. This procedure may be repeated in the inferior hemisphere to produce an inferior retinal detachment. This technique results in the exposure of approximately 70% of neurosensory retina and RPE to the vector suspension. For intravitreal injections, the needle tip may be advanced through the sclera 1 mm posterior to the corneoscleral limbus and 2 μl of vector suspension injected into the vitreous cavity. For intracameral injections, the needle tip may be advanced through a corneoscleral limbal paracentesis, directed towards the central cornea, and 2 μl of vector suspension may be injected. For intracameral injections, the needle tip may be advanced through a corneoscleral limbal paracentesis, directed towards the central cornea, and 2 μl of vector suspension may be injected. These vectors may be injected at titers of either 1.0-1.4×10¹⁰ or 1.0-1.4×10⁹ transducing units (TU)/ml.

In some embodiments, for administration to the eye, lentiviral vectors. In some embodiments, the lentiviral vector is an equine infectious anemia virus (EIAV) vector. Exemplary EIAV vectors for eye delivery are described in Balagaan, J Gene Med 2006; 8: 275-285, Published online 21 Nov. 2005 in Wiley InterScience (www.interscience.wiley.com). DOI: 10.1002/jgm.845; Binley et al., HUMAN GENE THERAPY 23:980-991 (September 2012), which can be adapted for use with the engineered nucleic acid modification system of the present invention or component(s) thereof. In some embodiments, the dosage can be 1.1×10⁵ transducing units per eye (TU/eye) in a total volume of 100 μl.

Other viral vectors can also be used for delivery to the eye, such as AAV vectors, such as those described in Campochiaro et al., Human Gene Therapy 17:167-176 (February 2006), Millington-Ward et al. (Molecular Therapy, vol. 19 no. 4, 642-649 April 2011; Dalkara et al. (Sci Transl Med 5, 189ra76 (2013)), which can be adapted for use with the engineered nucleic acid modification system of the present invention or component(s) thereof. In some embodiments, the dose can range from about 10⁶ to 109.5 particle units. In the context of the Millington-Ward AAV vectors, a dose of about 2×10¹¹ to about 6×10¹³ virus particles can be administered. In the context of Dalkara vectors, a dose of about 1×10¹⁵ to about 1×10¹⁶ vg/ml administered to a human and can be used for human administration of the engineered nucleic acid modification system of the present invention or component(s) thereof.

In some embodiments, the sd-rxRNA® system of RXi Pharmaceuticals may be used/and or adapted for delivering engineered nucleic acid modification system of the present invention or component(s) thereof to the eye. In this system, a single intravitreal administration of 3 μg of sd-rxRNA results in sequence-specific reduction of PPIB mRNA levels for 14 days. The sd-rxRNA® system may be applied to the nucleic acid-targeting system of the present invention, contemplating a dose of about 3 to 20 mg of the engineered nucleic acid modification system of the present invention or component(s) thereof to a human.

In other embodiments, the methods of U.S. Publication No. 2013/0183282, which is directed to methods of cleaving a target sequence from the human rhodopsin gene, may also be modified for the engineered nucleic acid modification system of the present invention or component(s) thereof.

In other embodiments, the methods of U.S. Patent Publication No. 2013/0202678 for treating retinopathies and sight-threatening ophthalmologic disorders relating to delivering of the Puf-A gene (which is expressed in retinal ganglion and pigmented cells of eye tissues and displays a unique anti-apoptotic activity) to the sub-retinal or intravitreal space in the eye. In particular, desirable targets are zgc:193933, prdm1a, spata2, tex10, rbb4, ddx3, zp2.2, Blimp-1 and HtrA2, all of which may be targeted by the engineered nucleic acid modification system of the present invention and/or component(s) thereof.

Wu (Cell Stem Cell, 13:659-62, 2013) designed a guide RNA that led Cas9 to a single base pair mutation that causes cataracts in mice, where it induced DNA cleavage. Then using either the other wild-type allele or oligos given to the zygotes repair mechanisms corrected the sequence of the broken allele and corrected the cataract-causing genetic defect in mutant mouse. This approach can be adapted to and/or applied to the the engineered nucleic acid modification system of the present invention and/or component(s) thereof.

U.S. Patent Publication No. 2012/0159653, describes use of zinc finger nucleases to genetically modify cells, animals and proteins associated with macular degeneration (MD), the teachings of which can be applied to and/or adapted for the engineered nucleic acid modification system of the present invention and/or component(s) thereof.

One aspect of U.S. Patent Publication No. 2012/0159653 relates to editing of any chromosomal sequences that encode proteins associated with MD which may be applied to the engineered nucleic acid modification system of the present invention and/or component(s) thereof.

### Treating Muscle Diseases and Cardiovascular Diseases

In some embodiments, the engineered nucleic acid modification system of the present invention and/or component(s) thereof can be used to treat and/or prevent a muscle disease and associated circulatory or cardiovascular disease or disorder. The present invention also contemplates delivering the engineered nucleic acid modification system of the present invention and/or component(s) thereof to the heart. For the heart, a myocardium tropic adeno-associated virus (AAVM) is preferred, in particular AAVM41 which showed preferential gene transfer in the heart (see, e.g., Lin-Yanga et al., PNAS, Mar. 10, 2009, vol. 106, no. 10). Administration may be systemic or local. A dosage of about 1-10×1014 vector genomes are contemplated for systemic administration. See also, e.g., Eulalio et al. (2012) Nature 492: 376 and Somasuntharam et al. (2013) Biomaterials 34: 7790, the teachings of which can be adapted for and/or applied to the the engineered nucleic acid modification system of the present invention and/or component(s) thereof.

For example, U.S. Patent Publication No. 2011/0023139, the teachings of which can be adapted for and/or applied to the engineered nucleic acid modification system of the present invention and/or component(s) thereof describes use of zinc finger nucleases to genetically modify cells, animals and proteins associated with cardiovascular disease. Cardiovascular diseases generally include high blood pressure, heart attacks, heart failure, and stroke and TIA. Any chromosomal sequence involved in cardiovascular disease or the protein encoded by any chromosomal sequence involved in cardiovascular disease may be utilized in the methods described in this disclosure. The cardiovascular-related proteins are typically selected based on an experimental association of the cardiovascular-related protein to the development of cardiovascular disease. For example, the production rate or circulating concentration of a cardiovascular-related protein may be elevated or depressed in a population having a cardiovascular disorder relative to a population lacking the cardiovascular disorder. Differences in protein levels may be assessed using proteomic techniques including but not limited to Western blot, immunohistochemical staining, enzyme linked immunosorbent assay (ELISA), and mass spectrometry. Alternatively, the cardiovascular-related proteins may be identified by obtaining gene expression profiles of the genes encoding the proteins using genomic techniques including but not limited to DNA microarray analysis, serial analysis of gene expression (SAGE), and quantitative real-time polymerase chain reaction (Q-PCR). Exemplary chromosomal sequences can be found in Table 5.

The engineered nucleic acid modification system of the present invention and/or component(s) thereof can be used for treating diseases of the muscular system. The present invention also contemplates delivering the engineered nucleic acid modification system of the present invention and/or component(s) thereof to muscle(s).

In some embodiments, the muscle disease to be treated is a muscle dystrophy such as DMD. In some embodiments, the engineered nucleic acid modification system of the present invention and/or component(s) thereof, such as a system capable of RNA modification, described herein can be used to achieve exon skipping to achieve correction of the diseased gene. As used herein, the term “exon skipping” refers to the modification of pre-mRNA splicing by the targeting of splice donor and/or acceptor sites within a pre-mRNA with one or more complementary antisense oligonucleotide(s) (AONs). By blocking access of a spliceosome to one or more splice donor or acceptor site, an AON may prevent a splicing reaction thereby causing the deletion of one or more exons from a fully-processed mRNA. Exon skipping may be achieved in the nucleus during the maturation process of pre-mRNAs. In some examples, exon skipping may include the masking of key sequences involved in the splicing of targeted exons by using an engineered nucleic acid modification system of the present invention and/or component(s) thereof capable of RNA modification. In some embodiments, exon skipping can be achieved in dystrophin mRNA. In some embodiments, the engineered nucleic acid modification system of the present invention and/or component(s) thereof can induce exon skipping at exon 1, 2, 3, 4, 5, 6, 7, 8, 10, 11, 12, 13, 14, 15, 16, 17, 18, 19, 20, 21, 22, 23, 24, 25, 26, 27, 28, 29, 30, 31, 32, 33, 34, 45, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or any combination thereof of the dystrophin mRNA. In some embodiments, the engineered nucleic acid modification system of the present invention and/or component(s) thereof can induce exon skipping at exon 43, 44, 50, 51, 52, 55, or any combination thereof of the dystrophin mRNA. Mutations in these exons, can also be corrected using non-exon skipping polynucleotide modification methods.

In some embodiments, for treatment of a muscle disease, the method of Bortolanza et al. Molecular Therapy vol. 19 no. 11, 2055-264 November 2011) may be applied to an AAV expressing CRISPR Cas and injected into humans at a dosage of about 2×10¹⁵ or 2×10¹⁶ vg of vector. The teachings of Bortolanza et al., can be adapted for and/or applied to the engineered nucleic acid modification system of the present invention and/or component(s) thereof described herein.

In some embodiments, the method of Dumonceaux et al. (Molecular Therapy vol. 18 no. 5, 881-887 May 2010) may be applied to an AAV expressing CRISPR Cas and injected into humans, for example, at a dosage of about 10¹⁴ to about 10¹⁵ vg of vector. The teachings of Dumonceaux described herein can be adapted for and/or applied to the engineered nucleic acid modification system of the present invention and/or component(s) thereof.

In some embodiments, the method of Kinouchi et al. (Gene Therapy (2008) 15, 1126-1130) may be applied to the engineered nucleic acid modification system of the present invention and/or component(s) thereof, which can be injected into a human, for example, at a dosage of about 500 to 1000 ml of a 40 μM solution into the muscle for administration of the engineered nucleic acid modification system of the present invention and/or component(s) thereof.

In some embodiments, the method of Hagstrom et al. (Molecular Therapy Vol. 10, No. 2, August 2004) can be adapted for and/or applied to the engineered nucleic acid modification system of the present invention and/or component(s) thereof, which can be injected at a dose of about 15 to about 50 mg into the great saphenous vein of a human for administration of the engineered nucleic acid modification system of the present invention and/or component(s) thereof.

### Treating Diseases of the Liver and Kidney

In some embodiments, the engineered nucleic acid modification system of the present invention and/or component(s) thereof can be used to treat a disease of the kidney or liver. Thus, in some embodiments, delivery the engineered nucleic acid modification system of the present invention and/or component(s) thereof is to the liver or kidney.

Delivery strategies to induce cellular uptake of the therapeutic nucleic acid include physical force or vector systems such as viral-, lipid- or complex-based delivery, or nanocarriers. From the initial applications with less possible clinical relevance, when nucleic acids were addressed to renal cells with hydrodynamic high-pressure injection systemically, a wide range of gene therapeutic viral and non-viral carriers have been applied already to target posttranscriptional events in different animal kidney disease models in vivo (Csaba Revesz and Peter Hamar (2011). Delivery Methods to Target RNAs in the Kidney, Gene Therapy Applications, Prof Chunsheng Kang (Ed.), ISBN: 978-953-307-541-9, InTech, Available from: www.intechopen.com/books/gene-therapy-applications/delivery-methods-to-target-rnas-inthe-kidney). Delivery methods to the kidney may include those in Yuan et al. (Am J Physiol Renal Physiol 295: F605-F617, 2008). The method of Yuang et al. may be applied to the engineered nucleic acid modification system of the present invention and/or component(s) thereof. In some embodiments, about 1-2 g of the engineered nucleic acid modification system of the present invention and/or component(s) thereof conjugated with cholesterol can be administered via subcutaneous injection to a human for delivery to the kidneys.

In some embodiments, the method of Molitoris et al. (J Am Soc Nephrol 20: 1754-1764, 2009) can be adapted to the engineered nucleic acid modification system of the present invention and/or component(s) thereof. In some embodiments, a cumulative dose of 12-20 mg/kg of the x the engineered nucleic acid modification system of the present invention and/or component(s) thereof can be administered to a human for delivery to the proximal tubule cells of the kidneys.

In some embodiments, the methods of Thompson et al. (Nucleic Acid Therapeutics, Volume 22, Number 4, 2012) can be adapted for use and administration of the engineered nucleic acid modification system of the present invention and/or component(s) thereof. In some embodiments, a dose of up to 25 mg/kg of the engineered nucleic acid modification system of the present invention and/or component(s) thereof can be delivered via i.v. administration to a subject, such as a human. In some embodiments, the method of Shimizu et al. (J Am Soc Nephrol 21: 622-633, 2010) can be adapted for the engineered nucleic acid modification system of the present invention and/or component(s) thereof. In some embodiments, a dose of about of 10-20 μmol of the engineered nucleic acid modification system of the present invention and/or component(s) thereof complexed with nanocarriers in about 1-2 liters of a physiologic fluid can be administered i.p. to a subject, such as a human.

Other various delivery vehicles can be used to deliver the engineered nucleic acid modification system of the present invention and/or component(s) thereof to the kidney such as viral, hydrodynamic, lipid, polymer nanoparticles, aptamers and various combinations thereof (see e.g. Larson et al., Surgery, (August 2007), Vol. 142, No. 2, pp. (262-269); Hamar et al., Proc Natl Acad Sci, (October 2004), Vol. 101, No. 41, pp. (14883-14888); Zheng et al., Am J Pathol, (October 2008), Vol. 173, No. 4, pp. (973-980); Feng et al., Transplantation, (May 2009), Vol. 87, No. 9, pp. (1283-1289); Q. Zhang et al., PloS ONE, (July 2010), Vol. 5, No. 7, e11709, pp. (1-13); Kushibikia et al., J Controlled Release, (July 2005), Vol. 105, No. 3, pp. (318-331); Wang et al., Gene Therapy, (July 2006), Vol. 13, No. 14, pp. (1097-1103); Kobayashi et al., Journal of Pharmacology and Experimental Therapeutics, (February 2004), Vol. 308, No. 2, pp. (688-693); Wolfrum et al., Nature Biotechnology, (September 2007), Vol. 25, No. 10, pp. (1149-1157); Molitoris et al., J Am Soc Nephrol, (August 2009), Vol. 20, No. 8 pp. (1754-1764); Mikhaylova et al., Cancer Gene Therapy, (March 2011), Vol. 16, No. 3, pp. (217-226); Y. Zhang et al., J Am Soc Nephrol, (April 2006), Vol. 17, No. 4, pp. (1090-1101); Singhal et al., Cancer Res, (May 2009), Vol. 69, No. 10, pp. (4244-4251); Malek et al., Toxicology and Applied Pharmacology, (April 2009), Vol. 236, No. 1, pp. (97-108); Shimizu et al., J Am Soc Nephrology, (April 2010), Vol. 21, No. 4, pp. (622-633); Jiang et al., Molecular Pharmaceutics, (May-June 2009), Vol. 6, No. 3, pp. (727-737); Cao et al, J Controlled Release, (June 2010), Vol. 144, No. 2, pp. (203-212); Ninichuk et al., Am J Pathol, (March 2008), Vol. 172, No. 3, pp. (628-637); Purschke et al., Proc Natl Acad Sci, (March 2006), Vol. 103, No. 13, pp. (5173-5178).

In some embodiments, delivery is to liver cells. In some embodiments, the liver cell is a hepatocyte. Delivery of the engineered nucleic acid modification system of the present invention and/or component(s) thereof to liver cells may be via viral vectors, especially AAV (and in particular AAV2/6) vectors. These can be administered by intravenous injection. A preferred target for the liver, whether in vitro or in vivo, is the albumin gene. This is a so-called ‘safe harbor” as albumin is expressed at very high levels and so some reduction in the production of albumin following successful gene editing is tolerated. It is also preferred as the high levels of expression seen from the albumin promoter/enhancer allows for useful levels of correct or transgene production (from the inserted donor template) to be achieved even if only a small fraction of hepatocytes are edited. See sites identified by Wechsler et al. (reported at the 57th Annual Meeting and Exposition of the American Society of Hematology—abstract available online at https://ash.confex.com/ash/2015/webprogram/Paper86495.html and presented on 6 Dec. 2015) which can be adapted for use with the CRISPR-Cas systems herein.

Exemplary liver and kidney diseases that can be treated and/or prevented are described elsewhere herein.

### Treating Epithelial and Lung Diseases

In some embodiments, the disease treated or prevented by the engineered nucleic acid modification system of the present invention and/or component(s) thereof can be a lung or epithelial disease. The engineered nucleic acid modification system of the present invention and/or component(s) thereof can be used for treating epithelial and/or lung diseases. In some embodiments, the engineered nucleic acid modification system of the present invention and/or component(s) thereof are delivered to one or both lungs. Delivery to the lung(s) can be by any suitable method, such as i.v., internasal, and/or aerosol inhalation.

In some embodiments, as viral vector can be used to deliver the engineered nucleic acid modification system of the present invention and/or component(s) thereof to the lung(s). In some embodiments, the AAV is an AAV-1, AAV-2, AAV-5, AAV-6, and/or AAV-9 for delivery to the lungs (see e.g., Li et al., Molecular Therapy, vol. 17 no. 12, 2067-277 December 2009). In some embodiments, the MOI can vary from 1×103 to 4×105 vector genomes/cell. In some embodiments, the delivery vector can be an RSV vector as in Zamora et al. (Am J Respir Crit Care Med Vol 183. pp 531-538, 2011. The method of Zamora et al. may be applied to the nucleic acid-targeting system of the present invention and an aerosolized engineered nucleic acid modification system of the present invention and/or component(s) thereof, for example with a dosage of 0.6 mg/kg, can be used for delivery of the engineered nucleic acid modification system of the present invention and/or component(s) thereof to a subject.

Subjects treated for a lung disease may for example receive pharmaceutically effective amount of aerosolized AAV vector system per lung endobronchially delivered while spontaneously breathing. As such, aerosolized delivery is preferred for AAV delivery in general. An adenovirus or an AAV particle may be used for delivery. Suitable gene constructs, each operably linked to one or more regulatory sequences, may be cloned into the delivery vector. In this instance, the following constructs are provided as examples: Cbh or EF1a promoter for the effector complex of the engineered nucleic acid modification system and optionally a U6 or H1 promoter for a guide RNA: A preferred arrangement is to use a CFTRdelta508 targeting guide, a repair template for deltaF508 mutation and a codon optimized effector complex or protein therein (e.g., a site specific nuclease polypeptide (e.g., Cas polypeptide), DNA polymerase, and/or Vir D1 and/or Vir D2 polypeptide, with optionally one or more nuclear localization signal or sequence(s) (NLS(s)), e.g., two (2) NLSs.

### Treating Diseases of the Skin

The engineered nucleic acid modification system of the present invention and/or component(s) thereof can be used for the treatment of skin diseases. The present invention also contemplates delivering the engineered nucleic acid modification system of the present invention and/or component(s) thereof to the skin.

In some embodiments, delivery to the skin (intradermal delivery) of the engineered nucleic acid modification system of the present invention and/or component(s) thereof can be via one or more microneedles or microneedle containing device. For example, in some embodiments the device and methods of Hickerson et al. (Molecular Therapy-Nucleic Acids (2013) 2, e129) can be used and/or adapted to deliver the engineered nucleic acid modification system of the present invention and/or component(s) thereof, for example, at a dosage of up to 300 μl of 0.1 mg/ml the engineered nucleic acid modification system of the present invention and/or component(s) thereof system to the skin of a subject, such as a human.

In some embodiments, the methods and techniques of Leachman et al. (Molecular Therapy, vol. 18 no. 2, 442-446 February 2010) can be used and/or adapted for delivery of the engineered nucleic acid modification system of the present invention and/or component(s) thereof to the skin of a subject, such as a human.

In some embodiments, the methods and techniques of Zheng et al. (PNAS, Jul. 24, 2012, vol. 109, no. 30, 11975-11980) can be used and/or adapted for nanoparticle delivery of the engineered nucleic acid modification system of the present invention and/or component(s) thereof herein to the skin of a subject, such as a human. In some embodiments, as dosage of about 25 nM applied in a single application can achieve gene knockdown in the skin of the subject, such as a human.

### Treating Cancer

The engineered nucleic acid modification system of the present invention and/or component(s) thereof can be used for the treatment of cancer. The present invention also contemplates delivering the engineered nucleic acid modification system of the present invention and/or component(s) thereof to a cancer cell. Also, as is described elsewhere herein the engineered nucleic acid modification system of the present invention and/or component(s) thereof can be used to modify an immune cell, such as a CAR or CAR T cell, which can then in turn be used to treat and/or prevent cancer. This is also described in International Patent Application Publication WO2015161276, the disclosure of which is hereby incorporated by reference and described herein below.

Target genes suitable for the treatment or prophylaxis of cancer can include those set forth in Tables 5 and 6 and those identified at mitoMap.org. In some embodiments, target genes for cancer treatment and prevention can also include those described in WO2015048577 the disclosure of which is hereby incorporated by reference and can be adapted for and/or applied the engineered nucleic acid modification system of the present invention and/or component(s) thereof.

### Exemplary Diseases

Genetic Diseases and Diseases with a Genetic and/or Epigenetic Aspect

The engineered nucleic acid modification system of the present invention and/or component(s) thereof can be used to treat and/or prevent a genetic disease or a disease with a genetic and/or epigenetic aspect. The genes and conditions exemplified herein are not exhaustive. In some embodiments, a method of treating and/or preventing a genetic disease can include administering an engineered nucleic acid modification system of the present invention and/or component(s) thereof to a subject, where the engineered nucleic acid modification system of the present invention and/or component(s) thereof is capable of modifying one or more copies of one or more genes associated with the genetic disease or a disease with a genetic and/or epigenetic aspect in one or more cells of the subject. In some embodiments, modifying one or more copies of one or more genes associated with a genetic disease or a disease with a genetic and/or epigenetic aspect in the subject can eliminate a genetic disease or a symptom thereof in the subject. In some embodiments, modifying one or more copies of one or more genes associated with a genetic disease or a disease with a genetic and/or epigenetic aspect in the subject can decrease the severity of a genetic disease or a symptom thereof in the subject. In some embodiments, the engineered nucleic acid modification system of the present invention and/or component(s) thereof can modify one or more genes or polynucleotides associated with one or more diseases, including genetic diseases and/or those having a genetic aspect and/or epigenetic aspect, including but not limited to, any one or more set forth in Table 5. It will be appreciated that those diseases and associated genes listed herein are non-exhaustive and non-limiting. Further some genes play roles in the development of multiple diseases.

TABLE 5
Exemplary Genetic and Other Diseases and Associated Genes

| | Primary | Additional | |
|---|---|---|---|
| | Tissues or | Tissues/ | |
| | System | Systems | |
| Disease Name | Affected | Affected | Genes |
| Achondroplasia | Bone and | | fibroblast growth factor receptor 3 |
| | Muscle | | (FGFR3) |
| Achromatopsia | eye | | CNGA3, CNGB3, GNAT2, PDE6C, |
| | | | PDE6H, ACHM2, ACHM3, |
| Acute Renal Injury | kidney | | NFkappaB, AATF, p85alpha, FAS, |
| | | | Apoptosis cascade elements (e.g. |
| | | | FASR, Caspase 2, 3, 4, 6, 7, 8, 9, 10, |
| | | | AKT, TNF alpha, IGF1, IGF1R, |
| | | | RIPK1), p53 |
| Age Related Macular | eye | | Abcr; CCL2; CC2; CP |
| Degeneration | | | (ceruloplasmin); Timp3; cathepsinD; |
| | | | VLDLR, CCR2 |
| AIDS | Immune System | | KIR3DL1, NKAT3, NKB1, AMB11, |
| | | | KIR3DS1, IFNG, CXCL12, SDF1 |
| Albinism (including | Skin, hair, eyes, | | TYR, OCA2, TYRP1, and SLC45A2, |
| oculocutaneous albinism (types | | | SLC24A5 and C10orf11 |
| 1-7) and ocular albinism) | | | |
| Alkaptonuria | Metabolism of | Tissues/organs | HGD |
| | amino acids | where | |
| | | homogentisic | |
| | | acid | |
| | | accumulates, | |
| | | particularly | |
| | | cartilage (joints), | |
| | | heart valves, | |
| | | kidneys | |
| alpha-1 antitrypsin deficiency | Lung | Liver, skin, | SERPINA1, those set forth in |
| (AATD or A1AD) | | vascular system, | WO2017165862, PiZ allele |
| | | kidneys, GI | |
| ALS | CNS | | SOD1; ALS2; ALS3; ALS5; |
| | | | ALS7; STEX; FUS; TARDBP; VEGF |
| | | | (VEGF-a; VEGF-b; VEGF-c); DPP6; NEFH, |
| | | | PTGS1, SLC1A2, TNFRSF10B, |
| | | | PRPH, HSP90AA1, CRIA2, IFNG, |
| | | | AMPA2 S100B, FGF2, AOX1, CS, |
| | | | TXN, RAPHJ1, MAP3K5, NBEAL1, |
| | | | GPX1, ICA1L, RAC1, MAPT, ITPR2, |
| | | | ALS2CR4, GLS, ALS2CR8, CNTFR, |
| | | | ALS2CR11, FOLH1, FAM117B, |
| | | | P4HB, CNTF, SQSTM1, STRADB, |
| | | | NAIP, NLR, YWHAQ, SLC33A1, |
| | | | TRAK2, SCA1, NIF3L1, NIF3, |
| | | | PARD3B, COX8A, CDK15, HECW1, |
| | | | HECT, C2, WW 15, NOS1, MET, |
| | | | SOD2, HSPB1, NEFL, CTSB, ANG, |
| | | | HSPA8, RNase A, VAPB, VAMP, |
| | | | SNCA, alpha HGF, CAT, ACTB, |
| | | | NEFM, TH, BCL2, FAS, CASP3, |
| | | | CLU, SMN1, G6PD, BAX, HSF1, |
| | | | RNF19A, JUN, ALS2CR12, HSPA5, |
| | | | MAPK14, APEX1, TXNRD1, NOS2, |
| | | | TIMP1, CASP9, XIAP, GLG1, EPO, |
| | | | VEGFA, ELN, GDNF, NFE2L2, |
| | | | SLC6A3, HSPA4, APOE, PSMB8, |
| | | | DCTN2, TIMP3, KIFAP3, SLC1A1, |
| | | | SMN2, CCNC, STUB1, ALS2, |
| | | | PRDX6, SYP, CABIN1, CASP1, |
| | | | GART, CDK5, ATXN3, RTN4, |
| | | | C1QB, VEGFC, HTT, PARK7, XDH, |
| | | | GFAP, MAP2, CYCS, FCGR3B, CCS, |
| | | | UBL5, MMP9m SLC18A3, TRPM7, |
| | | | HSPB2, AKT1, DEERL1, CCL2, |
| | | | NGRN, GSR, TPPP3, APAF1, |
| | | | BTBD10, GLUD1, CXCR4, S:C1A3, |
| | | | FLT1, PON1, AR, LIF, ERBB3, :GA:S1, |
| | | | CD44, TP53, TLR3, GRIA1, |
| | | | GAPDH, AMPA, GRIK1, DES, |
| | | | CHAT, FLT4, CHMP2B, BAG1, |
| | | | CHRNA4, GSS, BAK1, KDR, GSTP1, |
| | | | OGG1, IL6 |
| Alzheimer's Disease | Brain | | E1; CHIP; UCH; UBB; Tau; LRP; |
| | | | PICALM; CLU; PS1; |
| | | | SORL1; CR1; VLDLR; UBA1; |
| | | | UBA3; CHIP28; AQP1; UCHL1; |
| | | | UCHL3; APP, AAA, CVAP, AD1, |
| | | | APOE, AD2, DCP1, ACE1, MPO, |
| | | | PACIP1, PAXIP1L, PTIP, A2M, |
| | | | BLMH, BMH, PSEN1, AD3, ALAS2, |
| | | | ABCA1, BIN1, BDNF, BTNL8, |
| | | | C1ORF49, CDH4, CHRNB2, |
| | | | CKLFSF2, CLEC4E, CR1L, CSF3R, |
| | | | CST3, CYP2C, DAPK1, ESR1, |
| | | | FCAR, FCGR3B, FFA2, FGA, GAB2, |
| | | | GALP, GAPDHS, GMPB, HP, HTR7, |
| | | | IDE, IF127, IFI6, IFIT2, IL1RN, IL- |
| | | | 1RA, IL8RA, IL8RB, JAG1, KCNJ15, |
| | | | LRP6, MAPT, MARK4, MPHOSPH1, |
| | | | MTHFR, NBN, NCSTN, NIACR2, |
| | | | NMNAT3, NTM, ORM1, P2RY13, |
| | | | PBEF1, PCK1, PICALM, PLAU, |
| | | | PLXNC1, PRNP, PSEN1, PSEN2, |
| | | | PTPRA, RALGPS2, RGSL2, |
| | | | SELENBP1, SLC25A37, SORL1, |
| | | | Mitoferrin-1, TF, TFAM, TNF, |
| | | | TNFRSF10C, UBE1C |
| Amyloidosis | | | APOA1, APP, AAA, CVAP, AD1, |
| | | | GSN, FGA, LYZ, TTR, PALB |
| Amyloid neuropathy | | | TTR, PALB |
| Anemia | Blood | | CDAN1, CDA1, RPS19, DBA, PKLR, |
| | | | PK1, NT5C3, UMPH1, PSN1, RHAG, |
| | | | RH50A, NRAMP2, SPTB, ALAS2, |
| | | | ANH1, ASB, ABCB7, ABC7, ASAT |
| Angelman Syndrome | Nervous system, | | UBE3A |
| | brain | | |
| Attention Deficit Hyperactivity | Brain | | PTCHD1 |
| Disorder (ADHD) | | | |
| Autoimmune lymphoproliferative | Immune system | | TNFRSF6, APT1, FAS, CD95, |
| syndrome | | | ALPS1A |
| Autism, Autism spectrum | Brain | | PTCHD1; Mecp2; BZRAP1; MDGA2; |
| disorders (ASDs), including | | | Sema5A; Neurexin 1; GLO1, RTT, |
| Asperger's and a general | | | PPMX, MRX16, RX79, NLGN3, |
| diagnostic category called | | | NLGN4, KIAA1260, AUTSX2, |
| Pervasive Developmental | | | FMRI, FMR2; FXR1; FXR2; |
| Disorders (PDDs) | | | MGLUR5, ATP10C, CDH10, GRM6, |
| | | | MGLUR6, CDH9, CNTN4, NLGN2, |
| | | | CNTNAP2, SEMA5A, DHCR7, |
| | | | NLGN4X, NLGN4Y, DPP6, NLGN5, |
| | | | EN2, NRCAM, MDGA2, NRXN1, |
| | | | FMR2, AFF2, FOXP2, OR4M2, |
| | | | OXTR, FXR1, FXR2, PAH, |
| | | | GABRA1, PTEN, GABRA5, PTPRZ1, |
| | | | GABRB3, GABRG1, HIRIP3, |
| | | | SEZ6L2, HOXA1, SHANK3, IL6, |
| | | | SHBZRAP1, LAMB1, SLC6A4, |
| | | | SERT, MAPK3, TAS2R1, MAZ, |
| | | | TSC1, MDGA2, TSC2, MECP2, |
| | | | UBE3A, WNT2, see also |
| | | | 20110023145 |
| autosomal dominant polycystic | kidney | liver | PKD1, PKD2 |
| kidney disease (ADPKD) - | | | |
| (includes diseases such as von | | | |
| Hippel-Lindau disease and | | | |
| tubreous sclerosis complex | | | |
| disease) | | | |
| Autosomal Recessive Polycystic | kidney | liver | PKDH1 |
| Kidney Disease (ARPKD) | | | |
| Ataxia-Telangiectasia (a.k.a | Nervous system, | various | ATM |
| Louis Bar syndrome) | immune system | | |
| B-Cell Non-Hodgkin Lymphoma | | | BCL7A, BCL7 |
| Bardet-Biedl syndrome | Eye, | Liver, ear, | ARL6, BBS1, BBS2, BBS4, BBS5, |
| | musculoskeletal | gastrointestinal | BBS7, BBS9, BBS10, BBS12, |
| | system, kidney, | system, brain | CEP290, INPP5E, LZTFL1, MKKS, |
| | reproductive | | MKS1, SDCCAG8, TRIM32, TTC8 |
| | organs | | |
| Bare Lymphocyte Syndrome | blood | | TAPBP, TPSN, TAP2, ABCB3, PSF2, |
| | | | RING11, MHC2TA, C2TA, RFX5, |
| | | | RFXAP, RFX5 |
| Barter's Syndrome (types I, II, | kidney | | SLC12A1 (type I), KCNJ1 (type II), |
| III, IVA and B, and V) | | | CLCNKB (type III), BSND (type IV |
| | | | A), or both the CLCNKA CLCNKB |
| | | | genes (type IV B), CASR (type V). |
| Becker muscular dystrophy | Muscle | | DMD, BMD, MYF6 |
| Best Disease (Vitelliform | eye | | VMD2 |
| Macular Dystrophy type 2) | | | |
| Bleeding Disorders | blood | | TBXA2R, P2RX1, P2X1 |
| Blue Cone Monochromacy | eye | | OPN1LW, OPN1MW, and LCR |
| Breast Cancer | Breast tissue | | BRCA1, BRCA2, COX-2 |
| Bruton's Disease (aka X-linked | Immune system, | | BTK |
| Agammglobulinemia) | specifically B | | |
| | cells | | |
| Cancers (e.g., lymphoma, chronic | Various | | FAS, BID, CTLA4, PDCD1, CBLB, |
| lymphocytic leukemia (CLL), B | | | PTPN6, TRAC, TRBC, those |
| cell acute lymphocytic leukemia | | | described in WO2015048577 |
| (B-ALL), acute lymphoblastic | | | |
| leukemia, acute myeloid | | | |
| leukemia, non-Hodgkin's | | | |
| lymphoma (NHL), diffuse large | | | |
| cell lymphoma (DLCL), multiple | | | |
| myeloma, renal cell carcinoma | | | |
| (RCC), neuroblastoma, colorectal | | | |
| cancer, breast cancer, ovarian | | | |
| cancer, melanoma, sarcoma, | | | |
| prostate cancer, lung cancer, | | | |
| esophageal cancer, hepatocellular | | | |
| carcinoma, pancreatic cancer, | | | |
| astrocytoma, mesothelioma, head | | | |
| and neck cancer, and | | | |
| medulloblastoma | | | |
| Cardiovascular Diseases | heart | Vascular system | IL1B, XDH, TP53, PTGS, MB, IL4, |
| | | | ANGPT1, ABCGu8, CTSK, PTGIR, |
| | | | KCNJ11, INS, CRP, PDGFRB, |
| | | | CCNA2, PDGFB, KCNJ5, KCNN3, |
| | | | CAPN10, ADRA2B, ABCG5, |
| | | | PRDX2, CPAN5, PARP14, MEX3C, |
| | | | ACE, RNF, IL6, TNF, STN, |
| | | | SERPINE1, ALB, ADIPOQ, APOB, |
| | | | APOE, LEP, MTHFR, APOA1, |
| | | | EDN1, NPPB, NOS3, PPARG, PLAT, |
| | | | PTGS2, CETP, AGTR1, HMGCR, |
| | | | IGF1, SELE, REN, PPARA, PON1, |
| | | | KNG1, CCL2, LPL, VWF, F2, |
| | | | ICAM1, TGFB, NPPA, IL10, EPO, |
| | | | SOD1, VCAM1, IFNG, LPA, MPO, |
| | | | ESR1, MAPK, HP, F3, CST3, COG2, |
| | | | MMP9, SERPINC1, F8, HMOX1, |
| | | | APOC3, IL8, PROL1, CBS, NOS2, |
| | | | TLR4, SELP, ABCA1, AGT, LDLR, |
| | | | GPT, VEGFA, NR3C2, IL18, NOS1, |
| | | | NR3C1, FGB, HGF, ILIA, AKT1, |
| | | | LIPC, HSPD1, MAPK14, SPP1, |
| | | | ITGB3, CAT, UTS2, THBD, F10, CP, |
| | | | TNFRSF11B, EGFR, MMP2, PLG, |
| | | | NPY, RHOD, MAPK8, MYC, FN1, |
| | | | CMA1, PLAU, GNB3, ADRB2, |
| | | | SOD2, F5, VDR, ALOX5, HLA- |
| | | | DRB1, PARP1, CD40LG, PON2, |
| | | | AGER, IRS1, PTGS1, ECE1, F7, |
| | | | IRMN, EPHX2, IGFBP1, MAPK10, |
| | | | FAS, ABCB1, JUN, IGFBP3, CD14, |
| | | | PDE5A, AGTR2, CD40, LCAT, |
| | | | CCR5, MMP1, TIMP1, ADM, |
| | | | DYT10, STAT3, MMP3, ELN, USF1, |
| | | | CFH, HSPA4, MMP12, MME, F2R, |
| | | | SELL, CTSB, ANXA5, ADRB1, |
| | | | CYBA, FGA, GGT1, LIPG, HIF1A, |
| | | | CXCR4, PROC, SCARB1, CD79A, |
| | | | PLTP, ADD1, FGG, SAA1, KCNH2, |
| | | | DPP4, NPR1, VTN, KIAA0101, FOS, |
| | | | TLR2, PPIG, IL1R1, AR, CYP1A1, |
| | | | SERPINA1, MTR, RBP4, APOA4, |
| | | | CDKN2A, FGF2, EDNRB, ITGA2, |
| | | | VLA-2, CABIN1, SHBG, HMGB1, |
| | | | HSP90B2P, CYP3A4, GJA1, CAV1, |
| | | | ESR2, LTA, GDF15, BDNF, |
| | | | CYP2D6, NGF, SP1, TGIF1, SRC, |
| | | | EGF, PIK3CG, HLA-A, KCNQ1, |
| | | | CNR1, FBN1, CHKA, BEST1, |
| | | | CTNNB1, IL2, CD36, PRKAB1, TPO, |
| | | | ALDH7A1, CX3CR1, TH, F9, CH1, |
| | | | TF, HFE, IL17A, PTEN, GSTM1, |
| | | | DMD, GATA4, F13A1, TTR, FABP4, |
| | | | PON3, APOC1, INSR, TNFRSF1B, |
| | | | HTR2A, CSF3, CYP2C9, TXN, |
| | | | CYP11B2, PTH, CSF2, KDR, |
| | | | PLA2G2A, THBS1, GCG, RHOA, |
| | | | ALDH2, TCF7L2, NFE2L2, |
| | | | NOTCH1, UGT1A1, IFNA1, PPARD, |
| | | | SIRT1, GNHR1, PAPPA, ARR3, |
| | | | NPPC, AHSP, PTK2, IL13, MTOR, |
| | | | ITGB2, GSTT1, IL6ST, CPB2, |
| | | | CYP1A2, HNF4A, SLC64A, |
| | | | PLA2G6, TNFSF11, SLC8A1, F2RL1, |
| | | | AKR1A1, ALDH9A1, BGLAP, |
| | | | MTTP, MTRR, SULT1A3, RAGE, |
| | | | C4B, P2RY12, RNLS, CREB1, |
| | | | POMC, RAC1, LMNA, CD59, |
| | | | SCM5A, CYP1B1, MIF, MMP13, |
| | | | TIMP2, CYP19A1, CUP21A2, |
| | | | PTPN22, MYH14, MBL2, SELPLG, |
| | | | AOC3, CTSL1, PCNA, IGF2, ITGB1, |
| | | | CAST, CXCL12, IGHE, KCNE1, |
| | | | TFRC, COL1A1, COL1A2, IL2RB, |
| | | | PLA2G10, ANGPT2, PROCR, NOX4, |
| | | | HAMP, PTPN11, SLCA1, IL2RA, |
| | | | CCL5, IRF1, CF:AR, CA:CA, EIF4E, |
| | | | GSTP1, JAK2, CYP3A5, HSPG2, |
| | | | CCL3, MYD88, VIP, SOAT1, |
| | | | ADRBK1, NR4A2, MMP8, NPR2, |
| | | | GCH1, EPRS, PPARGC1A, F12, |
| | | | PECAM1, CCL4, CERPINA34, |
| | | | CASR, FABP2, TTF2, PROS1, CTF1, |
| | | | SGCB, YME1L1, CAMP, ZC3H12A, |
| | | | AKR1B1, MMP7, AHR, CSF1, |
| | | | HDAC9, CTGF, KCNMA1, UGT1A, |
| | | | PRKCA, COMT, S100B, EGR1, PRL, |
| | | | IL15, DRD4, CAMK2G, SLC22A2, |
| | | | CCL11, PGF, THPO, GP6, TACR1, |
| | | | NTS, HNF1A, SST, KCDN1, |
| | | | LOC646627, TBXAS1, CUP2J2, |
| | | | TBXA2R, ADH1C, ALOX12, AHSG, |
| | | | BHMT, GJA4, SLC25A4, ACLY, |
| | | | ALOX5AP, NUMA1, CYP27B1, |
| | | | CYSLTR2, SOD3, LTC4S, UCN, |
| | | | GHRL, APOC2, CLEC4A, |
| | | | KBTBD10, TNC, TYMS, SHC1, |
| | | | LRP1, SOCS3, ADH1B, KLK3, |
| | | | HSD11B1, VKORC1, SERPINB2, |
| | | | TNS1, RNF19A, EPOR, ITGAM, |
| | | | PITX2, MAPK7, FCGR3A, LEEPR, |
| | | | ENG, GPX1, GOT2, HRH1, NR112, |
| | | | CRH, HTR1A, VDAC1, HPSE, |
| | | | SFTPD, TAP2, RMF123, PTK2Bm |
| | | | NTRK2, IL6R, ACHE, GLP1R, GHR, |
| | | | GSR, NQO1, NR5A1, GJB2, |
| | | | SLC9A1, MAOA, PCSK9, FCGR2A, |
| | | | SERPINF1, EDN3, UCP2, TFAP2A, |
| | | | C4BPA, SERPINF2, TYMP, ALPP, |
| | | | CXCR2, SLC3A3, ABCG2, ADA, |
| | | | JAK3, HSPA1A, FASN, FGF1, F11, |
| | | | ATP7A, CR1, GFPA, ROCK1, |
| | | | MECP2, MYLK, BCHE, LIPE, |
| | | | ADORA1, WRN, CXCR3, CD81, |
| | | | SMAD7, LAMC2, MAP3K5, CHGA, |
| | | | IAPP, RHO, ENPP1, PTHLH, NRG1, |
| | | | VEGFC, ENPEP, CEBPB, NAGLU,. |
| | | | F2RL3, CX3CL1, BDKRB1, |
| | | | ADAMTS13, ELANE, ENPP2, CISH, |
| | | | GAST, MYOC, ATP1A2, NF1, GJB1, |
| | | | MEF2A, VCL, BMPR2, TUBB, |
| | | | CDC42, KRT18, HSF1, MYB, |
| | | | PRKAA2, ROCK2, TFP1, PRKG1, |
| | | | BMP2, CTNND1, CTH, CTSS, |
| | | | VAV2, NPY2R, IGFBP2, CD28, |
| | | | GSTA1, PPIA, APOH, S100A8, IL11, |
| | | | ALOX15, FBLN1, NR1H3, SCD, GIP, |
| | | | CHGB, PRKCB, SRD5A1, HSD11B2, |
| | | | CALCRL, GALNT2, ANGPTL4, |
| | | | KCNN4, PIK3C2A, HBEGF, |
| | | | CYP7A1, HLA-DRB5, BNIP3, |
| | | | GCKR, S100A12, PADI4, HSPA14, |
| | | | CXCR1, H19, KRTAP19-3, IDDM2, |
| | | | RAC2, YRY1, CLOCK, NGFR, DBH, |
| | | | CHRNA4, CACNA1C, PRKAG2, |
| | | | CHAT, PTGDS, NR1H2, TEK, |
| | | | VEGFB, MEF2C, MAPKAPK2, |
| | | | TNFRSF11A, HSPA9, CYSLTR1, |
| | | | MATIA, OPRL1, IMPA1, CLCN2, |
| | | | DLD, PSMA6, PSMB8, CHI3L1, |
| | | | ALDH1B1, PARP2, STAR, LBP, |
| | | | ABCC6, RGS2, EFNB2, GJB6, |
| | | | APOA2, AMPD1, DYSF, |
| | | | FDFT1, EMD2, CCR6, GJB3, IL1RL1, |
| | | | ENTPD1, BBS4, CELSR2, F11R, |
| | | | RAPGEF3, HYAL1, ZNF259, |
| | | | ATOX1, ATF6, KHK, SAT1, GGH, |
| | | | TIMP4, SLC4A4, PDE2A, PDE3B, |
| | | | FADS1, FADS2, TMSB4X, TXNIP, |
| | | | LIMS1, RHOB, LY96, FOXO1, |
| | | | PNPLA2, TRH, GJC1, S:C17A5, FTO, |
| | | | GJD2, PRSC1, CASP12, GPBAR1, |
| | | | PXK, IL33, TRIB1, PBX4, NUPR1, |
| | | | 15-SEP, CILP2, TERC, GGT2, |
| | | | MTCO1, UOX, AVP |
| Cataract | eye | | CRYAA, CRYA1, CRYBB2, CRYB2, |
| | | | PITX3, BFSP2, CP49, CP47, CRYAA, |
| | | | CRYA1, PAX6, AN2, MGDA, |
| | | | CRYBA1, CRYB1, CRYGC, CRYG3, |
| | | | CCL, LIM2, MP19, CRYGD, CRYG4, |
| | | | BFSP2, CP49, CP47, HSF4, CTM, |
| | | | HSF4, CTM, MIP, AQP0, CRYAB, |
| | | | CRYA2, CTPP2, CRYBB1, CRYGD, |
| | | | CRYG4, CRYBB2, CRYB2, CRYGC, |
| | | | CRYG3, CCL, CRYAA, CRYA1, |
| | | | GJA8, CX50, CAE1, GJA3, CX46, |
| | | | CZP3, CAE3, CCM1, CAM, KRIT1 |
| CDKL-5 Deficiencies or | Brain, CNS | | CDKL5 |
| Mediated Diseases | | | |
| Charcot-Marie-Tooth (CMT) | Nervous system | Muscles | PMP22 (CMT1A and E), MPZ |
| disease (Types 1, 2, 3, 4,) | | (dystrophy) | (CMT1B), LITAF (CMT1C), EGR2 |
| | | | (CMT1D), NEFL (CMT1F), GJB1 |
| | | | (CMT1X), MFN2 (CMT2A), KIF1B |
| | | | (CMT2A2B), RAB7A (CMT2B), |
| | | | TRPV4 (CMT2C), GARS (CMT2D), |
| | | | NEFL (CMT2E), GAPD1 (CMT2K), |
| | | | HSPB8 (CMT2L), DYNC1H1, |
| | | | CMT20), LRSAM1 (CMT2P), |
| | | | IGHMBP2 (CMT2S), MORC2 |
| | | | (CMT2Z), GDAP1 (CMT4A), |
| | | | MTMR2 or SBF2/MTMR13 |
| | | | (CMT4B), SH3TC2 (CMT4C), |
| | | | NDRG1 (CMT4D), PRX (CMT4F), |
| | | | FIG4 (CMT4J), NT-3 |
| Chédiak-Higashi Syndrome | Immune system | Skin, hair, eyes, | LYST |
| | | neurons | |
| Choroidermia | | | CHM, REP1, |
| Chorioretinal atrophy | eye | | PRDM13, RGR, TEAD1 |
| Chronic Granulomatous Disease | Immune system | | CYBA, CYBB, NCF1, NCF2, NCF4 |
| Chronic Mucocutaneous | Immune system | | AIRE, CARD9, CLEC7A IL12B, |
| Candidiasis | | | IL12B1, IL1F, IL17RA, IL17RC, |
| | | | RORC, STAT1, STAT3, TRAF31P2 |
| Cirrhosis | liver | | KRT18, KRT8, CIRH1A, NAIC, |
| | | | TEX292, KIAA1988 |
| Colon cancer (Familial | Gastrointestinal | | FAP: APC HNPCC: |
| adenomatous polyposis (FAP) | | | MSH2, MLH1, PMS2, SH6, PMS1 |
| and hereditary nonpolyposis | | | |
| colon cancer (HNPCC)) | | | |
| Combined Immunodeficiency | Immune System | | IL2RG, SCIDX1, SCIDX, IMD4); |
| | | | HIV-1 (CCL5, SCYA5, D17S136E, |
| | | | TCP228 |
| Cone(-rod) dystrophy | eye | | AIPL1, CRX, GUA1A, GUCY2D, |
| | | | PITPM3, PROM1, PRPH2, RIMS1, |
| | | | SEMA4A, ABCA4, ADAM9, ATF6, |
| | | | C21ORF2, C8ORF37, CACNA2D4, |
| | | | CDHR1, CERKL, CNGA3, CNGB3, |
| | | | CNNM4, CNAT2, IFT81, KCNV2, |
| | | | PDE6C, PDE6H, POC1B, RAX2, |
| | | | RDH5, RPGRIP1, TTLL5, RetCG1, |
| | | | GUCY2E |
| Congenital Stationary Night | eye | | CABP4, CACNA1F, CACNA2D4, |
| Blindness | | | GNAT1, CPR179, GRK1, GRM6, |
| | | | LRIT3, NYX, PDE6B, RDH5, RHO, |
| | | | RLBP1, RPE65, SAG, SLC24A1, |
| | | | TRPM1, |
| Congenital Fructose Intolerance | Metabolism | | ALDOB |
| Cori's Disease (Glycogen Storage | Various- | | AGL |
| Disease Type III) | wherever | | |
| | glycogen | | |
| | accumulates, | | |
| | particularly | | |
| | liver, heart, | | |
| | skeletal muscle | | |
| Corneal clouding and dystrophy | eye | | APOA1, TGFBI, CSD2, CDGG1, |
| | | | CSD, BIGH3, CDG2, TACSTD2, |
| | | | TROP2, M1S1, VSX1, RINX, PPCD, |
| | | | PPD, KTCN, COL8A2, FECD, |
| | | | PPCD2, PIP5K3, CFD |
| Cornea plana congenital | | | KERA, CNA2 |
| Cri du chat Syndrome, also | | | Deletions involving only band 5p15.2 |
| known as 5p syndrome and cat | | | to the entire short arm of chromosome |
| cry syndrome | | | 5, e.g. CTNND2, TERT, |
| Cystic Fibrosis (CF) | Lungs and | Pancreas, liver, | CTFR, ABCC7, CF, MRP7, SCNN1A, |
| | respiratory | digestive | those described in WO2015157070 |
| | system | system, | |
| | | reproductive | |
| | | system, | |
| | | exocrine, glands, | |
| Diabetic nephropathy | kidney | | Gremlin, 12/15- lipoxygenase, TIM44, |
| Dent Disease (Types 1 and 2) | Kidney | | Type 1: CLCN5, Type 2: ORCL |
| Dentatorubro-Pallidoluysian | CNS, brain, | | Atrophin-1 and Atn1 |
| Atrophy (DRPLA) (aka Haw | muscle | | |
| River and Naito-Oyanagi | | | |
| Disease) | | | |
| Down Syndrome | various | | Chromosome 21 trisomy |
| Drug Addiction | Brain | | Prkce; Drd2; Drd4; ABAT; |
| | | | GRIA2; Grm5; Grin1; Htr1b; Grin2a; |
| | | | Drd3; Pdyn; Gria1 |
| Duane syndrome (Types 1, 2, and | eye | | CHN1, indels on chromosomes 4 and 8 |
| 3, including subgroups A, B and | | | |
| C). Other names for this | | | |
| condition include: Duane's | | | |
| Retraction Syndrome (or DR | | | |
| syndrome), Eye Retraction | | | |
| Syndrome, Retraction Syndrome, | | | |
| Congenital retraction syndrome | | | |
| and Stilling-Turk-Duane | | | |
| Syndrome | | | |
| Duchenne muscular dystrophy | muscle | Cardiovascular, | DMD, BMD, dystrophin gene, intron |
| (DMD) | | respiratory | flanking exon 51 of DMD gene, exon |
| | | | 51 mutations in DMD gene, see also |
| | | | WO2013163628 and US Pat. Pub. |
| | | | 20130145487 |
| Edward's Syndrome | | | Complete or partial trisomy of |
| (Trisomy 18) | | | chromosome 18 |
| Ehlers-Danlos Syndrome (Types | Various | | COL5A1, COL5A2, COL1A1, |
| I-VI) | depending on | | COL3A1, TNXB, PLOD1, COL1A2, |
| | type: including | | FKBP14 and ADAMTS2 |
| | musculoskeletal, | | |
| | eye, vasculature, | | |
| | immune, and | | |
| | skin | | |
| Emery-Dreifuss muscular | muscle | | LMNA, LMN1, EMD2, FPLD, |
| dystrophy | | | CMD1A, HGPS, LGMD1B, LMNA, |
| | | | LMN1, EMD2, FPLD, CMD1A |
| Enhanced S-Cone Syndrome | eye | | NR2E3, NRL |
| Fabry's Disease | Various - | | GLA |
| | including skin, | | |
| | eyes, and | | |
| | gastrointestinal | | |
| | system, kidney, | | |
| | heart, brain, | | |
| | nervous system | | |
| Facioscapulohumeral muscular | muscles | | FSHMD1A, FSHD1A, FRG1, |
| dystrophy | | | |
| Factor H and Factor H-like 1 | blood | | HF1, CFH, HUS |
| Factor V Leiden thrombophilia | blood | | Factor V (F5) |
| and Factor V deficiency | | | |
| Factor V and Factor VII | blood | | MCFD2 |
| deficiency | | | |
| Factor VII deficiency | blood | | F7 |
| Factor X deficiency | blood | | F10 |
| Factor XI deficiency | blood | | F11 |
| Factor XII deficiency | blood | | F12, HAF |
| Factor XIIIA deficiency | blood | | F13A1, F13A |
| Factor XIIIB deficiency | blood | | F13B |
| Familial Hypercholestereolemia | Cardiovascular | | APOB, LDLR, PCSK9 |
| | system | | |
| Familial Mediterranean Fever | Various- | Heart, kidney, | MEFV |
| (FMF) also called recurrent | organs/tissues | brain/CNS, | |
| polyserositis or familial | with serous or | reproductive | |
| paroxysmal polyserositis | synovial | organs | |
| | membranes, | | |
| | skin, joints | | |
| Fanconi Anemia | Various - blood | | FANCA, FACA, FA1, FA, FAA, |
| | (anemia), | | FAAP95, FAAP90, FLJ34064, |
| | immune system, | | FANCC, FANCG, RAD51, BRCA1, |
| | cognitive, | | BRCA2, BRIP1, BACH1, FANCJ, |
| | kidneys, eyes, | | FANCB, FANCD1, FANCD2, |
| | musculoskeletal | | FANCD, FAD, FANCE, FACE, |
| | | | FANCF, FANCI, ERCC4, FANCL, |
| | | | FANCM, PALB2, RAD51C, SLX4, |
| | | | UBE2T, FANCB, XRCC9, PHF9, |
| | | | KIAA1596 |
| Fanconi Syndrome Types I | kidneys | | FRTS1, GATM |
| (Childhood onset) and II (Adult | | | |
| Onset) | | | |
| Fragile X syndrome and related | brain | | FMR1, FMR2; FXR1; FXR2; |
| disorders | | | mGLUR5 |
| Fragile XE Mental Retardation | Brain, nervous | | FMR1 |
| (aka Martin Bell syndrome) | system | | |
| Friedreich Ataxia (FRDA) | Brain, nervous | heart | FXN/X25 |
| | system | | |
| Fuchs endothelial corneal | Eye | | TCF4; COL8A2 |
| dystrophy | | | |
| Galactosemia | Carbohydrate | Various-where | GALT, GALK1, and GALE |
| | metabolism | galactose | |
| | disorder | accumulates - | |
| | | liver, brain, eyes | |
| Gastrointestinal Epithelial | | | CISH |
| Cancer, GI cancer | | | |
| Gaucher Disease (Types 1, 2, and | Fat metabolism | Various-liver, | GBA |
| 3, as well as other unusual forms | disorder | spleen, blood, | |
| that may not fit into these types) | | CNS, skeletal | |
| | | system | |
| Griscelli syndrome | | | |
| Glaucoma | eye | | MYOC, TIGR, GLC1A, JOAG, |
| | | | GPOA, OPTN, GLC1E, FIP2, HYPL, |
| | | | NRP, CYP1B1, GLC3A, OPA1, NTG, |
| | | | NPG, CYP1B1, GLC3A, those |
| | | | described in WO2015153780 |
| Glomerulo sclerosis | kidney | | CC chemokine ligand 2 |
| Glycogen Storage Diseases | Metabolism | | SLC2A2, GLUT2, G6PC, G6PT, |
| Types I-VI -See also Cori's | Diseases | | G6PT1, GAA, LAMP2, LAMPB, |
| Disease, Pompe's Disease, | | | AGL, GDE, GBE1, GYS2, PYGL, |
| McArdle's disease, Hers Disease, | | | PFKM, see also Cori's Disease, |
| and Von Gierke's disease | | | Pompe's Disease, McArdle's disease, |
| | | | Hers Disease, and Von Gierke's |
| | | | disease |
| RBC Glycolytic enzyme | blood | | any mutations in a gene for an enzyme |
| deficiency | | | in the glycolysis pathway including |
| | | | mutations in genes for hexokinases I |
| | | | and II, glucokinase, phosphoglucose |
| | | | isomerase, phosphofructokinase, |
| | | | aldolase Bm triosephosphate |
| | | | isomerease, glyceraldehydee-3- |
| | | | phosphate dehydrogenase, |
| | | | phosphoglycerokinase, |
| | | | phosphoglycerate mutase, enolase I, |
| | | | pyruvate kinase |
| Hartnup's disease | Malabsorption | Various- brain, | SLC6A19 |
| | disease | gastrointestinal, | |
| | | skin, | |
| Hearing Loss | ear | | NOX3, Hes5, BDNF, |
| Hemochromatosis (HH) | Iron absorption | Various- | HFE and H63D |
| | regulation | wherever iron | |
| | disease | accumulates, | |
| | | liver, heart, | |
| | | pancreas, joints, | |
| | | pituitary gland | |
| Hemophagocytic | blood | | PRF1, HPLH2, UNC13D, MUNC13- |
| lymphohistiocytosis disorders | | | 4, HPLH3, HLH3, FHL3 |
| Hemorrhagic disorders | blood | | PI, ATT, F5 |
| Hers disease (Glycogen storage | liver | muscle | PYGL |
| disease Type VI) | | | |
| Hereditary angioedema (HAE) | | | kalikrein B1 |
| Hereditary Hemorrhagic | Skin and | | ACVRL1, ENG and SMAD4 |
| Telangiectasia (Osler-Weber- | mucous | | |
| Rendu Syndrome) | membranes | | |
| Hereditary Spherocytosis | blood | | NK1, EPB42, SLC4A1, SPTA1, and |
| | | | SPTB |
| Hereditary Persistence of Fetal | blood | | HBG1, HBG2, BCL11A, promoter |
| Hemoglobin | | | region of HBG 1 and/or 2 (in the |
| | | | CCAAT box) |
| Hemophilia (hemophilia A | blood | | A: FVIII, F8C, HEMA |
| (Classic) a B (aka Christmas | | | B: FVIX, HEMB, FIX |
| disease) and C) | | | C: F9, F11 |
| Hepatic adenoma | liver | | TCF1, HNF1A, MODY3 |
| Hepatic failure, early onset, and | liver | | SCOD1, SCO1 |
| neurologic disorder | | | |
| Hepatic lipase deficiency | liver | | LIPC |
| Hepatoblastoma, cancer and | liver | | CTNNB1, PDGFRL, PDGRL, PRLTS, |
| carcinomas | | | AXIN1, AXIN, CTNNB1, TP53, P53, |
| | | | LFS1, IGF2R, MPRI, MET, CASP8, |
| | | | MCH5 |
| Hermansky-Pudlak syndrome | Skin, eyes, | | HPS1, HPS3, HPS4, HPS5, HPS6, |
| | blood, lung, | | HPS7, DTNBP1, BLOC1, BLOC1S2, |
| | kidneys, | | BLOC3 |
| | intestine | | |
| HIV susceptibility or infection | Immune system | | IL10, CSIF, CMKBR2, CCR2, |
| | | | CMKBR5, CCCKR5 (CCR5), those in |
| | | | WO2015148670A1 |
| Holoprosencephaly (HPE) | brain | | ACVRL1, ENG, SMAD4 |
| (Alobar, Semilobar, and Lobar) | | | |
| Homocystinuria | Metabolic | Various- | CBS, MTHFR, MTR, MTRR, and |
| | disease | connective | MMADHC |
| | | tissue, muscles, | |
| | | CNS, | |
| | | cardiovascular | |
| | | system | |
| HPV | | | HPV16 and HPV18 E6/E7 |
| HSV1, HSV2, and related | eye | | HSV1 genes (immediate early and late |
| keratitis | | | HSV-1 genes (UL1, 1.5, 5, 6, 8, 9, 12, |
| | | | 15, 16, 18, 19, 22, 23, 26, 26.5, 27, 28, |
| | | | 29, 30, 31, 32, 33, 34, 35, 36, 37, 38, |
| | | | 42, 48, 49.5, 50, 52, 54, S6, RL2, RS1, |
| | | | those described in WO2015153789, |
| | | | WO2015153791 |
| Hunter's Syndrome (aka | Lysosomal | Various- liver, | IDS |
| Mucopolysaccharidosis type II) | storage disease | spleen, eye, | |
| | | joint, heart, | |
| | | brain, skeletal | |
| Huntington's disease (HD) and | Brain, nervous | | HD, HTT, IT15, PRNP, PRIP, JPH3, |
| HD-like disorders | system | | JP3, HDL2, TBP, SCA17, PRKCE; |
| | | | IGF1; EP300; RCOR1; PRKCZ; |
| | | | HDAC4; and TGM2, and those |
| | | | described in WO2013130824, |
| | | | WO2015089354 |
| Hurler's Syndrome (aka | Lysosomal | Various- liver, | IDUA, α-L-iduronidase |
| mucopolysaccharidosis type I H, | storage disease | spleen, eye, | |
| MPS IH) | | joint, heart, | |
| | | brain, skeletal | |
| Hurler-Scheie syndrome (aka | Lysosomal | Various- liver, | IDUA, α-L-iduronidase |
| mucopolysaccharidosis type I H- | storage disease | spleen, eye, | |
| S, MPS I H-S) | | joint, heart, | |
| | | brain, skeletal | |
| hyaluronidase deficiency (aka | Soft and | | HYAL1 |
| MPS IX) | connective | | |
| | tissues | | |
| Hyper IgM syndrome | Immune system | | CD40L |
| Hyper- tension caused renal | kidney | | Mineral corticoid receptor |
| damage | | | |
| Immunodeficiencies | Immune System | | CD3E, CD3G, AICDA, AID, HIGM2, |
| | | | TNFRSF5, CD40, UNG, DGU, |
| | | | HIGM4, TNFSF5, CD40LG, HIGM1, |
| | | | IGM, FOXP3, IPEX, AIID, XPID, |
| | | | PIDX, TNFRSF14B, TACI |
| Inborn errors of metabolism: | Metabolism | Various organs | See also: Carbohydrate metabolism |
| including urea cycle disorders, | diseases, liver | and cells | disorders (e.g. galactosemia), Amino |
| organic acidemias), fatty acid | | | acid Metabolism disorders (e.g. |
| oxidation defects, amino | | | phenylketonuria), Fatty acid |
| acidopathies, carbohydrate | | | metabolism (e.g. MCAD deficiency), |
| disorders, mitochondrial | | | Urea Cycle disorders (e.g. |
| disorders | | | Citrullinemia), Organic acidemias (e.g. |
| | | | Maple Syrup Urine disease), |
| | | | Mitochondrial disorders (e.g. |
| | | | MELAS), peroxisomal disorders (e.g. |
| | | | Zellweger syndrome) |
| Inflammation | Various | | IL-10; IL-1 (IL-1a; IL-1b); IL-13; IL- |
| | | | 17 (IL-17a (CTLA8); IL- |
| | | | 17b; IL-17c; IL-17d; IL-17f); II-23; |
| | | | Cx3cr1; ptpn22; TNFa; |
| | | | NOD2/CARD15 for IBD; IL-6; IL-12 |
| | | | (IL-12a; IL-12b); |
| | | | CTLA4; Cx3cl1 |
| Inflammatory Bowel Diseases | Gastrointestinal | Joints, skin | NOD2, IRGM, LRRK2, ATG5, |
| (e.g. Ulcerative Colitis and | | | ATG16L1, IRGM, GATM, ECM1, |
| Chron's Disease) | | | CDH1, LAMB1, HNF4A, GNA12, |
| | | | IL10, CARD9/15. CCR6, IL2RA, |
| | | | MST1, TNFSF15, REL, STAT3, |
| | | | IL23R, IL12B, FUT2 |
| Interstitial renal fibrosis | kidney | | TGF-β type II receptor |
| Job's Syndrome (aka Hyper IgE | Immune System | | STAT3, DOCK8 |
| Syndrome) | | | |
| Juvenile Retinoschisis | eye | | RS1, XLRS1 |
| Kabuki Syndrome 1 | | | MLL4, KMT2D |
| Kennedy Disease (aka | Muscles, brain, | | SBMA/SMAX1/AR |
| Spinobulbar Muscular Atrophy) | nervous system | | |
| Klinefelter syndrome | Various- | | Extra X chromosome in males |
| | particularly | | |
| | those involved | | |
| | in development | | |
| | of male | | |
| | characteristics | | |
| Lafora Disease | Brain, CNS | | EMP2A and EMP2B |
| Leber Congenital Amaurosis | eye | | CRB1, RP12, CORD2, CRD, CRX, |
| | | | IMPDH1, OTX2, AIPL1, CABP4, |
| | | | CCT2, CEP290, CLUAP1, CRB1, |
| | | | CRX, DTHD1, GDF6, GUCY2D, |
| | | | IFT140, IQCB1, KCNJ13, LCA5, |
| | | | LRAT, NMNAT1, PRPH2, RD3, |
| | | | RDH12, RPE65, RP20, RPGRIP1, |
| | | | SPATA7, TULP1, LCA1, LCA4, |
| | | | GUC2D, CORD6, LCA3, |
| Lesch-Nyhan Syndrome | Metabolism | Various - joints, | HPRT1 |
| | disease | cognitive, brain, | |
| | | nervous system | |
| Leukocyte deficiencies and | blood | | ITGB2, CD18, LCAMB, LAD, |
| disorders | | | EIF2B1, EIF2BA, EIF2B2, EIF2B3, |
| | | | EIF2B5, LVWM, CACH, CLE, |
| | | | EIF2B4 |
| Leukemia | Blood | | TAL1, TCL5, SCL, TAL2, FLT3, |
| | | | NBS1, NBS, ZNFN1A1, IK1, LYF1, |
| | | | HOXD4, HOX4B, BCR, CML, PHL, |
| | | | ALL, ARNT, KRAS2, RASK2, |
| | | | GMPS, AF10, ARHGEF12, LARG, |
| | | | KIAA0382, CALM, CLTH, CEBPA, |
| | | | CEBP, CHIC2, BTL, FLT3, KIT, |
| | | | PBT, LPP, NPM1, NUP214, D9S46E, |
| | | | CAN, CAIN, RUNX1, CBFA2, |
| | | | AML1, WHSC1L1, NSD3, FLT3, |
| | | | AF1Q, NPM1, NUMA1, ZNF145, |
| | | | PLZF, PML, MYL, STAT5B, AF10, |
| | | | CALM, CLTH, ARL11, ARLTS1, |
| | | | P2RX7, P2X7, BCR, CML, PHL, |
| | | | ALL, GRAF, NF1, VRNF, WSS, |
| | | | NFNS, PTPN11, PTP2C, SHP2, NS1, |
| | | | BCL2, CCND1, PRAD1, BCL1, |
| | | | TCRA, GATA1, GF1, ERYF1, NFE1, |
| | | | ABL1, NQO1, DIA4, NMOR1, |
| | | | NUP214, D9S46E, CAN, CAIN |
| Limb-girdle muscular dystrophy | muscle | | LGMD |
| diseases | | | |
| Lowe syndrome | brain, eyes, | | OCRL |
| | kidneys | | |
| Lupus glomerulo- nephritis | kidney | | MAPK1 |
| Machado- | Brain, CNS, | | ATX3 |
| Joseph's Disease (also known as | muscle | | |
| Spinocerebellar ataxia Type 3) | | | |
| Macular degeneration | eye | | ABC4, CBC1, CHM1, APOE, |
| | | | C1QTNF5, C2, C3, CCL2, CCR2, |
| | | | CD36, CFB, CFH, CFHR1, CFHR3, |
| | | | CNGB3, CP, CRP, CST3, CTSD, |
| | | | CX3CR1, ELOVL4, ERCC6, FBLN5, |
| | | | FBLN6, FSCN2, HMCN1, HIRAI, |
| | | | IL6, IL8, PLEKHA1, PROM1, |
| | | | PRPH2, RPGR, SERPING1, TCOF1, |
| | | | TIMP3, TLR3 |
| Macular Dystrophy | eye | | BEST1, C1QTNF5, CTNNA1, |
| | | | EFEMP1, ELOVL4, FSCN2, |
| | | | GUCA1B, HMCN1, IMPG1, OTX2, |
| | | | PRDM13, PROM1, PRPH2, RP1L1, |
| | | | TIMP3, ABCA4, CFH, DRAM2, |
| | | | IMG1, MFSD8, ADMD, STGD2, |
| | | | STGD3, RDS, RP7, PRPH, AVMD, |
| | | | AOFMD, VMD2 |
| Malattia Leventinesse | eye | | EFEMP1, FBLN3 |
| Maple Syrup Urine Disease | Metabolism | | BCKDHA, BCKDHB, and DBT |
| | disease | | |
| Marfan syndrome | Connective | Musculoskeletal | FBN1 |
| | tissue | | |
| Maroteaux-Lamy Syndrome (aka | Musculoskeletal | Liver, spleen | ARSB |
| MPS VI) | system, nervous | | |
| | system | | |
| McArdle's Disease (Glycogen | Glycogen | muscle | PYGM |
| Storage Disease Type V) | storage disease | | |
| Medullary cystic kidney disease | kidney | | UMOD, HNFJ, FJHN, MCKD2, |
| | | | ADMCKD2 |
| Metachromatic leukodystrophy | Lysosomal | Nervous system | ARSA |
| | storage disease | | |
| Methylmalonic acidemia (MMA) | Metabolism | | MMAA, MMAB, MUT, MMACHC, |
| | disease | | MMADHC, LMBRD1 |
| Morquio Syndrome (aka MPS IV | Connective | heart | GALNS |
| A and B) | tissue, skin, | | |
| | bone, eyes | | |
| Mucopolysaccharidosis diseases | Lysosomal | | See also Hurler/Scheie syndrome, |
| (Types I H/S, I H, II, III A B and | storage disease - | | Hurler disease, Sanfillipo syndrome, |
| C, I S, IVA and B, IX, VII, and | affects various | | Scheie syndrome, Morquio syndrome, |
| VI) | organs/tissues | | hyaluronidase deficiency, Sly |
| | | | syndrome, and Maroteaux-Lamy |
| | | | syndrome |
| Muscular Atrophy | muscle | | VAPB, VAPC, ALS8, SMN1, SMA1, |
| | | | SMA2, SMA3, SMA4, BSCL2, |
| | | | SPG17, GARS, SMAD1, CMT2D, |
| | | | HEXB, IGHMBP2, SMUBP2, |
| | | | CATF1, SMARD1 |
| Muscular dystrophy | muscle | | FKRP, MDC1C, LGMD2I, LAMA2, |
| | | | LAMM, LARGE, KIAA0609, |
| | | | MDC1D, FCMD, TTID, MYOT, |
| | | | CAPN3, CANP3, DYSF, LGMD2B, |
| | | | SGCG, LGMD2C, DMDA1, SCG3, |
| | | | SGCA, ADL, DAG2, LGMD2D, |
| | | | DMDA2, SGCB, LGMD2E, SGCD, |
| | | | SGD, LGMD2F, CMD1L, TCAP, |
| | | | LGMD2G, CMD1N, TRIM32, HT2A, |
| | | | LGMD2H, FKRP, MDC1C, LGMD2I, |
| | | | TTN, CMD1G, TMD, LGMD2J, |
| | | | POMT1, CAV3, LGMD1C, SEPN1, |
| | | | SELN, RSMD1, PLEC1, PLTN, EBS1 |
| Myotonic dystrophy (Type 1 and | Muscles | Eyes, heart, | CNBP (Type 2) and DMPK (Type 1) |
| Type 2) | | endocrine | |
| Neoplasia | | | PTEN; ATM; ATR; EGFR; ERBB2; |
| | | | ERBB3; ERBB4; |
| | | | Notch1; Notch2; Notch3; Notch4; |
| | | | AKT; AKT2; AKT3; HIF; |
| | | | HIF1a; HIF3a; Met; HRG; Bcl2; |
| | | | PPAR alpha; PPAR |
| | | | gamma; WT1 (Wilms Tumor); FGF |
| | | | Receptor Family |
| | | | members (5 members: 1, 2, 3, 4, 5); |
| | | | CDKN2a; APC; RB |
| | | | (retinoblastoma); MEN1; VHL; |
| | | | BRCA1; BRCA2; AR |
| | | | (Androgen Receptor); TSG101; IGF; |
| | | | IGF Receptor; Igf1 (4 |
| | | | variants); Igf2 (3 variants); Igf 1 |
| | | | Receptor; Igf 2 Receptor; |
| | | | Bax; Bcl2; caspases family (9 |
| | | | members: |
| | | | 1, 2, 3, 4, 6, 7, 8, 9, 12); Kras; Apc |
| Neurofibromatosis (NF) (NF1, | brain, spinal | | NF1, NF2 |
| formerly Recklinghausen's NF, | cord, nerves, | | |
| and NF2) | and skin | | |
| Niemann-Pick Lipidosis (Types | Lysosomal | Various- where | Types A and B: SMPD1; Type C: |
| A, B, and C) | Storage Disease | sphingomyelin | NPC1 or NPC2 |
| | | accumulates, | |
| | | particularly | |
| | | spleen, liver, | |
| | | blood, CNS | |
| Noonan Syndrome | Various - | | PTPN11, SOS1, RAF1 and KRAS |
| | musculoskeletal, | | |
| | heart, eyes, | | |
| | reproductive | | |
| | organs, blood | | |
| Norrie Disease or X-linked | eye | | NDP |
| Familial Exudative | | | |
| Vitreoretinopathy | | | |
| North Carolina Macular | eye | | MCDR1 |
| Dystrophy | | | |
| Osteogenesis imperfecta (OI) | bones, | | COL1A1, COL1A2, CRTAP, P3H |
| (Types I, II, III, IV, V, VI, VII) | musculoskeletal | | |
| Osteopetrosis | bones | | LRP5, BMND1, LRP7, LR3, OPPG, |
| | | | VBCH2, CLCN7, CLC7, OPTA2, |
| | | | OSTM1, GL, TCIRG1, TIRC7, |
| | | | OC116, OPTB1 |
| Patau's Syndrome | Brain, heart, | | Additional copy of chromosome 13 |
| (Trisomy 13) | skeletal system | | |
| Parkinson's disease (PD) | Brain, nervous | | SNCA (PARK1), UCHL1 (PARK 5), |
| | system | | and LRRK2 (PARK8), (PARK3), |
| | | | PARK2, PARK4, PARK7 (PARK7), |
| | | | PINK1 (PARK6); x-Synuclein, DJ-1, |
| | | | Parkin, NR4A2, NURR1, NOT, |
| | | | TINUR, SNCAIP, TBP, SCA17, |
| | | | NCAP, PRKN, PDJ, DBH, NDUFV2 |
| Pattern Dystrophy of the RPE | eye | | RDS/peripherin |
| Phenylketonuria (PKU) | Metabolism | Various due to | PAH, PKU1, QDPR, DHPR, PTS |
| | disorder | build-up of | |
| | | phenylalanine, | |
| | | phenyl ketones | |
| | | in tissues and | |
| | | CNS | |
| Polycystic kidney and hepatic | Kidney, liver | | FCYT, PKHD1, ARPKD, PKD1, |
| disease | | | PKD2, PKD4, PKDTS, PRKCSH, |
| | | | G19P1, PCLD, SEC63 |
| Pompe's Disease | Glycogen | Various - heart, | GAA |
| | storage disease | liver, spleen | |
| Porphyria (actually refers to a | Various- | | ALAD, ALAS2, CPOX, FECH, |
| group of different diseases all | wherever heme | | HMBS, PPOX, UROD, or UROS |
| having a specific heme | precursors | | |
| production process abnormality) | accumulate | | |
| posterior polymorphous corneal | eyes | | TCF4; COL8A2 |
| dystrophy | | | |
| Primary Hyperoxaluria (e.g. type | Various - eyes, | | LDHA (lactate dehydrogenase A) and |
| 1) | heart, kidneys, | | hydroxyacid oxidase 1 (HAO1) |
| | skeletal system | | |
| Primary Open Angle Glaucoma | eyes | | MYOC |
| (POAG) | | | |
| Primary sclerosing cholangitis | Liver, | | TCF4; COL8A2 |
| | gallbladder | | |
| Progeria (also called Hutchinson- | All | | LMNA |
| Gilford progeria syndrome) | | | |
| Prader-Willi Syndrome | Musculoskeletal | | Deletion of region of short arm of |
| | system, brain, | | chromosome 15, including UBE3A |
| | reproductive | | |
| | and endocrine | | |
| | system | | |
| Prostate Cancer | prostate | | HOXB13, MSMB, GPRC6A, TP53 |
| Pyruvate Dehydrogenase | Brain, nervous | | PDHA1 |
| Deficiency | system | | |
| Kidney/Renal carcinoma | kidney | | RLIP76, VEGF |
| Rett Syndrome | Brain | | MECP2, RTT, PPMX, MRX16, |
| | | | MRX79, CDKL5, STK9, MECP2, |
| | | | RTT, PPMX, MRX16, MRX79, x- |
| | | | Synuclein, DJ-1 |
| Retinitis pigmentosa (RP) | eye | | ADIPOR1, ABCA4, AGBL5, |
| | | | ARHGEF18, ARL2BP, ARL3, ARL6, |
| | | | BEST1, BBS1, BBS2, C2ORF71, |
| | | | C8ORF37, CA4, CERKL, CLRN1, |
| | | | CNGA1, CMGB1, CRB1, CRX, |
| | | | CYP4V2, DHDDS, DHX38, EMC1, |
| | | | EYS, FAM161A, FSCN2, GPR125, |
| | | | GUCA1B, HK1, HPRPF3, HGSNAT, |
| | | | IDH3B, IMPDH1, IMPG2, IFT140, |
| | | | IFT172, KLHL7, KIAA1549, KIZ, |
| | | | LRAT, MAK, MERTK, MVK, NEK2, |
| | | | NUROD1, NR2E3, NRL, OFD1, |
| | | | PDE6A, PDE6B, PDE6G, POMGNT1, |
| | | | PRCD, PROM1, PRPF3, PRPF4, |
| | | | PRPF6, PRPF8, PRPF31, PRPH2, |
| | | | RPB3, RDH12, REEP6, RP39, RGR, |
| | | | RHO, RLBP1, ROM1, RP1, RP1L1, |
| | | | RPY, RP2, RP9, RPE65, RPGR, |
| | | | SAMD11, SAG, SEMA4A, SLC7A14, |
| | | | SNRNP200, SPP2, SPATA7, TRNT1, |
| | | | TOPORS, TTC8, TULP1, USH2A, |
| | | | ZFN408, ZNF513, see also |
| | | | 20120204282 |
| Scheie syndrome (also known as | Various- liver, | | IDUA, α-L-iduronidase |
| mucopolysaccharidosis type I | spleen, eye, | | |
| S(MPS I-S)) | joint, heart, | | |
| | brain, skeletal | | |
| Schizophrenia | Brain | | Neuregulin1 (Nrg1); Erb4 (receptor for |
| | | | Neuregulin); |
| | | | Complexin1 (Cplx1); Tph1 |
| | | | Tryptophan hydroxylase; Tph2 |
| | | | Tryptophan hydroxylase 2; Neurexin |
| | | | 1; GSK3; GSK3a; |
| | | | GSK3b; 5-HTT (Slc6a4); COMT; |
| | | | DRD (Drd1a); SLC6A3; DAOA; |
| | | | DTNBP1; Dao (Dao1); TCF4; |
| | | | COL8A2 |
| Secretase Related Disorders | Various | | APH-1 (alpha and beta); PSEN1; |
| | | | NCSTN; PEN-2; Nos1, Parp1, Nat1, |
| | | | Nat2, CTSB, APP, APH1B, PSEN2, |
| | | | PSENEN, BACE1, ITM2B, CTSD, |
| | | | NOTCH1, TNF, INS, DYT10, |
| | | | ADAM17, APOE, ACE, STN, TP53, |
| | | | IL6, NGFR, IL1B, ACHE, CTNNB1, |
| | | | IGF1, IFNG, NRG1, CASP3, MAPK1, |
| | | | CDH1, APBB1, HMGCR, CREB1, |
| | | | PTGS2, HES1, CAT, TGFB1, ENO2, |
| | | | ERBB4, TRAPPC10, MAOB, NGF, |
| | | | MMP12, JAG1, CD40LG, PPARG, |
| | | | FGF2, LRP1, NOTCH4, MAPK8, |
| | | | PREP, NOTCH3, PRNP, CTSG, EGF, |
| | | | REN, CD44, SELP, GHR, ADCYAP1, |
| | | | INSR, GFAP, MMP3, MAPK10, SP1, |
| | | | MYC, CTSE, PPARA, JUN, TIMP1, |
| | | | IL5, IL1A, MMP9, HTR4, HSPG2, |
| | | | KRAS, CYCS, SMG1, IL1R1, |
| | | | PROK1, MAPK3, NTRK1, IL13, |
| | | | MME, TKT, CXCR2, CHRM1, |
| | | | ATXN1, PAWR, NOTCJ2, M6PR, |
| | | | CYP46A1, CSNK1D, MAPK14, |
| | | | PRG2, PRKCA, L1 CAM, CD40, |
| | | | NR1I2, JAG2, CTNND1, CMA1, |
| | | | SORT1, DLK1, THEM4, JUP, CD46, |
| | | | CCL11, CAV3, RNASE3, HSPA8, |
| | | | CASP9, CYP3A4, CCR3, TFAP2A, |
| | | | SCP2, CDK4, JOF1A, TCF7L2, |
| | | | B3GALTL, MDM2, RELA, CASP7, |
| | | | IDE, FANP4, CASK, ADCYAP1R1, |
| | | | ATF4, PDGFA, C21ORF33, SCG5, |
| | | | RMF123, NKFB1, ERBB2, CAV1, |
| | | | MMP7, TGFA, RXRA, STX1A, |
| | | | PSMC4, P2RY2, TNFRSF21, DLG1, |
| | | | NUMBL, SPN, PLSCR1, UBQLN2, |
| | | | UBQLN1, PCSK7, SPON1, SILV, |
| | | | QPCT, HESS, GCC1 |
| Selective IgA Deficiency | Immune system | | Type 1: MSH5; Type 2: TNFRSF13B |
| Severe Combined | Immune system | | JAK3, JAKL, DCLRE1C, ARTEMIS, |
| Immunodeficiency (SCID) and | | | SCIDA, RAG1, RAG2, ADA, PTPRC, |
| SCID-XI, and ADA-SCID | | | CD45, LCA, IL7R, CD3D, T3D, |
| | | | IL2RG, SCIDX1, SCIDX, IMD4, |
| | | | those identified in US Pat. App. Pub. |
| | | | 20110225664, 20110091441, |
| | | | 20100229252, 20090271881 and |
| | | | 20090222937; |
| Sickle cell disease | blood | | HBB, BCL11A, BCL11Ae, cis- |
| | | | regulatory elements of the B-globin |
| | | | locus, HBG 1/2 promoter, HBG distal |
| | | | CCAAT box region between −92 and −130 |
| | | | of the HBG Transcription Start |
| | | | Site, those described in |
| | | | WO2015148863, WO 2013/126794, |
| | | | US Pat. Pub. 20110182867 |
| Sly Syndrome (aka MPS VII) | | | GUSB |
| Spinocerebellar Ataxias (SCA | | | ATXN1, ATXN2, ATX3 |
| types 1, 2, 3, 6, 7, 8, 12 and 17) | | | |
| Sorsby Fundus Dystrophy | eye | | TIMP3 |
| Stargardt disease | eye | | ABCR, ELOVL4, ABCA4, PROM1 |
| Tay-Sachs Disease | Lysosomal | Various - CNS, | HEX-A |
| | Storage disease | brain, eye | |
| Thalassemia (Alpha, Beta, Delta) | blood | | HBA1, HBA2 (Alpha), HBB (Beta), |
| | | | HBB and HBD (delta), LCRB, |
| | | | BCL11A, BCL11Ae, cis-regulatory |
| | | | elements of the B-globin locus, HBG |
| | | | 1/2 promoter, those described in |
| | | | WO2015148860, US Pat. Pub. |
| | | | 20110182867, 2015/148860 |
| Thymic Aplasia (DiGeorge | Immune system, | | deletion of 30 to 40 genes in the |
| Syndrome; 22q11.2 deletion | thymus | | middle of chromosome 22 at |
| syndrome) | | | a location known as 22q11.2, including |
| | | | TBX1, DGCR8 |
| Transthyretin amyloidosis | liver | | TTR (transthyretin) |
| (ATTR) | | | |
| trimethylaminuria | Metabolism | | FMO3 |
| | disease | | |
| Trinucleotide Repeat Disorders | Various | | HTT; SBMA/SMAX1/AR; |
| (generally) | | | FXN/X25 ATX3; |
| | | | ATXN1; ATXN2; |
| | | | DMPK; Atrophin-1 and Atn1 |
| | | | (DRPLA Dx); CBP (Creb-BP - global |
| | | | instability); VLDLR; Atxn7; Atxn10; |
| | | | FEN1, TNRC6A, PABPN1, JPH3, |
| | | | MED15, ATXN1, ATXN3, TBP, |
| | | | CACNA1A, ATXN80S, PPP2R2B, |
| | | | ATXN7, TNRC6B, TNRC6C, CELF3, |
| | | | MAB21L1, MSH2, TMEM185A, |
| | | | SIX5, CNPY3, RAXE, GNB2, RPL14, |
| | | | ATXN8, ISR, TTR, EP400, GIGYF2, |
| | | | OGG1, STC1, CNDP1, C10ORF2, |
| | | | MAML3, DKC1, PAXIP1, CASK, |
| | | | MAPT, SP1, POLG, AFF2, THBS1, |
| | | | TP53, ESR1, CGGBP1, ABT1, KLK3, |
| | | | PRNP, JUN, KCNN3, BAX, FRAXA, |
| | | | KBTBD10, MBNL1, RAD51, |
| | | | NCOA3, ERDA1, TSC1, COMP, |
| | | | GGLC, RRAD, MSH3, DRD2, CD44, |
| | | | CTCF, CCND1, CLSPN, MEF2A, |
| | | | PTPRU, GAPDH, TRIM22, WT1, |
| | | | AHR, GPX1, TPMT, NDP, ARX, |
| | | | TYR, EGR1, UNG, NUMBL, FABP2, |
| | | | EN2, CRYGC, SRP14, CRYGB, |
| | | | PDCD1, HOXA1, ATXN2L, PMS2, |
| | | | GLA, CBL, FTH1, IL12RB2, OTX2, |
| | | | HOXA5, POLG2, DLX2, AHRR, |
| | | | MANF, RMEM158, see also |
| | | | 20110016540 |
| Turner's Syndrome (XO) | Various - | | Monosomy X |
| | reproductive | | |
| | organs, and sex | | |
| | characteristics, | | |
| | vasculature | | |
| Tuberous Sclerosis | CNS, heart, | | TSC1, TSC2 |
| | kidneys | | |
| Usher syndrome (Types I, II, and | Ears, eyes | | ABHD12, CDH23, CIB2, CLRN1, |
| III) | | | DFNB31, GPR98, HARS, MYO7A, |
| | | | PCDH15, USH1C, USH1G, USH2A, |
| | | | USH11A, those described in |
| | | | WO2015134812A1 |
| Velocardiofacial syndrome (aka | Various - | | Many genes are deleted, COM, TBX1, |
| 22q11.2 deletion syndrome, | skeletal, heart, | | and other are associated with |
| DiGeorge syndrome, conotruncal | kidney, immune | | symptoms |
| anomaly face syndrome (CTAF), | system, brain | | |
| autosomal dominant Opitz G/BB | | | |
| syndrome or Cayler cardiofacial | | | |
| syndrome) | | | |
| Von Gierke's Disease (Glycogen | Glycogen | Various - liver, | G6PC and SLC37A4 |
| Storage Disease type I) | Storage disease | kidney | |
| Von Hippel-Lindau Syndrome | Various - cell | CNS, Kidney, | VHL |
| | growth | Eye, visceral | |
| | regulation | organs | |
| | disorder | | |
| Von Willebrand Disease (Types | blood | | VWF |
| I, II and III) | | | |
| Wilson Disease | Various - | Liver, brains, | ATP7B |
| | Copper Storage | eyes, other | |
| | Disease | tissues where | |
| | | copper builds up | |
| Wiskott-Aldrich Syndrome | Immune System | | WAS |
| Xeroderma Pigmentosum | Skin | Nervous system | POLH |
| XXX Syndrome | Endocrine, brain | | X chromosome trisomy |

In some embodiments, the engineered nucleic acid modification systems or components thereof of the present invention can be used treat or prevent a disease in a subject by modifying one or more genes associated with one or more cellular functions, such as any one or more of those in Table 6. In some embodiments, the disease is a genetic disease or disorder. In some of embodiments, the engineered nucleic acid modification systems or component thereof of the present invention can modify one or more genes or polynucleotides associated with one or more genetic diseases such as any set forth in Table 6.

TABLE 6
Exemplary Genes controlling Cellular Functions

| CELLULAR FUNCTION | GENES |
|---|---|
| PI3K/AKT Signaling | PRKCE; ITGAM; ITGA5; IRAK1; PRKAA2; EIF2AK2; |
| | PTEN; EIF4E; PRKCZ; GRK6; MAPK1; TSC1; PLK1; |
| | AKT2; IKBKB; PIK3CA; CDK8; CDKN1B; NFKB2; BCL2; |
| | PIK3CB; PPP2R1A; MAPK8; BCL2L1; MAPK3; TSC2; |
| | ITGA1; KRAS; EIF4EBP1; RELA; PRKCD; NOS3; |
| | PRKAA1; MAPK9; CDK2; PPP2CA; PIM1; ITGB7; |
| | YWHAZ; ILK; TP53; RAF1; IKBKG; RELB; DYRK1A; |
| | CDKN1A; ITGB1; MAP2K2; JAK1; AKT1; JAK2; PIK3R1; |
| | CHUK; PDPK1; PPP2R5C; CTNNB1; MAP2K1; NFKB1; |
| | PAK3; ITGB3; CCND1; GSK3A; FRAP1; SFN; ITGA2; |
| | TTK; CSNK1A1; BRAF; GSK3B; AKT3; FOXO1; SGK; |
| | HSP90AA1; RPS6KB1 |
| ERK/MAPK Signaling | PRKCE; ITGAM; ITGA5; HSPB1; IRAK1; PRKAA2; |
| | EIF2AK2; RAC1; RAP1A; TLN1; EIF4E; ELK1; GRK6; |
| | MAPK1; RAC2; PLK1; AKT2; PIK3CA; CDK8; CREB1; |
| | PRKCI; PTK2; FOS; RPS6KA4; PIK3CB; PPP2R1A; |
| | PIK3C3; MAPK8; MAPK3; ITGA1; ETS1; KRAS; MYCN; |
| | EIF4EBP1; PPARG; PRKCD; PRKAA1; MAPK9; SRC; |
| | CDK2; PPP2CA; PIM1; PIK3C2A; ITGB7; YWHAZ; |
| | PPP1CC; KSR1; PXN; RAF1; FYN; DYRK1A; ITGB1; |
| | MAP2K2; PAK4; PIK3R1; STAT3; PPP2R5C; MAP2K1; |
| | PAK3; ITGB3; ESR1; ITGA2; MYC; TTK; CSNK1A1; |
| | CRKL; BRAF; ATF4; PRKCA; SRF; STAT1; SGK |
| Glucocorticoid Receptor | RAC1; TAF4B; EP300; SMAD2; TRAF6; PCAF; ELK1; |
| Signaling | MAPK1; SMAD3; AKT2; IKBKB; NCOR2; UBE2I; |
| | PIK3CA; CREB1; FOS; HSPA5; NFKB2; BCL2; |
| | MAP3K14; STAT5B; PIK3CB; PIK3C3; MAPK8; BCL2L1; |
| | MAPK3; TSC22D3; MAPK10; NRIP1; KRAS; MAPK13; |
| | RELA; STAT5A; MAPK9; NOS2A; PBX1; NR3C1; |
| | PIK3C2A; CDKN1C; TRAF2; SERPINE1; NCOA3; |
| | MAPK14; TNF; RAF1; IKBKG; MAP3K7; CREBBP; |
| | CDKN1A; MAP2K2; JAK1; IL8; NCOA2; AKT1; JAK2; |
| | PIK3R1; CHUK; STAT3; MAP2K1; NFKB1; TGFBR1; |
| | ESR1; SMAD4; CEBPB; JUN; AR; AKT3; CCL2; MMP1; |
| | STAT1; IL6; HSP90AA1 |
| Axonal Guidance Signaling | PRKCE; ITGAM; ROCK1; ITGA5; CXCR4; ADAM12; |
| | IGF1; RAC1; RAP1A; EIF4E; PRKCZ; NRP1; NTRK2; |
| | ARHGEF7; SMO; ROCK2; MAPK1; PGF; RAC2; |
| | PTPN11; GNAS; AKT2; PIK3CA; ERBB2; PRKCI; PTK2; |
| | CFL1; GNAQ; PIK3CB; CXCL12; PIK3C3; WNT11; |
| | PRKD1; GNB2L1; ABL1; MAPK3; ITGA1; KRAS; RHOA; |
| | PRKCD; PIK3C2A; ITGB7; GLI2; PXN; VASP; RAF1; |
| | FYN; ITGB1; MAP2K2; PAK4; ADAM17; AKT1; PIK3R1; |
| | GLI1; WNT5A; ADAM10; MAP2K1; PAK3; ITGB3; |
| | CDC42; VEGFA; ITGA2; EPHA8; CRKL; RND1; GSK3B; |
| | AKT3; PRKCA |
| Ephrin Receptor Signaling | PRKCE; ITGAM; ROCK1; ITGA5; CXCR4; IRAK1; |
| Actin Cytoskeleton | PRKAA2; EIF2AK2; RAC1; RAP1A; GRK6; ROCK2; |
| Signaling | MAPK1; PGF; RAC2; PTPN11; GNAS; PLK1; AKT2; |
| | DOK1; CDK8; CREB1; PTK2; CFL1; GNAQ; MAP3K14; |
| | CXCL12; MAPK8; GNB2L1; ABL1; MAPK3; ITGA1; |
| | KRAS; RHOA; PRKCD; PRKAA1; MAPK9; SRC; CDK2; |
| | PIM1; ITGB7; PXN; RAF1; FYN; DYRK1A; ITGB1; |
| | MAP2K2; PAK4; AKT1; JAK2; STAT3; ADAM10; |
| | MAP2K1; PAK3; ITGB3; CDC42; VEGFA; ITGA2; |
| | EPHA8; TTK; CSNK1A1; CRKL; BRAF; PTPN13; ATF4; |
| | AKT3; SGK |
| | ACTN4; PRKCE; ITGAM; ROCK1; ITGA5; IRAK1; |
| | PRKAA2; EIF2AK2; RAC1; INS; ARHGEF7; GRK6; |
| | ROCK2; MAPK1; RAC2; PLK1; AKT2; PIK3CA; CDK8; |
| | PTK2; CFL1; PIK3CB; MYH9; DIAPH1; PIK3C3; MAPK8; |
| | F2R; MAPK3; SLC9A1; ITGA1; KRAS; RHOA; PRKCD; |
| | PRKAA1; MAPK9; CDK2; PIM1; PIK3C2A; ITGB7; |
| | PPP1CC; PXN; VIL2; RAF1; GSN; DYRK1A; ITGB1; |
| | MAP2K2; PAK4; PIP5K1A; PIK3R1; MAP2K1; PAK3; |
| | ITGB3; CDC42; APC; ITGA2; TTK; CSNK1A1; CRKL; |
| | BRAF; VAV3; SGK |
| Huntington's Disease | PRKCE; IGF1; EP300; RCOR1; PRKCZ; HDAC4; TGM2; |
| Signaling | MAPK1; CAPNS1; AKT2; EGFR; NCOR2; SP1; CAPN2; |
| | PIK3CA; HDAC5; CREB1; PRKCI; HSPA5; REST; |
| | GNAQ; PIK3CB; PIK3C3; MAPK8; IGF1R; PRKD1; |
| | GNB2L1; BCL2L1; CAPN1; MAPK3; CASP8; HDAC2; |
| | HDAC7A; PRKCD; HDAC11; MAPK9; HDAC9; PIK3C2A; |
| | HDAC3; TP53; CASP9; CREBBP; AKT1; PIK3R1; |
| | PDPK1; CASP1; APAF1; FRAP1; CASP2; JUN; BAX; |
| | ATF4; AKT3; PRKCA; CLTC; SGK; HDAC6; CASP3 |
| Apoptosis Signaling | PRKCE; ROCK1; BID; IRAK1; PRKAA2; EIF2AK2; BAK1; |
| | BIRC4; GRK6; MAPK1; CAPNS1; PLK1; AKT2; IKBKB; |
| | CAPN2; CDK8; FAS; NFKB2; BCL2; MAP3K14; MAPK8; |
| | BCL2L1; CAPN1; MAPK3; CASP8; KRAS; RELA; |
| | PRKCD; PRKAA1; MAPK9; CDK2; PIM1; TP53; TNF; |
| | RAF1; IKBKG; RELB; CASP9; DYRK1A; MAP2K2; |
| | CHUK; APAF1; MAP2K1; NFKB1; PAK3; LMNA; CASP2; |
| | BIRC2; TTK; CSNK1A1; BRAF; BAX; PRKCA; SGK; |
| | CASP3; BIRC3; PARP1 |
| B Cell Receptor Signaling | RAC1; PTEN; LYN; ELK1; MAPK1; RAC2; PTPN11; |
| | AKT2; IKBKB; PIK3CA; CREB1; SYK; NFKB2; CAMK2A; |
| | MAP3K14; PIK3CB; PIK3C3; MAPK8; BCL2L1; ABL1; |
| | MAPK3; ETS1; KRAS; MAPK13; RELA; PTPN6; MAPK9; |
| | EGR1; PIK3C2A; BTK; MAPK14; RAF1; IKBKG; RELB; |
| | MAP3K7; MAP2K2; AKT1; PIK3R1; CHUK; MAP2K1; |
| | NFKB1; CDC42; GSK3A; FRAP1; BCL6; BCL10; JUN; |
| | GSK3B; ATF4; AKT3; VAV3; RPS6KB1 |
| Leukocyte Extravasation | ACTN4; CD44; PRKCE; ITGAM; ROCK1; CXCR4; CYBA; |
| Signaling | RAC1; RAP1A; PRKCZ; ROCK2; RAC2; PTPN11; |
| | MMP14; PIK3CA; PRKCI; PTK2; PIK3CB; CXCL12; |
| | PIK3C3; MAPK8; PRKD1; ABL1; MAPK10; CYBB; |
| | MAPK13; RHOA; PRKCD; MAPK9; SRC; PIK3C2A; BTK; |
| | MAPK14; NOX1; PXN; VIL2; VASP; ITGB1; MAP2K2; |
| | CTNND1; PIK3R1; CTNNB1; CLDN1; CDC42; F11R; ITK; |
| | CRKL; VAV3; CTTN; PRKCA; MMP1; MMP9 |
| Integrin Signaling | ACTN4; ITGAM; ROCK1; ITGA5; RAC1; PTEN; RAP1A; |
| | TLN1; ARHGEF7; MAPK1; RAC2; CAPNS1; AKT2; |
| | CAPN2; PIK3CA; PTK2; PIK3CB; PIK3C3; MAPK8; |
| | CAV1; CAPN1; ABL1; MAPK3; ITGA1; KRAS; RHOA; |
| | SRC; PIK3C2A; ITGB7; PPP1CC; ILK; PXN; VASP; |
| | RAF1; FYN; ITGB1; MAP2K2; PAK4; AKT1; PIK3R1; |
| | TNK2; MAP2K1; PAK3; ITGB3; CDC42; RND3; ITGA2; |
| | CRKL; BRAF; GSK3B; AKT3 |
| Acute Phase Response | IRAK1; SOD2; MYD88; TRAF6; ELK1; MAPK1; PTPN11; |
| Signaling | AKT2; IKBKB; PIK3CA; FOS; NFKB2; MAP3K14; |
| | PIK3CB; MAPK8; RIPK1; MAPK3; IL6ST; KRAS; |
| | MAPK13; IL6R; RELA; SOCS1; MAPK9; FTL; NR3C1; |
| | TRAF2; SERPINE1; MAPK14; TNF; RAF1; PDK1; |
| | IKBKG; RELB; MAP3K7; MAP2K2; AKT1; JAK2; PIK3R1; |
| | CHUK; STAT3; MAP2K1; NFKB1; FRAP1; CEBPB; JUN; |
| | AKT3; IL1R1; IL6 |
| PTEN Signaling | ITGAM; ITGA5; RAC1; PTEN; PRKCZ; BCL2L11; |
| | MAPK1; RAC2; AKT2; EGFR; IKBKB; CBL; PIK3CA; |
| | CDKN1B; PTK2; NFKB2; BCL2; PIK3CB; BCL2L1; |
| | MAPK3; ITGA1; KRAS; ITGB7; ILK; PDGFRB; INSR; |
| | RAF1; IKBKG; CASP9; CDKN1A; ITGB1; MAP2K2; |
| | AKT1; PIK3R1; CHUK; PDGFRA; PDPK1; MAP2K1; |
| | NFKB1; ITGB3; CDC42; CCND1; GSK3A; ITGA2; |
| | GSK3B; AKT3; FOXO1; CASP3; RPS6KB1 |
| p53 Signaling | PTEN; EP300; BBC3; PCAF; FASN; BRCA1; GADD45A; |
| Aryl Hydrocarbon Receptor | BIRC5; AKT2; PIK3CA; CHEK1; TP53INP1; BCL2; |
| Signaling | PIK3CB; PIK3C3; MAPK8; THBS1; ATR; BCL2L1; E2F1; |
| | PMAIP1; CHEK2; TNFRSF10B; TP73; RB1; HDAC9; |
| | CDK2; PIK3C2A; MAPK14; TP53; LRDD; CDKN1A; |
| | HIPK2; AKT1; PIK3R1; RRM2B; APAF1; CTNNB1; |
| | SIRT1; CCND1; PRKDC; ATM; SFN; CDKN2A; JUN; |
| | SNAI2; GSK3B; BAX; AKT3 |
| | HSPB1; EP300; FASN; TGM2; RXRA; MAPK1; NQO1; |
| | NCOR2; SP1; ARNT; CDKN1B; FOS; CHEK1; |
| | SMARCA4; NFKB2; MAPK8; ALDH1A1; ATR; E2F1; |
| | MAPK3; NRIP1; CHEK2; RELA; TP73; GSTP1; RB1; |
| | SRC; CDK2; AHR; NFE2L2; NCOA3; TP53; TNF; |
| | CDKN1A; NCOA2; APAF1; NFKB1; CCND1; ATM; ESR1; |
| | CDKN2A; MYC; JUN; ESR2; BAX; IL6; CYP1B1; |
| | HSP90AA1 |
| Xenobiotic Metabolism | PRKCE; EP300; PRKCZ; RXRA; MAPK1; NQO1; |
| Signaling | NCOR2; PIK3CA; ARNT; PRKCI; NFKB2; CAMK2A; |
| | PIK3CB; PPP2R1A; PIK3C3; MAPK8; PRKD1; |
| | ALDH1A1; MAPK3; NRIP1; KRAS; MAPK13; PRKCD; |
| | GSTP1; MAPK9; NOS2A; ABCB1; AHR; PPP2CA; FTL; |
| | NFE2L2; PIK3C2A; PPARGC1A; MAPK14; TNF; RAF1; |
| | CREBBP; MAP2K2; PIK3R1; PPP2R5C; MAP2K1; |
| | NFKB1; KEAP1; PRKCA; EIF2AK3; IL6; CYP1B1; |
| | HSP90AA1 |
| SAPK/JNK Signaling | PRKCE; IRAK1; PRKAA2; EIF2AK2; RAC1; ELK1; |
| | GRK6; MAPK1; GADD45A; RAC2; PLK1; AKT2; PIK3CA; |
| | FADD; CDK8; PIK3CB; PIK3C3; MAPK8; RIPK1; |
| | GNB2L1; IRS1; MAPK3; MAPK10; DAXX; KRAS; |
| | PRKCD; PRKAA1; MAPK9; CDK2; PIM1; PIK3C2A; |
| | TRAF2; TP53; LCK; MAP3K7; DYRK1A; MAP2K2; |
| | PIK3R1; MAP2K1; PAK3; CDC42; JUN; TTK; CSNK1A1; |
| | CRKL; BRAF; SGK |
| PPAr/RXR Signaling | PRKAA2; EP300; INS; SMAD2; TRAF6; PPARA; FASN; |
| | RXRA; MAPK1; SMAD3; GNAS; IKBKB; NCOR2; |
| | ABCA1; GNAQ; NFKB2; MAP3K14; STAT5B; MAPK8; |
| | IRS1; MAPK3; KRAS; RELA; PRKAA1; PPARGC1A; |
| | NCOA3; MAPK14; INSR; RAF1; IKBKG; RELB; MAP3K7; |
| | CREBBP; MAP2K2; JAK2; CHUK; MAP2K1; NFKB1; |
| | TGFBR1; SMAD4; JUN; IL1R1; PRKCA; IL6; HSP90AA1; |
| | ADIPOQ |
| NF-KB Signaling | IRAK1; EIF2AK2; EP300; INS; MYD88; PRKCZ; TRAF6; |
| | TBK1; AKT2; EGFR; IKBKB; PIK3CA; BTRC; NFKB2; |
| | MAP3K14; PIK3CB; PIK3C3; MAPK8; RIPK1; HDAC2; |
| | KRAS; RELA; PIK3C2A; TRAF2; TLR4; PDGFRB; TNF; |
| | INSR; LCK; IKBKG; RELB; MAP3K7; CREBBP; AKT1; |
| | PIK3R1; CHUK; PDGFRA; NFKB1; TLR2; BCL10; |
| | GSK3B; AKT3; TNFAIP3; IL1R1 |
| Neuregulin Signaling | ERBB4; PRKCE; ITGAM; ITGA5; PTEN; PRKCZ; ELK1; |
| Wnt & Beta catenin | MAPK1; PTPN11; AKT2; EGFR; ERBB2; PRKCI; |
| Signaling | CDKN1B; STAT5B; PRKD1; MAPK3; ITGA1; KRAS; |
| | PRKCD; STAT5A; SRC; ITGB7; RAF1; ITGB1; MAP2K2; |
| | ADAM17; AKT1; PIK3R1; PDPK1; MAP2K1; ITGB3; |
| | EREG; FRAP1; PSEN1; ITGA2; MYC; NRG1; CRKL; |
| | AKT3; PRKCA; HSP90AA1; RPS6KB1 |
| | CD44; EP300; LRP6; DVL3; CSNK1E; GJA1; SMO; |
| | AKT2; PIN1; CDH1; BTRC; GNAQ; MARK2; PPP2R1A; |
| | WNT11; SRC; DKK1; PPP2CA; SOX6; SFRP2; ILK; |
| | LEF1; SOX9; TP53; MAP3K7; CREBBP; TCF7L2; AKT1; |
| | PPP2R5C; WNT5A; LRP5; CTNNB1; TGFBR1; CCND1; |
| | GSK3A; DVL1; APC; CDKN2A; MYC; CSNK1A1; GSK3B; |
| | AKT3; SOX2 |
| Insulin Receptor Signaling | PTEN; INS; EIF4E; PTPN1; PRKCZ; MAPK1; TSC1; |
| | PTPN11; AKT2; CBL; PIK3CA; PRKCI; PIK3CB; PIK3C3; |
| | MAPK8; IRS1; MAPK3; TSC2; KRAS; EIF4EBP1; |
| | SLC2A4; PIK3C2A; PPP1CC; INSR; RAF1; FYN; |
| | MAP2K2; JAK1; AKT1; JAK2; PIK3R1; PDPK1; MAP2K1; |
| | GSK3A; FRAP1; CRKL; GSK3B; AKT3; FOXO1; SGK; |
| | RPS6KB1 |
| IL-6 Signaling | HSPB1; TRAF6; MAPKAPK2; ELK1; MAPK1; PTPN11; |
| | IKBKB; FOS; NFKB2; MAP3K14; MAPK8; MAPK3; |
| | MAPK10; IL6ST; KRAS; MAPK13; IL6R; RELA; SOCS1; |
| | MAPK9; ABCB1; TRAF2; MAPK14; TNF; RAF1; IKBKG; |
| | RELB; MAP3K7; MAP2K2; IL8; JAK2; CHUK; STAT3; |
| | MAP2K1; NFKB1; CEBPB; JUN; IL1R1; SRF; IL6 |
| Hepatic Cholestasis | PRKCE; IRAK1; INS; MYD88; PRKCZ; TRAF6; PPARA; |
| | RXRA; IKBKB; PRKCI; NFKB2; MAP3K14; MAPK8; |
| | PRKD1; MAPK10; RELA; PRKCD; MAPK9; ABCB1; |
| | TRAF2; TLR4; TNF; INSR; IKBKG; RELB; MAP3K7; IL8; |
| | CHUK; NR1H2; TJP2; NFKB1; ESR1; SREBF1; FGFR4; |
| | JUN; IL1R1; PRKCA; IL6 |
| IGF-1 Signaling | IGF1; PRKCZ; ELK1; MAPK1; PTPN11; NEDD4; AKT2; |
| | PIK3CA; PRKCI; PTK2; FOS; PIK3CB; PIK3C3; MAPK8; |
| | IGF1R; IRS1; MAPK3; IGFBP7; KRAS; PIK3C2A; |
| | YWHAZ; PXN; RAF1; CASP9; MAP2K2; AKT1; PIK3R1; |
| | PDPK1; MAP2K1; IGFBP2; SFN; JUN; CYR61; AKT3; |
| | FOXO1; SRF; CTGF; RPS6KB1 |
| NRF2-mediated Oxidative | PRKCE; EP300; SOD2; PRKCZ; MAPK1; SQSTM1; |
| Stress Response | NQO1; PIK3CA; PRKCI; FOS; PIK3CB; PIK3C3; MAPK8; |
| | PRKD1; MAPK3; KRAS; PRKCD; GSTP1; MAPK9; FTL; |
| | NFE2L2; PIK3C2A; MAPK14; RAF1; MAP3K7; CREBBP; |
| | MAP2K2; AKT1; PIK3R1; MAP2K1; PPIB; JUN; KEAP1; |
| | GSK3B; ATF4; PRKCA; EIF2AK3; HSP90AA1 |
| Hepatic Fibrosis/Hepatic | EDN1; IGF1; KDR; FLT1; SMAD2; FGFR1; MET; PGF; |
| Stellate Cell Activation | SMAD3; EGFR; FAS; CSF1; NFKB2; BCL2; MYH9; |
| | IGF1R; IL6R; RELA; TLR4; PDGFRB; TNF; RELB; IL8; |
| | PDGFRA; NFKB1; TGFBR1; SMAD4; VEGFA; BAX; |
| | IL1R1; CCL2; HGF; MMP1; STAT1; IL6; CTGF; MMP9 |
| PPAR Signaling | EP300; INS; TRAF6; PPARA; RXRA; MAPK1; IKBKB; |
| | NCOR2; FOS; NFKB2; MAP3K14; STAT5B; MAPK3; |
| | NRIP1; KRAS; PPARG; RELA; STAT5A; TRAF2; |
| | PPARGC1A; PDGFRB; TNF; INSR; RAF1; IKBKG; |
| | RELB; MAP3K7; CREBBP; MAP2K2; CHUK; PDGFRA; |
| | MAP2K1; NFKB1; JUN; IL1R1; HSP90AA1 |
| Fc Epsilon RI Signaling | PRKCE; RAC1; PRKCZ; LYN; MAPK1; RAC2; PTPN11; |
| | AKT2; PIK3CA; SYK; PRKCI; PIK3CB; PIK3C3; MAPK8; |
| | PRKD1; MAPK3; MAPK10; KRAS; MAPK13; PRKCD; |
| | MAPK9; PIK3C2A; BTK; MAPK14; TNF; RAF1; FYN; |
| | MAP2K2; AKT1; PIK3R1; PDPK1; MAP2K1; AKT3; |
| | VAV3; PRKCA |
| G-Protein Coupled | PRKCE; RAP1A; RGS16; MAPK1; GNAS; AKT2; IKBKB; |
| Receptor Signaling | PIK3CA; CREB1; GNAQ; NFKB2; CAMK2A; PIK3CB; |
| | PIK3C3; MAPK3; KRAS; RELA; SRC; PIK3C2A; RAF1; |
| | IKBKG; RELB; FYN; MAP2K2; AKT1; PIK3R1; CHUK; |
| | PDPK1; STAT3; MAP2K1; NFKB1; BRAF; ATF4; AKT3; |
| | PRKCA |
| Inositol Phosphate | PRKCE; IRAK1; PRKAA2; EIF2AK2; PTEN; GRK6; |
| Metabolism | MAPK1; PLK1; AKT2; PIK3CA; CDK8; PIK3CB; PIK3C3; |
| | MAPK8; MAPK3; PRKCD; PRKAA1; MAPK9; CDK2; |
| | PIM1; PIK3C2A; DYRK1A; MAP2K2; PIP5K1A; PIK3R1; |
| | MAP2K1; PAK3; ATM; TTK; CSNK1A1; BRAF; SGK |
| PDGF Signaling | EIF2AK2; ELK1; ABL2; MAPK1; PIK3CA; FOS; PIK3CB; |
| | PIK3C3; MAPK8; CAV1; ABL1; MAPK3; KRAS; SRC; |
| | PIK3C2A; PDGFRB; RAF1; MAP2K2; JAK1; JAK2; |
| | PIK3R1; PDGFRA; STAT3; SPHK1; MAP2K1; MYC; |
| | JUN; CRKL; PRKCA; SRF; STAT1; SPHK2 |
| VEGF Signaling | ACTN4; ROCK1; KDR; FLT1; ROCK2; MAPK1; PGF; |
| | AKT2; PIK3CA; ARNT; PTK2; BCL2; PIK3CB; PIK3C3; |
| | BCL2L1; MAPK3; KRAS; HIF1A; NOS3; PIK3C2A; PXN; |
| | RAF1; MAP2K2; ELAVL1; AKT1; PIK3R1; MAP2K1; SFN; |
| | VEGFA; AKT3; FOXO1; PRKCA |
| Natural Killer Cell Signaling | PRKCE; RAC1; PRKCZ; MAPK1; RAC2; PTPN11; |
| | KIR2DL3; AKT2; PIK3CA; SYK; PRKCI; PIK3CB; |
| | PIK3C3; PRKD1; MAPK3; KRAS; PRKCD; PTPN6; |
| | PIK3C2A; LCK; RAF1; FYN; MAP2K2; PAK4; AKT1; |
| | PIK3R1; MAP2K1; PAK3; AKT3; VAV3; PRKCA |
| Cell Cycle: G1/S | HDAC4; SMAD3; SUV39H1; HDAC5; CDKN1B; BTRC; |
| Checkpoint Regulation | ATR; ABL1; E2F1; HDAC2; HDAC7A; RB1; HDAC11; |
| | HDAC9; CDK2; E2F2; HDAC3; TP53; CDKN1A; CCND1; |
| | E2F4; ATM; RBL2; SMAD4; CDKN2A; MYC; NRG1; |
| | GSK3B; RBL1; HDAC6 |
| T Cell Receptor Signaling | RAC1; ELK1; MAPK1; IKBKB; CBL; PIK3CA; FOS; |
| | NFKB2; PIK3CB; PIK3C3; MAPK8; MAPK3; KRAS; |
| | RELA; PIK3C2A; BTK; LCK; RAF1; IKBKG; RELB; FYN; |
| | MAP2K2; PIK3R1; CHUK; MAP2K1; NFKB1; ITK; BCL10; |
| | JUN; VAV3 |
| Death Receptor Signaling | CRADD; HSPB1; BID; BIRC4; TBK1; IKBKB; FADD; |
| | FAS; NFKB2; BCL2; MAP3K14; MAPK8; RIPK1; CASP8; |
| | DAXX; TNFRSF10B; RELA; TRAF2; TNF; IKBKG; RELB; |
| | CASP9; CHUK; APAF1; NFKB1; CASP2; BIRC2; CASP3; |
| | BIRC3 |
| FGF Signaling | RAC1; FGFR1; MET; MAPKAPK2; MAPK1; PTPN11; |
| | AKT2; PIK3CA; CREB1; PIK3CB; PIK3C3; MAPK8; |
| | MAPK3; MAPK13; PTPN6; PIK3C2A; MAPK14; RAF1; |
| | AKT1; PIK3R1; STAT3; MAP2K1; FGFR4; CRKL; ATF4; |
| | AKT3; PRKCA; HGF |
| GM-CSF Signaling | LYN; ELK1; MAPK1; PTPN11; AKT2; PIK3CA; CAMK2A; |
| | STAT5B; PIK3CB; PIK3C3; GNB2L1; BCL2L1; MAPK3; |
| | ETS1; KRAS; RUNX1; PIM1; PIK3C2A; RAF1; MAP2K2; |
| | AKT1; JAK2; PIK3R1; STAT3; MAP2K1; CCND1; AKT3; |
| | STAT1 |
| Amyotrophic Lateral | BID; IGF1; RAC1; BIRC4; PGF; CAPNS1; CAPN2; |
| Sclerosis Signaling | PIK3CA; BCL2; PIK3CB; PIK3C3; BCL2L1; CAPN1; |
| | PIK3C2A; TP53; CASP9; PIK3R1; RAB5A; CASP1; |
| | APAF1; VEGFA; BIRC2; BAX; AKT3; CASP3; BIRC3 |
| JAK/Stat Signaling | PTPN1; MAPK1; PTPN11; AKT2; PIK3CA; STAT5B; |
| | PIK3CB; PIK3C3; MAPK3; KRAS; SOCS1; STAT5A; |
| | PTPN6; PIK3C2A; RAF1; CDKN1A; MAP2K2; JAK1; |
| | AKT1; JAK2; PIK3R1; STAT3; MAP2K1; FRAP1; AKT3; |
| | STAT1 |
| Nicotinate and Nicotinamide | PRKCE; IRAK1; PRKAA2; EIF2AK2; GRK6; MAPK1; |
| Metabolism | PLK1; AKT2; CDK8; MAPK8; MAPK3; PRKCD; PRKAA1; |
| | PBEF1; MAPK9; CDK2; PIM1; DYRK1A; MAP2K2; |
| | MAP2K1; PAK3; NT5E; TTK; CSNK1A1; BRAF; SGK |
| Chemokine Signaling | CXCR4; ROCK2; MAPK1; PTK2; FOS; CFL1; GNAQ; |
| | CAMK2A; CXCL12; MAPK8; MAPK3; KRAS; MAPK13; |
| | RHOA; CCR3; SRC; PPP1CC; MAPK14; NOX1; RAF1; |
| | MAP2K2; MAP2K1; JUN; CCL2; PRKCA |
| IL-2 Signaling | ELK1; MAPK1; PTPN11; AKT2; PIK3CA; SYK; FOS; |
| | STAT5B; PIK3CB; PIK3C3; MAPK8; MAPK3; KRAS; |
| | SOCS1; STAT5A; PIK3C2A; LCK; RAF1; MAP2K2; |
| | JAK1; AKT1; PIK3R1; MAP2K1; JUN; AKT3 |
| Synaptic Long Term | PRKCE; IGF1; PRKCZ; PRDX6; LYN; MAPK1; GNAS; |
| Depression | PRKCI; GNAQ; PPP2R1A; IGF1R; PRKD1; MAPK3; |
| | KRAS; GRN; PRKCD; NOS3; NOS2A; PPP2CA; |
| | YWHAZ; RAF1; MAP2K2; PPP2R5C; MAP2K1; PRKCA |
| Estrogen Receptor | TAF4B; EP300; CARMI; PCAF; MAPK1; NCOR2; |
| Signaling | SMARCA4; MAPK3; NRIP1; KRAS; SRC; NR3C1; |
| | HDAC3; PPARGC1A; RBM9; NCOA3; RAF1; CREBBP; |
| | MAP2K2; NCOA2; MAP2K1; PRKDC; ESR1; ESR2 |
| Protein Ubiquitination | TRAF6; SMURF1; BIRC4; BRCA1; UCHL1; NEDD4; |
| Pathway | CBL; UBE2I; BTRC; HSPA5; USP7; USP10; FBXW7; |
| | USP9X; STUB1; USP22; B2M; BIRC2; PARK2; USP8; |
| | USP1; VHL; HSP90AA1; BIRC3 |
| IL-10 Signaling | TRAF6; CCR1; ELK1; IKBKB; SP1; FOS; NFKB2; |
| | MAP3K14; MAPK8; MAPK13; RELA; MAPK14; TNF; |
| | IKBKG; RELB; MAP3K7; JAK1; CHUK; STAT3; NFKB1; |
| | JUN; IL1R1; IL6 |
| VDR/RXR Activation | PRKCE; EP300; PRKCZ; RXRA; GADD45A; HES1; |
| | NCOR2; SP1; PRKCI; CDKN1B; PRKD1; PRKCD; |
| | RUNX2; KLF4; YY1; NCOA3; CDKN1A; NCOA2; SPP1; |
| | LRP5; CEBPB; FOXO1; PRKCA |
| TGF-beta Signaling | EP300; SMAD2; SMURF1; MAPK1; SMAD3; SMAD1; |
| | FOS; MAPK8; MAPK3; KRAS; MAPK9; RUNX2; |
| | SERPINE1; RAF1; MAP3K7; CREBBP; MAP2K2; |
| | MAP2K1; TGFBR1; SMAD4; JUN; SMAD5 |
| Toll-like Receptor Signaling | IRAK1; EIF2AK2; MYD88; TRAF6; PPARA; ELK1; |
| | IKBKB; FOS; NFKB2; MAP3K14; MAPK8; MAPK13; |
| | RELA; TLR4; MAPK14; IKBKG; RELB; MAP3K7; CHUK; |
| | NFKB1; TLR2; JUN |
| p38 MAPK Signaling | HSPB1; IRAK1; TRAF6; MAPKAPK2; ELK1; FADD; FAS; |
| | CREB1; DDIT3; RPS6KA4; DAXX; MAPK13; TRAF2; |
| | MAPK14; TNF; MAP3K7; TGFBR1; MYC; ATF4; IL1R1; |
| | SRF; STAT1 |
| Neurotrophin/TRK Signaling | NTRK2; MAPK1; PTPN11; PIK3CA; CREB1; FOS; |
| | PIK3CB; PIK3C3; MAPK8; MAPK3; KRAS; PIK3C2A; |
| | RAF1; MAP2K2; AKT1; PIK3R1; PDPK1; MAP2K1; |
| | CDC42; JUN; ATF4 |
| FXR/RXR Activation | INS; PPARA; FASN; RXRA; AKT2; SDC1; MAPK8; |
| | APOB; MAPK10; PPARG; MTTP; MAPK9; PPARGC1A; |
| | TNF; CREBBP; AKT1; SREBF1; FGFR4; AKT3; FOXO1 |
| Synaptic Long Term | PRKCE; RAP1A; EP300; PRKCZ; MAPK1; CREB1; |
| Potentiation | PRKCI; GNAQ; CAMK2A; PRKD1; MAPK3; KRAS; |
| | PRKCD; PPP1CC; RAF1; CREBBP; MAP2K2; MAP2K1; |
| | ATF4; PRKCA |
| Calcium Signaling | RAP1A; EP300; HDAC4; MAPK1; HDAC5; CREB1; |
| | CAMK2A; MYH9; MAPK3; HDAC2; HDAC7A; HDAC11; |
| | HDAC9; HDAC3; CREBBP; CALR; CAMKK2; ATF4; |
| | HDAC6 |
| EGF Signaling | ELK1; MAPK1; EGFR; PIK3CA; FOS; PIK3CB; PIK3C3; |
| | MAPK8; MAPK3; PIK3C2A; RAF1; JAK1; PIK3R1; |
| | STAT3; MAP2K1; JUN; PRKCA; SRF; STAT1 |
| Hypoxia Signaling in the | EDN1; PTEN; EP300; NQO1; UBE2I; CREB1; ARNT; |
| Cardiovascular System | HIF1A; SLC2A4; NOS3; TP53; LDHA; AKT1; ATM; |
| | VEGFA; JUN; ATF4; VHL; HSP90AA1 |
| LPS/IL-1 Mediated Inhibition | IRAK1; MYD88; TRAF6; PPARA; RXRA; ABCA1; |
| of RXR Function | MAPK8; ALDH1A1; GSTP1; MAPK9; ABCB1; TRAF2; |
| | TLR4; TNF; MAP3K7; NR1H2; SREBF1; JUN; IL1R1 |
| LXR/RXR Activation | FASN; RXRA; NCOR2; ABCA1; NFKB2; IRF3; RELA; |
| | NOS2A; TLR4; TNF; RELB; LDLR; NR1H2; NFKB1; |
| | SREBF1; IL1R1; CCL2; IL6; MMP9 |
| Amyloid Processing | PRKCE; CSNK1E; MAPK1; CAPNS1; AKT2; CAPN2; |
| | CAPN1; MAPK3; MAPK13; MAPT; MAPK14; AKT1; |
| | PSEN1; CSNK1A1; GSK3B; AKT3; APP |
| IL-4 Signaling | AKT2; PIK3CA; PIK3CB; PIK3C3; IRS1; KRAS; SOCS1; |
| | PTPN6; NR3C1; PIK3C2A; JAK1; AKT1; JAK2; PIK3R1; |
| | FRAP1; AKT3; RPS6KB1 |
| Cell Cycle: G2/M DNA | EP300; PCAF; BRCA1; GADD45A; PLK1; BTRC; |
| Damage Checkpoint | CHEK1; ATR; CHEK2; YWHAZ; TP53; CDKN1A; |
| Regulation | PRKDC; ATM; SFN; CDKN2A |
| Nitric Oxide Signaling in the | KDR; FLT1; PGF; AKT2; PIK3CA; PIK3CB; PIK3C3; |
| Cardiovascular System | CAV1; PRKCD; NOS3; PIK3C2A; AKT1; PIK3R1; |
| | VEGFA; AKT3; HSP90AA1 |
| Purine Metabolism | NME2; SMARCA4; MYH9; RRM2; ADAR; EIF2AK4; |
| | PKM2; ENTPD1; RAD51; RRM2B; TJP2; RAD51C; |
| | NT5E; POLDI; NME1 |
| cAMP-mediated Signaling | RAP1A; MAPK1; GNAS; CREB1; CAMK2A; MAPK3; |
| | SRC; RAF1; MAP2K2; STAT3; MAP2K1; BRAF; ATF4 |
| Mitochondrial Dysfunction | SOD2; MAPK8; CASP8; MAPK10; MAPK9; CASP9; |
| Notch Signaling | PARK7; PSEN1; PARK2; APP; CASP3 |
| | HES1; JAG1; NUMB; NOTCH4; ADAM17; NOTCH2; |
| | PSEN1; NOTCH3; NOTCH1; DLL4 |
| Endoplasmic Reticulum | HSPA5; MAPK8; XBP1; TRAF2; ATF6; CASP9; ATF4; |
| Stress Pathway | EIF2AK3; CASP3 |
| Pyrimidine Metabolism | NME2; AICDA; RRM2; EIF2AK4; ENTPD1; RRM2B; |
| | NT5E; POLD1; NME1 |
| Parkinson's Signaling | UCHL1; MAPK8; MAPK13; MAPK14; CASP9; PARK7; |
| | PARK2; CASP3 |
| Cardiac & Beta Adrenergic | GNAS; GNAQ; PPP2R1A; GNB2L1; PPP2CA; PPP1CC; |
| Signaling | PPP2R5C |
| Glycolysis/Gluconeogenesis | HK2; GCK; GPI; ALDH1A1; PKM2; LDHA; HK1 |
| Interferon Signaling | IRF1; SOCS1; JAK1; JAK2; IFITM1; STAT1; IFIT3 |
| Sonic Hedgehog Signaling | ARRB2; SMO; GLI2; DYRK1A; GLI1; GSK3B; DYRK1B |
| Glycerophospholipid | PLD1; GRN; GPAM; YWHAZ; SPHK1; SPHK2 |
| Metabolism | |
| Phospholipid Degradation | PRDX6; PLD1; GRN; YWHAZ; SPHK1; SPHK2 |
| Tryptophan Metabolism | SIAH2; PRMT5; NEDD4; ALDH1A1; CYP1B1; SIAH1 |
| Lysine Degradation | SUV39H1; EHMT2; NSD1; SETD7; PPP2R5C |
| Nucleotide Excision Repair | ERCC5; ERCC4; XPA; XPC; ERCC1 |
| Pathway | |
| Starch and Sucrose | UCHL1; HK2; GCK; GPI; HK1 |
| Metabolism | |
| Aminosugars Metabolism | NQO1; HK2; GCK; HK1 |
| Arachidonic Acid | PRDX6; GRN; YWHAZ; CYP1B1 |
| Metabolism | |
| Circadian Rhythm Signaling | CSNK1E; CREB1; ATF4; NR1D1 |
| Coagulation System | BDKRB1; F2R; SERPINE1; F3 |
| Dopamine Receptor | PPP2R1A; PPP2CA; PPP1CC; PPP2R5C |
| Signaling | |
| Glutathione Metabolism | IDH2; GSTP1; ANPEP; IDH1 |
| Glycerolipid Metabolism | ALDH1A1; GPAM; SPHK1; SPHK2 |
| Linoleic Acid Metabolism | PRDX6; GRN; YWHAZ; CYP1B1 |
| Methionine Metabolism | DNMT1; DNMT3B; AHCY; DNMT3A |
| Pyruvate Metabolism | GLO1; ALDH1A1; PKM2; LDHA |
| Arginine and Proline | ALDH1A1; NOS3; NOS2A |
| Metabolism | |
| Eicosanoid Signaling | PRDX6; GRN; YWHAZ |
| Fructose and Mannose | HK2; GCK; HK1 |
| Metabolism | |
| Galactose Metabolism | HK2; GCK; HK1 |
| Stilbene, Coumarine and | PRDX6; PRDX1; TYR |
| Lignin Biosynthesis | |
| Antigen Presentation | CALR; B2M |
| Pathway | |
| Biosynthesis of Steroids | NQO1; DHCR7 |
| Butanoate Metabolism | ALDH1A1; NLGN1 |
| Citrate Cycle | IDH2; IDH1 |
| Fatty Acid Metabolism | ALDH1A1; CYP1B1 |
| Glycerophospholipid | PRDX6; CHKA |
| Metabolism | |
| Histidine Metabolism | PRMT5; ALDH1A1 |
| Inositol Metabolism | ERO1L; APEX1 |
| Metabolism of Xenobiotics | GSTP1; CYP1B1 |
| by Cytochrome p450 | |
| Methane Metabolism | PRDX6; PRDX1 |
| Phenylalanine Metabolism | PRDX6; PRDX1 |
| Propanoate Metabolism | ALDH1A1; LDHA |
| Selenoamino Acid | PRMT5; AHCY |
| Metabolism | |
| Sphingolipid Metabolism | SPHK1; SPHK2 |
| Aminophosphonate | PRMT5 |
| Metabolism | |
| Androgen and Estrogen | PRMT5 |
| Metabolism | |
| Ascorbate and Aldarate | ALDH1A1 |
| Metabolism | |
| Bile Acid Biosynthesis | ALDH1A1 |
| Cysteine Metabolism | LDHA |
| Fatty Acid Biosynthesis | FASN |
| Glutamate Receptor | GNB2L1 |
| Signaling | |
| NRF2-mediated Oxidative | PRDX1 |
| Stress Response | |
| Pentose Phosphate | GPI |
| Pathway | |
| Pentose and Glucuronate | UCHL1 |
| Interconversions | |
| Retinol Metabolism | ALDH1A1 |
| Riboflavin Metabolism | TYR |
| Tyrosine Metabolism | PRMT5, TYR |
| Ubiquinone Biosynthesis | PRMT5 |
| Valine, Leucine and | ALDH1A1 |
| Isoleucine Degradation | |
| Glycine, Serine and | CHKA |
| Threonine Metabolism | |
| Lysine Degradation | ALDH1A1 |
| Pain/Taste | TRPM5; TRPA1 |
| Pain | TRPM7; TRPC5; TRPC6; TRPC1; Cnr1; cnr2; Grk2; |
| | Trpa1; Pomc; Cgrp; Crf; Pka; Era; Nr2b; TRPM5; Prkaca; |
| | Prkacb; Prkar1a; Prkar2a |
| Mitochondrial Function | AIF; CytC; SMAC (Diablo); Aifm-1; Aifm-2 |
| Developmental Neurology | BMP-4; Chordin (Chrd); Noggin (Nog); WNT (Wnt2; |
| | Wnt2b; Wnt3a; Wnt4; Wnt5a; Wnt6; Wnt7b; Wnt8b; |
| | Wnt9a; Wnt9b; Wnt10a; Wnt10b; Wnt16); beta-catenin; |
| | Dkk-1; Frizzled related proteins; Otx-2; Gbx2; FGF-8; |
| | Reelin; Dab1; unc-86 (Pou4f1 or Brn3a); Numb; Reln |

Further non-limiting examples of disease-associated genes and polynucleotides amd disease specific information is available from McKusick-Nathans Institute of Genetic Medicine, Johns Hopkins University (Baltimore, Md.) and National Center for Biotechnology Information, National Library of Medicine (Bethesda, Md.), available on the World Wide Web.

In an aspect, the invention provides a method of individualized or personalized treatment of a genetic disease in a subject in need of such treatment comprising: (a) introducing one or more mutations ex vivo in a tissue, organ or a cell line, or in vivo in a transgenic non-human mammal, comprising delivering to cell(s) of the tissue, organ, cell or mammal a composition comprising the engineered nucleic acid modification system of the present invention and/or component(s) thereof, a formulation thereof, modified cell, or other delivery particle, vector, etc. described elsewhere herein, wherein the specific mutations or precise sequence substitutions are or have been correlated to the genetic disease; (b) testing treatment(s) for the genetic disease on the cells to which the vector has been delivered that have the specific mutations or precise sequence substitutions correlated to the genetic disease; and (c) treating the subject based on results from the testing of treatment(s) of step (b).

### Infectious Diseases

In some embodiments, the engineered nucleic acid modification system of the present invention and/or component(s) thereof can be used to diagnose, prognose, treat, and/or prevent an infectious disease caused by a microorganism, such as bacteria, virus, fungi, parasites, or combinations thereof.

In some embodiments, the engineered nucleic acid modification system of the present invention and/or component(s) thereof can be capable of targeting specific microorganism within a mixed population. Exemplary methods of such techniques are described in e.g., Gomaa A A, Klumpe H E, Luo M L, Selle K, Barrangou R, Beisel C L. 2014. Programmable removal of bacterial strains by use of genome-targeting CRISPR-Cas systems. mBio 5:e00928-13; Citorik R J, Mimee M, Lu T K. 2014. Sequence-specific antimicrobials using efficiently delivered RNA-guided nucleases. Nat Biotechnol 32:1141-1145, the teachings of which can be adapted for use with t the engineered nucleic acid modification system of the present invention and/or component(s) thereof.

In some embodiments, the engineered nucleic acid modification system of the present invention and/or component(s) thereof can be capable of targeting pathogenic and/or drug-resistant microorganisms, such as bacteria, virus, parasites, and fungi. In some embodiments, the engineered nucleic acid modification system of the present invention and/or component(s) thereof can be capable of targeting and modifying one or more polynucleotides in a pathogenic microorganism such that the microorganism is less virulent, killed, inhibited, or is otherwise rendered incapable of causing disease and/or infecting and/or replicating in a host cell.

In some embodiments, the pathogenic bacteria that can be targeted and/or modified by the engineered nucleic acid modification system of the present invention and/or component(s) thereof described herein include, but are not limited to, those of the genus Actinomyces (e.g. A. israelii), Bacillus (e.g. B. anthracis, B. cereus), Bactereoides (e.g. B. fragilis), Bartonella (B. henselae, B. quintana), Bordetella (B. pertussis), Borrelia (e.g. B. burgdorferi, B. garinii, B. afzelii, and B. recurreentis), Brucella (e.g. B. abortus, B. canis, B. melitensis, and B. suis), Campylobacter (e.g. C. jejuni), Chlamydia (e.g. C. pneumoniae and C. trachomatis), Chlamydophila (e.g. C. psittaci), Clostridium (e.g. C. botulinum, C. difficile, C. perfringens. C. tetani), Corynebacterium (e.g. C. diptheriae), Enterococcus (e.g. E. faecalis, E. faecium), Ehrlichia (E. canis and E. chaffensis) Escherichia (e.g. E. coli), Francisella (e.g. F. tularensis), Haemophilus (e.g. H. influenzae), Helicobacter (H. pylori), Klebsiella (E.g. K. pneumoniae), Legionella (e.g. L. pneumophila), Leptospira (e.g. L. interrogans, L. santarosai, L. weilii, L. noguchii), Listereia (e.g. L. monocytogeenes), Mycobacterium (e.g. M. leprae, M. tuberculosis, M. ulcerans), Mycoplasma (M. pneumoniae), Neisseria (N. gonorrhoeae and N. menigitidis), Nocardia (e.g. N. asteeroides), Pseudomonas (P. aeruginosa), Rickettsia (R. rickettsia), Salmonella (S. typhi and S. typhimurium), Shigella (S. sonnei and S. dysenteriae), Staphylococcus (S. aureus, S. epidermidis, and S. saprophyticus), Streeptococcus (S. agalactiaee, S. pneumoniae, S. pyogenes), Treponema (T. pallidum), Ureeaplasma (e.g. U. urealyticum), Vibrio (e.g. V. cholerae), Yersinia (e.g. Y pestis, Y, enteerocolitica, and Y, pseudotuberculosis).

In some embodiments, the pathogenic virus that can be targeted and/or modified by the engineered nucleic acid modification system of the present invention and/or component(s) thereof described herein include, but are not limited to, a double-stranded DNA virus, a partly double-stranded DNA virus, a single-stranded DNA virus, a positive single-stranded RNA virus, a negative single-stranded RNA virus, or a double stranded RNA virus. In some embodiments, the pathogenic virus can be from the family Adenoviridae (e.g. Adenovirus), Herpesviridae (e.g. Herpes simplex, type 1, Herpes simplex, type 2, Varicella-zoster virus, Epstein-Barr virus, Human cytomegalovirus, Human herpesvirus, type 8), Papillomaviridae (e.g. Human papillomavirus), Polyomaviridae (e.g. BK virus, JC virus), Poxviridae (e.g. smallpox), Hepadnaviridae (e.g. Hepatitis B), Parvoviridae (e.g. Parvovirus B19), Astroviridae (e.g. Human astrovirus), Caliciviridae (e.g. Norwalk virus), Picornaviridae (e.g. coxsackievirus, hepatitis A virus, poliovirus, rhinovirus), Coronaviridae (e.g. Severe acute respiratory syndrome-related coronavirus, strains: Severe acute respiratory syndrome virus, Severe acute respiratory syndrome coronavirus 2 (COVID-19)), Flaviviridae (e.g. Hepatitis C virus, yellow fever virus, dengue virus, West Nile virus, TBE virus), Togaviridae (e.g. Rubella virus), Hepeviridae (e.g. Hepatitis E virus), Retroviridae (Human immunodeficiency virus (HIV)), Orthomyxoviridae (e.g. Influenza virus), Arenaviridae (e.g. Lassa virus), Bunyaviridae (e.g. Crimean-Congo hemorrhagic fever virus, Hantaan virus), Filoviridae (e.g. Ebola virus and Marburg virus), Paramyxoviridae (e.g. Measles virus, Mumps virus, Parainfluenza virus, Respiratory syncytial virus), Rhabdoviridae (Rabies virus), Hepatits D virus, Reoviridae (e.g. Rotavirus, Orbivirus, Coltivirus, Banna virus).

In some embodiments, the pathogenic fungi that can be targeted and/or modified by the engineered nucleic acid modification system of the present invention and/or component(s) thereof include, but are not limited to, those of the genus Candida (e.g., C. albicans), Aspergillus (e.g., A. fumigatus, A. flavus, A. clavatus), Cryptococcus (e.g., C. neoformans, C. gattii), Histoplasma (H. capsulatum), Pneumocystis (e.g., P. jiroveecii), Stachybotrys (e.g., S. chartarum).

In some embodiments, the pathogenic parasites that can be targeted and/or modified by the engineered nucleic acid modification system of the present invention and/or component(s) thereof include, but are not limited to, protozoa, helminths, and ectoparasites. In some embodiments, the pathogenic protozoa that can be targeted and/or modified by the engineered nucleic acid modification system of the present invention and/or component(s) thereof include, but are not limited to, those from the groups Sarcodina (e.g., ameba such as Entamoeba), Mastigophora (e.g., flagellates such as Giardia and Leishmania), Cilophora (e.g., ciliates such as Balantidum), and sporozoa (e.g., plasmodium and cryptosporidium). In some embodiments, the pathogenic helminths that can be targeted and/or modified by the engineered nucleic acid modification system of the present invention and/or component(s) thereof include, but are not limited to, flatworms (platyhelminths), thorny-headed worms (acanthoceephalins), and roundworms (nematodes). In some embodiments, the pathogenic ectoparasites that can be targeted and/or modified by t the engineered nucleic acid modification system of the present invention and/or component(s) thereof include, but are not limited to, ticks, fleas, lice, and mites.

In some embodiments, the pathogenic parasite that can be targeted and/or modified by the engineered nucleic acid modification system of the present invention and/or component(s) thereof include, but are not limited to, Acanthamoeba spp., Balamuthia mandrillaris, Babesiosis spp. (e.g., Babesia B. divergens, B. bigemina, B. equi, B. microfti, B. duncani), Balantidiasis spp. (e.g., Balantidium coli), Blastocystis spp., Cryptosporidium spp., Cyclosporiasis spp. (e.g., Cyclospora cayetanensis), Dientamoebiasis spp. (e.g., Dientamoeba fragilis), Amoebiasis spp. (e.g., Entamoeba histolytica), Giardiasis spp. (e.g., Giardia lamblia), Isosporiasis spp. (e.g., Isospora belli), Leishmania spp., Naegleria spp. (e.g., Naegleria fowleri), Plasmodium spp. (e.g., Plasmodium falciparum, Plasmodium vivax, Plasmodium ovale curtisi, Plasmodium ovale wallikeri, Plasmodium malariae, Plasmodium knowlesi), Rhinosporidiosis spp. (e.g., Rhinosporidium seeberi), Sarcocystosis spp. (e.g., Sarcocystis bovihominis, Sarcocystis suihominis), Toxoplasma spp. (e.g., Toxoplasma gondii), Trichomonas spp. (e.g., Trichomonas vaginalis), Trypanosoma spp. (e.g., Trypanosoma brucei), Trypanosoma spp. (e.g., Trypanosoma cruzi), Tapeworm (e.g., Cestoda, Taenia multiceps, Taenia saginata, Taenia solium), Diphyllobothrium latum spp., Echinococcus spp. (e.g., Echinococcus granulosus, Echinococcus multilocularis, E. vogeli, E. oligarthrus), Hymenolepis spp. (e.g., Hymenolepis nana, Hymenolepis diminuta), Bertiella spp. (e.g., Bertiella mucronata, Bertiella studeri), Spirometra (e.g., Spirometra erinaceieuropaei), Clonorchis spp. (e.g., Clonorchis sinensis; Clonorchis viverrini), Dicrocoelium spp. (e.g., Dicrocoelium dendriticum), Fasciola spp. (e.g., Fasciola hepatica, Fasciola gigantica), Fasciolopsis spp. (e.g. Fasciolopsis buski), Metagonimus spp. (e.g., Metagonimus yokogawai), Metorchis spp. (e.g., Metorchis conjunctus), Opisthorchis spp. (e.g., Opisthorchis viverrini, Opisthorchis felineus), Clonorchis spp. (e.g., Clonorchis sinensis), Paragonimus spp. (e.g., Paragonimus westermani; Paragonimus africanus; Paragonimus caliensis; Paragonimus kellicotti; Paragonimus skrjabini; Paragonimus uterobilateralis), Schistosoma sp., Schistosoma spp. (e.g., Schistosoma mansoni, Schistosoma haematobium, Schistosoma japonicum, Schistosoma mekongi, and Schistosoma intercalatum), Echinostoma spp. (e.g., E. echinatum), Trichobilharzia spp. (e.g., Trichobilharzia regent), Ancylostoma spp. (e.g., Ancylostoma duodenale), Necator spp. (e.g., Necator americanus), Angiostrongylus spp., Anisakis spp., Ascaris spp. (e.g., Ascaris lumbricoides), Baylisascaris spp. (e.g., Baylisascaris procyonis), Brugia spp. (e.g., Brugia malayi, Brugia timori), Dioctophyme spp. (e.g., Dioctophyme renale), Dracunculus spp. (e.g., Dracunculus medinensis), Enterobius spp. (e.g., Enterobius vermicularis, Enterobius gregorii), Gnathostoma spp. (e.g., Gnathostoma spinigerum, Gnathostoma hispidum), Halicephalobus spp. (e.g., Halicephalobus gingivalis), Loa loa spp. (e.g., Loa loa filaria), Mansonella spp. (e.g., Mansonella streptocerca), Onchocerca spp. (e.g., Onchocerca volvulus), Strongyloides spp. (e.g., Strongyloides stercoralis), Thelazia spp. (e.g., Thelazia californiensis, Thelazia callipaeda), Toxocara spp. (e.g., Toxocara canis, Toxocara cati, Toxascaris leonine), Trichinella spp. (e.g., Trichinella spiralis, Trichinella britovi, Trichinella nelsoni, Trichinella nativa), Trichuris spp. (e.g., Trichuris trichiura, Trichuris vulpis), Wuchereria spp. (e.g., Wuchereria bancrofti), Dermatobia spp. (e.g., Dermatobia hominis), Tunga spp. (e.g., Tunga penetrans), Cochliomyia spp. (e.g., Cochliomyia hominivorax), Linguatula spp. (e.g., Linguatula serrata), Archiacanthocephala sp., Moniliformis sp. (e.g., Moniliformis moniliformis), Pediculus spp. (e.g., Pediculus humanus capitis, Pediculus humanus humanus), Pthirus spp. (e.g., Pthirus pubis), Arachnida spp. (e.g., Trombiculidae, Ixodidae, Argaside), Siphonaptera spp (e.g., Siphonaptera: Pulicinae), Cimicidae spp. (e.g., Cimex lectularius and Cimex hemipterus), Diptera spp., Demodex spp. (e.g., Demodex folliculorum/brevis/canis), Sarcoptes spp. (e.g., Sarcoptes scabiei), Dermanyssus spp. (e.g., Dermanyssus gallinae), Ornithonyssus spp. (e.g., Ornithonyssus sylviarum, Ornithonyssus bursa, Ornithonyssus bacoti), Laelaps spp. (e.g., Laelaps echidnina), Liponyssoides spp. (e.g., Liponyssoides sanguineus).

In some embodiments the gene targets can be any of those as set forth in Table 1 of Strich and Chertow. 2019. J. Clin. Microbio. 57:4 e01307-18, which is incorporated herein as if expressed in its entirety herein.

In some embodiments, the method can include delivering an engineered nucleic acid modification system of the present invention and/or component(s) thereof to a pathogenic organism described herein, allowing the engineered nucleic acid modification system of the present invention and/or component(s) thereof to specifically bind and modify one or more targets in the pathogenic organism, whereby the modification kills, inhibits, reduces the pathogenicity of the pathogenic organism, or otherwise renders the pathogenic organism non-pathogenic. In some embodiments, delivery of the engineered nucleic acid modification system of the present invention and/or component(s) thereof occurs in vivo (i.e., in the subject being treated). In some embodiments occurs by an intermediary, such as microorganism or phage that is non-pathogenic to the subject but is capable of transferring polynucleotides and/or infecting the pathogenic microorganism. In some embodiments, the intermediary microorganism can be an engineered bacteria, virus, or phage that contains the engineered nucleic acid modification system of the present invention and/or component(s) thereof and/or vectors and/or vector systems thereof. The method can include administering an intermediary microorganism containing the engineered nucleic acid modification system of the present invention and/or component(s) thereof and/vectors and/or vector systems thereof to the subject to be treated. The intermediary microorganism can then produce t the engineered nucleic acid modification system of the present invention and/or component(s) thereof and/or transfer a engineered nucleic acid modification system polynucleotide to the pathogenic organism. In embodiments, where the engineered nucleic acid modification system of the present invention and/or component(s) thereof, vector, or vector system is transferred to the pathogenic microorganism, the engineered nucleic acid modification system of the present invention and/or component(s) thereof is then produced in the pathogenic microorganism and modifies the pathogenic microorganism such that it is less virulent, killed, inhibited, or is otherwise rendered incapable of causing disease and/or infecting and/or replicating in a host or cell thereof.

In some embodiments, where the pathogenic microorganism inserts its genetic material into the host cell's genome (e.g., a virus), the engineered nucleic acid modification system of the present invention and/or component(s) thereof can be designed such that it modifies the host cell's genome such that the viral DNA or cDNA cannot be replicated by the host cell's machinery into a functional virus. In some embodiments, where the pathogenic microorganism inserts its genetic material into the host cell's genome (e.g., a virus), the engineered nucleic acid modification system of the present invention and/or component(s) thereof can be designed such that it modifies the host cell's genome such that the viral DNA or cDNA is deleted from the host cell's genome.

It will be appreciated that inhibiting or killing the pathogenic microorganism, the disease and/or condition that its infection causes in the subject can be treated or prevented. Thus, also provided herein are methods of treating and/or preventing one or more diseases or symptoms thereof caused by any one or more pathogenic microorganisms, such as any of those described herein.

### Mitochondrial Diseases

Some of the most challenging mitochondrial disorders arise from mutations in mitochondrial DNA (mtDNA), a high copy number genome that is maternally inherited. In some embodiments, mtDNA mutations can be modified using the engineered nucleic acid modification system of the present invention and/or component(s) thereof. In some embodiments, the mitochondrial disease that can be diagnosed, prognosed, treated, and/or prevented can be MELAS (mitochondrial myopathy encephalopathy, and lactic acidosis and stroke-like episodes), CPEO/PEO (chronic progressive external ophthalmoplegia syndrome/progressive external ophthalmoplegia), KSS (Kearns-Sayre syndrome), MIDD (maternally inherited diabetes and deafness), MERRF (myoclonic epilepsy associated with ragged red fibers), NIDDM (noninsulin-dependent diabetes mellitus), LHON (Leber hereditary optic neuropathy), LS (Leigh Syndrome) an aminoglycoside induced hearing disorder, NARP (neuropathy, ataxia, and pigmentary retinopathy), Extrapyramidal disorder with akinesia-rigidity, psychosis and SNHL, Nonsyndromic hearing loss a cardiomyopathy, an encephalomyopathy, Pearson's syndrome, a disease identified as being caused or attributed to a mtDNA mutation set forth at mitomap.org, or a combination thereof.

In some embodiments, the mtDNA of a subject can be modified in vivo or ex vivo. In some embodiments, where the mtDNA is modified ex vivo, after modification the cells containing the modified mitochondria can be administered back to the subject. In some embodiments, the engineered nucleic acid modification system of the present invention and/or component(s) thereof can be capable of correcting an mtDNA mutation such as any one or more of those that can be found at mitomap.org.

In some embodiments, at least one of the one or more mtDNA mutations is selected from the group consisting of. A3243G, C3256T, T3271C, G1019A, A1304T, A15533G, C1494T, C4467A, T1658C, G12315A, A3421G, A8344G, T8356C, G8363A, A13042T, T3200C, G3242A, A3252G, T3264C, G3316A, T3394C, T14577C, A4833G, G3460A, G9804A, G11778A, G14459A, A14484G, G15257A, T8993C, T8993G, G10197A, G13513A, T1095C, C1494T, A1555G, G1541A, C1634T, A3260G, A4269G, T7587C, A8296G, A8348G, G8363A, T9957C, T9997C, G12192A, C12297T, A14484G, G15059A, duplication of CCCCCTCCCC-tandem repeats at positions 305-314 and/or 956-965, deletion at positions from 8,469-13,447, 4,308-14,874, and/or 4,398-14,822, 961ins/delC, the mitochondrial common deletion (e.g. mtDNA 4,977 bp deletion), and combinations thereof.

In some embodiments, the mitochondrial mutation can be any mutation as set forth in or as identified by use of one or more bioinformatic tools available at Mitomap available at mitomap.org. Such tools include, but are not limited to, “Variant Search, aka Market Finder”, Find Sequences for Any Haplogroup, aka “Sequence Finder”, “Variant Info”, “POLG Pathogenicity Prediction Server”, “MITOMASTER”, “Allele Search”, “Sequence and Variant Downloads”, “Data Downloads”. MitoMap contains reports of mutations in mtDNA that can be associated with disease and maintains a database of reported mitochondrial DNA Base Substitution Diseases: rRNA/tRNA mutations.

In some embodiments, the method includes delivering an engineered nucleic acid modification system of the present invention and/or component(s) thereof to a cell, and more specifically one or more mitochondria in a cell, allowing the engineered nucleic acid modification system of the present invention and/or component(s) thereof to modify one or more target polynucleotides in the cell, and more specifically one or more mitochondria in the cell. The target polynucleotides can correspond to a mutation in the mtDNA, such as any one or more of those described herein. In some embodiments, the modification can alter a function of the mitochondria such that the mitochondria functions normally or at least is/are less dysfunctional as compared to an unmodified mitochondria. Modification can occur in vivo or ex vivo. Where modification is performed ex vivo, cells containing modified mitochondria can be administered to a subject in need thereof in an autologous or allogenic manner.

### Microbiome Modification

Microbiomes play important roles in health and disease. For example, the gut microbiome can play a role in health by controlling digestion, preventing growth of pathogenic microorganisms and have been suggested to influence mood and emotion. Imbalanced microbiomes can promote disease and are suggested to contribute to weight gain, unregulated blood sugar, high cholesterol, cancer, and other disorders. A healthy microbiome has a series of joint characteristics that can be distinguished from non-healthy individuals; thus, detection and identification of the disease-associated microbiome can be used to diagnose and detect disease in an individual. The engineered nucleic acid modification system of the present invention and/or component(s) thereof can be used to screen the microbiome cell population and be used to identify a disease associated microbiome. Cell screening methods utilizing the engineered nucleic acid modification system of the present invention and/or component(s) thereof are described elsewhere herein and can be applied to screening a microbiome, such as a gut, skin, vagina, and/or oral microbiome, of a subject.

In some embodiments, the microbe population of a microbiome in a subject can be modified using an engineered nucleic acid modification system of the present invention and/or component(s) thereof. In some embodiments, the engineered nucleic acid modification system of the present invention and/or component(s) thereof can be used to identify and select one or more cell types in the microbiome and remove them from the microbiome population. Exemplary methods of selecting cells using the engineered nucleic acid modification system of the present invention and/or component(s) thereof are described elsewhere herein. In this way the make-up or microorganism profile of the microbiome can be altered. In some embodiments, the alteration causes a change from a diseased microbiome composition to a healthy microbiome composition. In this way the ratio of one type or species of microorganism to another can be modified, such as going from a diseased ratio to a healthy ratio. In some embodiments, the cells selected are pathogenic microorganisms.

In some embodiments, the engineered nucleic acid modification system of the present invention and/or component(s) thereof can be used to modify a polynucleotide in a microorganism of a microbiome in a subject. In some embodiments, the microorganism is a pathogenic microorganism. In some embodiments, the microorganism is a commensal and non-pathogenic microorganism. Methods of modifying polynucleotides in a cell in the subject are described elsewhere herein and can be applied to these embodiments.

### Adoptive Therapy

The engineered nucleic acid modification system of the present invention and/or component(s) thereof can be used to modify cells for an adoptive cell therapy.

Aspects of the invention accordingly involve the adoptive transfer of immune system cells, such as T cells, specific for selected antigens, such as tumor associated antigens (see Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; and, Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144). Various strategies may for example be employed to genetically modify T cells by altering the specificity of the T cell receptor (TCR) for example by introducing new TCR a and R chains with selected peptide specificity (see U.S. Pat. No. 8,697,854; International Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and PCT Publication WO9215322). Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8a hinge domain and a CD8a transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GD11a-CD18, CD2, ICOS, CD27, CD154, CDS, OX40, 4-1BB, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects.

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV.

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with 7-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to threat tumor xenografts.

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoreponsive cell, thereby treating or preventing the disease (such as a neoplasia, a pathogen infection, an autoimmune disorder, or an allogeneic transplant reaction). Dosing in CAR T cell therapies may for example involve administration of from 106 to 109 cells/kg, with or without a course of lymphodepletion, for example with cyclophosphamide.

In one embodiment, the treatment can be administrated into patients undergoing an immunosuppressive treatment. The cells or population of cells may be made resistant to at least one immunosuppressive agent due to the inactivation of a gene encoding a receptor for such immunosuppressive agent. Not being bound by a theory, the immunosuppressive treatment should help the selection and expansion of the immunoresponsive or T cells according to the invention within the patient.

The administration of the cells or population of cells according to the present invention may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, by intravenous or intralymphatic injection, or intraperitoneally. In one embodiment, the cell compositions of the present invention are preferably administered by intravenous injection.

The administration of the cells or population of cells can consist of the administration of 10⁴-10⁹ cells per kg body weight, preferably 105 to 106 cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing and other modifications with the engineered nucleic acid modification system of the present invention and/or component(s) thereof may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853). For example, immunoresponsive cells may be edited to delete expression of some or all of the class of HLA type II and/or type I molecules, or to knockout selected genes that may inhibit the desired immune response, such as the PD1 gene.

Cells may be edited using any of the engineered nucleic acid modification system of the present invention and/or component(s) thereof and method of use thereof as described herein. The engineered nucleic acid modification system of the present invention and/or component(s) thereof may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed to eliminate potential alloreactive T-cell receptors (TCR), disrupt the target of a chemotherapeutic agent, block an immune checkpoint, activate a T cell, and/or increase the differentiation and/or proliferation of functionally exhausted or dysfunctional CD8+ T-cells (see PCT Patent Publications: WO2013176915, WO2014059173, WO2014172606, WO2014184744, and WO2014191128). Editing may result in inactivation of a gene.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SHP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT1 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SIP-1 or TIM-3. In preferred embodiments, the gene locus involved in the expression of PD-1 or CTLA-4 genes is targeted. In other preferred embodiments, combinations of genes are targeted, such as but not limited to PD-1 and TIGIT.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ.

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

The practice of the present invention employs, unless otherwise indicated, conventional techniques of immunology, biochemistry, chemistry, molecular biology, microbiology, cell biology, genomics and recombinant DNA, which are within the skill of the art. See MOLECULAR CLONING: A LABORATORY MANUAL, 2nd edition (1989) (Sambrook, Fritsch and Maniatis); MOLECULAR CLONING: A LABORATORY MANUAL, 4th edition (2012) (Green and Sambrook); CURRENT PROTOCOLS IN MOLECULAR BIOLOGY (1987) (F. M. Ausubel, et al. eds.); the series METHODS IN ENZYMOLOGY (Academic Press, Inc.); PCR 2: A PRACTICAL APPROACH (1995) (M. J. MacPherson, B. D. Hames and G. R. Taylor eds.); ANTIBODIES, A LABORATORY MANUAL (1988) (Harlow and Lane, eds.); ANTIBODIES A LABORATORY MANUAL, 2nd edition (2013) (E. A. Greenfield ed.); and ANIMAL CELL CULTURE (1987) (R.I. Freshney, ed.).

The practice of the present invention employs, unless otherwise indicated, conventional techniques for generation of genetically modified mice. See Marten H. Hofker and Jan van Deursen, TRANSGENIC MOUSE METHODS AND PROTOCOLS, 2nd edition (2011).

In some embodiments, the invention described herein relates to a method for adoptive immunotherapy, in which T cells are edited ex vivo by CRISPR to modulate at least one gene and subsequently administered to a patient in need thereof. In some embodiments, the CRISPR editing comprising knocking-out or knocking-down the expression of a target gene in the edited T cells. In some embodiments, in addition to modulating the target gene, the T cells are also edited ex vivo by CRISPR to (1) knock-in an exogenous gene encoding a chimeric antigen receptor (CAR) or a T-cell receptor (TCR), (2) knock-out or knock-down expression of an immune checkpoint receptor, (3) knock-out or knock-down expression of an endogenous TCR, (4) knock-out or knock-down expression of a human leukocyte antigen class I (HLA-I) proteins, and/or (5) knock-out or knock-down expression of an endogenous gene encoding an antigen targeted by an exogenous CAR or TCR.

In some embodiments, the T cells are contacted ex vivo with an adeno-associated virus (AAV) vector encoding a the engineered nucleic acid modification system of the present invention and/or component(s) thereof, optionally a donor construct, and/or a guide molecule comprising a guide sequence hybridizable to a target sequence, a tracr mate sequence, and a tracr sequence hybridizable to the tracr mate sequence. In some embodiments, the T cells are contacted ex vivo (e.g., by electroporation) with a ribonucleoprotein (RNP) comprising an engineered nucleic acid effector protein(s) (e.g., a site-specific nuclease (such as a Cas polypeptide), VirD1 and/or VirD2, and/or DNA polymerase) complexed with a guide molecule, wherein the guide molecule comprising a guide sequence hybridizable to a target sequence, a tracr mate sequence, and a tracr sequence hybridizable to the tracr mate sequence. See Rupp et al., Scientific Reports 7:737 (2017); Liu et al., Cell Research 27:154-157 (2017). In some embodiments, the T cells are contacted ex vivo (e.g., by electroporation) with an mRNA encoding a protein of the engineered nucleic acid modification system of the present invention, a donor construct, and/or and a guide molecule comprising a guide sequence hybridizable to a target sequence, a tracr mate sequence, and a tracr sequence hybridizable to the tracr mate sequence. See Eyquem et al., Nature 543:113-117 (2017). In some embodiments, the T cells are not contacted ex vivo with a lentivirus or retrovirus vector.

In some embodiments, the method comprises editing T cells ex vivo via engineered nucleic acid modification system mediated knock-in an exogenous gene encoding a CAR, thereby allowing the edited T cells to recognize cancer cells based on the expression of specific proteins located on the cell surface. In some embodiments, T cells are edited ex vivo by using an engineered nucleic acid modification system of the present invention to knock-in an exogenous gene encoding a TCR, thereby allowing the edited T cells to recognize proteins derived from either the surface or inside of the cancer cells. In some embodiments, the method comprising providing an exogenous CAR-encoding or TCR-encoding sequence as a donor sequence, which can be integrated by homology-directed repair (HDR) into a genomic locus targeted by a guide molecule sequence. In some embodiments, targeting the exogenous CAR or TCR to an endogenous TCR a constant (TRAC) locus can reduce tonic CAR signaling and facilitate effective internalization and re-expression of the CAR following single or repeated exposure to antigen, thereby delaying effector T-cell differentiation and exhaustion. See Eyquem et al., Nature 543:113-117 (2017).

In some embodiments, the method comprises editing T cells ex vivo by using an engineered nucleic acid modification system mediated to block one or more immune checkpoint receptors to reduce immunosuppression by cancer cells. In some embodiments, T cells are edited ex vivo by the engineered nucleic acid modification system of the present invention and/or component(s) thereof to knock-out or knock-down an endogenous gene involved in the programmed death-1 (PD-1) signaling pathway, such as PD-1 and PD-L1. In some embodiments, T cells are edited ex vivo by CRISPR to mutate the Pdcd1 locus or the CD274 locus. In some embodiments, T cells are edited ex vivo the engineered nucleic acid modification system of the present invention and/or component(s) thereof using one or more guide sequences targeting the first exon of PD-1. See Rupp et al., Scientific Reports 7:737 (2017); Liu et al., Cell Research 27:154-157 (2017).

In some embodiments, the method comprises editing T cells ex vivo by the engineered nucleic acid modification system of the present invention and/or component(s) thereof to eliminate potential alloreactive TCRs to allow allogeneic adoptive transfer. In some embodiments, T cells are edited ex vivo by the engineered nucleic acid modification system of the present invention and/or component(s) thereof to knock-out or knock-down an endogenous gene encoding a TCR (e.g., an αβ TCR) to avoid graft-versus-host-disease (GVHD). In some embodiments, T cells are edited ex vivo by the engineered nucleic acid modification system of the present invention and/or component(s) thereof to mutate the TRAC locus. In some embodiments, T cells are edited ex vivo by the engineered nucleic acid modification system of the present invention and/or component(s) thereof using one or more guide sequences targeting the first exon of TRAC. See Liu et al., Cell Research 27:154-157 (2017). In some embodiments, the method comprises use of the engineered nucleic acid modification system of the present invention and/or component(s) thereof to knock-in an exogenous gene encoding a CAR or a TCR into the TRAC locus, while simultaneously knocking-out the endogenous TCR (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous TCR promoter.

In some embodiments, the method comprises editing T cells ex vivo by the engineered nucleic acid modification system of the present invention and/or component(s) thereof to knock-out or knock-down an endogenous gene encoding an HLA-I protein to minimize immunogenicity of the edited T cells. In some embodiments, T cells are edited ex vivo by the engineered nucleic acid modification system of the present invention and/or component(s) thereof to mutate the beta-2 microglobulin (B2M) locus. In some embodiments, T cells are edited ex vivo by the engineered nucleic acid modification system of the present invention and/or component(s) thereof using one or more guide sequences targeting the first exon of B2M. See Liu et al., Cell Research 27:154-157 (2017). In some embodiments, the method comprises use of the engineered nucleic acid modification system of the present invention and/or component(s) thereof to knock-in an exogenous gene encoding a CAR or a TCR into the B2M locus, while simultaneously knocking-out the endogenous B2M (e.g., with a donor sequence encoding a self-cleaving P2A peptide following the CAR cDNA). See Eyquem et al., Nature 543:113-117 (2017). In some embodiments, the exogenous gene comprises a promoter-less CAR-encoding or TCR-encoding sequence which is inserted operably downstream of an endogenous B2M promoter.

In some embodiments, the method comprises editing T cells ex vivo by the engineered nucleic acid modification system of the present invention and/or component(s) thereof to knock-out or knock-down an endogenous gene encoding an antigen targeted by an exogenous CAR or TCR. In some embodiments, the T cells are edited ex vivo by the engineered nucleic acid modification system of the present invention and/or component(s) thereof to knock-out or knock-down the expression of a tumor antigen selected from human telomerase reverse transcriptase (hTERT), survivin, mouse double minute 2 homolog (MDM2), cytochrome P450 1B 1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53 or cyclin (DI) (see WO2016/011210). In some embodiments, the T cells are edited ex vivo by the engineered nucleic acid modification system of the present invention and/or component(s) thereof to knock-out or knock-down the expression of an antigen selected from B cell maturation antigen (BCMA), transmembrane activator and CAML Interactor (TACI), or B-cell activating factor receptor (BAFF-R), CD38, CD138, CS-1, CD33, CD26, CD30, CD53, CD92, CD100, CD148, CD150, CD200, CD261, CD262, or CD362 (see WO2017/011804).

### Treating and Preventing Diseases Using RNA Editing

In some embodiments, the disease, disorder, and/condition or symptom thereof can be treated or prevented using the engineered nucleic acid modification system of the present invention and/or component(s) thereof, where the system is capable of editing or otherwise modifying RNA. In some embodiments, the engineered nucleic acid modification system of the present invention and/or component(s) thereof described herein is an RNA editing system (i.e., is capable of editing and/or otherwise modifying RNA). In some embodiments, treatment or prevention using the engineered nucleic acid modification system of the present invention and/or component(s) thereof capable of RNA editing or modification described herein can have the advantage of less immunogenicity than a DNA editing the engineered nucleic acid modification system and is not as hindered by limitations on viral vector packaging size. Further, as the effect is transient, the effect can be better controlled over time and can potentially be reversible. Thus, they pose less risk of causing permeant detrimental effects than DNA editing/modification-based preventatives and treatments.

In some of these embodiments, the engineered nucleic acid modification system of the present invention and/or component(s) thereof contains an ADAR enzyme or effector domain thereof. Such systems are described elsewhere herein. In some embodiments, the engineered nucleic acid modification system of the present invention and/or component(s) thereof includes a Cas13 or Cas13d effector.

Any disease involving a dysfunctional RNA molecule, where the dysfunction is the result of a mutation in the RNA sequence can be treated or prevented by modifying its sequence using an engineered nucleic acid modification system of the present invention and/or component(s) thereof capable of RNA editing/modification described elsewhere herein. In some embodiments, the disease that can be treated or prevented using an engineered nucleic acid modification system of the present invention and/or component(s) thereof capable of RNA modification can be one or more of those listed in Tables 5-6, one or more of those set forth in any of a disease identified as being caused or attributed to a mtDNA mutation set forth at mitomap.org, or a combination thereof. In some embodiments, the coding sequence for the gene involved in the disease is greater than the packaging capacity of a viral vector system, particularly an AAV vector system.

The potential for RNA editing/modification has now been demonstrated in vitro and in vivo for pathogenic mutations in genes related to cystic fibrosis, Duchenne's muscular dystrophy, Hurler's syndrome, and Ornithine transcarbamylase (OTC) deficiency, among others. See e.g., Katrekar et al. Nat. Methods. 2019. 16:239-242; Montieel-Gonzalez et al. 2013. PNAS USA. 110: 18285-18290; Sinnamon et al. PNAS USA 2017; Wettengel et al. Curr. Gene Ther. 2018, 18:31-39; Qu et al. BioRxiv. 2019, 605972; and Fry et al. 2020. Int. J. Mol. Sci. 12:777, which are incorporated by reference as if expressed in their entirety here and the teachings of which can be adapted in view of the description herein to the engineered nucleic acid modification system of the present invention and/or component(s) thereof.

In some embodiments, the disease is an inherited retinal degeneration disease. In some embodiments, gene whose transcript can be modified using a engineered nucleic acid modification system of the present invention and/or component(s) thereof capable of RNA modification that is associated with inherited retinal degeneration and whose coding sequence is too large for packaging in a single AAV can be ABC4, USH2A, CEP290, MYO7A, EYS, and CDH23.

### Models of Diseases and Conditions

In an aspect, the invention provides a method of modeling a disease associated with a genomic locus in a eukaryotic organism or a non-human organism comprising manipulation of a target sequence within a coding, non-coding or regulatory element of said genomic locus comprising delivering a non-naturally occurring or engineered composition comprising a viral vector system comprising one or more viral vectors operably encoding a composition for expression thereof, wherein the composition comprises particle delivery system or the delivery system or the virus particle of any one of the above embodiments or the cell of any one of the above embodiment.

In one aspect, the invention provides a method of generating a model eukaryotic cell that can include one or more a mutated disease genes and/or infectious microorganisms. In some embodiments, a disease gene is any gene associated an increase in the risk of having or developing a disease. In some embodiments, the method includes (a) introducing one or more vectors into a eukaryotic cell, wherein the one or more vectors comprise a the engineered nucleic acid modification system of the present invention and/or component(s) thereof and/or vector or vector system that is capable of driving expression of the engineered nucleic acid modification system of the present invention and/or component(s) thereof including, but not limited to: a guide sequence optionally linked to a tracr mate sequence, a tracr sequence, one or more Cas effectors included in the engineered nucleic acid modification system of the present invention, and combinations thereof and (b) allowing a CRISPR-Cas complex to bind to one or more target polynucleotides, e.g., to effect binding, cleavage, nicking, or other modification of the target polynucleotide (such as introduction of a donor polynucleotide) within said disease gene, wherein the CRISPR-Cas complex is composed of one or more Cas polypeptides of the engineered nucleic acid modification system complexed with (1) one or more guide sequences that is/are hybridized to the target sequence(s) within the target polynucleotide(s), and optionally (2) the tracr mate sequence(s) that is/are hybridized to the tracr sequence(s), thereby generating a model eukaryotic cell comprising one or more mutated disease gene(s). Thus, in some embodiments the engineered nucleic acid modification system of the present invention comprises nucleic acid molecules for and drives expression of one or more of: a Cas polypeptide, Vir polypeptide (e.g., VirD1 and/or VirD2) a guide sequence linked to a tracr mate sequence, and a tracr sequence, a donor polynucleotide, and/or optionally a Homologous Recombination template and/or a stabilizing ligand if the Cas effector has a destabilization domain. In some embodiments, said cleavage comprises cleaving or nicking one or two strands at the location of the target sequence by the Cas polypeptide(s). In some embodiments, nicking comprises nicking one or two strands at the location of the target sequence by the Cas polypeptide(s). In some embodiments, said cleavage or nicking results in modified transcription of a target polynucleotide. In some embodiments, modification results in decreased transcription of the target polynucleotide. In some embodiments, the method further comprises repairing said cleaved or nicked target polynucleotide by homologous recombination with an exogenous template polynucleotide, wherein said repair results in a mutation comprising an insertion, deletion, or substitution of one or more nucleotides of said target polynucleotide. In some embodiments, said mutation results in one or more amino acid changes in a protein expression from a gene comprising the target sequence.

The disease modeled can be any disease with a genetic or epigenetic component. In some embodiments, the disease modeled can be any as discussed elsewhere herein, including but not limited to any as set forth in Tables 5 and 6 herein or any as set forth in any one or more of a disease identified as being caused or attributed to a mtDNA mutation set forth at mitomap.org.

### In Situ Disease Detection

The engineered nucleic acid modification system of the present invention and/or component(s) thereof and/or components thereof can be used for diagnostic methods of detection such as in methods analogous to CASFISH (see e.g., Deng et al. 2015. PNAS USA 112(38): 11870-11875), CRISPR-Live FISH (see e.g., Wang et al. 2020. Science; 365(6459):1301-1305), sm-FISH (Lee and Jefcoate. 2017. Front. Endocrinol. doi.org/10.3389/fendo.2017.00289), sequential FISH CRISPRainbow (Ma et al. Nat Biotechnol, 34 (2016), pp. 528-530), CRISPR-Sirius (Nat Methods, 15 (2018), pp. 928-931), Casilio (Cheng et al. Cell Res, 26 (2016), pp. 254-257), Halo-Tag based genomic loci visualization techniques (e.g., Deng et al. 2015. PNAS USA 112(38): 11870-11875; Knight et al., Science, 350 (2015), pp. 823-826), RNA-aptamer based methods (e.g. Ma et al., J Cell Biol, 214 (2016), pp. 529-537), molecular beacon-based methods (e.g. Zhao et al. Biomaterials, 100 (2016), pp. 172-183; Wu et al. Nucleic Acids Res (2018)), Quantum Dot-based systems (e.g. Ma et al. Anal Chem, 89 (2017), pp. 12896-12901), multiplexed methods (e.g. Ma et al., Proc Natl Acad Sci USA, 112 (2015), pp. 3002-3007; Fu et al. Nat Commun, 7 (2016), p. 11707; Ma et al. Nat Biotechnol, 34 (2016), pp. 528-530; Shao et al. Nucleic Acids Res, 44 (2016), Article e86); Wang et al. Sci Rep, 6 (2016), p. 26857), 9, and other in situ CRISPR-hybridization based methods (e.g. Chen et al. Cell, 155 (2013), pp. 1479-1491; Gu et al. Science, 359 (2018), pp. 1050-1055; Tanebaum et al. Cell, 159 (2014), pp. 635-646; Ye et al. Protein Cell, 8 (2017), pp. 853-855; Chen et al. Nat Commun, 9 (2018), p. 5065; Shao et al. ACS Synth Biol (2017); Fu et al. Nat Commun, 7 (2016), p. 11707; Shao et al. Nucleic Acids Res, 44 (2016), Article e86; Wang et al., Sci Rep, 6 (2016), p. 26857), all of which are incorporated by reference herein as if expressed in their entirety and whose teachings can be adapted to the engineered nucleic acid modification system of the present invention and/or component(s) thereof in view of the description herein.

In some embodiments, the engineered nucleic acid modification system of the present invention and/or component(s) thereof can be used in a detection method, such as an in situ detection method described herein. In some embodiments, the engineered nucleic acid modification system of the present invention and/or component(s) thereof can include a catalytically inactivate Cas effector described herein, preferably an inactivated Cas9 (dCas9) and/or inactivated Cas12 (dCas12) protein(s) and use this system in detection methods such as fluorescence in situ hybridization (FISH) or any other described herein. In some embodiments, the inactivated Cas polypeptide, which lacks the ability to produce DNA double-strand breaks may be fused with a marker, such as fluorescent protein, such as the enhanced green fluorescent protein (eEGFP) and co-expressed with small guide RNAs to target pericentric, centric and telomeric repeats in vivo. The dCas polypeptide or system thereof can be used to visualize both repetitive sequences and individual genes in the human genome. Such new applications of labelled dCas polypeptide and the engineered nucleic acid modification system of the present invention and/or component(s) thereof incorporating such polypeptides can be important in imaging cells and studying the functional nuclear architecture, especially in cases with a small nucleus volume or complex 3-D structures.

### Cell Selection

In some embodiments, the engineered nucleic acid modification system of the present invention and/or component(s) thereof can be used in a method to screen and/or select cells. In some embodiments, engineered nucleic acid modification system-based screening/selection method can be used to identify diseased cells in a cell population. In some embodiments, selection of the cells results in a modification in the cells such that the selected cells die. In this way, diseased cells can be identified, and removed from the healthy cell population. In some embodiments, the diseased cells can be a cancer cell, pre-cancerous cell, a virus or other pathogenic organism infected cells, or otherwise abnormal cell. In some embodiments, the modification can impart another detectable change in the cells to be selected (e.g., a functional change and/or genomic barcode) that facilitates selection of the desired cells. In some embodiments a negative selection scheme can be used to obtain a desired cell population. In these embodiments, the cells to be selected against are modified, thus can be removed from the cell population based on their death or identification or sorting based the detectable change imparted on the cells. Thus, in these embodiments, the remaining cells after selection are the desired cell population.

In some embodiments, a method of selecting one or more cell(s) containing a polynucleotide modification can include: introducing one or more the engineered nucleic acid modification system of the present invention and/or component(s) thereof and/or vectors or vector systems thereof into the cell(s), wherein the engineered nucleic acid modification system of the present invention and/or component(s) thereof and/or vectors or vector systems contains and/or is capable of expressing one or more of: a site-specific nuclease (e.g., Cas polypeptide), VirD1 and/or VirD2, and/or DNA polymerase, a donor polynucleotide, a guide sequence optionally linked to a tracr mate sequence, a tracr sequence, and optionally an editing template; wherein, for example that which is being expressed is within and expressed in vivo by the engineered nucleic acid modification system of the present invention and/or component(s) thereof, vector or vector system, and/or the donor polynucleotide and/or editing template comprises the one or more mutations that abolish site specific nuclease (e.g., Cas polypeptide) cleavage; allowing insertion of a donor polynucleotide and/or homologous recombination of the editing template with the target polynucleotide in the cell(s) to be selected; allowing a engineered nucleic acid medication system complex to bind to a target polynucleotide to effect cleavage and/or nicking of the target polynucleotide within said gene and insertion of a donor polynucleotide from a donor construct, wherein the engineered nucleic acid modification system complex comprises a site specific nuclease (e.g., a Cas polypeptide) complexed with (1) the guide sequence that is hybridized to the target sequence within the target polynucleotide, and (2) the tracr mate sequence that is hybridized to the tracr sequence, wherein binding of the complex to the target polynucleotide results in a modification to the target polynucleotide via insertion of a donor polynucleotide and induces cell death or imparts some other detectable change to the cell, thereby allowing one or more cell(s) in which one or more mutations have been introduced to be selected. In a preferred embodiment, the site-specific nuclease is a Cas polypeptide, optionally a Cas 9, dCas9, Cas12, or dCas12. In some embodiments, the cell to be selected may be a eukaryotic cell. In some embodiments, the cell to be selected may be a prokaryotic cell. Selection of specific cells via the methods herein can be performed without requiring a selection marker or a two-step process that may include a counter-selection system.

### Therapeutic Agent Development

The engineered nucleic acid modification system of the present invention and/or component(s) thereof can be used to develop engineered nucleic acid modification system-based and non-engineered nucleic acid modification system based biologically active agents, such as small molecule therapeutics. As used herein, “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. As used herein, “agent” refers to any substance, compound, molecule, and the like, which can be biologically active or otherwise can induce a biological and/or physiological effect on a subject to which it is administered to. An agent can be a primary active agent, or in other words, the component(s) of a composition to which the whole or part of the effect of the composition is attributed. An agent can be a secondary agent, or in other words, the component(s) of a composition to which an additional part and/or other effect of the composition is attributed. Thus, described herein are methods for developing a biologically active agent that modulates a cell function and/or signaling event associated with a disease and/or disease gene. In some embodiments, the method comprises (a) contacting a test compound with a diseased cell and/or a cell containing a disease gene cell; and (b) detecting a change in a readout that is indicative of a reduction or an augmentation of a cell signaling event or other cell functionality associated with said disease or disease gene, thereby developing said biologically active agent that modulates said cell signaling event or other functionality associated with said disease gene. In some embodiments, the diseased cell is a model cell described elsewhere herein. In some embodiments, the diseased cell is a diseased cell isolated from a subject in need of treatment. In some embodiments, the test compound is a small molecule agent. In some embodiments, test compound is a small molecule agent. In some embodiments, the test compound is a biologic molecule agent.

In some embodiments, the method involves developing a therapeutic based on the engineered nucleic acid modification system of the present invention and/or component(s) thereof. In particular embodiments, the therapeutic engineered nucleic acid modification system comprises a Cas polypeptide and/or a guide RNA capable of hybridizing to a target sequence of interest. In particular embodiments, the therapeutic is a vector or vector system that can contain a) a first regulatory element operably linked to a nucleotide sequence encoding the Cas effector protein(s) and other effector proteins of the engineered system of the present invention; and b) a second regulatory element operably linked to one or more nucleotide sequences encoding one or more nucleic acid molecules comprising a guide RNA comprising a guide sequence, a direct repeat sequence; wherein components (a) and (b) are located on same or different vectors. In particular embodiments, the biologically active agent is a composition comprising a delivery system operably configured to deliver the engineered nucleic acid modification system of the present invention and/or component(s) thereof, and/or or one or more polynucleotide sequences, vectors, or vector systems containing or encoding said components into a cell and capable of forming a complex such as a CRISPR-Cas complex between a Cas effector of the engineered nucleic acid modification system, and wherein said CRISPR-Cas complex is operable in the cell to guide the system to a target polynucleotide and facilitate introduction of a donor polynucleotide from a donor construct into the target polynucleotide. In some embodiments, the CRISPR-Cas complex can include the Cas effector protein(s) as described herein, guide RNA comprising the guide sequence, and a direct repeat sequence. In any such compositions, the delivery system can be a yeast system, a lipofection system, a microinjection system, a biolistic system, virosomes, liposomes, immunoliposomes, polycations, lipid:nucleic acid conjugates or artificial virions, or any other system as described herein. In particular embodiments, the delivery is via a particle, a nanoparticle, a lipid or a cell penetrating peptide (CPP).

Also described herein are methods for developing or designing a the engineered nucleic acid modification system of the present invention, optionally an engineered nucleic acid modification system based therapy or therapeutic, comprising (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, and optionally validating one or more of the (sub)selected target sites for an individual subject, optionally designing one or more gRNA recognizing one or more of said (sub)selected target sites.

In some embodiments, the method for developing or designing a gRNA for use in a the engineered nucleic acid modification system of the present invention, optionally a the engineered nucleic acid modification system based therapy or therapeutic, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, thee method for developing or designing a the engineered nucleic acid modification system of the present invention, optionally an engineered nucleic acid modification system based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments the method for developing or designing a gRNA for use in a the engineered nucleic acid modification system, optionally an engineered nucleic acid modification system based therapy or therapeutic in a population, can include (a) selecting for a (therapeutic) locus of interest gRNA target sites, wherein said target sites have minimal sequence variation across a population, and from said selected target sites subselecting target sites, wherein a gRNA directed against said target sites recognizes a minimal number of off-target sites across said population, or (b) selecting for a (therapeutic) locus of interest gRNA target sites, wherein said target sites have minimal sequence variation across a population, or selecting for a (therapeutic) locus of interest gRNA target sites, wherein a gRNA directed against said target sites recognizes a minimal number of off-target sites across said population, and optionally estimating the number of (sub)selected target sites needed to treat or otherwise modulate or manipulate a population, optionally validating one or more of the (sub)selected target sites for an individual subject, optionally designing one or more gRNA recognizing one or more of said (sub)selected target sites.

In some embodiments, the method for developing or designing an engineered nucleic acid modification system of the present invention, such as an engineered nucleic acid modification system based therapy or therapeutic, optionally in a population; or for developing or designing a gRNA for use in an the engineered nucleic acid modification system of the present invention, optionally an engineered nucleic acid modification system based therapy or therapeutic, optionally in a population, can include: selecting a set of target sequences for one or more loci in a target population, wherein the target sequences do not contain variants occurring above a threshold allele frequency in the target population (i.e. platinum target sequences); removing from said selected (platinum) target sequences any target sequences having high frequency off-target candidates (relative to other (platinum) targets in the set) to define a final target sequence set; preparing one or more, such as a set of the engineered nucleic acid modification systems based on the final target sequence set, optionally wherein a number of the engineered nucleic acid modification systems prepared is based (at least in part) on the size of a target population.

In certain embodiments, off-target candidates/off-targets, PAM restrictiveness, target cleavage efficiency, or effector protein specificity is identified or determined using a sequencing-based double-strand break (DSB) detection assay, such as described herein elsewhere. In certain embodiments, off-target candidates/off-targets are identified or determined using a sequencing-based double-strand break (DSB) detection assay, such as described herein elsewhere. In certain embodiments, off-targets, or off target candidates have at least 1, preferably 1-3, mismatches or (distal) PAM mismatches, such as 1 or more, such as 1, 2, 3, or more (distal) PAM mismatches. In certain embodiments, sequencing-based DSB detection assay comprises labeling a site of a DSB with an adapter comprising a primer binding site, labeling a site of a DSB with a barcode or unique molecular identifier, or combination thereof, as described herein elsewhere.

It will be understood that the guide sequence of the gRNA is 100% complementary to the target site, i.e., does not comprise any mismatch with the target site. It will be further understood that “recognition” of an (off-)target site by a gRNA presupposes engineered nucleic acid modification system functionality, i.e., an (off-)target site is only recognized by a gRNA if binding of the gRNA to the (off-)target site leads to engineered nucleic acid modification system activity (such as induction of single or double strand DNA cleavage, transcriptional modulation, etc.).

In certain embodiments, the target sites having minimal sequence variation across a population are characterized by absence of sequence variation in at least 99%, preferably at least 99.9%, more preferably at least 99.99% of the population. In certain embodiments, optimizing target location comprises selecting target sequences or loci having an absence of sequence variation in at least 99%, %, preferably at least 99.9%, more preferably at least 99.99% of a population. These targets are referred to herein elsewhere also as “platinum targets”. In certain embodiments, said population comprises at least 1000 individuals, such as at least 5000 individuals, such as at least 10000 individuals, such as at least 50000 individuals.

In certain embodiments, the off-target sites are characterized by at least one mismatch between the off-target site and the gRNA. In certain embodiments, the off-target sites are characterized by at most five, preferably at most four, more preferably at most three mismatches between the off-target site and the gRNA. In certain embodiments, the off-target sites are characterized by at least one mismatch between the off-target site and the gRNA and by at most five, preferably at most four, more preferably at most three mismatches between the off-target site and the gRNA.

In certain embodiments, said minimal number of off-target sites across said population is determined for high-frequency haplotypes in said population. In certain embodiments, said minimal number of off-target sites across said population is determined for high-frequency haplotypes of the off-target site locus in said population. In certain embodiments, said minimal number of off-target sites across said population is determined for high-frequency haplotypes of the target site locus in said population. In certain embodiments, the high-frequency haplotypes are characterized by occurrence in at least 0.1% of the population.

In certain embodiments, the number of (sub)selected target sites needed to treat a population is estimated based on based low frequency sequence variation, such as low frequency sequence variation captured in large scale sequencing datasets. In certain embodiments, the number of (sub)selected target sites needed to treat a population of a given size is estimated.

In certain embodiments, the method further comprises obtaining genome sequencing data of a subject to be treated; and treating the subject with an engineered nucleic acid modification system of the present invention selected from the set of the engineered nucleic acid modification systems, wherein the engineered nucleic acid modification system selected is based (at least in part) on the genome sequencing data of the individual. In certain embodiments, the ((sub)selected) target is validated by genome sequencing, preferably whole genome sequencing.

In certain embodiments, target sequences or loci as described herein are (further) selected based on optimization of one or more parameters, such as PAM type (natural or modified), PAM nucleotide content, PAM length, target sequence length, PAM restrictiveness, target cleavage efficiency, and target sequence position within a gene, a locus or other genomic region. Methods of optimization are discussed in greater detail elsewhere herein.

In certain embodiments, target sequences or loci as described herein are (further) selected based on optimization of one or more of target loci location, target length, target specificity, and PAM characteristics. As used herein, PAM characteristics may comprise for instance PAM sequence, PAM length, and/or PAM GC contents. In certain embodiments, optimizing PAM characteristics comprises optimizing nucleotide content of a PAM. In certain embodiments, optimizing nucleotide content of PAM is selecting a PAM with a motif that maximizes abundance in the one or more target loci, minimizes mutation frequency, or both. Minimizing mutation frequency can for instance be achieved by selecting PAM sequences devoid of or having low or minimal CpG.

In certain embodiments, the effector protein for each engineered nucleic acid modification system in the set of the engineered nucleic acid modification systems of the present invention is selected based on optimization of one or more parameters selected from the group consisting of, effector protein size, ability of effector protein to access regions of high chromatin accessibility, degree of uniform enzyme activity across genomic targets, epigenetic tolerance, mismatch/budge tolerance, effector protein specificity, effector protein stability or half-life, effector protein immunogenicity or toxicity. Methods of optimization are discussed in greater detail elsewhere herein.

### Gene Drives

In some embodiments, the engineered nucleic acid modification system of the present invention and/or component(s) thereof can be used to provide RNA-guided gene drives, for example in systems analogous to gene drives described in PCT Patent Publication WO 2015/105928. Systems of this kind may for example provide methods for altering eukaryotic germline cells, by introducing into the germline cell a nucleic acid sequence encoding an RNA-guided DNA nuclease and one or more guide RNAs. The guide RNAs may be designed to be complementary to one or more target locations on genomic DNA of the germline cell. The nucleic acid sequence encoding the RNA guided DNA nuclease and the nucleic acid sequence encoding the guide RNAs may be provided on constructs between flanking sequences, with promoters arranged such that the germline cell may express the RNA guided DNA nuclease and the guide RNAs, together with any desired cargo-encoding sequences that are also situated between the flanking sequences. The flanking sequences will typically include a sequence which is identical to a corresponding sequence on a selected target chromosome, so that the flanking sequences work with the components encoded by the construct to facilitate insertion of the foreign nucleic acid construct sequences into genomic DNA at a target cut site by mechanisms such as homologous recombination, to render the germline cell homozygous for the foreign nucleic acid sequence. In this way, gene-drive systems are capable of introgressing desired cargo genes throughout a breeding population (Gantz et al., 2015, Highly efficient Cas9-mediated gene drive for population modification of the malaria vector mosquito Anopheles stephensi, PNAS 2015, published ahead of print Nov. 23, 2015, doi:10.1073/pnas.1521077112; Esvelt et al., 2014, Concerning RNA-guided gene drives for the alteration of wild populations eLife 2014; 3:e03401). In select embodiments, target sequences may be selected which have few potential off-target sites in a genome. Targeting multiple sites within a target locus, using multiple guide RNAs, may increase the cutting frequency and hinder the evolution of drive resistant alleles. Truncated guide RNAs may reduce off-target cutting. Paired nickases may be used instead of a single nuclease, to further increase specificity. Gene drive constructs may include cargo sequences encoding transcriptional regulators, for example to activate homologous recombination genes and/or repress non-homologous end-joining. Target sites may be chosen within an essential gene, so that non-homologous end-joining events may cause lethality rather than creating a drive-resistant allele. The gene drive constructs can be engineered to function in a range of hosts at a range of temperatures (Cho et al. 2013, Rapid and Tunable Control of Protein Stability in Caenorhabditis elegans Using a Small Molecule, PLoS ONE 8(8): e72393. doi:10.1371/journal.pone.0072393).

### Xenotransplantation

In some embodiments, the engineered nucleic acid modification system of the present invention and/or component(s) thereof can be used to provide RNA-guided DNA nucleases adapted to be used to provide modified tissues for transplantation. For example, RNA-guided DNA nucleases may be used to knockout, knockdown or disrupt selected genes in an animal, such as a transgenic pig (such as the human heme oxygenase-1 transgenic pig line), for example by disrupting expression of genes that encode epitopes recognized by the human immune system, i.e., xenoantigen genes. Candidate porcine genes for disruption may for example include α(1,3)-galactosyltransferase and cytidine monophosphate-N-acetylneuraminic acid hydroxylase genes (see PCT Patent Publication WO 2014/066505). In addition, genes encoding endogenous retroviruses may be disrupted, for example the genes encoding all porcine endogenous retroviruses (see Yang et al., 2015, Genome-wide inactivation of porcine endogenous retroviruses (PERVs), Science 27 Nov. 2015: Vol. 350 no. 6264 pp. 1101-1104). In addition, RNA-guided DNA nucleases may be used to target a site for integration of additional genes in xenotransplant donor animals, such as a human CD55 gene to improve protection against hyperacute rejection.

### Optimization of CRISPR-Cas Systems

The methods of the present invention can involve optimization of selected parameters or variables associated with the engineered nucleic acid modification system of the present invention and/or component(s) thereof and/or its functionality, as described herein further elsewhere. Optimization of the engineered nucleic acid modification system of the present invention and/or component(s) thereof in the methods as described herein may depend on the target(s), such as the therapeutic target or therapeutic targets, the mode or type of engineered nucleic acid modification system, such as engineered nucleic acid modification system based therapeutic target(s) modulation, modification, or manipulation, as well as the delivery of the engineered nucleic acid modification system components. One or more targets may be selected, depending on the genotypic and/or phenotypic outcome. For instance, one or more therapeutic targets may be selected, depending on (genetic) disease etiology or the desired therapeutic outcome. The (therapeutic) target(s) may be a single gene, locus, or other genomic site, or may be multiple genes, loci or other genomic sites. As is known in the art, a single gene, locus, or other genomic site may be targeted more than once, such as by use of multiple gRNAs.

Engineered nucleic acid modification system activity, such as engineered nucleic acid modification system-based therapy or therapeutics may involve target disruption, such as target mutation, such as leading to gene knockout. Engineered nucleic acid modification system activity, such engineered nucleic acid modification system-based therapy or therapeutics may involve replacement of particular target sites, such as leading to target correction. Engineered nucleic acid modification system-based therapy or therapeutics may involve removal of particular target sites, such as leading to target deletion. Engineered nucleic acid modification system activity, such as Engineered nucleic acid modification system-based therapy or therapeutics may involve modulation of target site functionality, such as target site activity or accessibility, leading for instance to (transcriptional and/or epigenetic) gene or genomic region activation or gene or genomic region silencing. The skilled person will understand that modulation of target site functionality may involve engineered nucleic acid modification system mutation (such as for instance generation of a catalytically inactive site specific nuclease (e.g., Cas effector) and/or functionalization (such as for instance fusion of an engineered nucleic acid system protein (e.g., a Cas, Vir polypeptide (e.g., VirD1 and/or virD2, and/or DNA polymerase) with a heterologous functional domain, such as a transcriptional activator or repressor), as described herein elsewhere.

Accordingly, in an aspect, the invention relates to a method as described herein, comprising selection of one or more (therapeutic) target, selecting one or more engineered nucleic acid modification system functionality, and optimization of selected parameters or variables associated with the engineered nucleic acid modification system and/or its functionality. In a related aspect, the invention relates to a method as described herein, comprising (a) selecting one or more (therapeutic) target loci, (b) selecting one or more engineered nucleic acid modification system functionalities, (c) optionally selecting one or more modes of delivery, and preparing, developing, or designing an engineered nucleic acid modification system selected based on steps (a)-(c).

In certain embodiments engineered nucleic acid modification system functionality comprises genomic mutation. In certain embodiments, engineered nucleic acid modification system functionality comprises single genomic mutation. In certain embodiments, engineered nucleic acid modification system functionality comprises multiple genomic mutation. In certain embodiments, engineered nucleic acid modification system functionality comprises gene knockout. In certain embodiments, engineered nucleic acid modification system functionality comprises single gene knockout. In certain embodiments, engineered nucleic acid modification system functionality comprises multiple gene knockout. In certain embodiments, engineered nucleic acid modification system functionality comprises gene correction. In certain embodiments, engineered nucleic acid modification system functionality comprises single gene correction. In certain embodiments, engineered nucleic acid modification system functionality comprises multiple gene correction. In certain embodiments, engineered nucleic acid modification system functionality comprises genomic region correction. In certain embodiments, engineered nucleic acid modification system functionality comprises single genomic region correction. In certain embodiments, engineered nucleic acid modification system functionality comprises multiple genomic region correction. In certain embodiments, engineered nucleic acid modification system functionality comprises gene deletion. In certain embodiments, engineered nucleic acid modification system functionality comprises single gene deletion. In certain embodiments, engineered nucleic acid modification system functionality comprises multiple gene deletion. In certain embodiments, engineered nucleic acid modification system functionality comprises genomic region deletion. In certain embodiments, engineered nucleic acid modification system functionality comprises single genomic region deletion. In certain embodiments, engineered nucleic acid modification system functionality comprises multiple genomic region deletion. In certain embodiments, engineered nucleic acid modification system functionality comprises modulation of gene or genomic region functionality. In certain embodiments, engineered nucleic acid modification system functionality comprises modulation of single gene or genomic region functionality. In certain embodiments, engineered nucleic acid modification system functionality comprises modulation of multiple gene or genomic region functionality. In certain embodiments, engineered nucleic acid modification system functionality comprises gene or genomic region functionality, such as gene or genomic region activity. In certain embodiments, engineered nucleic acid modification system functionality comprises single gene or genomic region functionality, such as gene or genomic region activity. In certain embodiments, engineered nucleic acid modification system functionality comprises multiple gene or genomic region functionality, such as gene or genomic region activity. In certain embodiments, engineered nucleic acid modification system functionality comprises modulation gene activity or accessibility optionally leading to transcriptional and/or epigenetic gene or genomic region activation or gene or genomic region silencing. In certain embodiments, engineered nucleic acid modification system functionality comprises modulation single gene activity or accessibility optionally leading to transcriptional and/or epigenetic gene or genomic region activation or gene or genomic region silencing. In certain embodiments, engineered nucleic acid modification system functionality comprises modulation multiple gene activity or accessibility optionally leading to transcriptional and/or epigenetic gene or genomic region activation or gene or genomic region silencing.

Optimization of selected parameters or variables in the methods as described herein may result in optimized or improved engineered nucleic acid modification system, such as an engineered nucleic acid modification system-based therapy or therapeutic, specificity, efficacy, and/or safety. In certain embodiments, one or more of the following parameters or variables are taken into account, are selected, or are optimized in the methods of the invention as described herein: Cas protein allosteric interactions, Cas protein functional domains and functional domain interactions, CRISPR-Cas effector specificity, gRNA specificity, CRISPR-Cas complex specificity, PAM restrictiveness, PAM type (natural or modified), PAM nucleotide content, PAM length, CRISPR-Cas effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR effector stability, CRISPR-Cas effector mRNA stability, gRNA stability, CRISPR-Cas complex stability, CRISPR-Cas effector protein or mRNA immunogenicity or toxicity, gRNA immunogenicity or toxicity, CRISPR-Cas complex immunogenicity or toxicity, CRISPR-Cas effector protein or mRNA dose or titer, gRNA dose or titer, CRISPR-Cas complex dose or titer, CRISPR effector protein size, CRISPR effector expression level, gRNA expression level, CRISPR-Cas complex expression level, CRISPR-Cas effector spatiotemporal expression, gRNA spatiotemporal expression, CRISPR-Cas complex spatiotemporal expression.

By means of example, and without limitation, parameter or variable optimization may be achieved as follows. CRISPR-Cas effector specificity may be optimized by selecting the most specific CRISPR-Cas effector. This may be achieved for instance by selecting the most specific CRISPR-Cas effector orthologue or by specific CRISPR-Cas effector mutations which increase specificity. gRNA specificity may be optimized by selecting the most specific gRNA. This can be achieved for instance by selecting gRNA having low homology, i.e., at least one or preferably more, such as at least 2, or preferably at least 3, mismatches to off-target sites. CRISPR-Cas complex specificity may be optimized by increasing CRISPR effector specificity and/or gRNA specificity as above. PAM restrictiveness may be optimized by selecting a CRISPR-Cas effector having to most restrictive PAM recognition. This can be achieved for instance by selecting a CRISPR-Cas effector orthologue having more restrictive PAM recognition or by specific CRISPR-Cas effector mutations which increase or alter PAM restrictiveness. PAM type may be optimized for instance by selecting the appropriate CRISPR-Cas effector, such as the appropriate CRISPR-Cas effector recognizing a desired PAM type. The CRISPR-Cas effector or PAM type may be naturally occurring or may for instance be optimized based on CRISPR-Cas effector mutants having an altered PAM recognition, or PAM recognition repertoire. PAM nucleotide content may for instance be optimized by selecting the appropriate CRISPR-Cas effector, such as the appropriate CRISPR-Cas effector recognizing a desired PAM nucleotide content. The CRISPR-Cas effector or PAM type may be naturally occurring or may for instance be optimized based on CRISPR-Cas effector mutants having an altered PAM recognition, or PAM recognition repertoire. PAM length may for instance be optimized by selecting the appropriate CRISPR-Cas effector, such as the appropriate CRISPR-Cas effector recognizing a desired PAM nucleotide length. The CRISPR-Cas effector or PAM type may be naturally occurring or may for instance be optimized based on CRISPR-Cas effector mutants having an altered PAM recognition, or PAM recognition repertoire.

Target length or target sequence length may for instance be optimized by selecting the appropriate CRISPR-Cas effector, such as the appropriate CRISPR-Cas effector recognizing a desired target or target sequence nucleotide length. Alternatively, or in addition, the target (sequence) length may be optimized by providing a target having a length deviating from the target (sequence) length typically associated with the CRISPR-Cas effector, such as the naturally occurring CRISPR-Cas effector. The CRISPR-Cas effector or target (sequence) length may be naturally occurring or may for instance be optimized based on CRISPR-Cas effector mutants having an altered target (sequence) length recognition, or target (sequence) length recognition repertoire. For instance, increasing or decreasing target (sequence) length may influence target recognition and/or off-target recognition. CRISPR-Cas effector activity may be optimized by selecting the most active CRISPR-Cas effector. This may be achieved for instance by selecting the most active CRISPR-Cas effector orthologue or by specific CRISPR-Cas effector mutations which increase activity. The ability of the CRISPR-Cas effector protein to access regions of high chromatin accessibility, may be optimized by selecting the appropriate CRISPR-Cas effector or mutant thereof, and can consider the size of the CRISPR-Cas effector, charge, or other dimensional variables etc. The degree of uniform CRISPR-Cas effector activity may be optimized by selecting the appropriate CRISPR effector or mutant thereof, and can consider CRISPR-Cas effector specificity and/or activity, PAM specificity, target length, mismatch tolerance, epigenetic tolerance, CRISPR-Cas effector and/or gRNA stability and/or half-life, CRISPR-Cas effector and/or gRNA immunogenicity and/or toxicity, etc. gRNA activity may be optimized by selecting the most active gRNA. In some embodiments, this can be achieved by increasing gRNA stability through RNA modification. CRISPR-Cas complex activity may be optimized by increasing CRISPR-Cas effector activity and/or gRNA activity as above.

The target site selection may be optimized by selecting the optimal position of the target site within a gene, locus or other genomic region. The target site selection may be optimized by optimizing target location comprises selecting a target sequence with a gene, locus, or other genomic region having low variability. This may be achieved for instance by selecting a target site in an early and/or conserved exon or domain (i.e., having low variability, such as polymorphisms, within a population).

In certain embodiments, optimizing target (sequence) length comprises selecting a target sequence within one or more target loci between 5 and 25 nucleotides. In certain embodiments, a target sequence is 20 nucleotides.

In certain embodiments, optimizing target specificity comprises selecting targets loci that minimize off-target candidates.

In some embodiments, the target site may be selected by minimization of off-target effects (e.g., off-targets qualified as having 1-5, 1-4, or preferably 1-3 mismatches compared to target and/or having one or more PAM mismatches, such as distal PAM mismatches), preferably also considering variability within a population. CRISPR-Cas effector stability may be optimized by selecting CRISPR-Cas effector having appropriate half-life, such as preferably a short half-life while still capable of maintaining sufficient activity. In some embodiments, this can be achieved by selecting an appropriate CRISPR-Cas effector orthologue having a specific half-life or by specific CRISPR-Cas effector mutations or modifications which affect half-life or stability, such as inclusion (e.g., fusion) of stabilizing or destabilizing domains or sequences. CRISPR-Cas effector mRNA stability may be optimized by increasing or decreasing CRISPR-Cas effector mRNA stability. In some embodiments, this can be achieved by increasing or decreasing CRISPR-Cas effector mRNA stability through mRNA modification. gRNA stability may be optimized by increasing or decreasing gRNA stability. In some embodiments, this can be achieved by increasing or decreasing gRNA stability through RNA modification. CRISPR-Cas complex stability may be optimized by increasing or decreasing CRISPR-Cas effector stability and/or gRNA stability as above. CRISPR-Cas effector protein or mRNA immunogenicity or toxicity may be optimized by decreasing CRISPR-Cas effector protein or mRNA immunogenicity or toxicity. In some embodiments, this can be achieved by mRNA or protein modifications. Similarly, in case of DNA based expression systems, DNA immunogenicity or toxicity may be decreased. gRNA immunogenicity or toxicity may be optimized by decreasing gRNA immunogenicity or toxicity. In some embodiments, this can be achieved by gRNA modifications. Similarly, in case of DNA based expression systems, DNA immunogenicity or toxicity may be decreased. CRISPR-Cas complex immunogenicity or toxicity may be optimized by decreasing CRISPR-Cas effector immunogenicity or toxicity and/or gRNA immunogenicity or toxicity as above, or by selecting the least immunogenic or toxic CRISPR-Cas effector/gRNA combination. Similarly, in case of DNA based expression systems, DNA immunogenicity or toxicity may be decreased. CRISPR-Cas effector protein or mRNA dose or titer may be optimized by selecting dosage or titer to minimize toxicity and/or maximize specificity and/or efficacy. gRNA dose or titer may be optimized by selecting dosage or titer to minimize toxicity and/or maximize specificity and/or efficacy. CRISPR-Cas complex dose or titer may be optimized by selecting dosage or titer to minimize toxicity and/or maximize specificity and/or efficacy. CRISPR-Cas effector protein size may be optimized by selecting minimal protein size to increase efficiency of delivery, in particular for virus mediated delivery. CRISPR-Cas effector, gRNA, or CRISPR-Cas complex expression level may be optimized by limiting (or extending) the duration of expression and/or limiting (or increasing) expression level. This may be achieved for instance by using self-inactivating CRISPR-Cas systems, such as including a self-targeting (e.g. CRISPR-Cas effector targeting) gRNA, by using viral vectors having limited expression duration, by using appropriate promoters for low (or high) expression levels, by combining different delivery methods for individual CRISP-Cas system components, such as virus mediated delivery of CRISPR-Cas effector encoding nucleic acid combined with non-virus mediated delivery of gRNA, or virus mediated delivery of gRNA combined with non-virus mediated delivery of CRISPR-Cas effector protein or mRNA. CRISPR-Cas effector, gRNA, or CRISPR-Cas complex spatiotemporal expression may be optimized by appropriate choice of conditional and/or inducible expression systems, including controllable CRISPR effector activity optionally a destabilized CRISPR-Cas effector and/or a split CRISPR-Cas effector, and/or cell- or tissue-specific expression systems.

In an embodiment, the invention relates to a method as described herein, comprising selection of one or more (therapeutic) target, selecting engineered nucleic acid modification system functionalities, selecting engineered nucleic acid modification system mode of delivery, selecting engineered nucleic acid modification system delivery vehicle or expression system, and optimization of selected parameters or variables associated with the engineered nucleic acid modification system and/or its functionality, optionally wherein the parameters or variables are one or more selected from CRISPR-Cas effector specificity, gRNA specificity, CRISPR-Cas complex specificity, PAM restrictiveness, PAM type (natural or modified), PAM nucleotide content, PAM length, CRISPR-Cas effector activity, gRNA activity, CRISPR-Cas complex activity, target cleavage efficiency, target site selection, target sequence length, ability of effector protein to access regions of high chromatin accessibility, degree of uniform enzyme activity across genomic targets, epigenetic tolerance, mismatch/budge tolerance, CRISPR-Cas effector stability, CRISPR-Cas effector mRNA stability, gRNA stability, CRISPR-Cas complex stability, CRISPR-Cas effector protein or mRNA immunogenicity or toxicity, gRNA immunogenicity or toxicity, CRISPR-Cas complex immunogenicity or toxicity, CRISPR-Cas effector protein or mRNA dose or titer, gRNA dose or titer, CRISPR-Cas complex dose or titer, CRISPR-Cas effector protein size, CRISPR-Cas effector expression level, gRNA expression level, CRISPR-Cas complex expression level, CRISPR-Cas effector spatiotemporal expression, gRNA spatiotemporal expression, CRISPR-Cas complex spatiotemporal expression.

In an embodiment, the invention relates to a method as described herein, comprising selecting one or more (therapeutic) target, selecting one or more engineered nucleic acid modification system functionalities, selecting one or more engineered nucleic acid modification system mode of delivery, selecting one or more engineered nucleic acid modification system delivery vehicles or expression systems, and optimization of selected parameters or variables associated with the engineered nucleic acid modification system and/or its functionality, wherein specificity, efficacy, and/or safety are optimized, and optionally wherein optimization of specificity comprises optimizing one or more parameters or variables selected from CRISPR-Cas effector specificity, gRNA specificity, CRISPR-Cas complex specificity, PAM restrictiveness, PAM type (natural or modified), PAM nucleotide content, PAM length, wherein optimization of efficacy comprises optimizing one or more parameters or variables selected from CRISPR-Cas effector activity, gRNA activity, CRISPR-Cas complex activity, target cleavage efficiency, target site selection, target sequence length, CRISPR-Cas effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR-Cas effector stability, CRISPR-Cas effector mRNA stability, gRNA stability, CRISPR-Cas complex stability, CRISPR-Cas effector protein or mRNA immunogenicity or toxicity, gRNA immunogenicity or toxicity, CRISPR-Cas complex immunogenicity or toxicity, CRISPR-Cas effector protein or mRNA dose or titer, gRNA dose or titer, CRISPR-Cas complex dose or titer, CRISPR-Cas effector expression level, gRNA expression level, CRISPR-Cas complex expression level, CRISPR-Cas effector spatiotemporal expression, gRNA spatiotemporal expression, CRISPR-Cas complex spatiotemporal expression.

In an embodiment, the invention relates to a method as described herein, comprising optionally selecting one or more (therapeutic) target, optionally selecting one or more engineered nucleic acid modification system functionalities, optionally selecting one or more engineered nucleic acid modification system modes of delivery, optionally selecting one or more engineered nucleic acid modification system delivery vehicles or expression systems, and optimization of selected parameters or variables associated with the engineered nucleic acid modification system and/or its functionality, wherein specificity, efficacy, and/or safety are optimized, and optionally wherein optimization of specificity comprises optimizing one or more parameters or variables selected from CRISPR-Cas effector specificity, gRNA specificity, CRISPR-Cas complex specificity, PAM restrictiveness, PAM type (natural or modified), PAM nucleotide content, PAM length, wherein optimization of efficacy comprises optimizing one or more parameters or variables selected from CRISPR-Cas effector activity, gRNA activity, CRISPR-Cas complex activity, target cleavage efficiency, target site selection, target sequence length, CRISPR-Cas effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR-Cas effector stability, CRISPR-Cas effector mRNA stability, gRNA stability, CRISPR-Cas complex stability, CRISPR-Cas effector protein or mRNA immunogenicity or toxicity, gRNA immunogenicity or toxicity, CRISPR-Cas complex immunogenicity or toxicity, CRISPR effector protein or mRNA dose or titer, gRNA dose or titer, CRISPR-Cas complex dose or titer, CRISPR-Cas effector expression level, gRNA expression level, CRISPR-Cas complex expression level, CRISPR-Cas effector spatiotemporal expression, gRNA spatiotemporal expression, CRISPR-Cas complex spatiotemporal expression.

In an aspect, the invention relates to a method as described herein, comprising optimization of selected parameters or variables associated with the engineered nucleic acid modification system and/or its functionality, wherein specificity, efficacy, and/or safety are optimized, and optionally wherein optimization of specificity comprises optimizing one or more parameters or variables selected from CRISPR-Cas effector specificity, gRNA specificity, CRISPR-Cas complex specificity, PAM restrictiveness, PAM type (natural or modified), PAM nucleotide content, PAM length, wherein optimization of efficacy comprises optimizing one or more parameters or variables selected from CRISPR-Cas effector activity, gRNA activity, CRISPR-Cas complex activity, target cleavage efficiency, target site selection, target sequence length, CRISPR-Cas effector protein size, ability of effector protein to access regions of high chromatin accessibility, degree of uniform enzyme activity across genomic targets, epigenetic tolerance, mismatch/budge tolerance, and wherein optimization of safety comprises optimizing one or more parameters or variables selected from CRISPR-Cas effector stability, CRISPR-Cas effector mRNA stability, gRNA stability, CRISPR-Cas complex stability, CRISPR-Cas effector protein or mRNA immunogenicity or toxicity, gRNA immunogenicity or toxicity, CRISPR-Cas complex immunogenicity or toxicity, CRISPR-Cas effector protein or mRNA dose or titer, gRNA dose or titer, CRISPR-Cas complex dose or titer, CRISPR-Cas effector expression level, gRNA expression level, CRISPR-Cas complex expression level, CRISPR-Cas effector spatiotemporal expression, gRNA spatiotemporal expression, CRISPR-Cas complex spatiotemporal expression.

It will be understood that the parameters or variables to be optimized as well as the nature of optimization may depend on the (therapeutic) target, the engineered nucleic acid modification system functionality, the engineered nucleic acid modification system mode of delivery, and/or the engineered nucleic acid modification system delivery vehicle or expression system.

In an aspect, the invention relates to a method as described herein, comprising optimization of gRNA specificity at the population level. Preferably, said optimization of gRNA specificity comprises minimizing gRNA target site sequence variation across a population and/or minimizing gRNA off-target incidence across a population.

In some embodiments, optimization can result in selection of a CRISPR-Cas effector that is naturally occurring or is modified. In some embodiments, optimization can result in selection of a CRISPR-Cas effector that has nuclease, nickase, deaminase, transposase, and/or has one or more effector functionalities deactivated or eliminated. In some embodiments, optimizing a PAM specificity can include selecting a CRISPR-Cas effector with a modified PAM specificity. In some embodiments, optimizing can include selecting a CRISPR-Cas effector having a minimal size. In certain embodiments, optimizing effector protein stability comprises selecting an effector protein having a short half-life while maintaining sufficient activity, such as by selecting an appropriate CRISPR effector orthologue having a specific half-life or stability. In certain embodiments, optimizing immunogenicity or toxicity comprises minimizing effector protein immunogenicity or toxicity by protein modifications. In certain embodiments, optimizing functional specific comprises selecting a protein effector with reduced tolerance of mismatches and/or bulges between the guide RNA and one or more target loci.

In certain embodiments, optimizing efficacy comprises optimizing overall efficiency, epigenetic tolerance, or both. In certain embodiments, maximizing overall efficiency comprises selecting an effector protein with uniform enzyme activity across target loci with varying chromatin complexity, selecting an effector protein with enzyme activity limited to areas of open chromatin accessibility. In certain embodiments, chromatin accessibility is measured using one or more of ATAC-seq, or a DNA-proximity ligation assay. In certain embodiments, optimizing epigenetic tolerance comprises optimizing methylation tolerance, epigenetic mark competition, or both. In certain embodiments, optimizing methylation tolerance comprises selecting an effector protein that modify methylated DNA. In certain embodiments, optimizing epigenetic tolerance comprises selecting an effector protein unable to modify silenced regions of a chromosome, selecting an effector protein able to modify silenced regions of a chromosome, or selecting target loci not enriched for epigenetic markers

In certain embodiments, selecting an optimized guide RNA comprises optimizing gRNA stability, gRNA immunogenicity, or both, or other gRNA associated parameters or variables as described herein elsewhere.

In certain embodiments, optimizing gRNA stability and/or gRNA immunogenicity comprises RNA modification, or other gRNA associated parameters or variables as described herein elsewhere. In certain embodiments, the modification comprises removing 1-3 nucleotides form the 3′ end of a target complementarity region of the gRNA. In certain embodiments, modification comprises an extended gRNA and/or trans RNA/DNA element that create stable structures in the gRNA that compete with gRNA base pairing at a target of off-target loci, or extended complimentary nucleotides between the gRNA and target sequence, or both.

In certain embodiments, the mode of delivery comprises delivering gRNA and/or CRISPR effector protein or other component of a engineered nucleic acid modification system of the present invention, delivering gRNA and/or CRISPR effector mRNA or other mRNA of the engineered nucleic acid modification system of the present invention, or delivery gRNA and/or CRISPR-Cas effector as a DNA based expression system. In certain embodiments, the mode of delivery further comprises selecting a delivery vehicle and/or expression systems from the group consisting of liposomes, lipid particles, nanoparticles, biolistics, or viral-based expression/delivery systems. In certain embodiments, expression is spatiotemporal expression is optimized by choice of conditional and/or inducible expression systems, including controllable CRISPR-Cas effector activity, optionally a destabilized CRISPR-Cas effector and/or a split CRISPR-CAs effector, and/or cell- or tissue-specific expression system.

The methods as described herein may further involve selection of the engineered nucleic acid modification system mode of delivery. In certain embodiments, donor construct gRNA (and tracr, if and where needed, optionally provided as a sgRNA) and/or CRISPR-Cas effector protein are or are to be delivered. In certain embodiments, gRNA (and tracr, if and where needed, optionally provided as a sgRNA) and/or CRISPR-CAs effector mRNA are or are to be delivered. In certain embodiments, gRNA (and tracr, if and where needed, optionally provided as a sgRNA) and/or CRISPR-Cas effector provided in a DNA-based expression system are or are to be delivered. In certain embodiments, delivery of the individual engineered nucleic acid modification system components comprises a combination of the above modes of delivery. In certain embodiments, delivery comprises delivering a donor construct, gRNA and/or CRISPR-Cas and other engineered nucleic acid modification system effector proteins, delivering gRNA and/or CRISPR-Cas and other engineered nucleic acid modification system effector mRNA, or delivering a donor construct, gRNA and/or CRISPR-Cas or other system effector as a DNA based expression system.

The methods as described herein may further involve selection of the engineered nucleic acid modification system delivery vehicle and/or expression system. Delivery vehicles and expression systems are described herein elsewhere. By means of example, delivery vehicles of nucleic acids and/or proteins include nanoparticles, liposomes, etc. Delivery vehicles for DNA, such as DNA-based expression systems include for instance biolistics, viral based vector systems (e.g., adenoviral, AAV, lentiviral), etc. the skilled person will understand that selection of the mode of delivery, as well as delivery vehicle or expression system may depend on for instance the cell or tissues to be targeted. In certain embodiments, the delivery vehicle and/or expression system for delivering the CRISPR-Cas systems or components thereof comprises liposomes, lipid particles, nanoparticles, biolistics, or viral-based expression/delivery systems.

### Considerations for Therapeutic Applications

A consideration in genome editing therapy is the choice of sequence-specific nuclease, such as a variant of a Cas (e.g., Cas9, dCas9, Cas12 and/or dCas12) nuclease. Each nuclease variant may possess its own unique set of strengths and weaknesses, many of which must be balanced in the context of treatment to maximize therapeutic benefit. For a specific editing therapy to be efficacious, a sufficiently high level of modification must be achieved in target cell populations to reverse disease symptoms. This therapeutic modification ‘threshold’ is determined by the fitness of edited cells following treatment and the amount of gene product necessary to reverse symptoms. With regard to fitness, editing creates three potential outcomes for treated cells relative to their unedited counterparts: increased, neutral, or decreased fitness. In the case of increased fitness, corrected cells may be able and expand relative to their diseased counterparts to mediate therapy. In this case, where edited cells possess a selective advantage, even low numbers of edited cells can be amplified through expansion, providing a therapeutic benefit to the patient. Where the edited cells possess no change in fitness, an increase the therapeutic modification threshold can be warranted. As such, significantly greater levels of editing may be needed to treat diseases, where editing creates a neutral fitness advantage, relative to diseases where editing creates increased fitness for target cells. If editing imposes a fitness disadvantage, as would be the case for restoring function to a tumor suppressor gene in cancer cells, modified cells would be outcompeted by their diseased counterparts, causing the benefit of treatment to be low relative to editing rates. This may be overcome with supplemental therapies to increase the potency and/or fitness of the edited cells relative to the diseased counterparts.

In addition to cell fitness, the amount of gene product necessary to treat disease can also influence the minimal level of therapeutic genome editing that can treat or prevent a disease or a symptom thereof. In cases where a small change in the gene product levels can result in significant changes in clinical outcome, the minimal level of therapeutic genome editing is less relative to cases where a larger change in the gene product levels are needed to gain a clinically relevant response. In some embodiments, the minimal level of therapeutic genome editing can range from 0.1 to 1%, 1-5%, 5-10%, 10-15%, 15-20%, 20-25%, 25-30%, 30-35%, 35-40%, 40-45%. 45-50%, or 50-55%. Thus, where a small change in gene product levels can influence clinical outcomes and diseases where there is a fitness advantage for edited cells, are ideal targets for genome editing therapy, as the therapeutic modification threshold is low enough to permit a high chance of success.

The activity of NHEJ and HDR DSB repair can vary by cell type and cell state. NHEJ is not highly regulated by the cell cycle and is efficient across cell types, allowing for high levels of gene disruption in accessible target cell populations. In contrast, HDR acts primarily during S/G2 phase, and is therefore restricted to cells that are actively dividing, limiting treatments that require precise genome modifications to mitotic cells [Ciccia, A. & Elledge, S. J. Molecular cell 40, 179-204 (2010); Chapman, J. R., et al. Molecular cell 47, 497-510 (2012)].

The efficiency of correction via HDR may be controlled by the epigenetic state or sequence of the targeted locus, or the specific repair template configuration (single vs. double stranded, long vs. short homology arms) used [Hacein-Bey-Abina, S., et al. The New England journal of medicine 346, 1185-1193 (2002); Gaspar, H. B., et al. Lancet 364, 2181-2187 (2004); Beumer, K. J., et al. G3 (2013)]. The relative activity of NHEJ and HDR machineries in target cells may also affect gene correction efficiency, as these pathways may compete to resolve DSBs [Beumer, K. J., et al. Proceedings of the National Academy of Sciences of the United States of America 105, 19821-19826 (2008)]. HDR also imposes a delivery challenge not seen with NHEJ strategies, as it uses the concurrent delivery of nucleases and repair templates. Thus, these differences can be kept in mind when designing, optimizing, and/or selecting a engineered nucleic acid modification system based therapeutic as described in greater detail elsewhere herein.

Polynucleotide modification via the engineered nucleic acid modification system can include combinations of proteins, small RNA molecules, donor constructs, editing templates, and/or repair templates, and can make, in some embodiments, delivery of these multiple parts substantially more challenging than, for example, traditional small molecule therapeutics. Two main strategies for delivery of the engineered nucleic acid modification system and components thereof have been developed: ex vivo and in vivo. In some embodiments of ex vivo treatments, diseased cells are removed from a subject, edited and then transplanted back into the patient. In other embodiments, cells from a healthy allogeneic donor are collected, modified using a engineered nucleic acid modification system or component thereof, to impart various functionalities and/or reduce immunogenicity, and administered to an allogeneic recipient in need of treatment. Ex vivo editing has the advantage of allowing the target cell population to be well defined and the specific dosage of therapeutic molecules delivered to cells to be specified. The latter consideration may be particularly important when off-target modifications are a concern, as titrating the amount of nuclease may decrease such mutations (Hsu et al., 2013). Another advantage of ex vivo approaches is the typically high editing rates that can be achieved, due to the development of efficient delivery systems for proteins and nucleic acids into cells in culture for research and gene therapy applications.

In vivo polynucleotide modification via engineered nucleic acid modification systems and/or components thereof involves direct delivery of engineered nucleic acid modification systems and/or components thereof to cell types in their native tissues. In vivo polynucleotide modification via engineered nucleic acid modification systems and/or components thereof allows diseases in which the affected cell population is not amenable to ex vivo manipulation to be treated. Furthermore, engineered nucleic acid modification systems and/or components thereof to cells in situ allows for the treatment of multiple tissue and cell types.

In some embodiments, such as those where viral vector systems are used to generate viral particles to deliver the engineered nucleic acid modification system and/or component thereof to a cell, the total cargo size of the engineered nucleic acid modification system and/or component thereof should be considered as vector systems can have limits on the size of a polynucleotide that can be expressed therefrom and/or packaged into cargo inside of a viral particle. In some embodiments, the tropism of a vector system, such as a viral vector system, should be considered as it can impact the cell type to which the engineered nucleic acid modification system or component thereof can be efficiently and/or effectively delivered.

When delivering a engineered nucleic acid modification system or component thereof via a viral-based system, it can be important to consider the amount of viral particles that will be needed to achieve a therapeutic effect so as to account for the potential immune response that can be elicited by the viral particles when delivered to a subject or cell(s). When delivering a engineered nucleic acid modification system or component thereof via a viral based system, it can be important to consider mechanisms of controlling the distribution and/or dosage of the engineered nucleic acid modification system in vivo. Generally, to reduce the potential for off-target effects, it is optimal but not necessarily required, that the amount of the engineered nucleic acid modification system be as close to the minimum or least effective dose. In practice this can be challenging to do.

In some embodiments, it can be important to consider the immunogenicity of the engineered nucleic acid modification system or component thereof. In embodiments, where the immunogenicity of the engineered nucleic acid modification system or component thereof is of concern, the immunogenicity engineered nucleic acid modification system or component thereof can be reduced. By way of example only, the immunogenicity of the engineered nucleic acid modification system or component thereof can be reduced using the approach set out in Tangri et al. Accordingly, directed evolution or rational design may be used to reduce the immunogenicity of a polypeptide of the engineered nucleic acid modification system (for instance a Cas (e.g. Cas9, dCas9, Cas12 and/or dCas12)) in the host species (human or other species).

### Kits

In another aspect, the present disclosure provides kit and kit of parts. The terms “kit of parts” and “kit” as used throughout this specification refer to a product containing components necessary for carrying out the specified methods (e.g., methods for detecting, quantifying or isolating immune cells as taught herein), packed so as to allow their transport and storage. Materials suitable for packing the components comprised in a kit include crystal, plastic (e.g., polyethylene, polypropylene, polycarbonate), bottles, flasks, vials, ampules, paper, envelopes, or other types of containers, carriers or supports. Where a kit comprises a plurality of components, at least a subset of the components (e.g., two or more of the plurality of components) or all of the components may be physically separated, e.g., comprised in or on separate containers, carriers or supports. The components comprised in a kit may be sufficient or may not be sufficient for carrying out the specified methods, such that external reagents or substances may not be necessary or may be necessary for performing the methods, respectively. Typically, kits are employed in conjunction with standard laboratory equipment, such as liquid handling equipment, environment (e.g., temperature) controlling equipment, analytical instruments, etc. In addition to the recited binding agents(s) as taught herein, such as for example, antibodies, hybridization probes, amplification and/or sequencing primers, optionally provided on arrays or microarrays, the present kits may also include some or all of solvents, buffers (such as for example but without limitation histidine-buffers, citrate-buffers, succinate-buffers, acetate-buffers, phosphate-buffers, formate buffers, benzoate buffers, TRIS (Tris(hydroxymethyl)-aminomethan) buffers or maleate buffers, or mixtures thereof), enzymes (such as for example but without limitation thermostable DNA polymerase), detectable labels, detection reagents, and control formulations (positive and/or negative), useful in the specified methods. Typically, the kits may also include instructions for use thereof, such as on a printed insert or on a computer readable medium. The terms may be used interchangeably with the term “article of manufacture”, which broadly encompasses any man-made tangible structural product, when used in the present context.

### EXAMPLES

### Example 1

Agrobacterium transfer DNA (T-DNA) systems have been widely used in agriculture for gene delivery and integration into plant cells. See e.g., Gelvin et al. 2003. Microbiol. Molec. Biol. Rev. 67:16-37 and Nester E. W. 2015. Front. Plant. Sci. Art. 730. The system is typically applied using a two-vector system: (1) binary plasmid and (2) helper plasmid. The binary plasmid can contain the two border sequences flanking a gene of interest (GOI). The helper plasmid carries the Agrobacterium virulence genes (virA through virH). virD1 and virD2 are known to be involved in releasing the single-stranded T-DNA from the binary plasmid. When released, the 5′ end of the T-DNA is covalently linked with a molecule of virD2 (See e.g., Gelvin et al. 2003. Microbiol. Molec. Biol. Rev. 67:16-37 and Nester E. W. 2015. Front. Plant. Sci. Art. 730. and Singer DOI 10.1007/82_2018_98. When virD2 alone is incubated with ssDNA containing the full 25 nt RB, virD2 is able to cleave the RB sequence and covalently link to the 5′ of the cleaved ssDNA. See e.g., Pansegrau et al. 1993. PNAS. December 15; 90(24):11538-11542.

This Example describes compositions and systems for precise insertion of DNA into the target site. As shown in FIGS. 1-4 Cas9 (wildtype or nickase)-virD2 can be fused with a DNA polymerase, such as the E. coli DNA polymerase I. The free 3′ end of the cleaved non-targeted strand will be able to prime off of the single-stranded T-DNA (a donor polynucleotide sequence flanked by 5′ and/or 3′ boundaries) to allow the sequence in the T-DNA to be incorporated into the target site.

A vector based donor can be used to provide a double-stranded donor polynucleotide sequence (see e.g., FIG. 1). A fusion protein or complex including a Cas9-VirD2-DNA polymerase can be introduced to the double-stranded donor construct and a VirD1 protein. The VirD1 can, in some instance, be complexed or connected to the Cas9-VirD2-DNA polymerase fusion protein or complex. The VirD1 and VirD2 can work in concert to cleave the donor polynucleotide from the donor construct and conjugate it to the VirD2 protein. A guide RNA can be complexed with the Cas9-VirD2-DNA polymerase fusion protein or complex or with the resulting Cas9-VirD2-DNA polymerase-donor polynucleotide sequence and act to guide the complex a target polynucleotide. This can occur in vitro, ex vivo, or in vivo. In some embodiments, one or more or all components of the system can be introduced to a target polynucleotide in vitro. In some instances, one or more or all components of the system can be introduced to a cell containing the target polynucleotide. In some embodiments, one more or all of components of the system can be provided to a cell in a vector or vector, whereby the components are expressed from the vector and complex and go on to modify a target polynucleotide within the cell. In other instances, the Cas9-VirD2-DNA-polymerase can be conjugated to a donor polynucleotide then complexed with a guide RNA molecule to form an RNP, which can be delivered to a cell containing a target polynucleotide. In some of these instances, the guide RNA molecule can be produced from an expression vector, which expresses the guide RNA molecule and then is complexed to form the RNP.

In some instances, the donor construct can be a single stranded DNA as shown in FIG. 2. In these instances, VirD1 is not required as VirD2 can act in vitro to cleave and conjugate to the single stranded 5′ boundary sequence. A fusion protein or complex including a Cas9-VirD2-DNA polymerase can be introduced to single stranded donor construct and act to conjugate the donor polynucleotide to the VirD2. A guide RNA can be complexed with the Cas9-VirD2-DNA polymerase fusion protein or complex or with the resulting Cas9-VirD2-DNA polymerase-donor polynucleotide sequence and act to guide the complex a target polynucleotide. This can occur in vitro, ex vivo, or in vivo. In some embodiments, one or more or all components of the system can be introduced to a target polynucleotide in vitro. In some instances, one or more or all components of the system can be introduced to a cell containing the target polynucleotide. In some embodiments, one more or all of components of the system can be provided to a cell in a vector or vector, whereby the components are expressed from the vector and complex and go on to modify a target polynucleotide within the cell. In other instances, the Cas9-VirD2-DNA-polymerase can be conjugated to a donor polynucleotide then complexed with a guide RNA molecule to form an RNP, which can be delivered to a cell containing a target polynucleotide. In some of these instances, the guide RNA molecule can be produced from an expression vector, which expresses the guide RNA molecule and then is complexed to form the RNP.

Delivery of any of the previously discussed components can be delivered by any suitable delivery method or technique.

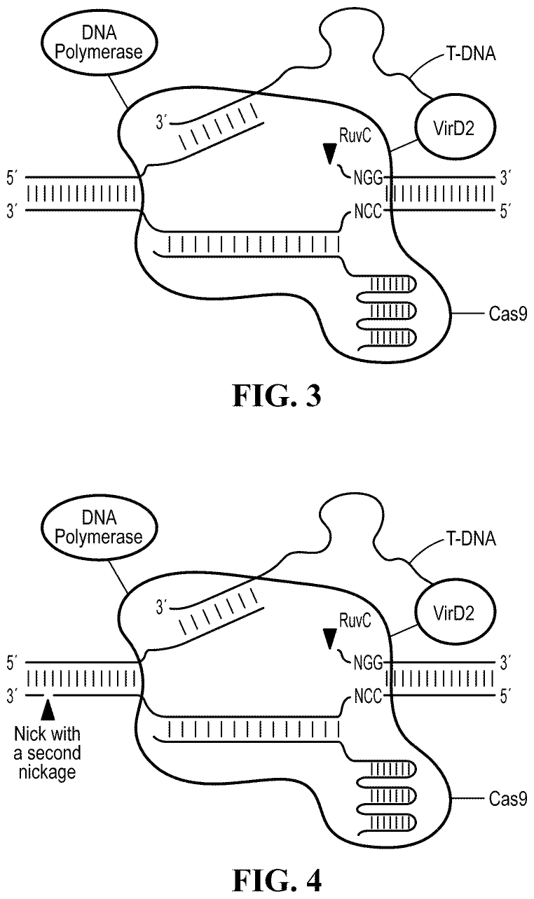

As shown in FIG. 3, the DNA polymerase can prime off the donor polynucleotide sequence to result in integration of the donor polynucleotide sequence into the target polynucleotide.

As shown in FIG. 4, efficiency of insertion by this system can be improved by incorporating a second nickase may be used to nick the targeted strand. This nickase could be another Cas9 or other Cas nickase designed to cleave the non-targeting strand. The nickase can be separate from the VirD2 containing fusion protein or can be connected to or otherwise complexed with the VirD2 containing fusion protein.

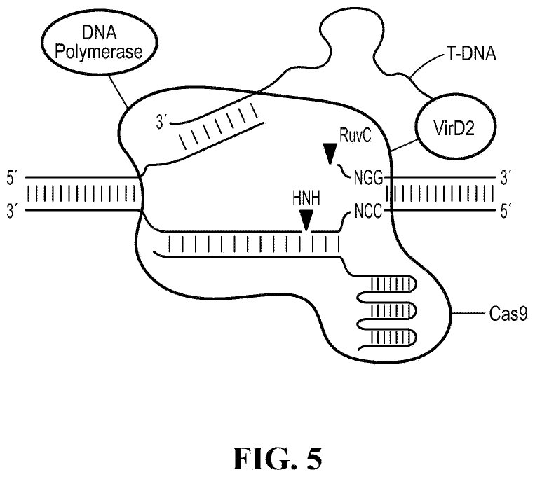

As shown in FIG. 5, a wtCas9 can be used in the system to deliver the donor polynucleotide and DNA polymerase to facilitate insertion of the donor polynucleotide sequence into the target sequence.

As shown in FIGS. 3-5, there is a homology region in the donor polynucleotide (e.g., T-DNA) that can have homology with a guide molecule and/or target polynucleotide. In some instances, the homology region is located at the 3′ end of the donor polynucleotide. In some instances, the homology region has homology complementary to the first region of the sgRNA guide sequence. In some instances, the homology region is about 17 nucleotides. Exemplary polypeptide and polynucleotide sequences are shown below.

VirD1 Agrobacterium fabrum (strain C58/ATCC 33970)
(SEQ ID NO: 1)
MSQGSRPTSSDIAVNQRECVKVEGFKVVSTRLRSAEYESFSHQARLLGLS
DSMAIRVAVRRIGGFLEIDAETRHRMEAILQSIGTLSSNIAALLSAYAEN
PTMDLEALRAERIAFGKSFADLDGLLRSILSVSRRRIDGCSLLKDAL
>VirD2 Agrobacterium fabrum (strain C58/ATCC 33970)
(SEQ ID NO: 2)
MPDRAQVIIRIVPGGGTKTLQQIINQLEYLSRKGRLELQRSARHLDIPLP
PDQIHELARSWVQETGTYDESQPDEERQQELTTHIIVSFPAGTSQVAAYA
ASREWAAEMFGSGAGGGRYNYLTAFHIDRDHPHLHVVVNRRELLGHGWLK
ISRRHPQLNYDALRIKMAEISLRHGIALDASRRAERGITERPITYAQYRR
LEREQARQIRFEDADLEQSSPQGDHPEFSQPFDTSPFEASAGGPEDMPRP
NNRQNESQVHLQEPAGVSNEAGVLVRVALETERLAQPFVSETILADDIGS
GSSRVAEGRVESANRTPDIPRAATEAATHTTHDRQRRAKRPHDDDGGPSG
AKRVTLEGIAVGPQANAGEQDGSSGPLVRQAGTSRPSPPTATTRASTATD
SLSATAHLQQRRGVLSKRPREDDDGEPSERKRERDERSKDGRGGNRR
>DNA polymerase I Escherichia coli (strain K12)
(SEQ ID NO: 3)
MVQIPQNPLILVDGSSYLYRAYHAFPPLTNSAGEPTGAMYGVLNMLRSLI
MQYKPTHAAVVFDAKGKTFRDELFEHYKSHRPPMPDDLRAQIEPLHAMVK
AMGLPLLAVSGVEADDVIGTLAREAEKAGRPVLISTGDKDMAQLVTPNIT
LINTMTNTILGPEEVVNKYGVPPELIIDFLALMGDSSDNIPGVPGVGEKT
AQALLQGLGGLDTLYAEPEKIAGLSFRGAKTMAAKLEQNKEVAYLSYQLA
TIKTDVELELTCEQLEVQQPAAEELLGLFKKYEFKRWTADVEAGKWLQAK
GAKPAAKPQETSVADEAPEVTATVISYDNYVTILDEETLKAWIAKLEKAP
VFAFDTETDSLDNISANLVGLSFAIEPGVAAYIPVAHDYLDAPDQISRER
ALELLKPLLEDEKALKVGQNLKYDRGILANYGIELRGIAFDTMLESYILN
SVAGRHDMDSLAERWLKHKTITFEEIAGKGKNQLTFNQIALEEAGRYAAE
DADVTLQLHLKMWPDLQKHKGPLNVFENIEMPLVPVLSRIERNGVKIDPK
VLHNHSEELTLRLAELEKKAHEIAGEEFNLSSTKQLQTILFEKQGIKPLK
KTPGGAPSTSEEVLEELALDYPLPKVILEYRGLAKLKSTYTDKLPLMINP
KTGRVHTSYHQAVTATGRLSSTDPNLQNIPVRNEEGRRIRQAFIAPEDYV
IVSADYSQIELRIMAHLSRDKGLLTAFAEGKDIHRATAAEVFGLPLETVT
SEQRRSAKAINFGLIYGMSAFGLARQLNIPRKEAQKYMDLYFERYPGVLE
YMERTRAQAKEQGYVETLDGRRLYLPDIKSSNGARRAAAERAAINAPMQG
TAADIIKRAMIAVDAWLQAEQPRVRMIMQVHDELVFEVHKDDVDAVAKQI
HQLMENCTRLDVPLLVEVGSGENWDQAH
>Border sequence (same for LB and RB)
(SEQ ID NO: 4)
GTTTACCCGCCAATATATCCTGTCA
* * *

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

What is claimed is:

1. An engineered composition comprising:
a. a site-specific nuclease polypeptide;
b. a DNA polymerase polypeptide connected to or otherwise capable of forming a complex with the site-specific nuclease polypeptide; and
c. a VirD2 polypeptide connected to or otherwise capable of forming a complex with the site-specific nuclease polypeptide;
wherein the site-specific nuclease polypeptide directs the DNA polymerase and VirD2 polypeptide to a target sequence in a target polynucleotide.

2. The engineered composition of claim 1, further comprising a donor construct capable of forming a complex with the VirD2 polypeptide and encoding a donor polynucleotide sequence, the donor polynucleotide sequence serving as a template for integration of the donor polynucleotide sequence into the target polynucleotide by the DNA polymerase.

3. The composition of claim 2, wherein the donor construct comprises a single-stranded donor polynucleotide sequence and a VirD2 binding sequence.

4. The composition of claim 2, wherein the donor construct is a double-stranded polynucleotide encoding a 5′ and a 3′ boundary sequence and a donor polynucleotide sequence located between the 5′ and the 3′ boundary sequences, and wherein the composition further comprises a VirD1 polypeptide that alone, or in combination with the VirD2 polypeptide, releases a single-stranded donor polynucleotide sequence from the donor construct, resulting in a complex of the single-stranded donor polynucleotide sequence with the VirD2 polypeptide.

5. The composition of claim 4, where the VirD1 polypeptide is connected to the VirD2 polypeptide.

6. The composition of any one of claims 2 to 4, wherein the donor construct further comprises a homology sequence complementary to at least a portion of the target sequence.

7. The composition of any one of claims 2 to 6, wherein the donor polynucleotide sequence is 10 bp to 20 kb bp in length.

8. The composition of any one of claims 1 to 7, wherein the site-specific nuclease is an IscB or TnpA polypeptide.

9. The composition of any one of the previous claims, wherein the site-specific nuclease polypeptide is a Cas polypeptide, and the composition further comprises a guide polynucleotide capable of forming a complex with the Cas-polypeptide and directing site specific binding of the complex to the target sequence.

10. The composition of claim 8, wherein the homology sequence is complementary to at least a portion of the guide polynucleotide.

11. The composition of claims 8 or 10, wherein the Cas polypeptide is a nickase that nicks a targeted strand of the target polynucleotide.

12. The composition of claim 11, further comprising a second guide molecule capable of forming a complex with the Cas nickase and directing nicking of a non-targeted strand of the target polynucleotide.

13. The engineered or non-naturally occurring composition of any one of the preceding claims, wherein the VirD2 and/or VirD1 polypeptides are derived from Agrobacterium tumefaceins or Rhizobium meliloti.

14. The engineered or non-naturally occurring composition of any one of the preceding claims, wherein the DNA polymerase is a DNA Pol I polymerase.

15. The engineered or non-naturally occurring composition of any one of the preceding claims, wherein the DNA polymerase is an E. coli DNA polymerase.

16. A vector system comprising one or more vectors encoding the site-specific nuclease, the DNA polymerase, the virD1 polypeptide, the virD2 polypeptide, and the donor on construct of any one of claims 1 to 15.

17. A method of inserting a donor polynucleotide sequence into a target polynucleotide comprising: introducing the composition of any one of claims 1 to 15, into a cell or cell population, wherein the complex of the site-specific nuclease polypeptide and the guide directs the single-stranded donor polynucleotide and the DNA polymerase to the target sequence, and wherein the DNA polymerase facilitates incorporation of the donor polynucleotide at or adjacent to the target sequence.

18. The method of any one of the preceding claims, wherein the donor polynucleotide is between 10 bases and 50 kb in length.

19. The method of any one of the preceding claims, wherein the polypeptide and/or nucleic acid components are provided via one or more polynucleotides encoding the polypeptides and/or nucleic acid component(s), and wherein the one or more polynucleotides are operably configured to express the polypeptides and/or nucleic acid component(s).

20. The method of any one of the preceding claims, wherein the donor polynucleotide is inserted into a region on the target polynucleotide that is 5′ or 3′ of a PAM.

21. The method of any one of the preceding claims, wherein the donor polynucleotide
a. introduces one or more mutations to the target polynucleotide,
b. inserts a functional gene or gene fragment at the target polynucleotide,
c. corrects or introduces a premature stop codon in the target polynucleotide,
d. disrupts or restores a splice cite in the target polynucleotide,
e. causes a shift in the open reading frame of the target polynucleotide, or
f. a combination thereof.

22. The method of any one of the preceding claims, wherein the one or more mutations include substitutions, deletions, and insertions.


SEQUENCE LISTING (40 sequences)

SEQ ID NO: 1 - protein, 147 aa, Agrobacterium fabrum
MSQGSRPTSSDIAVNQRECVKVEGFKVVSTRLRSAEYESFSHQARLLGLSDSMAIRVAVRRIGGFLEIDAETRHRMEAILQSIGTLSSNIAALLSAYAENPTMDLEALRAERIAFGKSFADLDGLLRSILSVSRRRIDGCSLLKDAL

SEQ ID NO: 2 - protein, 447 aa, Agrobacterium fabrum
MPDRAQVIIRIVPGGGTKTLQQIINQLEYLSRKGRLELQRSARHLDIPLPPDQIHELARSWVQETGTYDESQPDEERQQELTTHIIVSFPAGTSQVAAYAASREWAAEMFGSGAGGGRYNYLTAFHIDRDHPHLHVVVNRRELLGHGWLKISRRHPQLNYDALRIKMAEISLRHGIALDASRRAERGITERPITYAQYRRLEREQARQIRFEDADLEQSSPQGDHPEFSQPFDTSPFEASAGGPEDMPRPNNRQNESQVHLQEPAGVSNEAGVLVRVALETERLAQPFVSETILADDIGSGSSRVAEGRVESANRTPDIPRAATEAATHTTHDRQRRAKRPHDDDGGPSGAKRVTLEGIAVGPQANAGEQDGSSGPLVRQAGTSRPSPPTATTRASTATDSLSATAHLQQRRGVLSKRPREDDDGEPSERKRERDERSKDGRGGNRR

SEQ ID NO: 3 - protein, 928 aa, Escherichia coli [sequence omitted]

SEQ ID NO: 4 - DNA, 25 bp, Artificial Sequence
  Synthetic
gtttacccgccaatatatcctgtca

SEQ ID NO: 5 - protein, 15 aa, Artificial Sequence
  Synthetic
GGGGSGGGGSGGGGS

SEQ ID NO: 6 - protein, 4 aa, Artificial Sequence
  Synthetic
GGGS

SEQ ID NO: 7 - protein, 5 aa, Artificial Sequence
  Synthetic
GGGGS

SEQ ID NO: 8 - protein, 9 aa, Artificial Sequence
  Synthetic
GGSGGSGGS

SEQ ID NO: 9 - protein, 10 aa, Artificial Sequence
  Synthetic
GGGGSGGGGS

SEQ ID NO: 10 - protein, 20 aa, Artificial Sequence
  Synthetic
GGGGSGGGGSGGGGSGGGGS

SEQ ID NO: 11 - protein, 25 aa, Artificial Sequence
  Synthetic
GGGGSGGGGSGGGGSGGGGSGGGGS

SEQ ID NO: 12 - protein, 30 aa, Artificial Sequence
  Synthetic
GGGGSGGGGSGGGGSGGGGSGGGGSGGGGS

SEQ ID NO: 13 - protein, 35 aa, Artificial Sequence
  Synthetic
GGGGSGGGGSGGGGSGGGGSGGGGSGGGGSGGGGS

SEQ ID NO: 14 - protein, 40 aa, Artificial Sequence
  Synthetic
GGGGSGGGGSGGGGSGGGGSGGGGSGGGGSGGGGSGGGGS

SEQ ID NO: 15 - protein, 45 aa, Artificial Sequence
  Synthetic
GGGGSGGGGSGGGGSGGGGSGGGGSGGGGSGGGGSGGGGSGGGGS

SEQ ID NO: 16 - protein, 50 aa, Artificial Sequence
  Synthetic
GGGGSGGGGSGGGGSGGGGSGGGGSGGGGSGGGGSGGGGSGGGGSGGGGS

SEQ ID NO: 17 - protein, 55 aa, Artificial Sequence
  Synthetic
GGGGSGGGGSGGGGSGGGGSGGGGSGGGGSGGGGSGGGGSGGGGSGGGGSGGGGS

SEQ ID NO: 18 - protein, 60 aa, Artificial Sequence
  Synthetic
GGGGSGGGGSGGGGSGGGGSGGGGSGGGGSGGGGSGGGGSGGGGSGGGGSGGGGSGGGGS

SEQ ID NO: 19 - protein, 32 aa, Artificial Sequence
  Synthetic
LEPGEKPYKCPECGKSFSQSGALTRHQRTHTR

SEQ ID NO: 20 - protein, 20 aa, Artificial Sequence
  Synthetic
PKKKRKVEASSPKKRKVEAS

SEQ ID NO: 21 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 22 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

SEQ ID NO: 23 - protein, 7 aa, Simian virus 40
PKKKRKV

SEQ ID NO: 24 - protein, 16 aa, Artificial Sequence
  Synthetic
KRPAATKKAGQAKKKK

SEQ ID NO: 25 - protein, 9 aa, Homo sapiens
PAAKRVKLD

SEQ ID NO: 26 - protein, 11 aa, Homo sapiens
RQRRNELKRSP

SEQ ID NO: 27 - protein, 38 aa, Homo sapiens
NQSSNFGPMKGGNFGGRSSGPYGGGGQYFAKPRNQGGY

SEQ ID NO: 28 - protein, 42 aa, Homo sapiens
RMRIZFKNKGKDTAELRRRRVEVSVELRKAKKDEQILKRRNV

SEQ ID NO: 29 - protein, 8 aa, Homo sapiens
VSRKRPRP

SEQ ID NO: 30 - protein, 8 aa, Homo sapiens
PPKKARED

SEQ ID NO: 31 - protein, 8 aa, Homo sapiens
PQPKKKPL

SEQ ID NO: 32 - protein, 12 aa, Mus sp.
SALIKKKKKMAP

SEQ ID NO: 33 - protein, 5 aa, Influenza virus
DRLRR

SEQ ID NO: 34 - protein, 7 aa, Influenza virus
PKQKKRK

SEQ ID NO: 35 - protein, 10 aa, Hepatitis D virus
RKLKKKIKKL

SEQ ID NO: 36 - protein, 10 aa, Mus sp.
REKKKFLKRR

SEQ ID NO: 37 - protein, 20 aa, Homo sapiens
KRKGDEVDGVDEVAKKKSKK

SEQ ID NO: 38 - protein, 17 aa, Homo sapiens
RKCLQAGMNLEARKTKK

SEQ ID NO: 39 - protein, 15 aa, Artificial Sequence
  Synthetic
GGGGGGGGGGGGGGG

SEQ ID NO: 40 - protein, 8 aa, Artificial Sequence
  Synthetic
GPLGIAGQ